(19) United States
(12) Patent Application Publication
(10) Pub. No.: US 2019/0031673 A1
(43) Pub. Date: Jan. 31, 2019

(54) Organic Compound, Light-Emitting Element, Light-Emitting Device, Electronic Device, and Lighting Device

(71) Applicant: Semiconductor Energy Laboratory Co., Ltd., Kanagawa-ken (JP)

(72) Inventors: Tomoya Yamaguchi, Atsugi (JP); Hideko Yoshizumi, Isehara (JP); Hiromitsu Kido, Atsugi (JP); Satoshi Seo, Sagamihara (JP); Toshiki Sasaki, Isehara (JP)

(73) Assignee: Semiconductor Energy Laboratory Co., Ltd., Kanagawa-ken (JP)

(21) Appl. No.: 16/042,151

(22) Filed: Jul. 23, 2018

(30) Foreign Application Priority Data
Jul. 27, 2017 (JP) 2017-145790
Dec. 1, 2017 (JP) 2017-231510

(51) Int. Cl. C07D 491/048; H01L 51/52; H01L 51/50; C07D 495/04; H01L 51/00

(52) U.S. Cl. CPC C07D 491/048; H01L 51/5206; H01L 51/5221; H01L 51/5036; H01L 51/5092; H01L 51/508; C07D 495/04; H01L 51/0072; H01L 51/0073; H01L 51/0074; H01L 51/5064; H01L 51/5024

(57) ABSTRACT

A furopyrazine derivative that is a novel organic compound is provided. The organic compound has a furopyrazine skeleton and is represented by General Formula (G1).

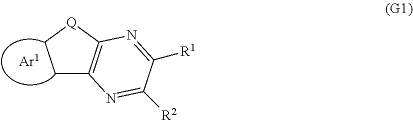

In General Formula (G1), Q represents oxygen or sulfur, Ar¹ represents a substituted or unsubstituted condensed aromatic ring, R¹ and R² independently represent hydrogen or a group having 1 to 100 total carbon atoms, and at least one of R¹ and R² has a hole-transport skeleton.

### BACKGROUND OF THE INVENTION

### 1. Field of the Invention

One embodiment of the present invention relates to an organic compound, a light-emitting element, a light-emitting device, an electronic device, and a lighting device. Note that one embodiment of the present invention is not limited to the above technical field. That is, one embodiment of the present invention relates to an object, a method, a manufacturing method, or a driving method. One embodiment of the present invention relates to a process, a machine, manufacture, or a composition of matter. Specific examples include a semiconductor device, a display device, and a liquid crystal display device.

### 2. Description of the Related Art

A light-emitting element including an EL layer between a pair of electrodes (also referred to as an organic EL element) has characteristics such as thinness, light weight, high-speed response to input signals, and low power consumption; thus, a display including such a light-emitting element has attracted attention as a next-generation flat panel display.

In a light-emitting element, voltage application between a pair of electrodes causes, in an EL layer, recombination of electrons and holes injected from the electrodes, which brings a light-emitting substance (organic compound) contained in the EL layer into an excited state. Light is emitted when the light-emitting substance returns to the ground state from the excited state. The excited state can be a singlet excited state (S*) and a triplet excited state (T*). Light emission from a singlet excited state is referred to as fluorescence, and light emission from a triplet excited state is referred to as phosphorescence. The statistical generation ratio thereof in the light-emitting element is considered to be S*:T*=1:3. Since the spectrum of light emitted from a light-emitting substance depends on the light-emitting substance, the use of different types of organic compounds as light-emitting substances makes it possible to obtain light-emitting elements that exhibit various colors.

Various kinds of substances have been developed as organic compounds and synthesis methods and the like of the substances have also been developed. The organic compounds have a wide variety of uses and development fields. In the field of biochemistry, a method for easily synthesizing a substance having a naphthofuropyrazine skeleton is reported (see Non-Patent Document 1, for example).

However, a novel substance containing, as a raw material, the substance having the naphthofuropyrazine skeleton has not been developed yet.

### REFERENCE

### Non-Patent Document

[Non-Patent Document 1] K. Shiva Kumar, Raju Adepu, Ravikumar Kapavarapu, D. Rambabu, G. Rama Krishna, C. Malla Reddy, K. Krishna Priya, Kishore V. L. Parsa, and Manojit Pal, “AlCl₃ Induced C-arylation/cyclization in a Single Pot: A New Route to Benzofuran Fused N-heterocycles of Pharmacological Interest”, Tetrahedron Letters, 2012, Vol. 53, pp. 1134-1138.

### SUMMARY OF THE INVENTION

Thus, an object of one embodiment of the present invention is to provide a novel organic compound containing, as a raw material, a substance having a furopyrazine skeleton (including naphthofuropyrazine). Another object of one embodiment of the present invention is to provide a furopyrazine derivative that is a novel organic compound. Another object of one embodiment of the present invention is to provide a novel organic compound that can be used in a light-emitting element. Another object of one embodiment of the present invention is to provide a novel organic compound that can be used in an EL layer of a light-emitting element. Another object is to provide a highly reliable and novel light-emitting element using a novel organic compound of one embodiment of the present invention. Another object is to provide a novel light-emitting device, a novel electronic device, or a novel lighting device. Note that the description of these objects does not disturb the existence of other objects. In one embodiment of the present invention, there is no need to achieve all the objects. Other objects will be apparent from and can be derived from the description of the specification, the drawings, the claims, and the like.

One embodiment of the present invention is an organic compound represented by General Formula (G1).

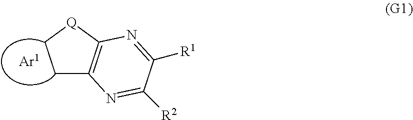

In General Formula (G1), Q represents oxygen or sulfur, Ar¹ represents a substituted or unsubstituted condensed aromatic ring, R¹ and R² independently represent hydrogen or a group having 1 to 100 total carbon atoms, and at least one of R¹ and R² has a hole-transport skeleton.

Another embodiment of the present invention is an organic compound represented by General Formula (G1).

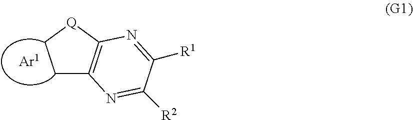

In General Formula (G1), Q represents oxygen or sulfur, Ar¹ represents any one of substituted or unsubstituted naphthalene, substituted or unsubstituted phenanthrene, and substituted or unsubstituted chrysene, R¹ and R² independently represent hydrogen or a group having 1 to 100 total carbon atoms, and at least one of R¹ and R² has a hole-transport skeleton.

Another embodiment of the present invention is an organic compound represented by General Formula (G1).

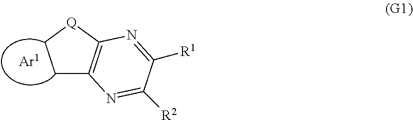

In General Formula (G1), Q represents oxygen or sulfur, Ar¹ represents a substituted or unsubstituted condensed aromatic ring, R¹ and R² independently represent hydrogen or a group having 1 to 100 total carbon atoms, and at least one of R¹ and R² is a group including a condensed ring.

Another embodiment of the present invention is an organic compound represented by General Formula (G1).

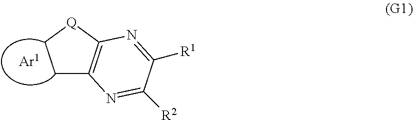

In General Formula (G1), Q represents oxygen or sulfur, Ar¹ represents any one of substituted or unsubstituted naphthalene, substituted or unsubstituted phenanthrene, and substituted or unsubstituted chrysene, R¹ and R² independently represent hydrogen or a group having 1 to 100 total carbon atoms, and at least one of R¹ and R² is a group including a condensed ring.

Note that in General Formula (G1), Ar¹ is represented by any one of General Formulae (t1) to (t3).

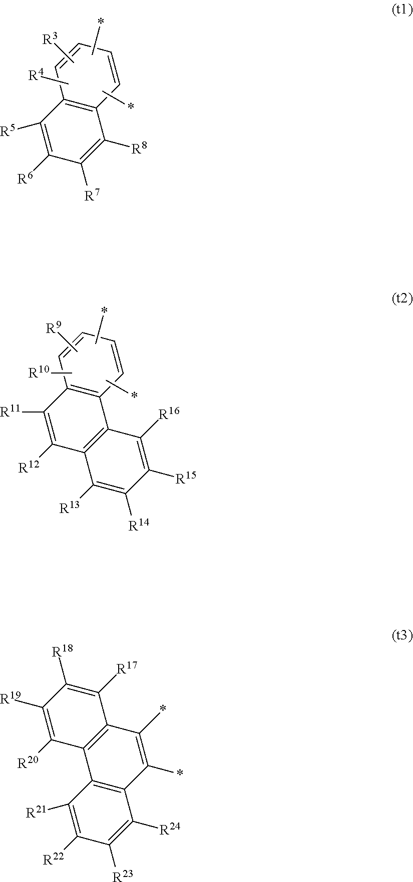

In General Formulae (t1) to (t3), R³ to R²⁴ independently represent any one of hydrogen, a substituted or unsubstituted alkyl group having 1 to 6 carbon atoms, a substituted or unsubstituted cycloalkyl group having 3 to 7 carbon atoms, and a substituted or unsubstituted aryl group having 6 to 30 carbon atoms. In addition, * represents a bonding portion in General Formula (G1).

In the above embodiments, General Formula (G1) is any one of General Formulae (G1-1) to (G1-4).

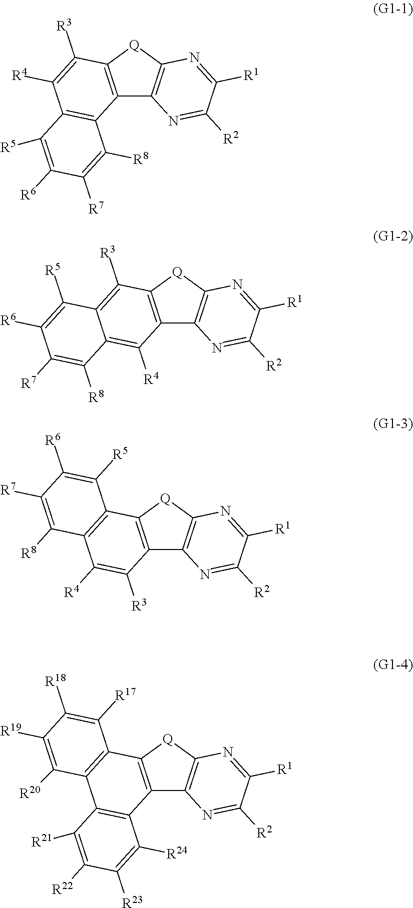

In General Formulae (G1-1) to (G1-4), Q represents oxygen or sulfur, R¹ and R² independently represent hydrogen or a group having 1 to 100 total carbon atoms, at least one of R¹ and R² has a hole-transport skeleton, and R³ to R⁸ and R¹⁷ to R²⁴ independently represent any one of hydrogen, a substituted or unsubstituted alkyl group having 1 to 6 carbon atoms, a substituted or unsubstituted cycloalkyl group having 3 to 7 carbon atoms, and a substituted or unsubstituted aryl group having 6 to 30 carbon atoms.

In the above embodiments, the hole-transport skeleton is any one of a substituted or unsubstituted diarylamino group, a substituted or unsubstituted condensed aromatic hydrocarbon ring, and a substituted or unsubstituted π-electron rich condensed heteroaromatic ring.

In some of the above embodiments, the condensed ring is any one of a substituted or unsubstituted condensed aromatic hydrocarbon ring and a substituted or unsubstituted π-electron rich condensed heteroaromatic ring. Alternatively, the condensed ring is a substituted or unsubstituted condensed heteroaromatic ring having any one of a dibenzothiophene skeleton, a dibenzofuran skeleton, and a carbazole skeleton. Alternatively, the condensed ring is a substituted or unsubstituted condensed aromatic hydrocarbon ring having any one of a naphthalene skeleton, a fluorene skeleton, a triphenylene skeleton, and a phenanthrene skeleton.

In the above embodiments, R¹ and R² in General Formula (G1) independently represent hydrogen or a group having 1 to 100 total carbon atoms. At least one of R¹ and R² is a group represented by General Formula (u1).

A¹-(α)ₙ-* (u1)

In General Formula (u1), α represents a substituted or unsubstituted arylene group having 6 to 25 carbon atoms, n represents an integer of 0 to 4, and A¹ represents a substituted or unsubstituted aryl group having 6 to 30 total carbon atoms or a substituted or unsubstituted heteroaryl group having 3 to 30 total carbon atoms. In addition, * represents a bonding portion in General Formula (G1).

In General Formula (u1), A¹ is any one of General Formulae (A¹-1) to (A¹-17).

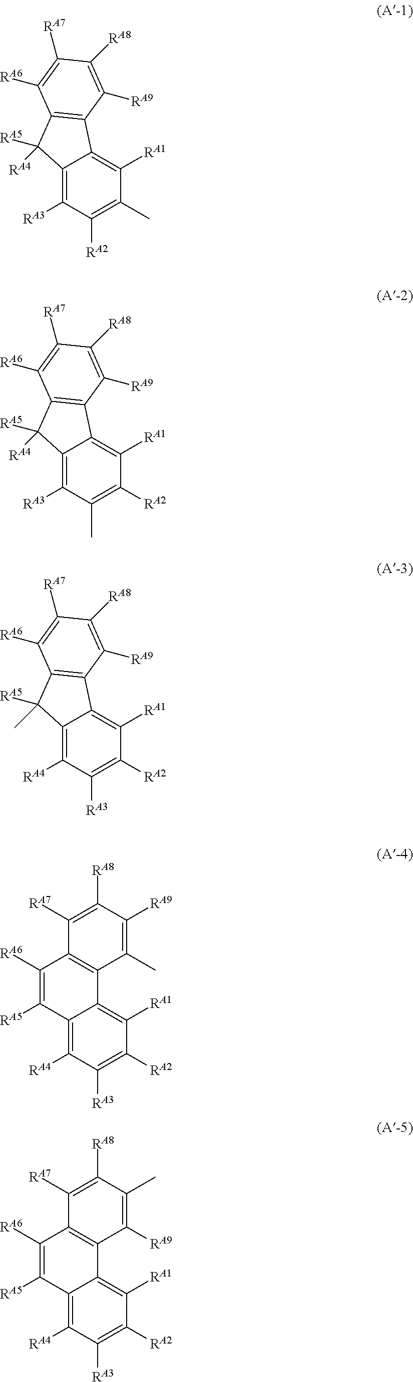

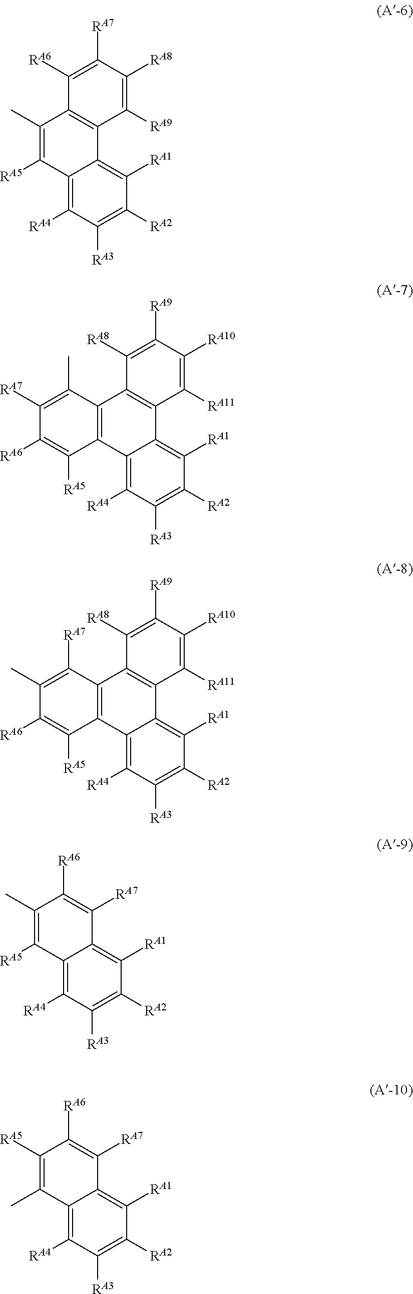

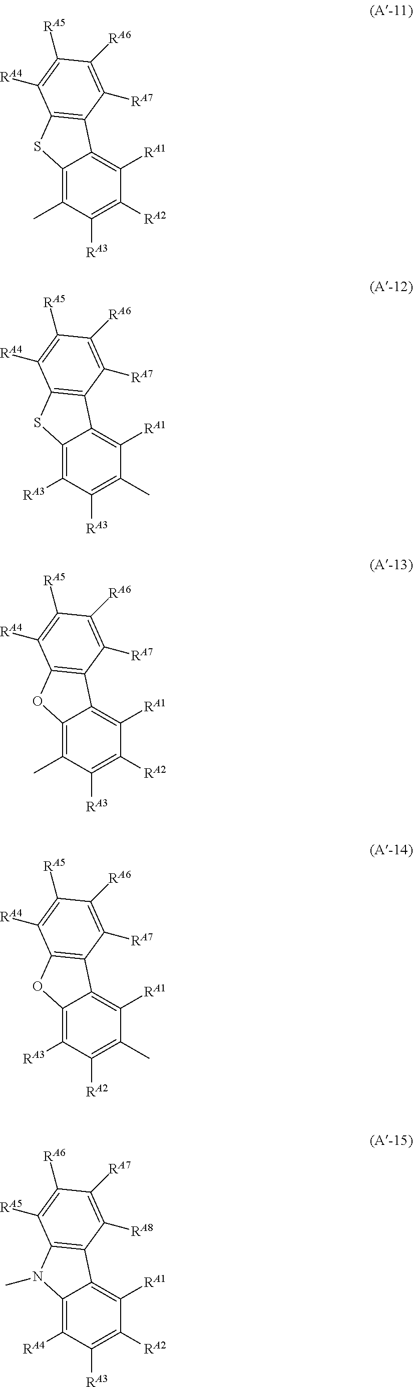

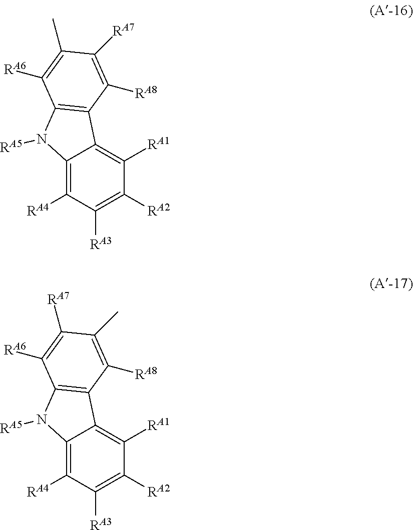

In General Formulae (A¹-1) to (A¹-17), R^{A1} to R^{A11} independently represent any one of hydrogen, a substituted or unsubstituted alkyl group having 1 to 6 carbon atoms, a substituted or unsubstituted cycloalkyl group having 3 to 7 carbon atoms, and a substituted or unsubstituted aryl group having 6 to 30 carbon atoms.

In General Formula (u1), ac is any one of General Formulae (Ar-1) to (Ar-14).

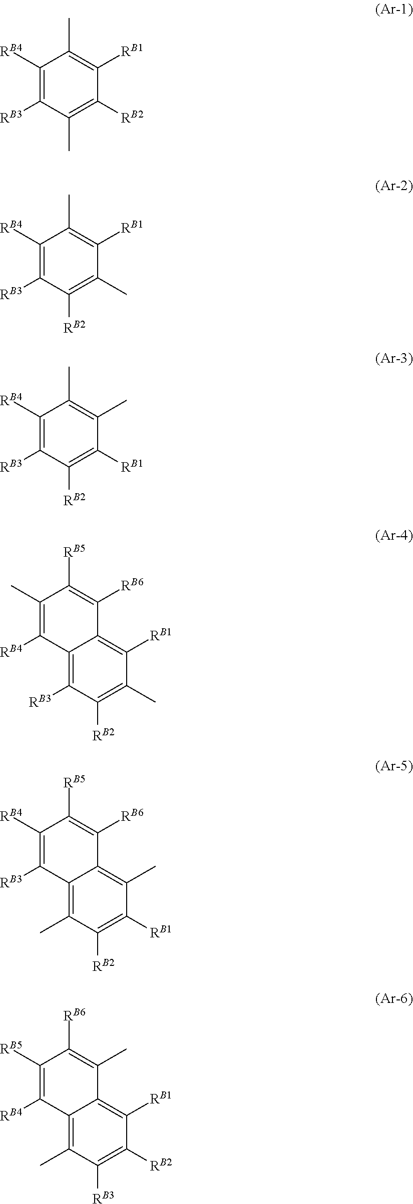

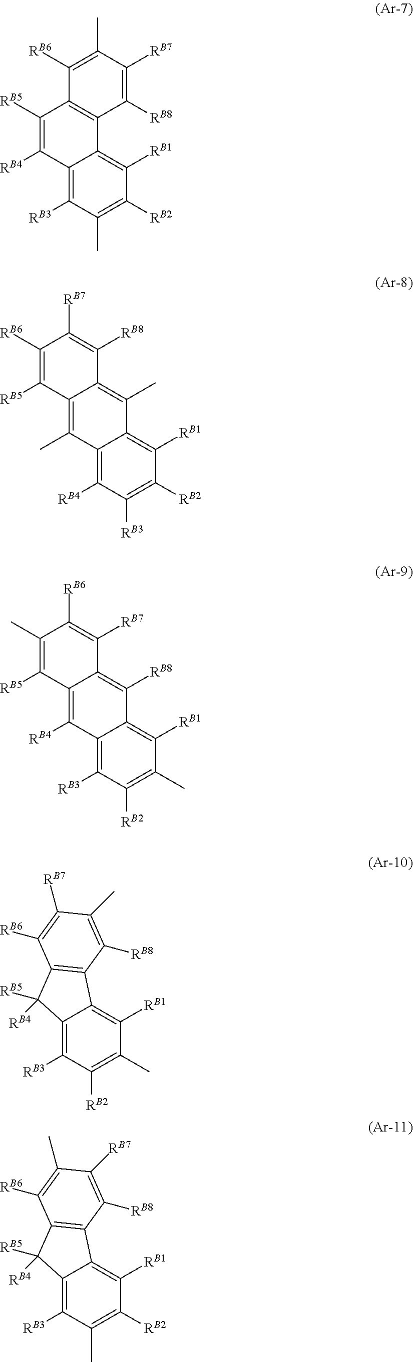

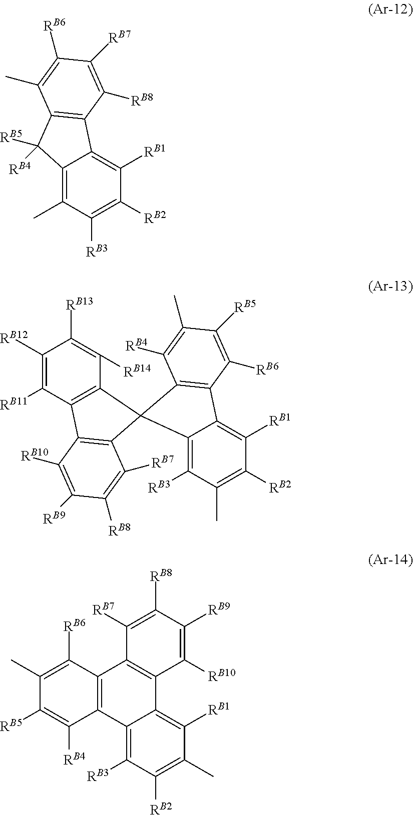

In General Formulae (Ar-1) to (Ar-14), R^{B1} to R^{B14} independently represent any one of hydrogen, a substituted or unsubstituted alkyl group having 1 to 6 carbon atoms, a substituted or unsubstituted cycloalkyl group having 3 to 7 carbon atoms, and a substituted or unsubstituted aryl group having 6 to 30 carbon atoms.

Another embodiment of the present invention is an organic compound represented by any one of Structural Formulae (100), (123), (125), (126), (133), (156), (208), (238), (239), (244), (245), and (246).

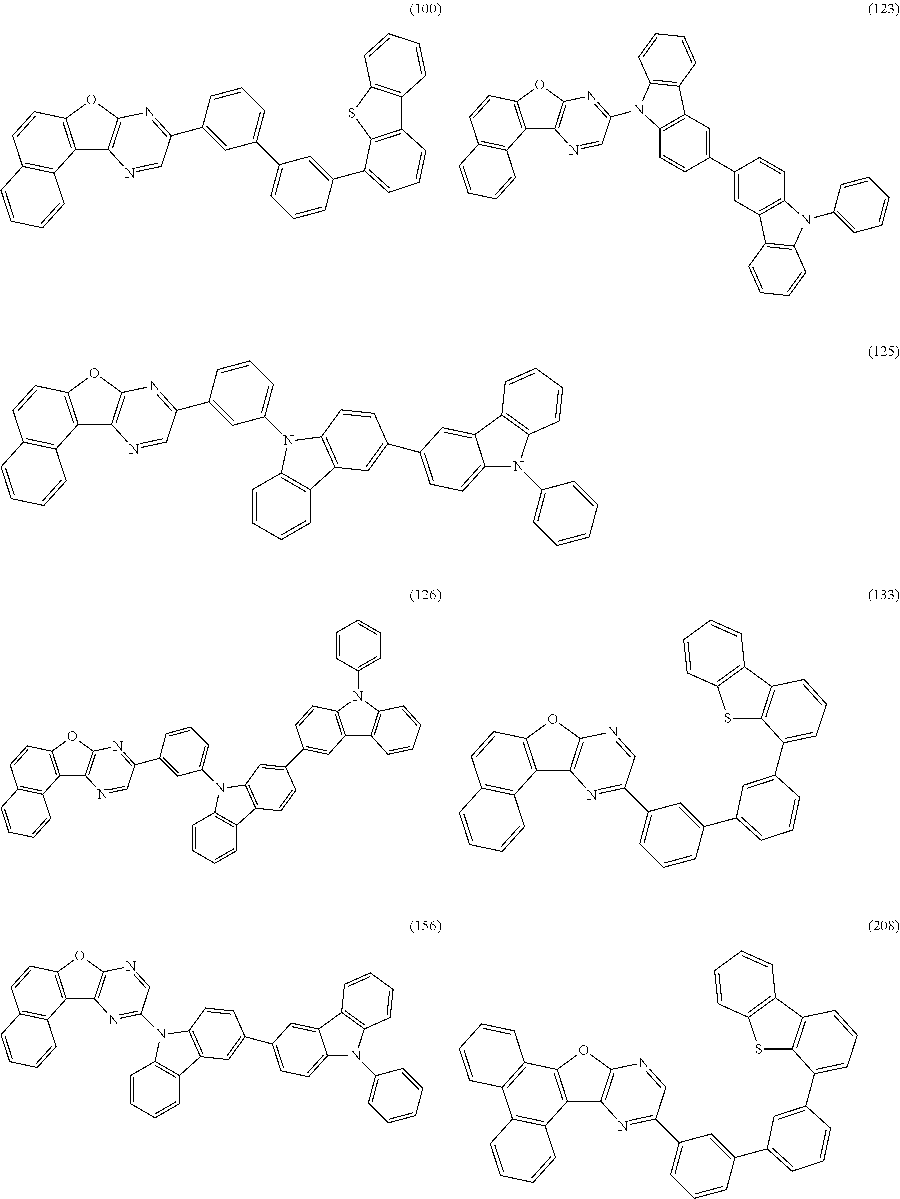

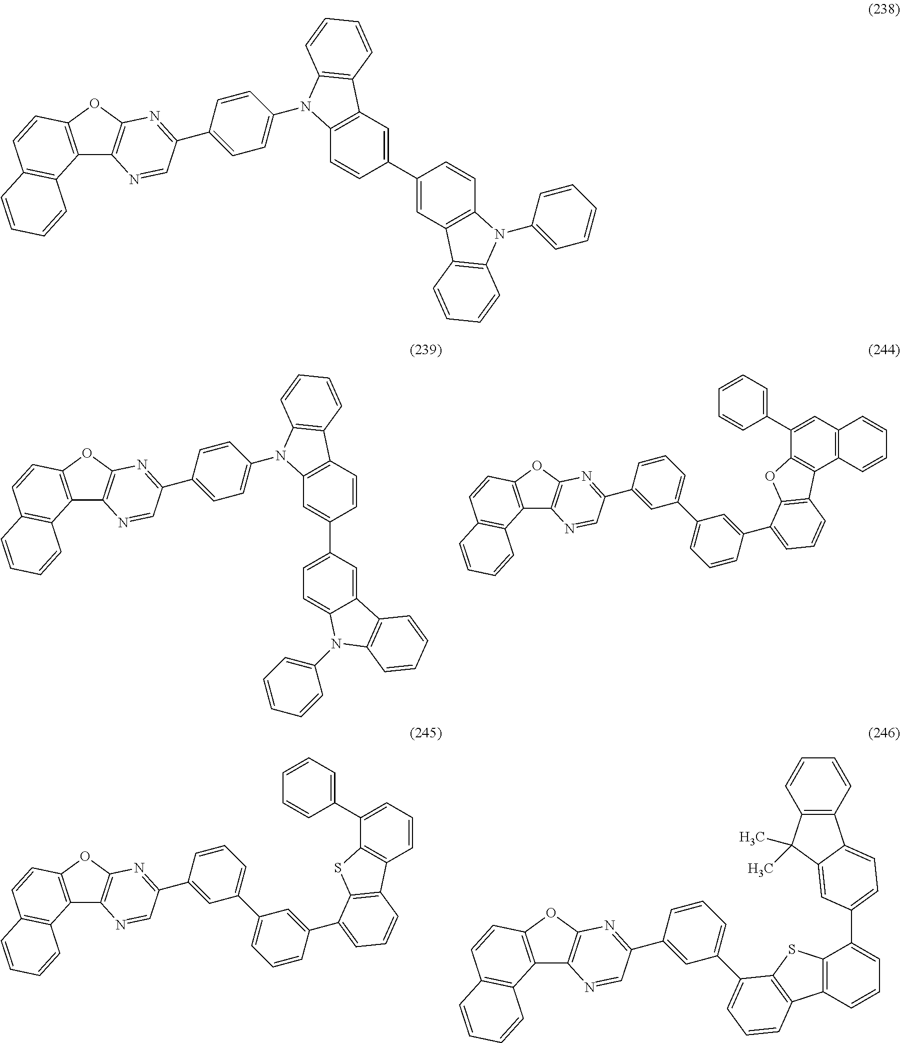

Note that the present invention also includes a novel organic compound (refer to Embodiment 1) serving as a raw material for synthesizing the aforementioned organic compound of one embodiment of the present invention. Another embodiment of the present invention is a light-emitting element including the aforementioned organic compound of one embodiment of the present invention. The present invention also includes a light-emitting element containing a guest material as well as the aforementioned organic compound.

Another embodiment of the present invention is a light-emitting element including the aforementioned organic compound of one embodiment of the present invention. Note that the present invention also includes a light-emitting element that uses the organic compound of one embodiment of the present invention for an EL layer between a pair of electrodes and a light-emitting layer in the EL layer. In addition to the aforementioned light-emitting elements, the present invention includes a light-emitting element including a layer (e.g., a cap layer) that is in contact with an electrode and contains an organic compound. In addition to the light-emitting element, a light-emitting device including a transistor, a substrate, and the like is also included in the scope of the invention. Furthermore, the scope of the invention includes, in addition to the light-emitting device, an electronic device and a lighting device that include a microphone, a camera, an operation button, an external connection portion, a housing, a cover, a support, a speaker, and the like.

In addition, the scope of one embodiment of the present invention includes a light-emitting device including a light-emitting element, and a lighting device including the light-emitting device. Accordingly, the light-emitting device in this specification refers to an image display device or a light source (including a lighting device). In addition, the light-emitting device includes the following in its category: a module in which a connector such as a flexible printed circuit (FPC) or a tape carrier package (TCP) is attached to a light-emitting device; a module in which a printed wiring board is provided at the end of a TCP; and a module in which an integrated circuit (IC) is directly mounted on a light-emitting element by a chip on glass (COG) method.

According to one embodiment of the present invention, a novel organic compound containing, as a raw material, a substance having a furopyrazine skeleton (including naphthofuropyrazine) can be provided. According to another embodiment of the present invention, a furopyrazine derivative that is a novel organic compound can be provided. According to another embodiment of the present invention, a novel organic compound that can be used in a light-emitting element can be provided. According to another embodiment of the present invention, a novel organic compound that can be used in an EL layer of a light-emitting element can be provided. According to another embodiment of the present invention, a highly reliable and novel light-emitting element using a novel organic compound of one embodiment of the present invention can be provided. Furthermore, a novel light-emitting device, a novel electronic device, or a novel lighting device can be provided. Note that the description of these effects does not disturb the existence of other effects. One embodiment of the present invention does not necessarily achieve all the effects. Other effects will be apparent from and can be derived from the description of the specification, the drawings, the claims, and the like.

### BRIEF DESCRIPTION OF THE DRAWINGS

In the accompanying drawings:

FIGS. 1A to 1E illustrate structures of light-emitting elements;

FIGS. 2A to 2C illustrate a light-emitting device;

FIGS. 3A and 3B illustrate a light-emitting device;

FIGS. 4A to 4G illustrate electronic devices;

FIGS. 5A to 5C illustrate an electronic device;

FIGS. 6A and 6B illustrate an automobile;

FIGS. 7A to 7D illustrate lighting devices;

FIG. 8 illustrates lighting devices;

FIG. 9 is a ¹H-NMR chart of an organic compound represented by Structural Formula (100);

FIGS. 10A and 10B show ultraviolet-visible absorption and emission spectra of the organic compound represented by Structural Formula (100);

FIG. 11 illustrates a light-emitting element;

FIG. 12 shows current density-luminance characteristics of a light-emitting element 1 and a comparative light-emitting element 2;

FIG. 13 shows voltage-luminance characteristics of the light-emitting element 1 and the comparative light-emitting element 2;

FIG. 14 shows luminance-current efficiency characteristics of the light-emitting element 1 and the comparative light-emitting element 2;

FIG. 15 shows voltage-current characteristics of the light-emitting element 1 and the comparative light-emitting element 2;

FIG. 16 shows emission spectra of the light-emitting element 1 and the comparative light-emitting element 2;

FIG. 17 shows reliability of the light-emitting element 1 and the comparative light-emitting element 2;

FIG. 18 shows current density-luminance characteristics of a light-emitting element 3;

FIG. 19 shows voltage-luminance characteristics of the light-emitting element 3;

FIG. 20 shows luminance-current efficiency characteristics of the light-emitting element 3;

FIG. 21 shows voltage-current characteristics of the light-emitting element 3;

FIG. 22 shows an emission spectrum of the light-emitting element 3;

FIG. 23 shows reliability of the light-emitting element 3;

FIG. 24 shows current density-luminance characteristics of a light-emitting element 4;

FIG. 25 shows voltage-luminance characteristics of the light-emitting element 4;

FIG. 26 shows luminance-current efficiency characteristics of the light-emitting element 4;

FIG. 27 shows voltage-current characteristics of the light-emitting element 4;

FIG. 28 shows an emission spectrum of the light-emitting element 4;

FIG. 29 shows reliability of the light-emitting element 4;

FIG. 30 shows current density-luminance characteristics of a light-emitting element 5;

FIG. 31 shows voltage-luminance characteristics of the light-emitting element 5;

FIG. 32 shows luminance-current efficiency characteristics of the light-emitting element 5;

FIG. 33 shows voltage-current characteristics of the light-emitting element 5;

FIG. 34 shows an emission spectrum of the light-emitting element 5;

FIG. 35 shows reliability of the light-emitting element 5;

FIG. 36 is a ¹H-NMR chart of an organic compound represented by Structural Formula (123);

FIG. 37 is a ¹H-NMR chart of an organic compound represented by Structural Formula (125);

FIG. 38 is a ¹H-NMR chart of an organic compound represented by Structural Formula (126);

FIG. 39 is a ¹H-NMR chart of an organic compound represented by Structural Formula (133);

FIG. 40 is a ¹H-NMR chart of an organic compound represented by Structural Formula (156);

FIG. 41 is a ¹H-NMR chart of an organic compound represented by Structural Formula (208);

FIG. 42 is a ¹H-NMR chart of an organic compound represented by Structural Formula (238);

FIG. 43 is a ¹H-NMR chart of an organic compound represented by Structural Formula (239);

FIG. 44 is a ¹H-NMR chart of an organic compound represented by Structural Formula (244);

FIG. 45 is a ¹H-NMR chart of an organic compound represented by Structural Formula (245);

FIG. 46 is a ¹H-NMR chart of an organic compound represented by Structural Formula (246);

FIG. 47 shows current density-luminance characteristics of a light-emitting element 8;

FIG. 48 shows voltage-luminance characteristics of the light-emitting element 8;

FIG. 49 shows luminance-current efficiency characteristics of the light-emitting element 8;

FIG. 50 shows voltage-current characteristics of the light-emitting element 8;

FIG. 51 shows an emission spectrum of the light-emitting element 8;

FIG. 52 shows reliability of the light-emitting element 8;

FIG. 53 shows current density-luminance characteristics of a light-emitting element 9;

FIG. 54 shows voltage-luminance characteristics of the light-emitting element 9;

FIG. 55 shows luminance-current efficiency characteristics of the light-emitting element 9;

FIG. 56 shows voltage-current characteristics of the light-emitting element 9;

FIG. 57 shows an emission spectrum of the light-emitting element 9;

FIG. 58 shows reliability of the light-emitting element 9;

FIG. 59 shows current density-luminance characteristics of light-emitting elements 10 to 15;

FIG. 60 shows voltage-luminance characteristics of the light-emitting elements 10 to 15;

FIG. 61 shows luminance-current efficiency characteristics of the light-emitting elements 10 to 15;

FIG. 62 shows voltage-current characteristics of the light-emitting elements 10 to 15;

FIG. 63 shows emission spectra of the light-emitting elements 10 to 15;

FIG. 64 shows reliability of the light-emitting elements 10 to 15;

FIG. 65 shows current density-luminance characteristics of a light-emitting element 16 and a comparative light-emitting element 17;

FIG. 66 shows voltage-luminance characteristics of the light-emitting element 16 and the comparative light-emitting element 17;

FIG. 67 shows luminance-current efficiency characteristics of the light-emitting element 16 and the comparative light-emitting element 17;

FIG. 68 shows voltage-current characteristics of the light-emitting element 16 and the comparative light-emitting element 17;

FIG. 69 shows emission spectra of the light-emitting element 16 and the comparative light-emitting element 17; and

FIG. 70 shows reliability of the light-emitting element 16 and the comparative light-emitting element 17.

### DETAILED DESCRIPTION OF THE INVENTION

Embodiments of the present invention will be described in detail below with reference to the drawings. Note that the present invention is not limited to the following description, and the modes and details of the present invention can be modified in various ways without departing from the spirit and scope of the present invention. Therefore, the present invention should not be construed as being limited to the description in the following embodiments.

Note that the position, size, range, or the like of each component illustrated in drawings and the like is not accurately represented in some cases for easy understanding. Therefore, the disclosed invention is not necessarily limited to the position, size, range, or the like disclosed in the drawings and the like.

In the description of modes of the present invention with reference to the drawings in this specification and the like, the same components in different drawings are commonly denoted by the same reference numeral.

### Embodiment 1

In this embodiment, an organic compound of one embodiment of the present invention will be described. Note that the organic compound of one embodiment of the present invention has a naphthofuropyrazine skeleton and is represented by General Formula (G1).

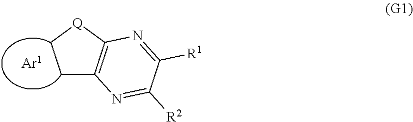

Note that in General Formula (G1), Q represents oxygen or sulfur, Ar¹ represents a substituted or unsubstituted condensed aromatic ring, R¹ and R² independently represent hydrogen or a group having 1 to 100 total carbon atoms, and at least one of R¹ and R² has a hole-transport skeleton.

Another embodiment of the present invention is an organic compound represented by General Formula (G1).

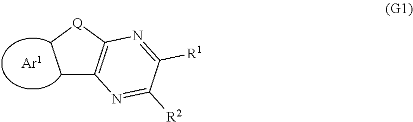

In General Formula (G1), Q represents oxygen or sulfur, Ar¹ represents any one of substituted or unsubstituted naphthalene, substituted or unsubstituted phenanthrene, and substituted or unsubstituted chrysene, R¹ and R² independently represent hydrogen or a group having 1 to 100 total carbon atoms, and at least one of R¹ and R² has a hole-transport skeleton.

Another embodiment of the present invention is an organic compound represented by General Formula (G1).

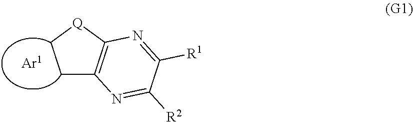

In General Formula (G1), Q represents oxygen or sulfur, Ar¹ represents a substituted or unsubstituted condensed aromatic ring, R¹ and R² independently represent hydrogen or a group having 1 to 100 total carbon atoms, and at least one of R¹ and R² is a group including a condensed ring.

Another embodiment of the present invention is an organic compound represented by General Formula (G1).

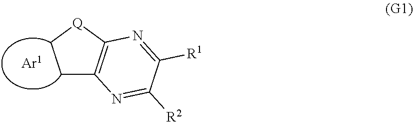

In General Formula (G1), Q represents oxygen or sulfur, Ar¹ represents any one of substituted or unsubstituted naphthalene, substituted or unsubstituted phenanthrene, and substituted or unsubstituted chrysene, R¹ and R² independently represent hydrogen or a group having 1 to 100 total carbon atoms, and at least one of R¹ and R² is a group including a condensed ring.

Note that in General Formula (G1), Ar¹ is represented by any one of General Formulae (t1) to (t3).

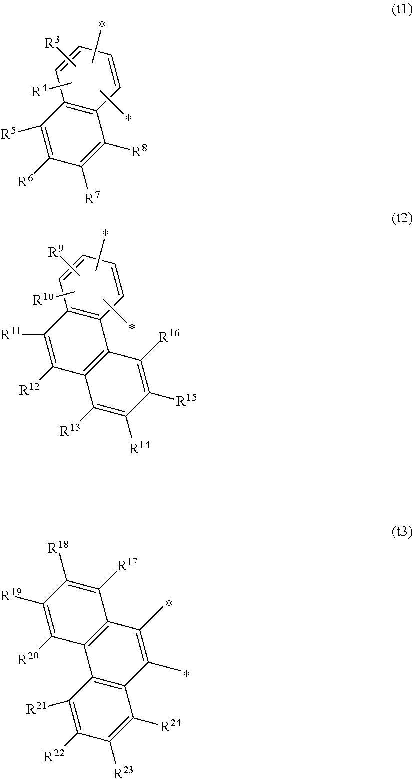

In General Formulae (t1) to (t3), R³ to R²⁴ independently represent any one of hydrogen, a substituted or unsubstituted alkyl group having 1 to 6 carbon atoms, a substituted or unsubstituted cycloalkyl group having 3 to 7 carbon atoms, and a substituted or unsubstituted aryl group having 6 to 30 carbon atoms. In addition, * represents a bonding portion in General Formula (G1).

In the above embodiments, General Formula (G1) is any one of General Formulae (G1-1) to (G1-4).

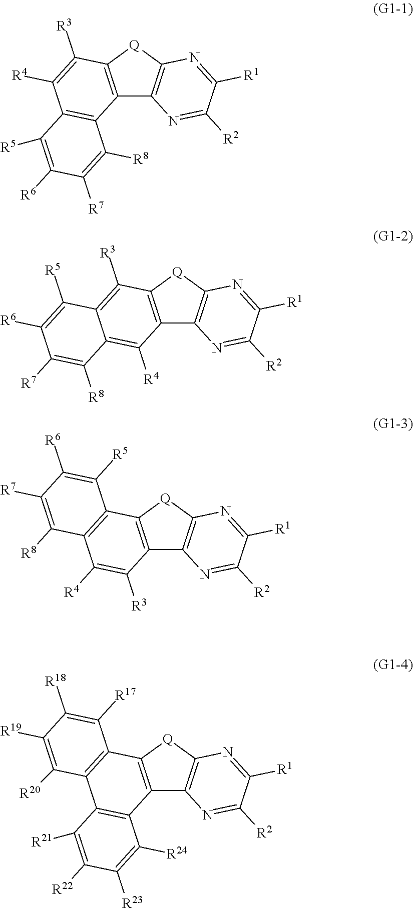

In General Formulae (G1-1) to (G1-4), Q represents oxygen or sulfur, R¹ and R² independently represent hydrogen or a group having 1 to 100 total carbon atoms, at least one of R¹ and R² has a hole-transport skeleton, and R³ to R⁸ and R¹⁷ to R²⁴ independently represent any one of hydrogen, a substituted or unsubstituted alkyl group having 1 to 6 carbon atoms, a substituted or unsubstituted cycloalkyl group having 3 to 7 carbon atoms, and a substituted or unsubstituted aryl group having 6 to 30 carbon atoms.

In the above embodiments, the hole-transport skeleton included in at least one of R¹ and R² is any one of a substituted or unsubstituted diarylamino group, a substituted or unsubstituted condensed aromatic hydrocarbon ring, and a substituted or unsubstituted π-electron rich condensed heteroaromatic ring. The condensed aromatic hydrocarbon ring preferably includes any one of a naphthalene skeleton, a fluorene skeleton, a triphenylene skeleton, and a phenanthrene skeleton. The π-electron rich condensed heteroaromatic ring is preferably a condensed heteroaromatic ring having any one of a dibenzothiophene skeleton, a dibenzofuran skeleton, and a carbazole skeleton. The condensed heteroaromatic ring can be carbazole, dibenzothiophene, or dibenzofuran, or can be a condensed ring having a carbazole skeleton, a dibenzothiophene skeleton, or a dibenzofuran skeleton in a ring structure (i.e., a condensed ring in which a ring is condensed with a carbazole skeleton, a dibenzothiophene skeleton, or a dibenzofuran skeleton), such as benzocarbazole, dibenzocarbazole, indolocarbazole, benzindolocarbazole, dibenzindolocarbazole, benzindolobenzocarbazole, benzonaphthothiophene, or benzonaphthofuran.

In the above embodiments, the condensed ring included in at least one of R¹ and R² is any one of a substituted or unsubstituted condensed aromatic hydrocarbon ring and a substituted or unsubstituted π-electron rich condensed heteroaromatic ring. The condensed ring is particularly preferably a substituted or unsubstituted condensed aromatic hydrocarbon ring having any one of a naphthalene skeleton, a fluorene skeleton, a triphenylene skeleton, and a phenanthrene skeleton. Alternatively, the condensed ring is particularly preferably a substituted or unsubstituted condensed heteroaromatic ring having any one of a dibenzothiophene skeleton, a dibenzofuran skeleton, and a carbazole skeleton. The condensed heteroaromatic ring can be carbazole, dibenzothiophene, or dibenzofuran, or can be a condensed ring having a carbazole skeleton, a dibenzothiophene skeleton, or a dibenzofuran skeleton in a ring structure (i.e., a condensed ring in which a ring is condensed with a carbazole skeleton, a dibenzothiophene skeleton, or a dibenzofuran skeleton), such as benzocarbazole, dibenzocarbazole, indolocarbazole, benzindolocarbazole, dibenzindolocarbazole, benzindolobenzocarbazole, benzonaphthothiophene, or benzonaphthofuran.

In the above embodiments, R¹ and R² in General Formula (G1) independently represent hydrogen or a group having 1 to 100 total carbon atoms. At least one of R¹ and R² is a group represented by General Formula (u1).

A¹-(α)ₙ-* (u1)

In General Formula (u1), α represents a substituted or unsubstituted arylene group having 6 to 25 carbon atoms, n represents an integer of 0 to 4, and A¹ represents a substituted or unsubstituted aryl group having 6 to 30 total carbon atoms or a substituted or unsubstituted heteroaryl group having 3 to 30 total carbon atoms.

In General Formula (u1), A¹ represents a substituted or unsubstituted aryl group having 6 to 30 total carbon atoms or a substituted or unsubstituted heteroaryl group having 3 to 30 total carbon atoms. Specifically, A¹ is any one of General Formulae (A¹-1) to (A¹-17).

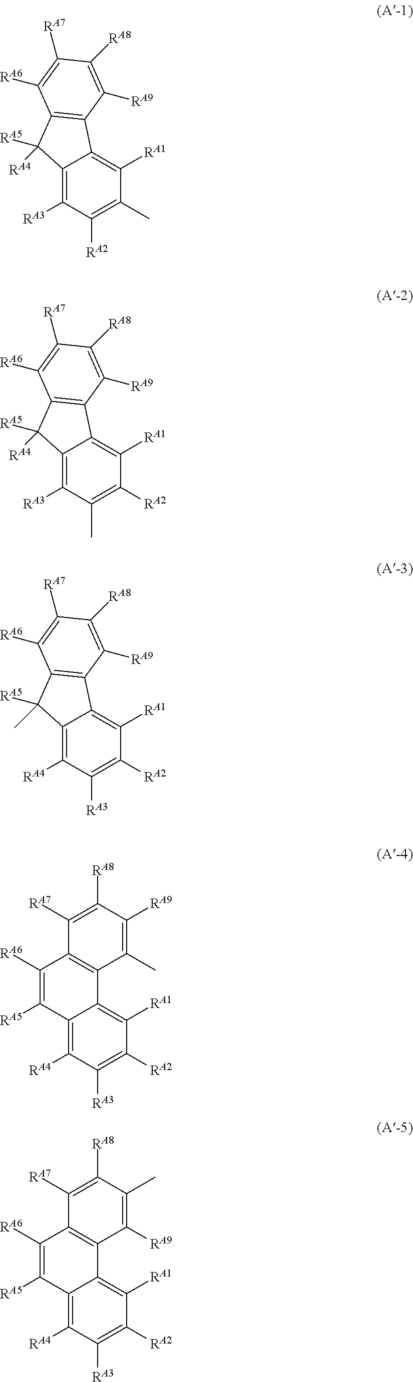

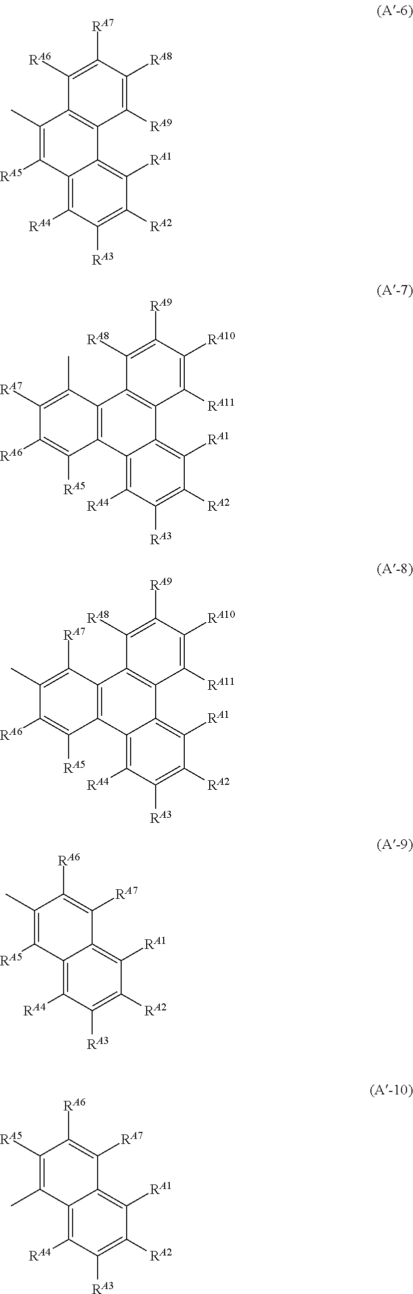

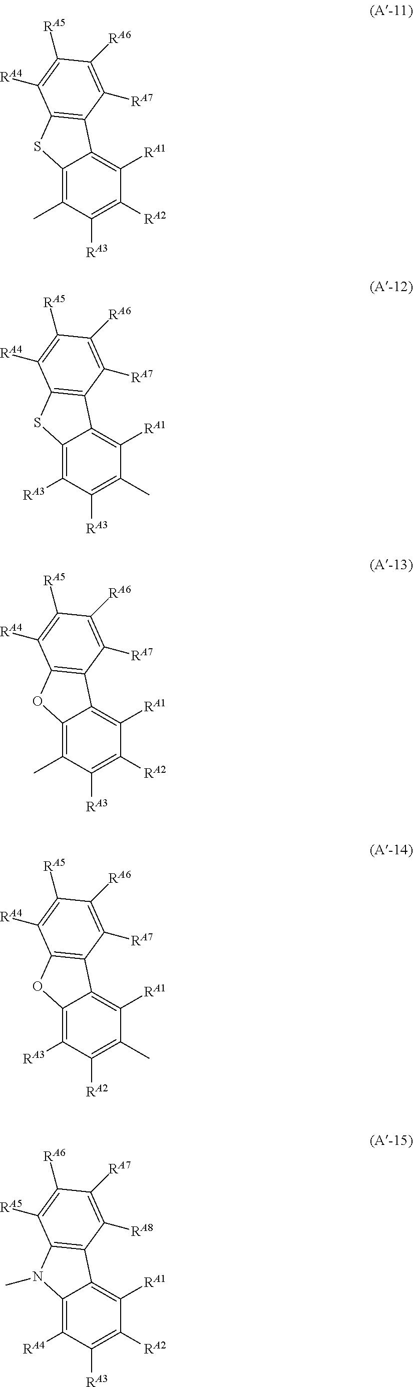

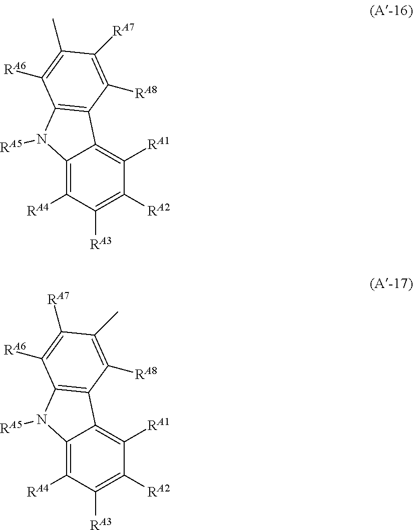

In General Formulae (A¹-1) to (A¹-17), R^{A1} to R^{A11} independently represent any one of hydrogen, a substituted or unsubstituted alkyl group having 1 to 6 carbon atoms, a substituted or unsubstituted cycloalkyl group having 3 to 7 carbon atoms, and a substituted or unsubstituted aryl group having 6 to 30 carbon atoms.

In General Formula (u1), α is any one of General Formulae (Ar-1) to (Ar-14).

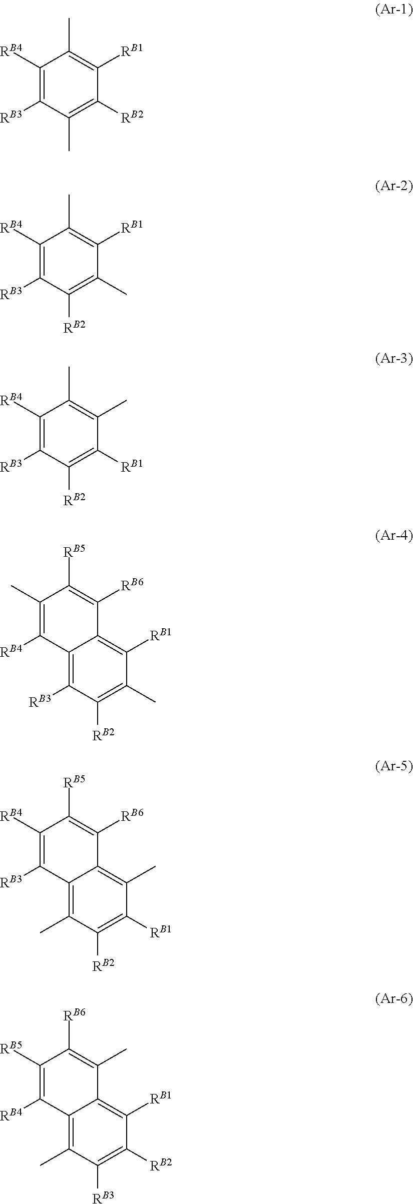

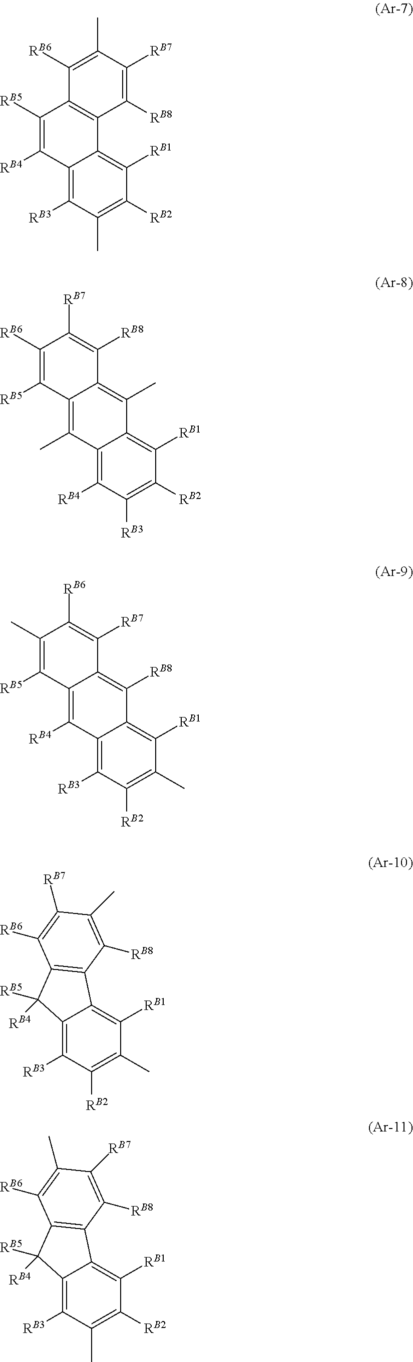

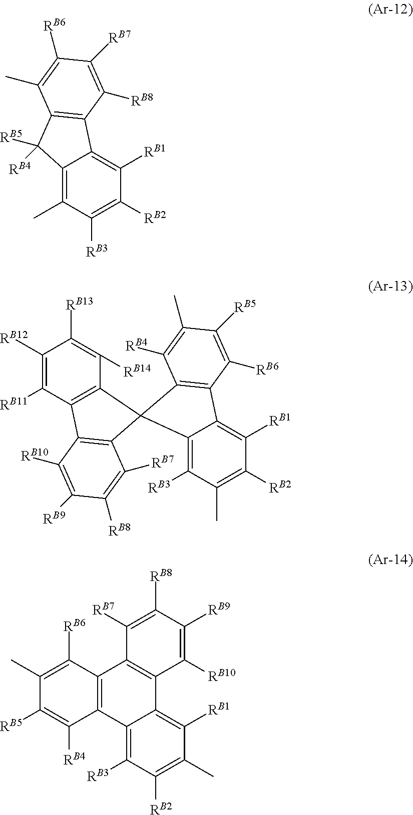

In General Formulae (Ar-1) to (Ar-14), R^{B1} to R^{B14} independently represent any one of hydrogen, a substituted or unsubstituted alkyl group having 1 to 6 carbon atoms, a substituted or unsubstituted cycloalkyl group having 3 to 7 carbon atoms, and a substituted or unsubstituted aryl group having 6 to 30 carbon atoms.

Examples of the group having 1 to 100 total carbon atoms that is included in R¹ and R² in General Formula (G1) and General Formulae (G1-1) to (G1-4) include a substituted or unsubstituted alkyl group having 1 to 6 carbon atoms, a substituted or unsubstituted cycloalkyl group having 3 to 7 carbon atoms, a substituted or unsubstituted aryl group having 6 to 30 carbon atoms, and a substituted or unsubstituted heteroaryl group having 3 to 30 carbon atoms. Note that at least one of R¹ and R² has the hole-transport skeleton or the condensed ring.

Note that in the case where any of the substances listed below includes a substituent, examples of the substituent include an alkyl group having 1 to 7 carbon atoms, such as a methyl group, an ethyl group, a propyl group, an isopropyl group, a butyl group, an isobutyl group, a sec-butyl group, a tert-butyl group, a pentyl group, or a hexyl group; a cycloalkyl group having 5 to 7 carbon atoms, such as a cyclopentyl group, a cyclohexyl group, a cycloheptyl group, or a 8,9,10-trinorbornanyl group; and an aryl group having 6 to 12 carbon atoms, such as a phenyl group, a naphthyl group, or a biphenyl group. The substances are as follows: the substituted or unsubstituted condensed aromatic ring in General Formula (G1); the substituted or unsubstituted naphthalene, the substituted or unsubstituted phenanthrene, and the substituted or unsubstituted chrysene in General Formula (G1); the substituted or unsubstituted alkyl group having 1 to 6 carbon atoms, the substituted or unsubstituted cycloalkyl group having 3 to 7 carbon atoms, and the substituted or unsubstituted aryl group having 6 to 30 carbon atoms in General Formulae (t1) to (t3); the substituted or unsubstituted alkyl group having 1 to 6 carbon atoms, the substituted or unsubstituted cycloalkyl group having 3 to 7 carbon atoms, and the substituted or unsubstituted aryl group having 6 to 30 carbon atoms in General Formulae (G1-1) to (G1-4); the substituted or unsubstituted condensed aromatic hydrocarbon ring and the substituted or unsubstituted π-electron rich condensed heteroaromatic ring in General Formula (G1); the substituted or unsubstituted arylene group having 6 to 25 carbon atoms, the substituted or unsubstituted aryl group having 6 to 30 total carbon atoms, and the substituted or unsubstituted heteroaryl group having 3 to 30 total carbon atoms in General Formula (u1); the substituted or unsubstituted alkyl group having 1 to 6 carbon atoms, the substituted or unsubstituted cycloalkyl group having 3 to 7 carbon atoms, and the substituted or unsubstituted aryl group having 6 to 30 carbon atoms in General Formulae (Ar-1) to (Ar-14); and the substituted or unsubstituted alkyl group having 1 to 6 carbon atoms, the substituted or unsubstituted cycloalkyl group having 3 to 7 carbon atoms, the substituted or unsubstituted aryl group having 6 to 30 carbon atoms, and the substituted or unsubstituted heteroaryl group having 3 to 30 carbon atoms in the group having 1 to 100 total carbon atoms that is included in R¹ and R² in General Formula (G1) and General Formulae (G1-1) to (G1-4).

Specific examples of the alkyl group having 1 to 6 carbon atoms in General Formulae (t1) to (t3), General Formulae (G1-1) to (G1-4), and General Formulae (A¹-1) to (A¹-17) include a methyl group, an ethyl group, a propyl group, an isopropyl group, a butyl group, a sec-butyl group, an isobutyl group, a tert-butyl group, a pentyl group, an isopentyl group, a sec-pentyl group, a tert-pentyl group, a neopentyl group, a hexyl group, an isohexyl group, a 3-methylpentyl group, a 2-methylpentyl group, a 2-ethylbutyl group, a 1,2-dimethylbutyl group, a 2,3-dimethylbutyl group, and an n-heptyl group.

Specific examples of the cycloalkyl group having 3 to 7 carbon atoms in General Formulae (t1) to (t3), General Formulae (G1-1) to (G1-4), and General Formulae (A¹-1) to (A¹-17) include a cyclopropyl group, a cyclobutyl group, a cyclopentyl group, a cyclohexyl group, a 1-methylcyclohexyl group, a 2,6-dimethylcyclohexyl group, a cycloheptyl group, and a cyclooctyl group.

Specific examples of the aryl group having 6 to 30 carbon atoms in General Formulae (t1) to (t3), General Formulae (G1-1) to (G1-4), and General Formulae (A¹-1) to (A¹-17) include a phenyl group, an o-tolyl group, an m-tolyl group, a p-tolyl group, a mesityl group, an o-biphenyl group, an m-biphenyl group, a p-biphenyl group, a 1-naphthyl group, a 2-naphthyl group, a fluorenyl group, a 9,9-dimethylfluorenyl group, a spirofluorenyl group, a phenanthrenyl group, an anthracenyl group, and a fluoranthenyl group.

Specific examples of the aryl group having 6 to 30 carbon atoms in the group having 1 to 100 total carbon atoms that is included in R¹ and R² in General Formula (G1) and General Formulae (G1-1) to (G1-4) include a phenyl group, an o-tolyl group, an m-tolyl group, a p-tolyl group, a mesityl group, an o-biphenyl group, an m-biphenyl group, a p-biphenyl group, a 1-naphthyl group, a 2-naphthyl group, a fluorenyl group, a 9,9-dimethylfluorenyl group, a spirofluorenyl group, a phenanthrenyl group, an anthracenyl group, and a fluoranthenyl group. In addition, specific examples of the heteroaryl group having 3 to 30 carbon atoms in the group having 1 to 100 total carbon atoms that is included in R¹ and R² include monovalent groups such as carbazole, benzocarbazole, dibenzocarbazole, indolocarbazole, benzindolocarbazole, dibenzindolocarbazole, benzindolobenzocarbazole, dibenzothiophene, benzonaphthothiophene, dibenzofuran, and benzonaphthofuran.

Next, specific structural formulae of the aforementioned organic compounds of embodiments of the present invention are shown below. Note that the present invention is not limited to these formulae.

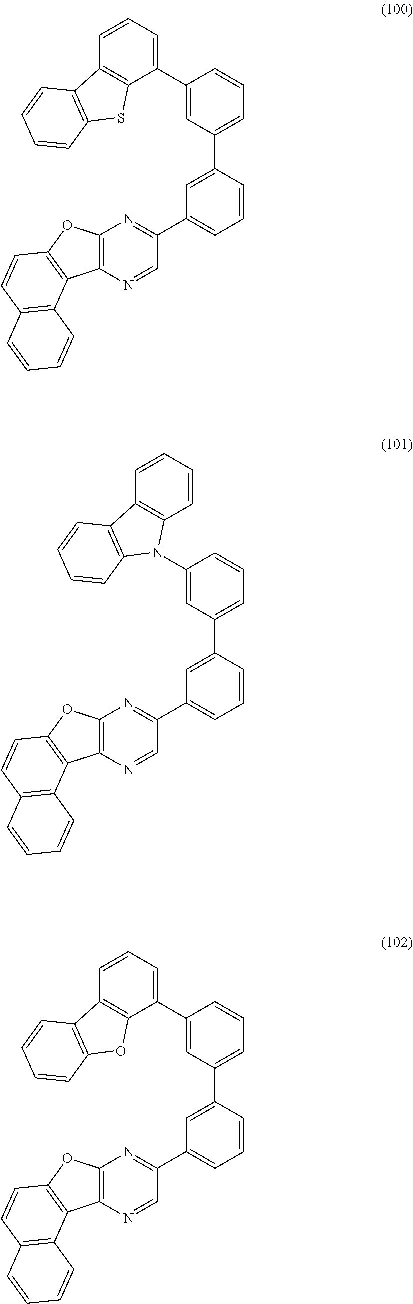 [SMILES: c1cc(-c2cccc(-c3cccc4c3sc3ccccc34)c2)cc(-c2cnc3c(n2)oc2ccc4ccccc4c23)c1 ; c1cc(-c2cccc(-n3c4ccccc4c4ccccc43)c2)cc(-c2cnc3c(n2)oc2ccc4ccccc4c23)c1 ; c1cc(-c2cccc(-c3cccc4c3oc3ccccc34)c2)cc(-c2cnc3c(n2)oc2ccc4ccccc4c23)c1]

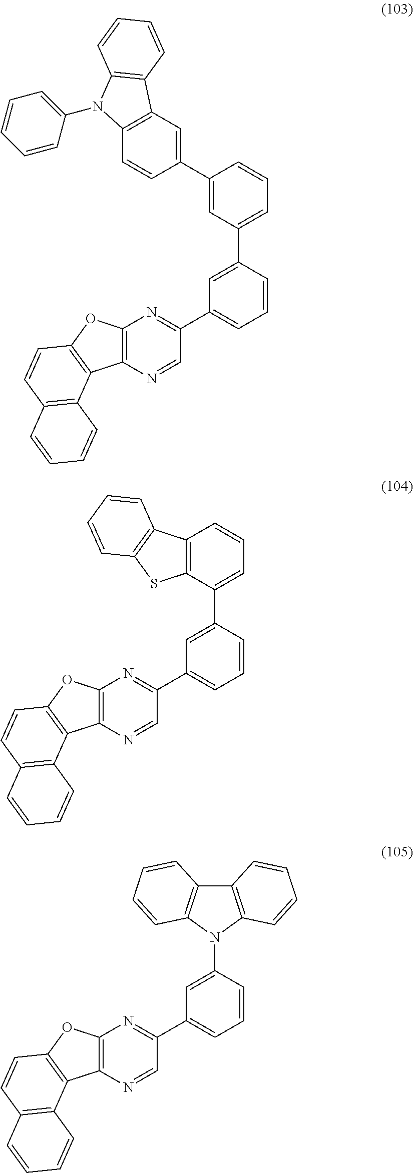

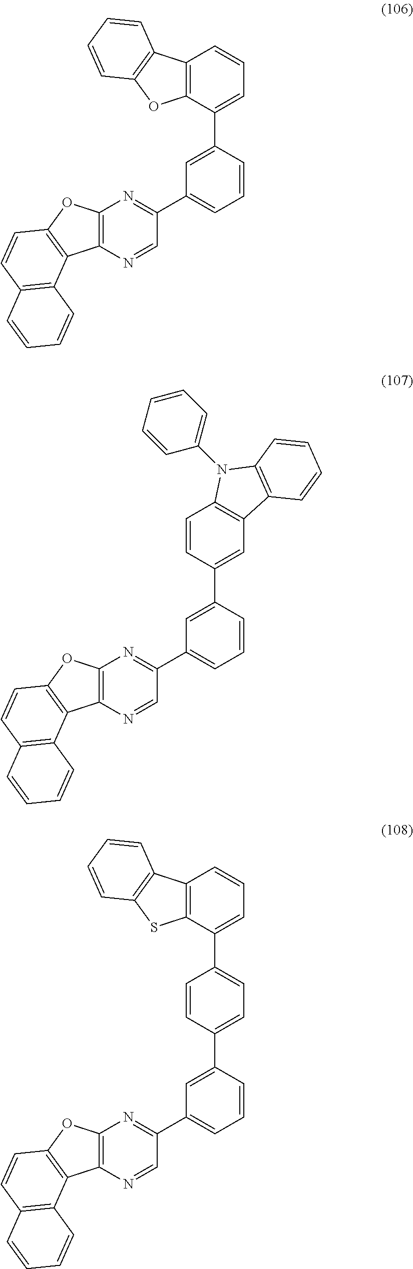 [SMILES: c1cc(-c2cnc3c(n2)oc2ccc4ccccc4c23)cc(-c2cccc3c2oc2ccccc23)c1 ; c1ccc(-n2c3ccccc3c3cc(-c4cccc(-c5cnc6c(n5)oc5ccc7ccccc7c56)c4)ccc32)cc1 ; c1cc(-c2ccc(-c3cccc4c3sc3ccccc34)cc2)cc(-c2cnc3c(n2)oc2ccc4ccccc4c23)c1]

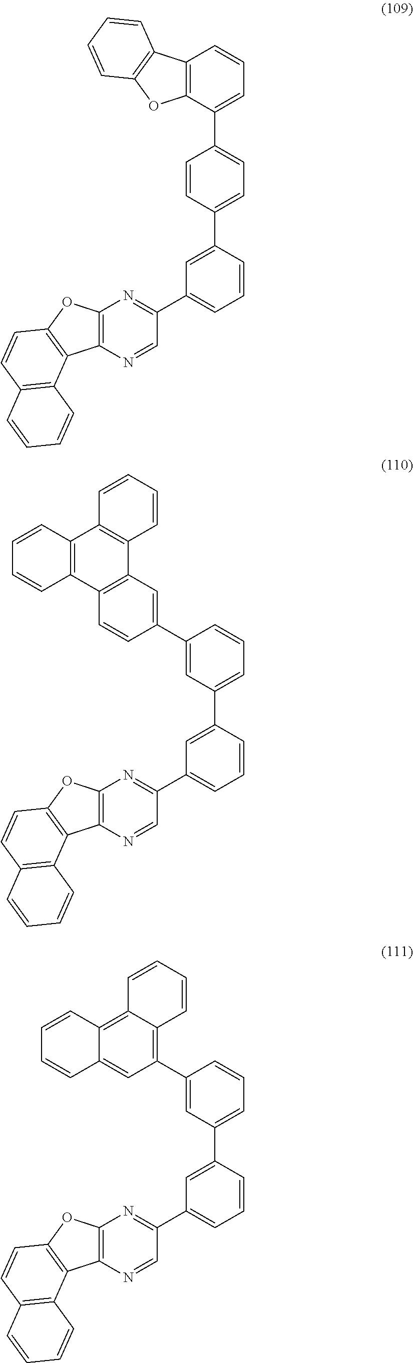 [SMILES: c1cc(-c2ccc(-c3cccc4c3oc3ccccc34)cc2)cc(-c2cnc3c(n2)oc2ccc4ccccc4c23)c1 ; c1cc(-c2cccc(-c3cnc4c(n3)oc3ccc5ccccc5c34)c2)cc(-c2ccc3c4ccccc4c4ccccc4c3c2)c1 ; c1cc(-c2cccc(-c3cc4ccccc4c4ccccc34)c2)cc(-c2cnc3c(n2)oc2ccc4ccccc4c23)c1]

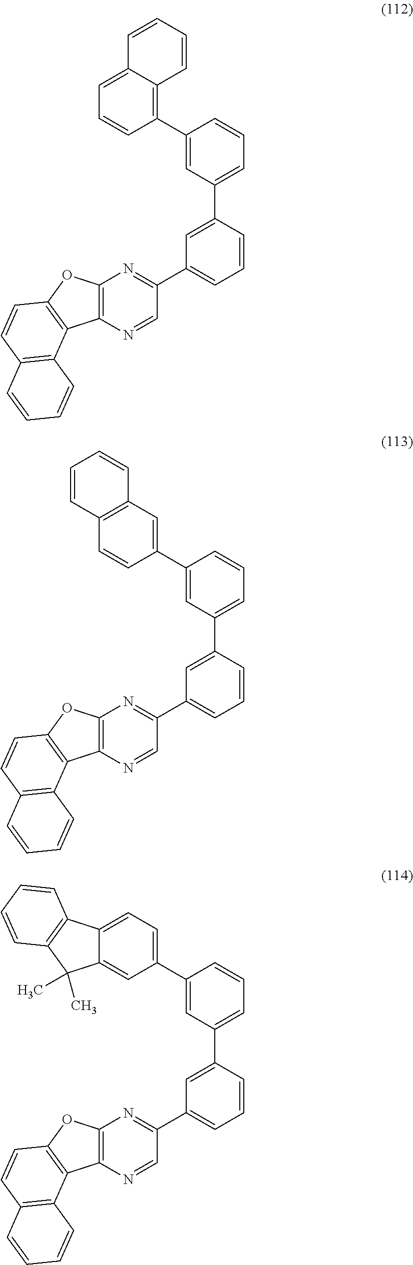

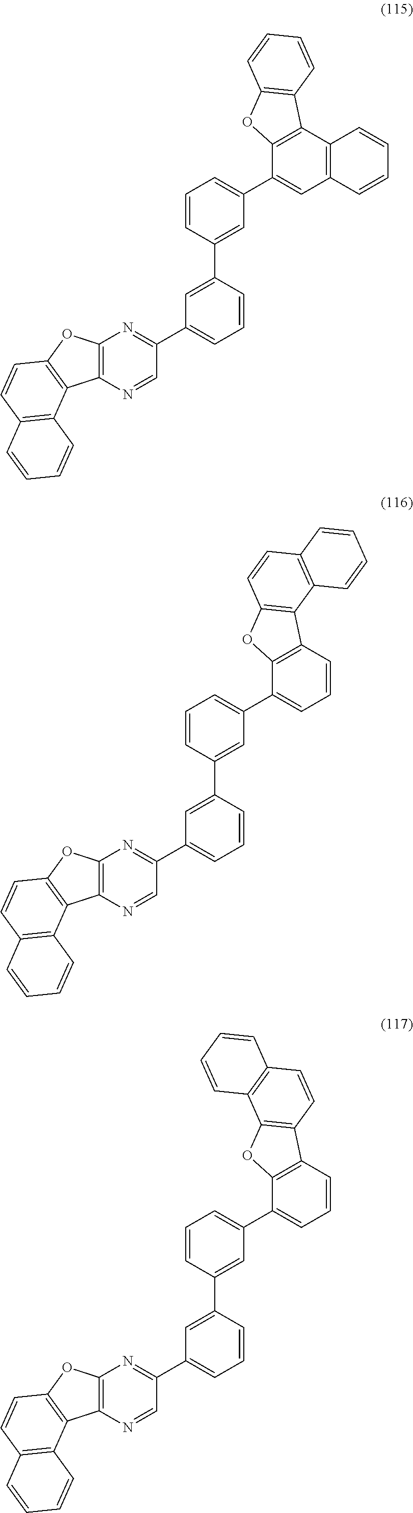 [SMILES: c1cc(-c2cccc(-c3cc4ccccc4c4c3oc3ccccc34)c2)cc(-c2cnc3c(n2)oc2ccc4ccccc4c23)c1 ; c1cc(-c2cccc(-c3cccc4c3oc3ccc5ccccc5c34)c2)cc(-c2cnc3c(n2)oc2ccc4ccccc4c23)c1 ; c1cc(-c2cccc(-c3cccc4c3oc3c5ccccc5ccc43)c2)cc(-c2cnc3c(n2)oc2ccc4ccccc4c23)c1]

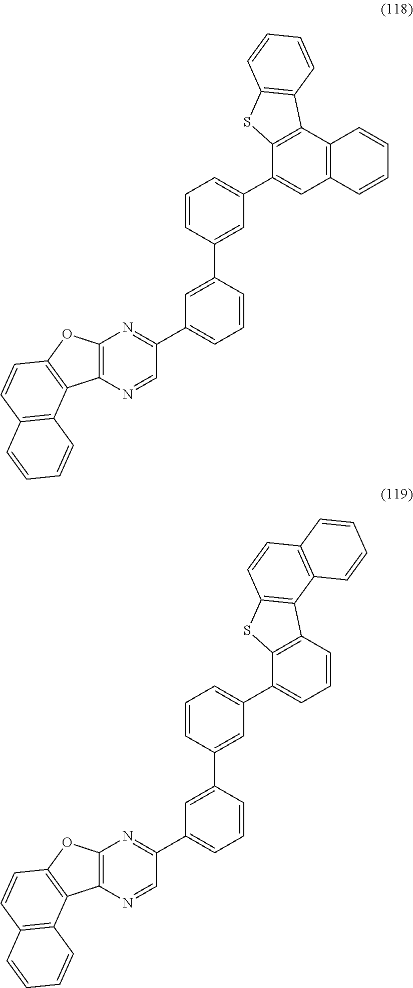 [SMILES: c1cc(-c2cccc(-c3cc4ccccc4c4c3sc3ccccc34)c2)cc(-c2cnc3c(n2)oc2ccc4ccccc4c23)c1 ; c1cc(-c2cccc(-c3cccc4c3sc3ccc5ccccc5c34)c2)cc(-c2cnc3c(n2)oc2ccc4ccccc4c23)c1]

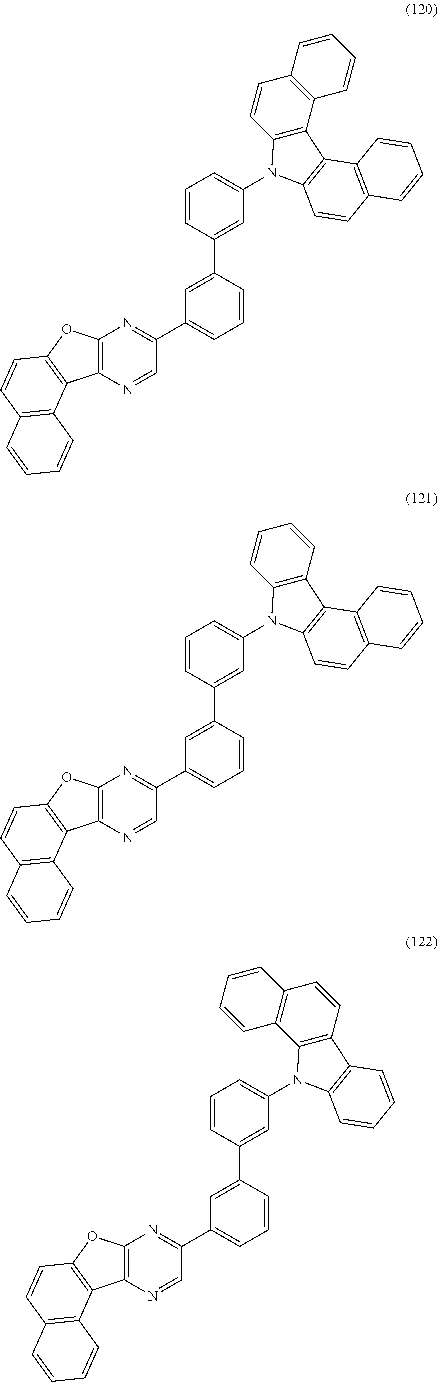 [SMILES: c1cc(-c2cccc(-n3c4ccc5ccccc5c4c4c5ccccc5ccc43)c2)cc(-c2cnc3c(n2)oc2ccc4ccccc4c23)c1 ; c1cc(-c2cccc(-n3c4ccccc4c4c5ccccc5ccc43)c2)cc(-c2cnc3c(n2)oc2ccc4ccccc4c23)c1 ; c1cc(-c2cccc(-n3c4ccccc4c4ccc5ccccc5c43)c2)cc(-c2cnc3c(n2)oc2ccc4ccccc4c23)c1]

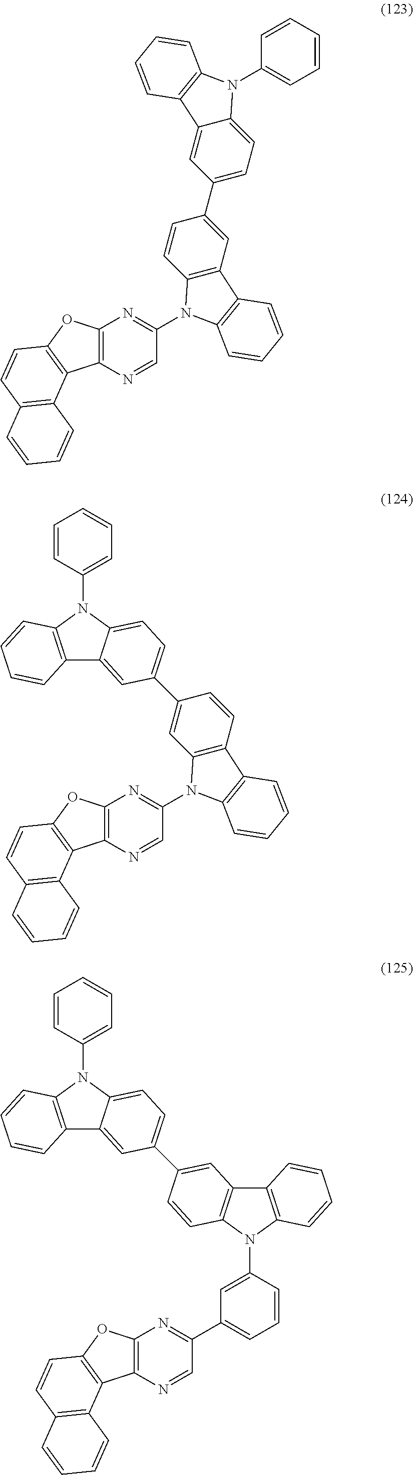

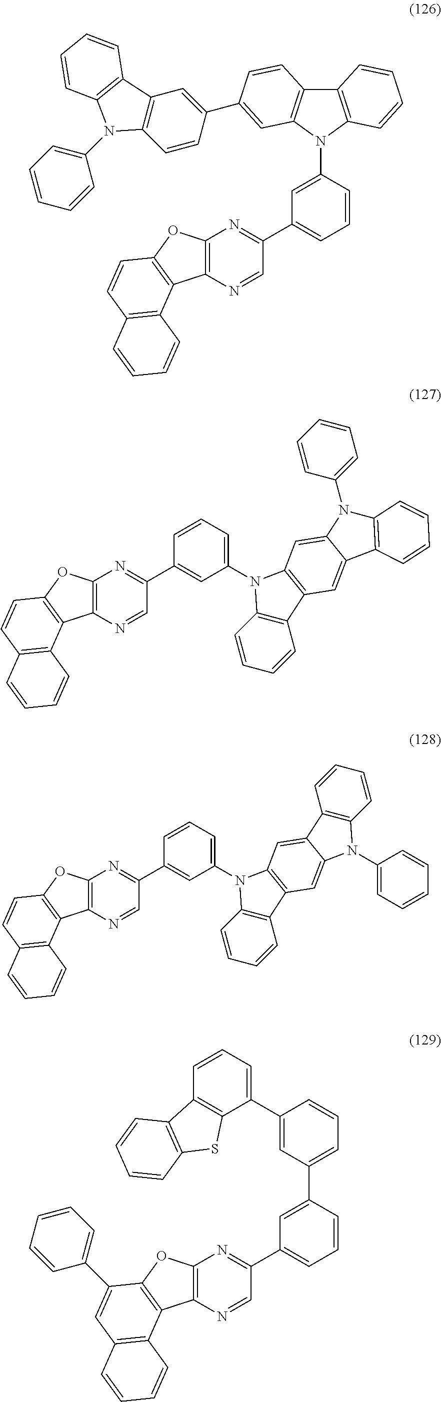 [SMILES: c1ccc(-n2c3ccccc3c3cc(-c4ccc5c6ccccc6n(-c6cccc(-c7cnc8c(n7)oc7ccc9ccccc9c78)c6)c5c4)ccc32)cc1 ; c1ccc(-n2c3ccccc3c3cc4c5ccccc5n(-c5cccc(-c6cnc7c(n6)oc6ccc8ccccc8c67)c5)c4cc32)cc1 ; c1ccc(-n2c3ccccc3c3cc4c(cc32)c2ccccc2n4-c2cccc(-c3cnc4c(n3)oc3ccc5ccccc5c34)c2)cc1 ; c1ccc(-c2cc3ccccc3c3c2oc2nc(-c4cccc(-c5cccc(-c6cccc7c6sc6ccccc67)c5)c4)cnc23)cc1]

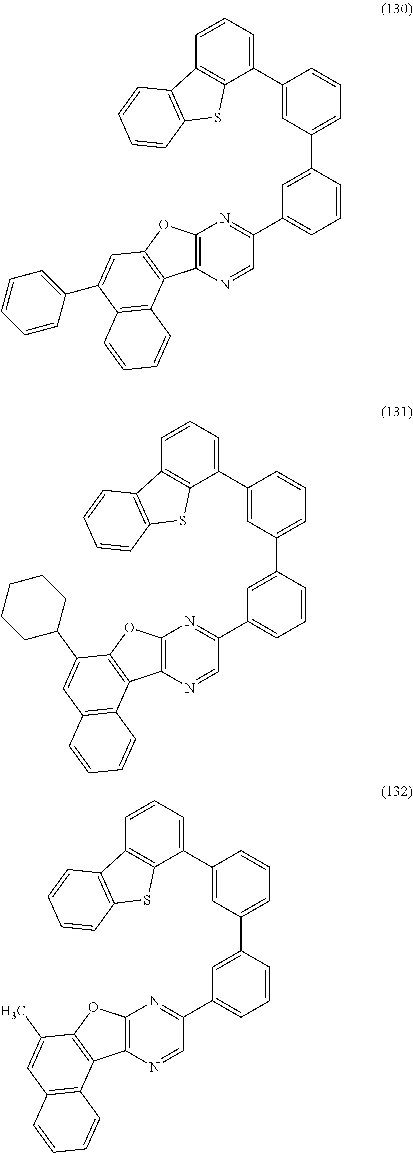

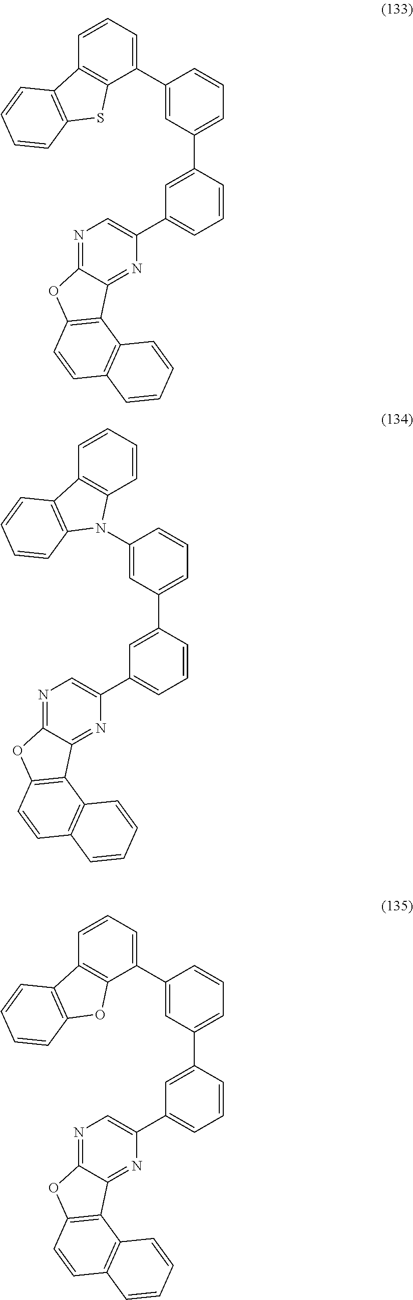

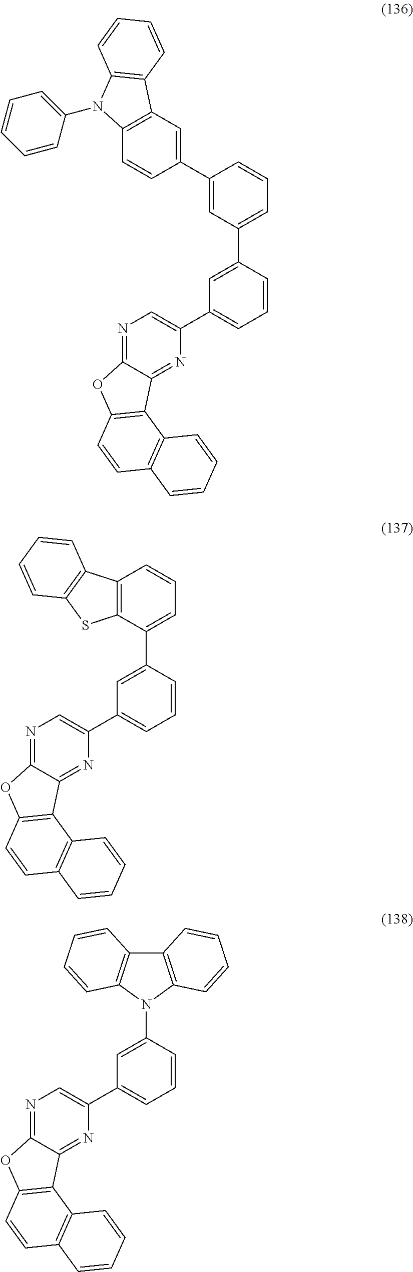

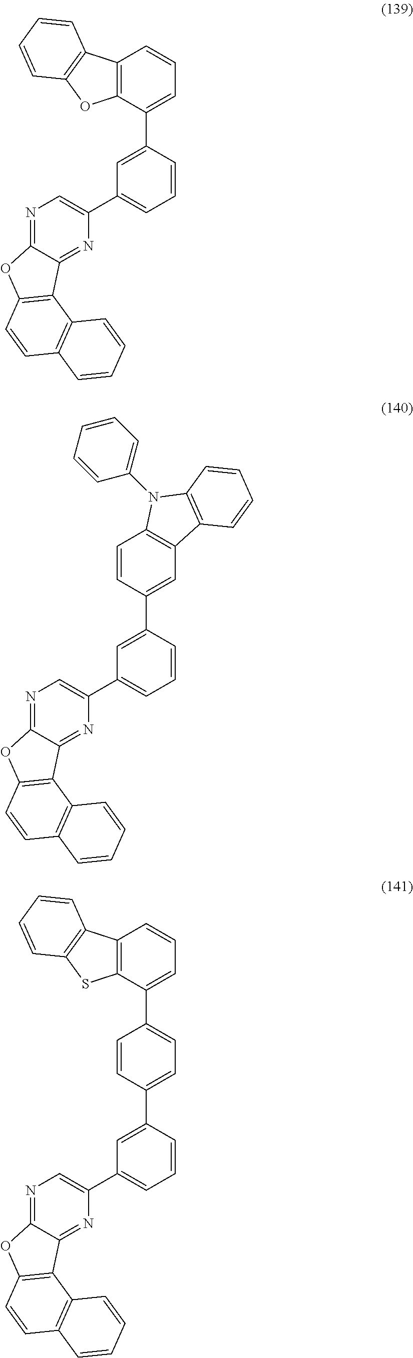

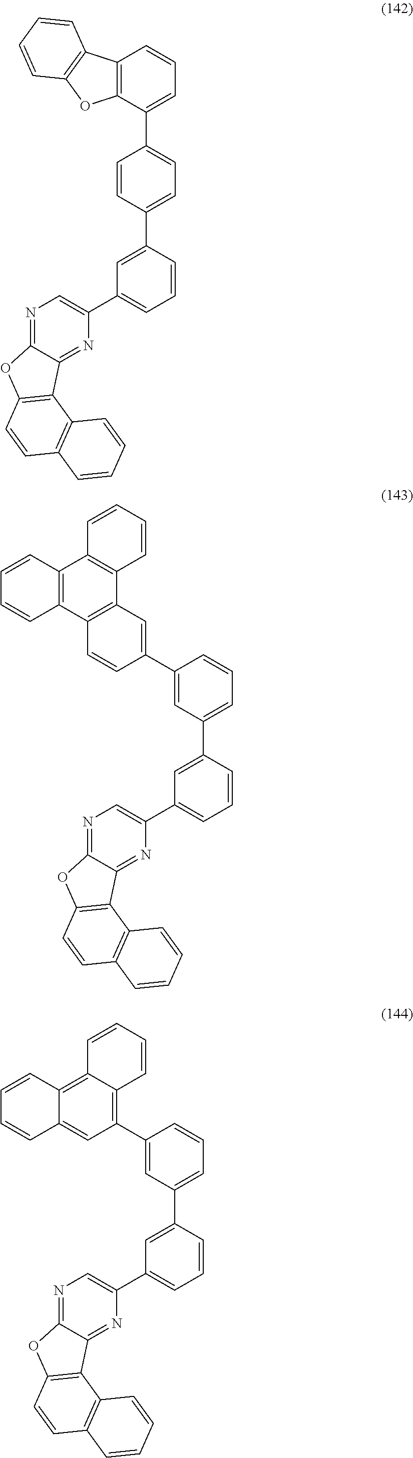

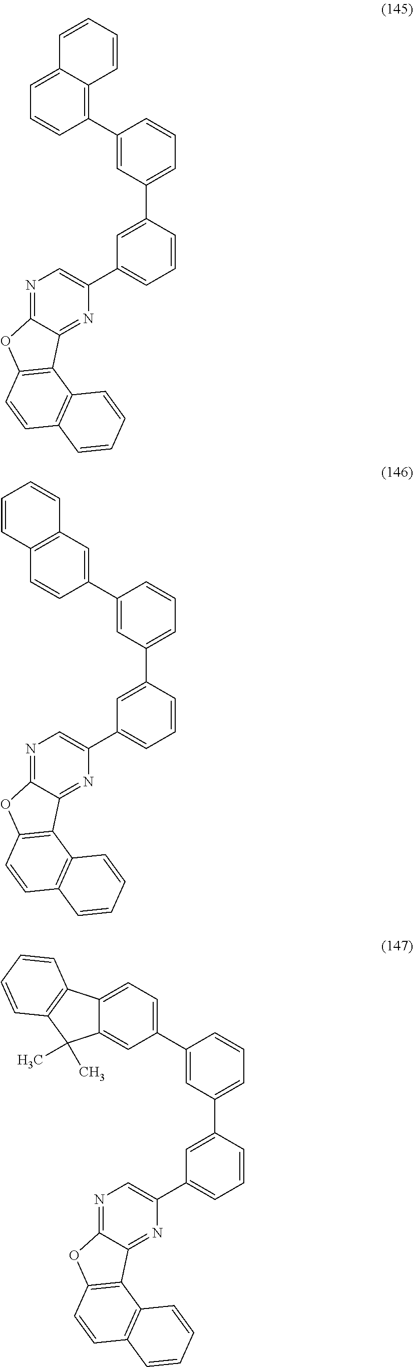 [SMILES: c1cc(-c2cccc(-c3cccc4ccccc34)c2)cc(-c2cnc3oc4ccc5ccccc5c4c3n2)c1 ; c1cc(-c2cccc(-c3cnc4oc5ccc6ccccc6c5c4n3)c2)cc(-c2ccc3ccccc3c2)c1 ; CC1(C)c2ccccc2-c2ccc(-c3cccc(-c4cccc(-c5cnc6oc7ccc8ccccc8c7c6n5)c4)c3)cc21]

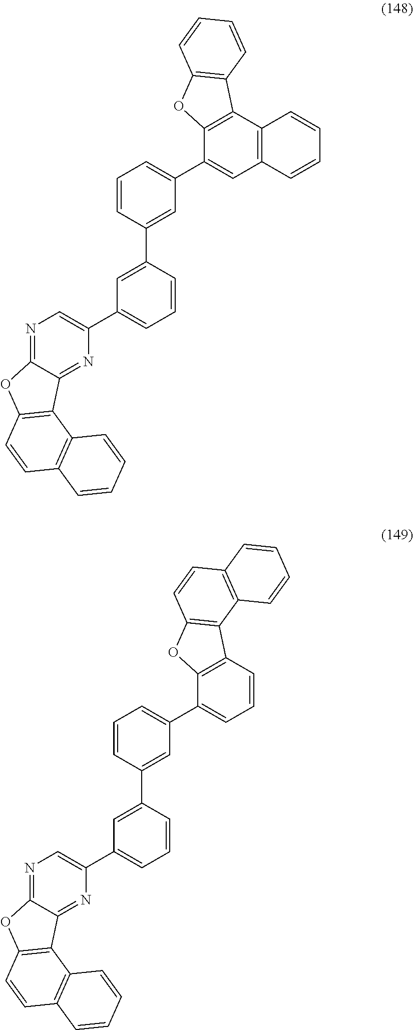 [SMILES: c1cc(-c2cccc(-c3cc4ccccc4c4c3oc3ccccc34)c2)cc(-c2cnc3oc4ccc5ccccc5c4c3n2)c1 ; c1cc(-c2cccc(-c3cccc4c3oc3ccc5ccccc5c34)c2)cc(-c2cnc3oc4ccc5ccccc5c4c3n2)c1]

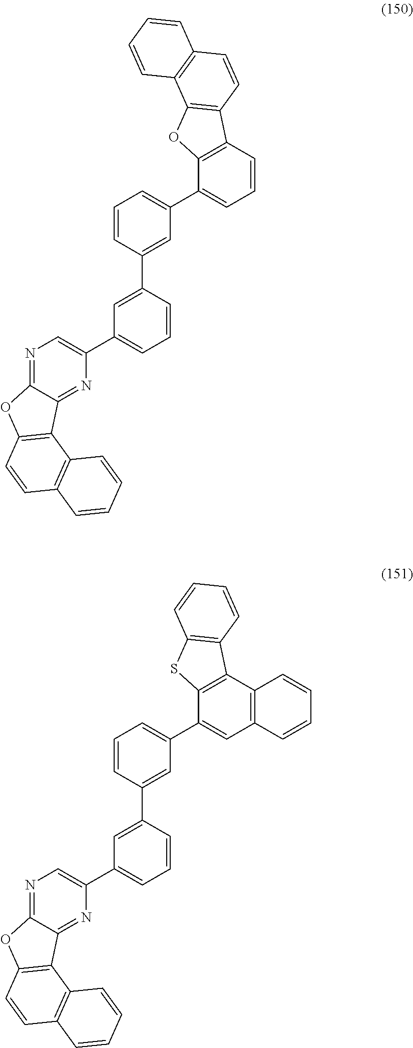 [SMILES: c1cc(-c2cccc(-c3cccc4c3oc3c5ccccc5ccc43)c2)cc(-c2cnc3oc4ccc5ccccc5c4c3n2)c1 ; c1cc(-c2cccc(-c3cc4ccccc4c4c3sc3ccccc34)c2)cc(-c2cnc3oc4ccc5ccccc5c4c3n2)c1]

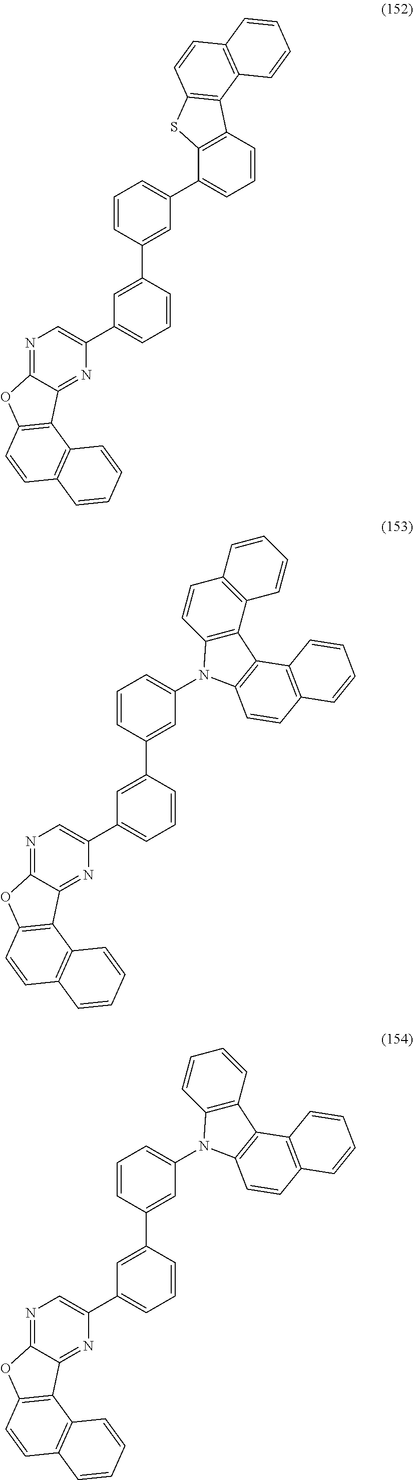 [SMILES: c1cc(-c2cccc(-c3cccc4c3sc3ccc5ccccc5c34)c2)cc(-c2cnc3oc4ccc5ccccc5c4c3n2)c1 ; c1cc(-c2cccc(-n3c4ccc5ccccc5c4c4c5ccccc5ccc43)c2)cc(-c2cnc3oc4ccc5ccccc5c4c3n2)c1 ; c1cc(-c2cccc(-n3c4ccccc4c4c5ccccc5ccc43)c2)cc(-c2cnc3oc4ccc5ccccc5c4c3n2)c1]

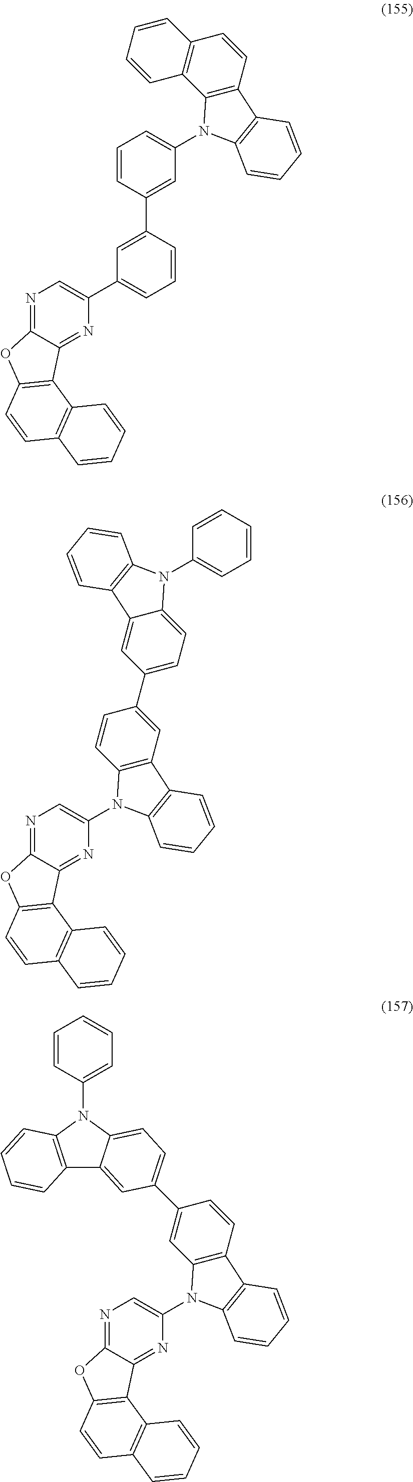

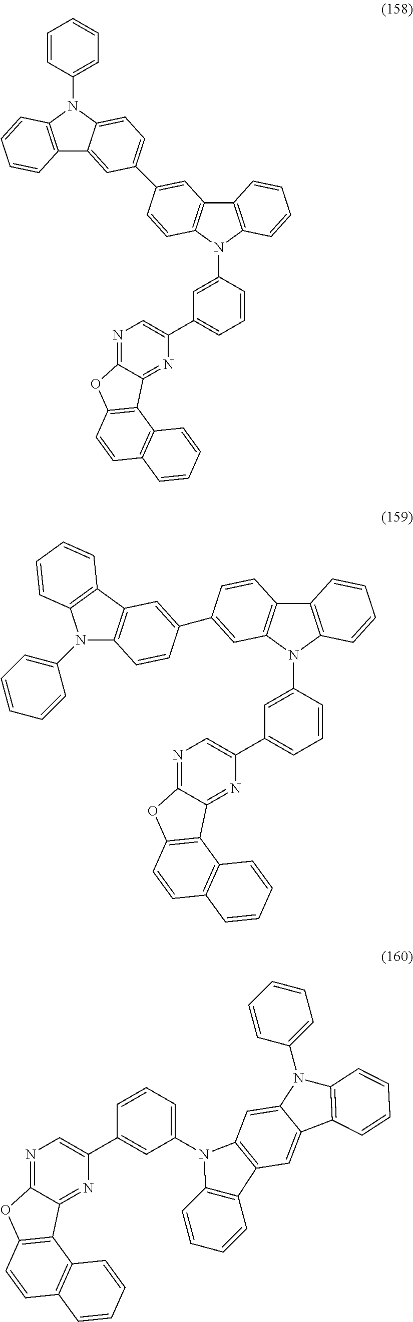 [SMILES: c1ccc(-n2c3ccccc3c3cc(-c4ccc5c(c4)c4ccccc4n5-c4cccc(-c5cnc6oc7ccc8ccccc8c7c6n5)c4)ccc32)cc1 ; c1ccc(-n2c3ccccc3c3cc(-c4ccc5c6ccccc6n(-c6cccc(-c7cnc8oc9ccc%10ccccc%10c9c8n7)c6)c5c4)ccc32)cc1 ; c1ccc(-n2c3ccccc3c3cc4c5ccccc5n(-c5cccc(-c6cnc7oc8ccc9ccccc9c8c7n6)c5)c4cc32)cc1]

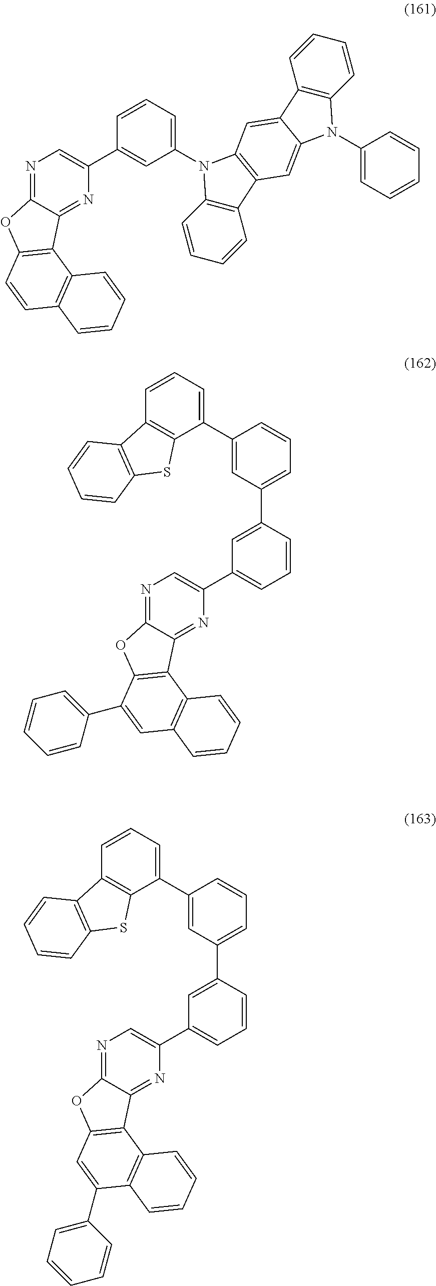 [SMILES: c1ccc(-n2c3ccccc3c3cc4c(cc32)c2ccccc2n4-c2cccc(-c3cnc4oc5ccc6ccccc6c5c4n3)c2)cc1 ; c1ccc(-c2cc3ccccc3c3c2oc2ncc(-c4cccc(-c5cccc(-c6cccc7c6sc6ccccc67)c5)c4)nc23)cc1 ; c1ccc(-c2cc3oc4ncc(-c5cccc(-c6cccc(-c7cccc8c7sc7ccccc78)c6)c5)nc4c3c3ccccc23)cc1]

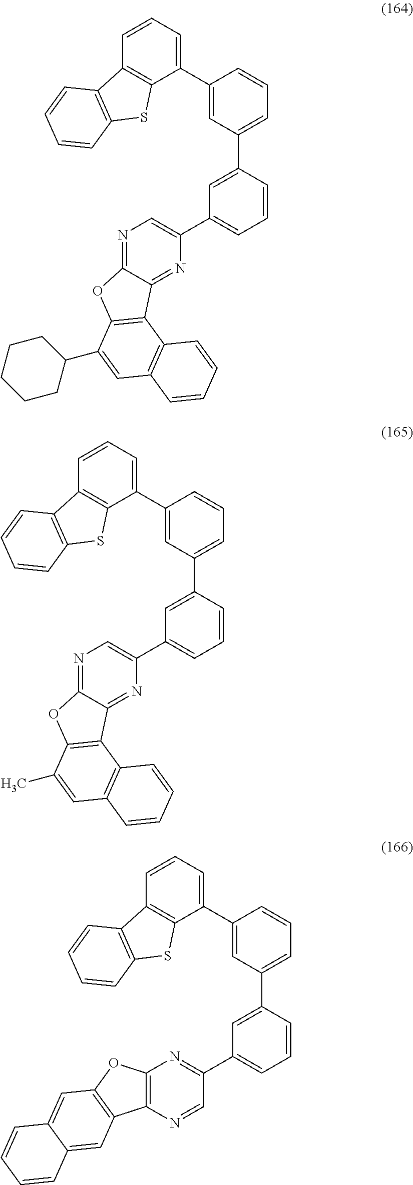

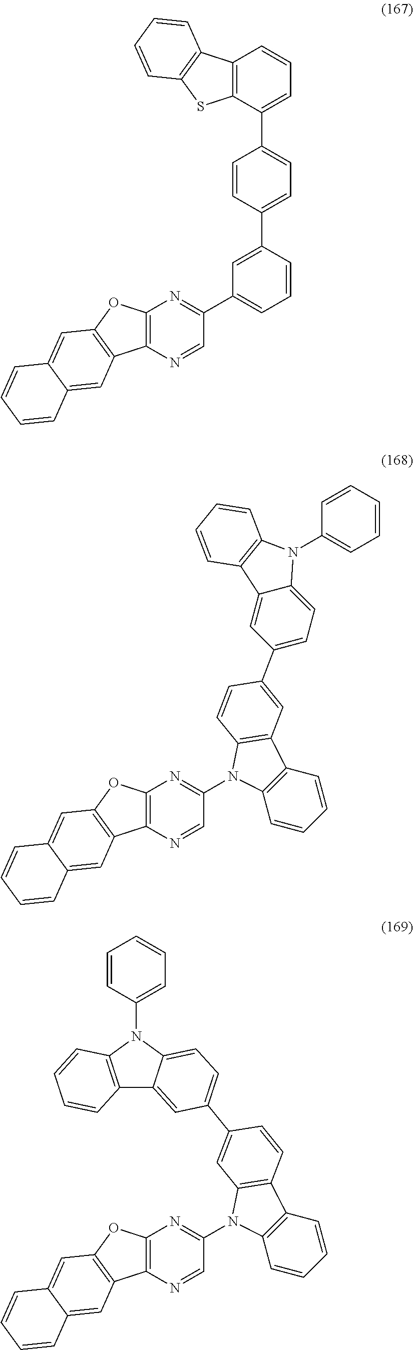 [SMILES: c1cc(-c2ccc(-c3cccc4c3sc3ccccc34)cc2)cc(-c2cnc3c(n2)oc2cc4ccccc4cc23)c1 ; c1ccc(-n2c3ccccc3c3cc(-c4ccc5c(c4)c4ccccc4n5-c4cnc5c(n4)oc4cc6ccccc6cc45)ccc32)cc1 ; c1ccc(-n2c3ccccc3c3cc(-c4ccc5c6ccccc6n(-c6cnc7c(n6)oc6cc8ccccc8cc67)c5c4)ccc32)cc1]

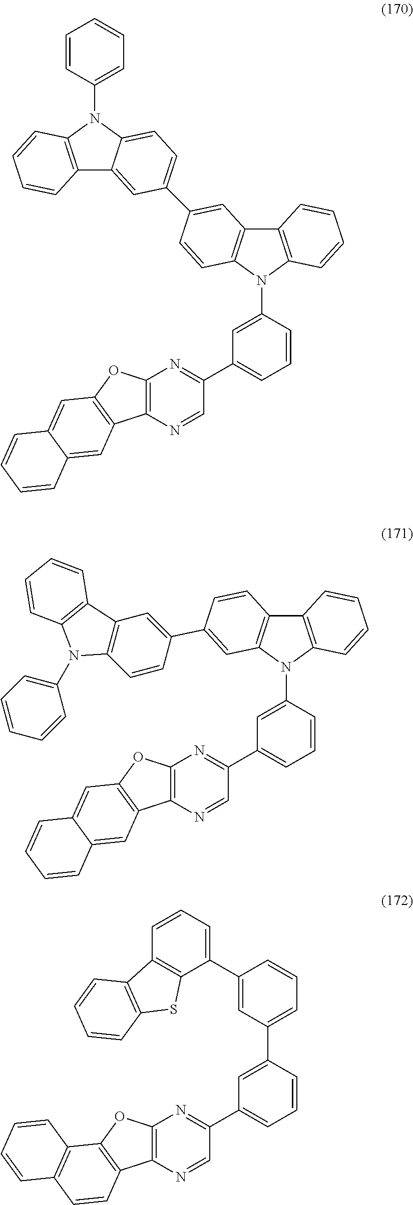 [SMILES: c1ccc(-n2c3ccccc3c3cc(-c4ccc5c(c4)c4ccccc4n5-c4cccc(-c5cnc6c(n5)oc5cc7ccccc7cc56)c4)ccc32)cc1 ; c1ccc(-n2c3ccccc3c3cc(-c4ccc5c6ccccc6n(-c6cccc(-c7cnc8c(n7)oc7cc9ccccc9cc78)c6)c5c4)ccc32)cc1 ; c1cc(-c2cccc(-c3cccc4c3sc3ccccc34)c2)cc(-c2cnc3c(n2)oc2c4ccccc4ccc32)c1]

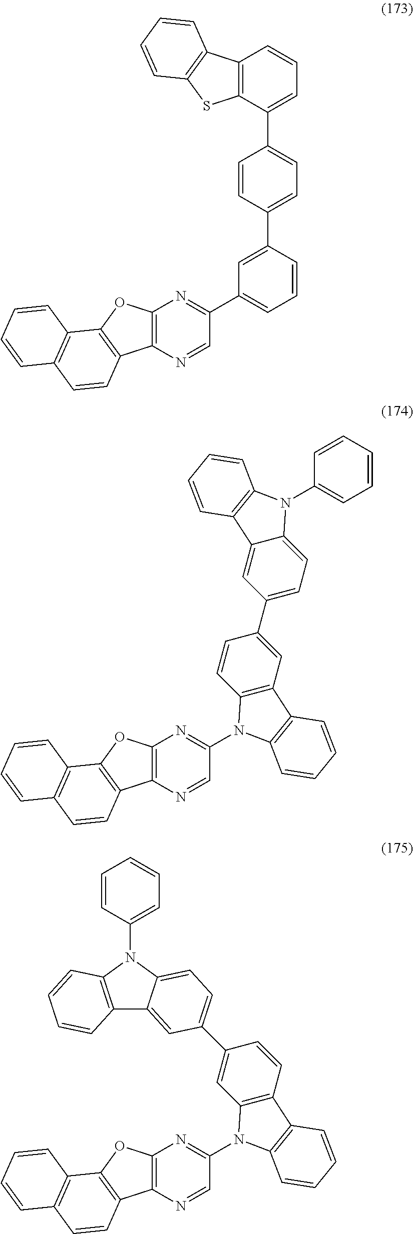

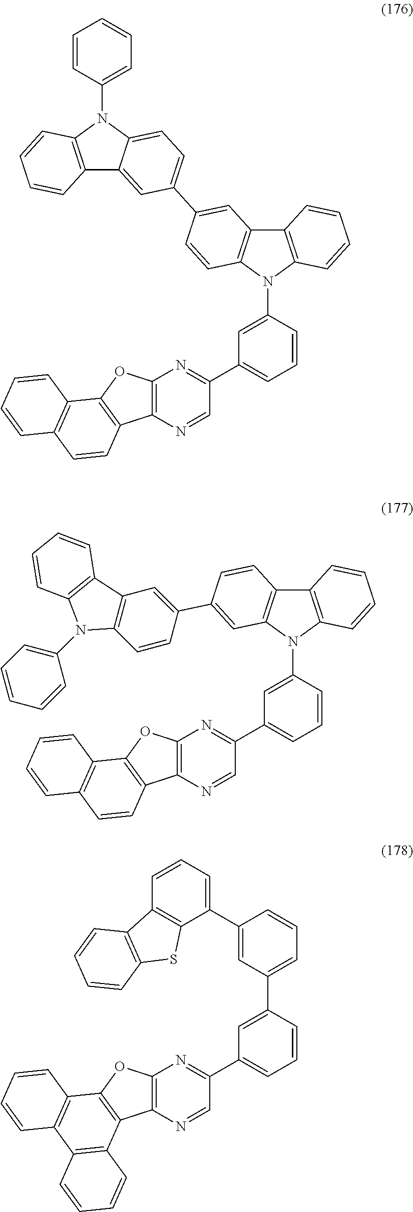 [SMILES: c1ccc(-n2c3ccccc3c3cc(-c4ccc5c(c4)c4ccccc4n5-c4cccc(-c5cnc6c(n5)oc5c7ccccc7ccc65)c4)ccc32)cc1 ; c1ccc(-n2c3ccccc3c3cc(-c4ccc5c6ccccc6n(-c6cccc(-c7cnc8c(n7)oc7c9ccccc9ccc87)c6)c5c4)ccc32)cc1 ; c1cc(-c2cccc(-c3cccc4c3sc3ccccc34)c2)cc(-c2cnc3c(n2)oc2c4ccccc4c4ccccc4c32)c1]

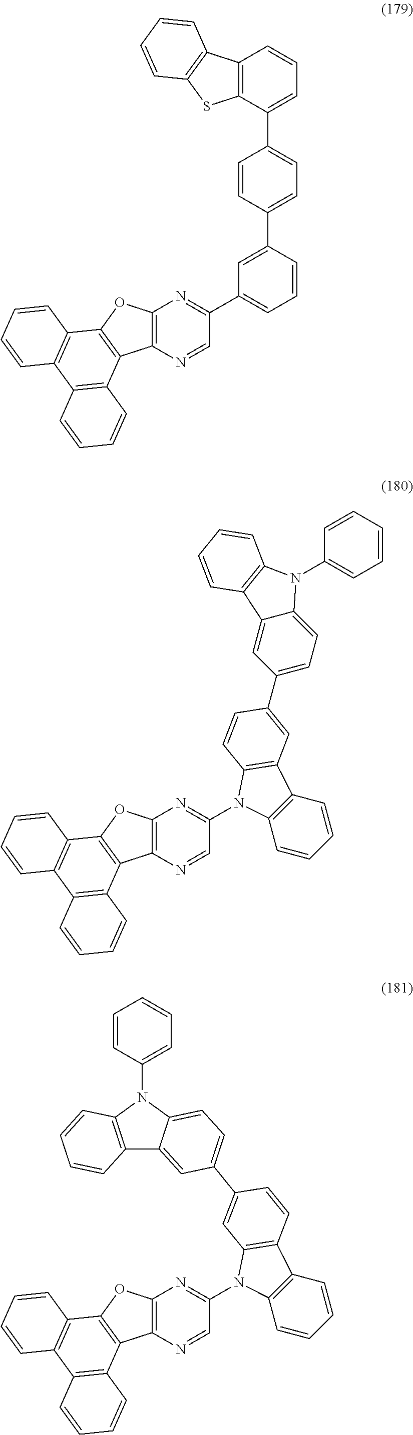 [SMILES: c1cc(-c2ccc(-c3cccc4c3sc3ccccc34)cc2)cc(-c2cnc3c(n2)oc2c4ccccc4c4ccccc4c32)c1 ; c1ccc(-n2c3ccccc3c3cc(-c4ccc5c(c4)c4ccccc4n5-c4cnc5c(n4)oc4c6ccccc6c6ccccc6c54)ccc32)cc1 ; c1ccc(-n2c3ccccc3c3cc(-c4ccc5c6ccccc6n(-c6cnc7c(n6)oc6c8ccccc8c8ccccc8c76)c5c4)ccc32)cc1]

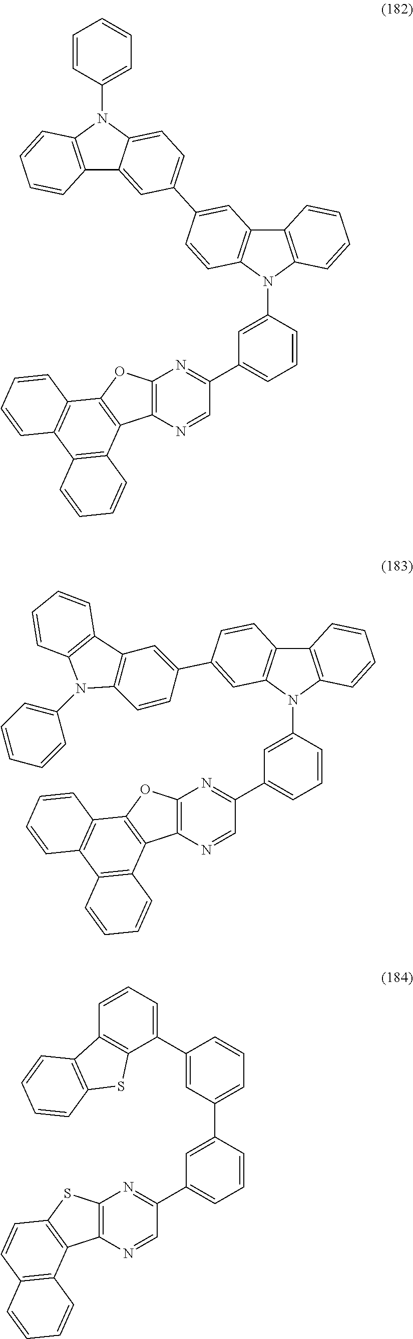 [SMILES: c1ccc(-n2c3ccccc3c3cc(-c4ccc5c(c4)c4ccccc4n5-c4cccc(-c5cnc6c(n5)oc5c7ccccc7c7ccccc7c65)c4)ccc32)cc1 ; c1ccc(-n2c3ccccc3c3cc(-c4ccc5c6ccccc6n(-c6cccc(-c7cnc8c(n7)oc7c9ccccc9c9ccccc9c87)c6)c5c4)ccc32)cc1 ; c1cc(-c2cccc(-c3cccc4c3sc3ccccc34)c2)cc(-c2cnc3c(n2)sc2ccc4ccccc4c23)c1]

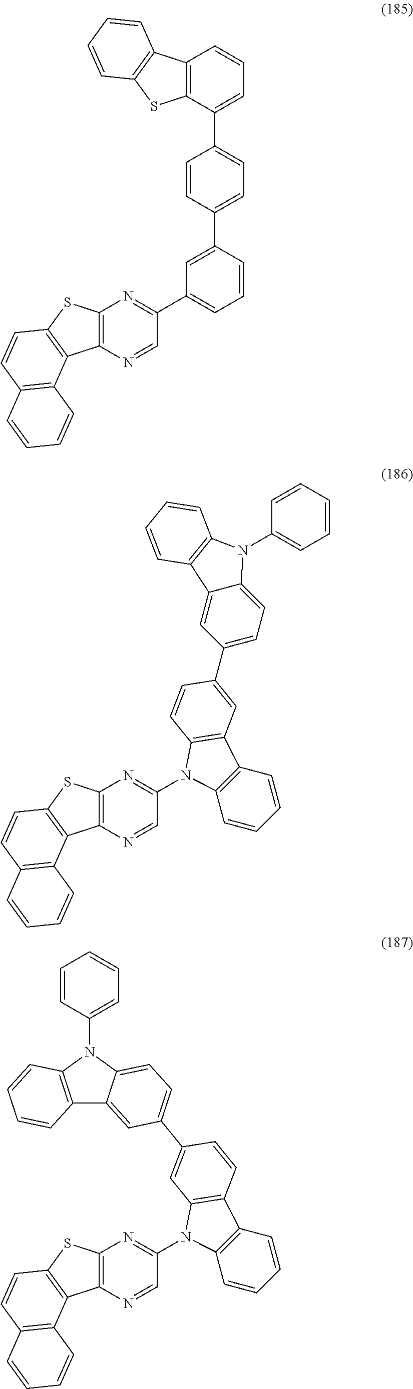

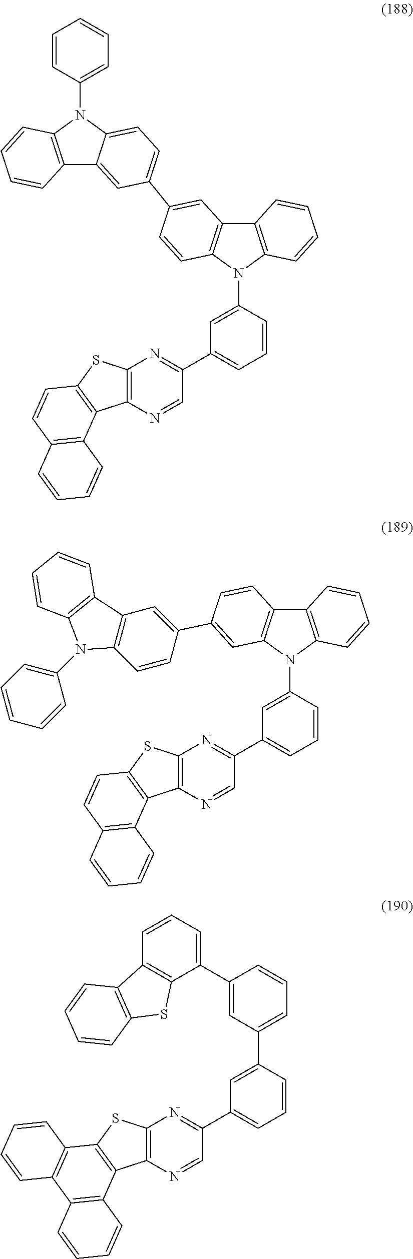

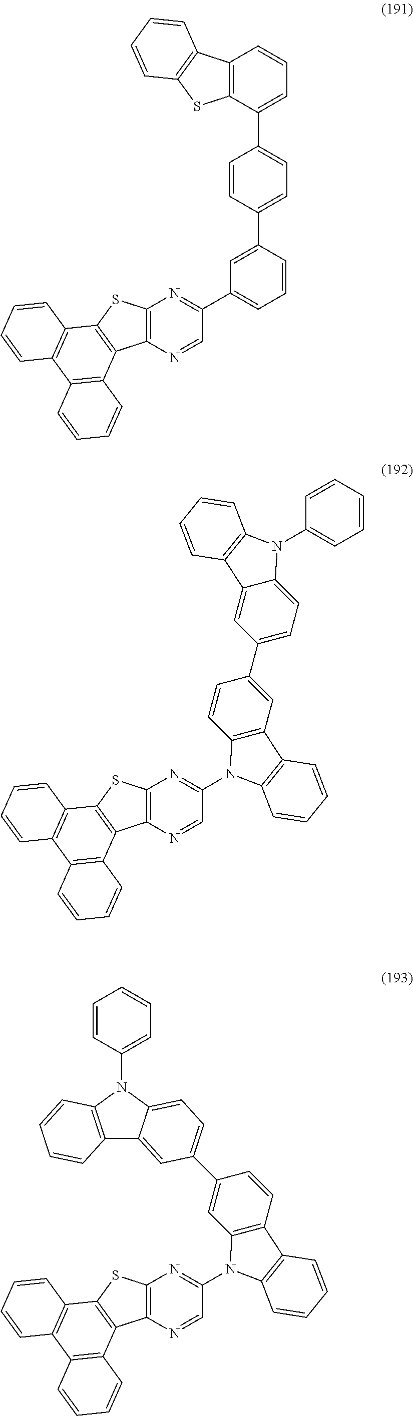 [SMILES: c1cc(-c2ccc(-c3cccc4c3sc3ccccc34)cc2)cc(-c2cnc3c(n2)sc2c4ccccc4c4ccccc4c32)c1 ; c1ccc(-n2c3ccccc3c3cc(-c4ccc5c(c4)c4ccccc4n5-c4cnc5c(n4)sc4c6ccccc6c6ccccc6c54)ccc32)cc1 ; c1ccc(-n2c3ccccc3c3cc(-c4ccc5c6ccccc6n(-c6cnc7c(n6)sc6c8ccccc8c8ccccc8c76)c5c4)ccc32)cc1]

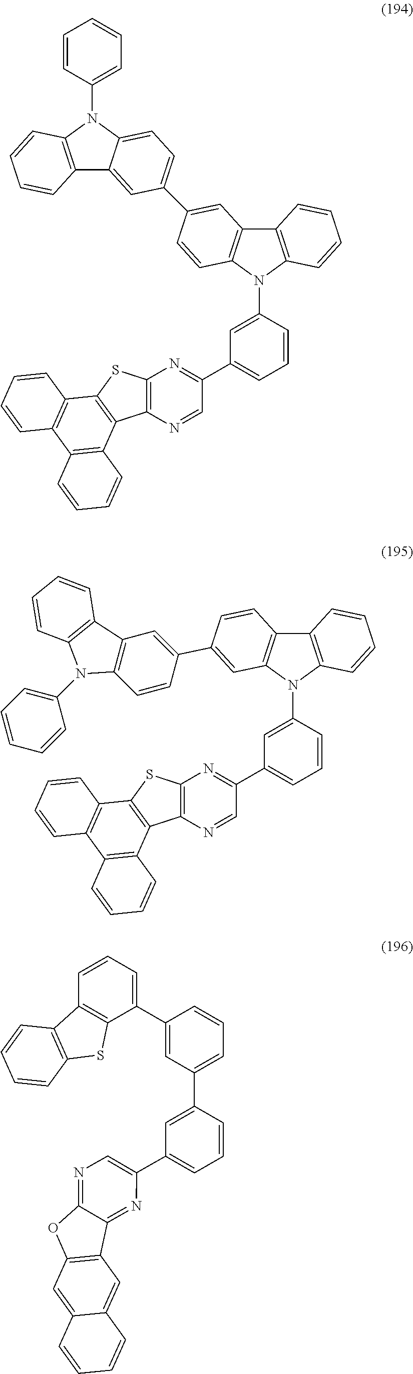

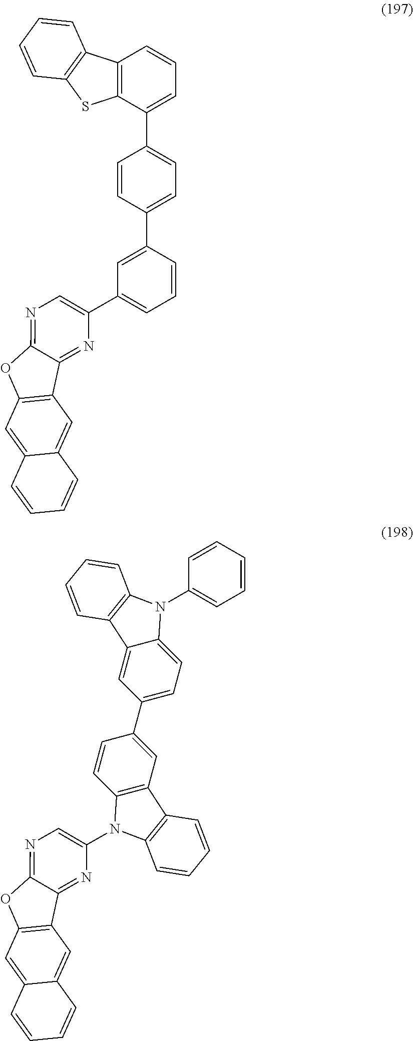 [SMILES: c1cc(-c2ccc(-c3cccc4c3sc3ccccc34)cc2)cc(-c2cnc3oc4cc5ccccc5cc4c3n2)c1 ; c1ccc(-n2c3ccccc3c3cc(-c4ccc5c(c4)c4ccccc4n5-c4cnc5oc6cc7ccccc7cc6c5n4)ccc32)cc1]

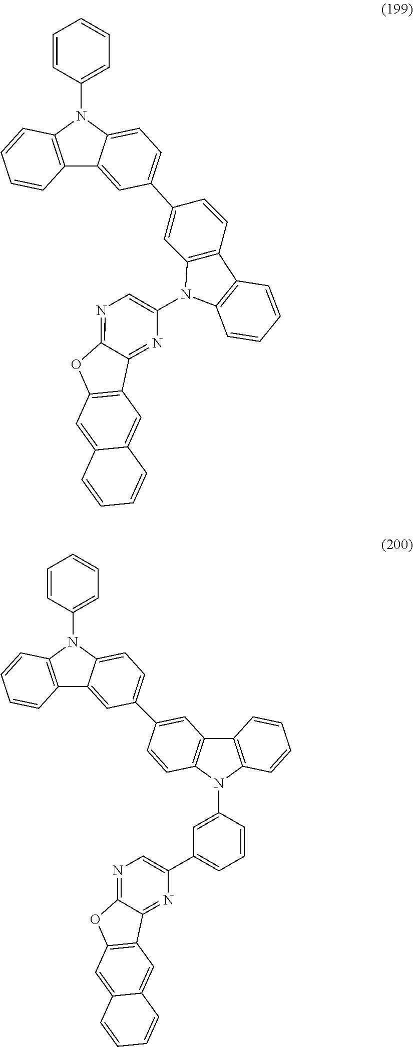 [SMILES: c1ccc(-n2c3ccccc3c3cc(-c4ccc5c6ccccc6n(-c6cnc7oc8cc9ccccc9cc8c7n6)c5c4)ccc32)cc1 ; c1ccc(-n2c3ccccc3c3cc(-c4ccc5c(c4)c4ccccc4n5-c4cccc(-c5cnc6oc7cc8ccccc8cc7c6n5)c4)ccc32)cc1]

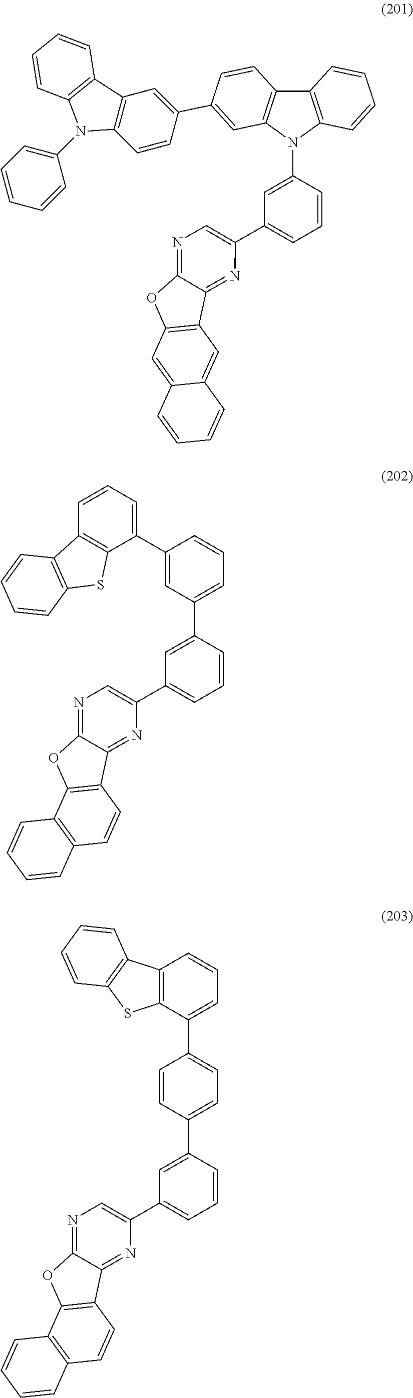 [SMILES: c1ccc(-n2c3ccccc3c3cc(-c4ccc5c6ccccc6n(-c6cccc(-c7cnc8oc9cc%10ccccc%10cc9c8n7)c6)c5c4)ccc32)cc1 ; c1cc(-c2cccc(-c3cccc4c3sc3ccccc34)c2)cc(-c2cnc3oc4c5ccccc5ccc4c3n2)c1 ; c1cc(-c2ccc(-c3cccc4c3sc3ccccc34)cc2)cc(-c2cnc3oc4c5ccccc5ccc4c3n2)c1]

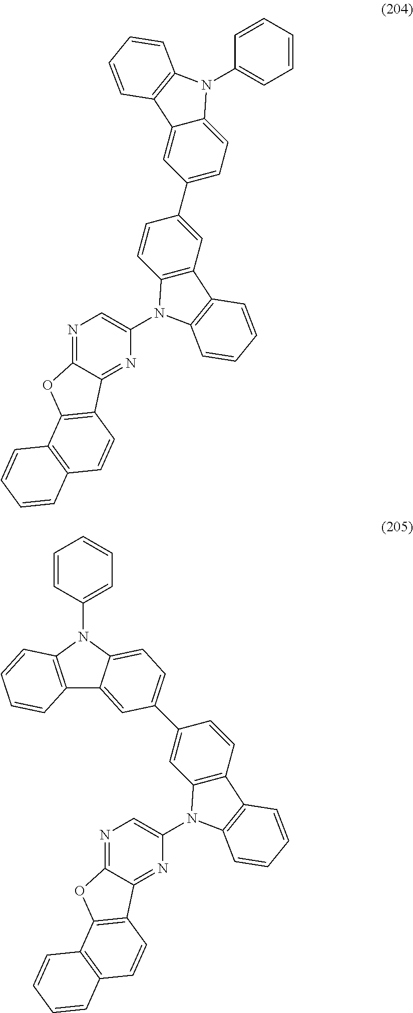

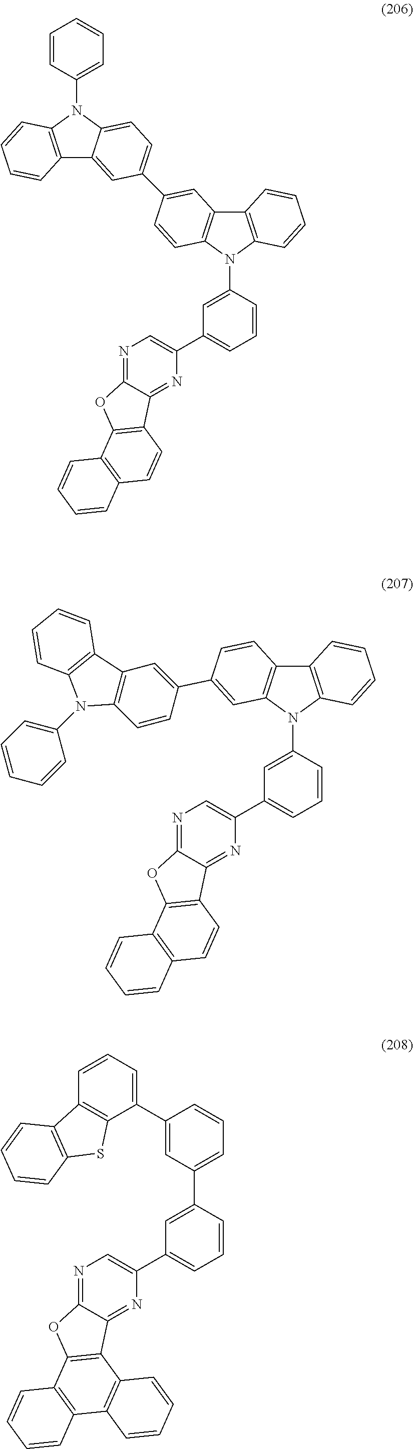 [SMILES: c1ccc(-n2c3ccccc3c3cc(-c4ccc5c(c4)c4ccccc4n5-c4cccc(-c5cnc6oc7c8ccccc8ccc7c6n5)c4)ccc32)cc1 ; c1ccc(-n2c3ccccc3c3cc(-c4ccc5c6ccccc6n(-c6cccc(-c7cnc8oc9c%10ccccc%10ccc9c8n7)c6)c5c4)ccc32)cc1 ; c1cc(-c2cccc(-c3cccc4c3sc3ccccc34)c2)cc(-c2cnc3oc4c5ccccc5c5ccccc5c4c3n2)c1]

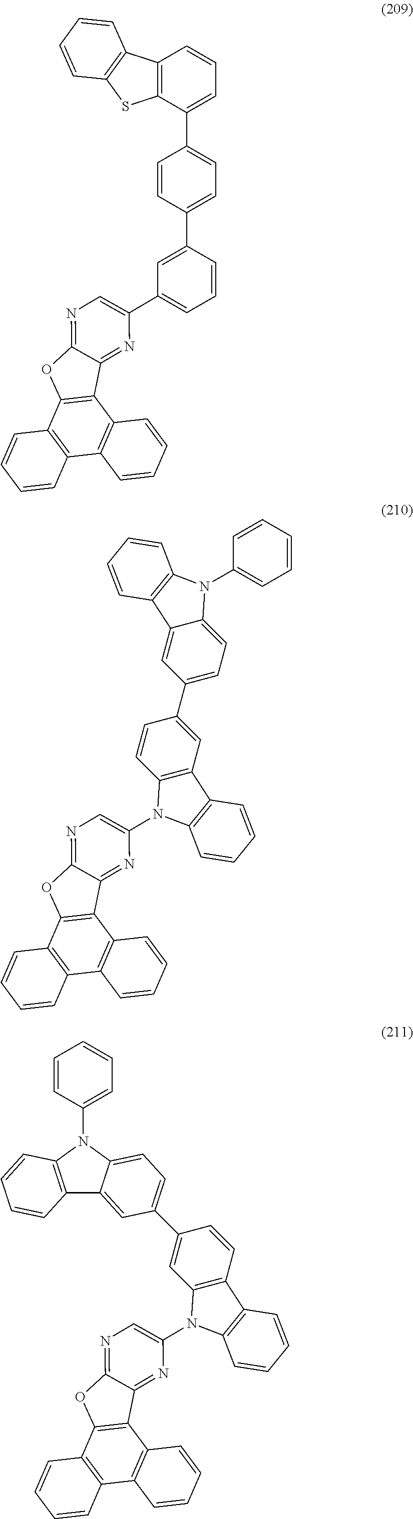

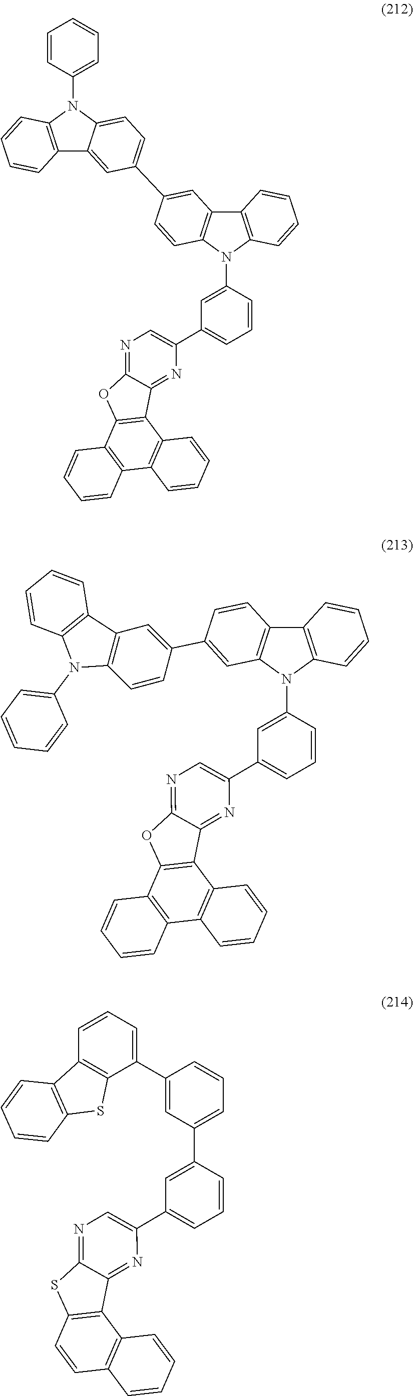 [SMILES: c1ccc(-n2c3ccccc3c3cc(-c4ccc5c(c4)c4ccccc4n5-c4cccc(-c5cnc6oc7c8ccccc8c8ccccc8c7c6n5)c4)ccc32)cc1 ; c1ccc(-n2c3ccccc3c3cc(-c4ccc5c6ccccc6n(-c6cccc(-c7cnc8oc9c%10ccccc%10c%10ccccc%10c9c8n7)c6)c5c4)ccc32)cc1 ; c1cc(-c2cccc(-c3cccc4c3sc3ccccc34)c2)cc(-c2cnc3sc4ccc5ccccc5c4c3n2)c1]

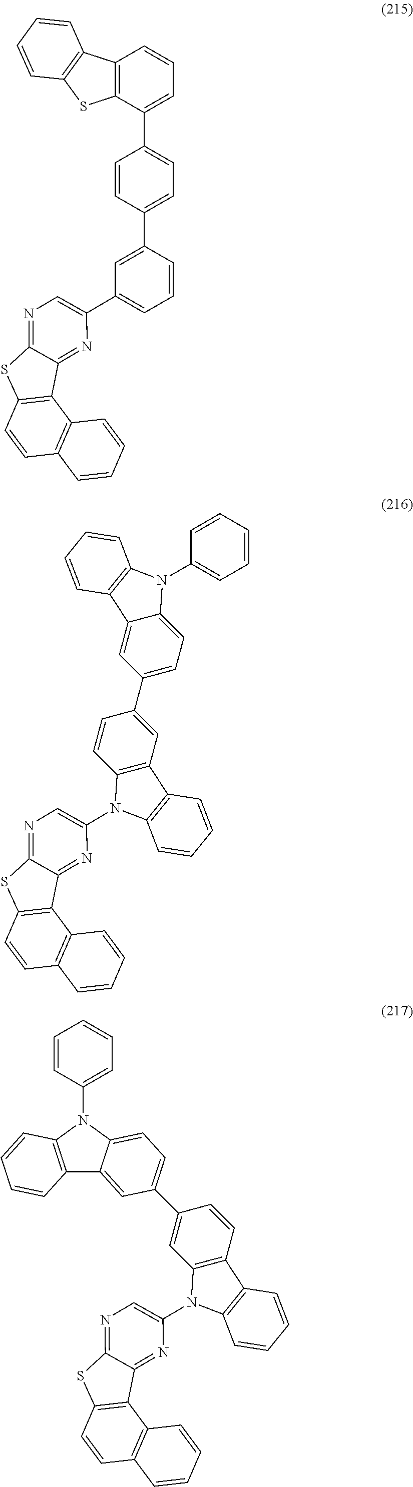

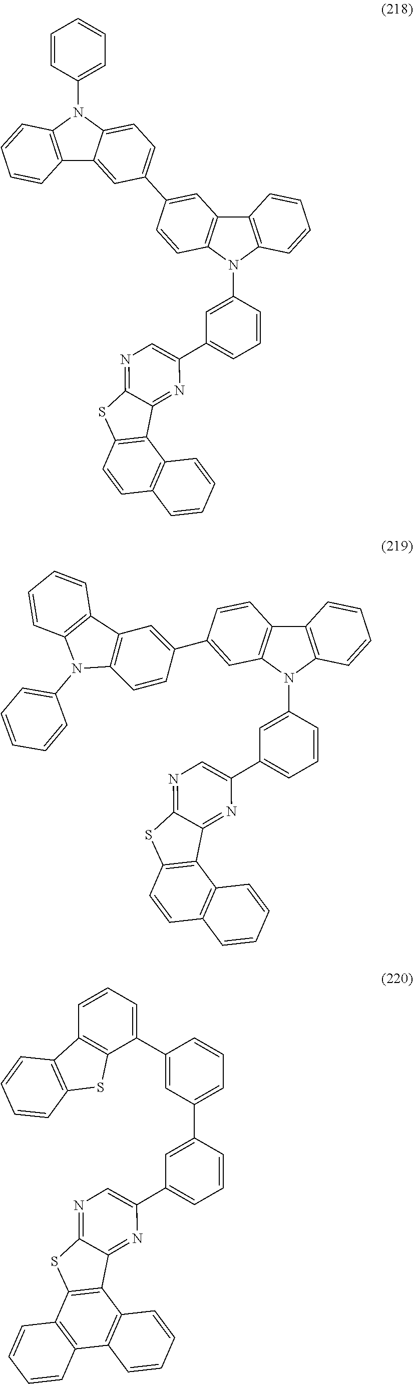 [SMILES: c1ccc(-n2c3ccccc3c3cc(-c4ccc5c(c4)c4ccccc4n5-c4cccc(-c5cnc6sc7ccc8ccccc8c7c6n5)c4)ccc32)cc1 ; c1ccc(-n2c3ccccc3c3cc(-c4ccc5c6ccccc6n(-c6cccc(-c7cnc8sc9ccc%10ccccc%10c9c8n7)c6)c5c4)ccc32)cc1 ; c1cc(-c2cccc(-c3cccc4c3sc3ccccc34)c2)cc(-c2cnc3sc4c5ccccc5c5ccccc5c4c3n2)c1]

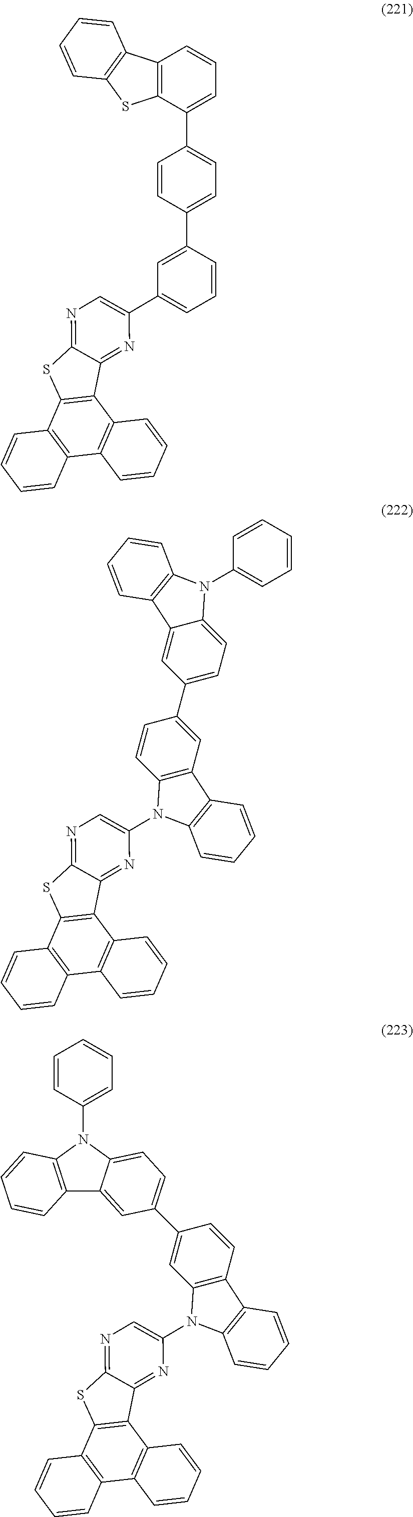

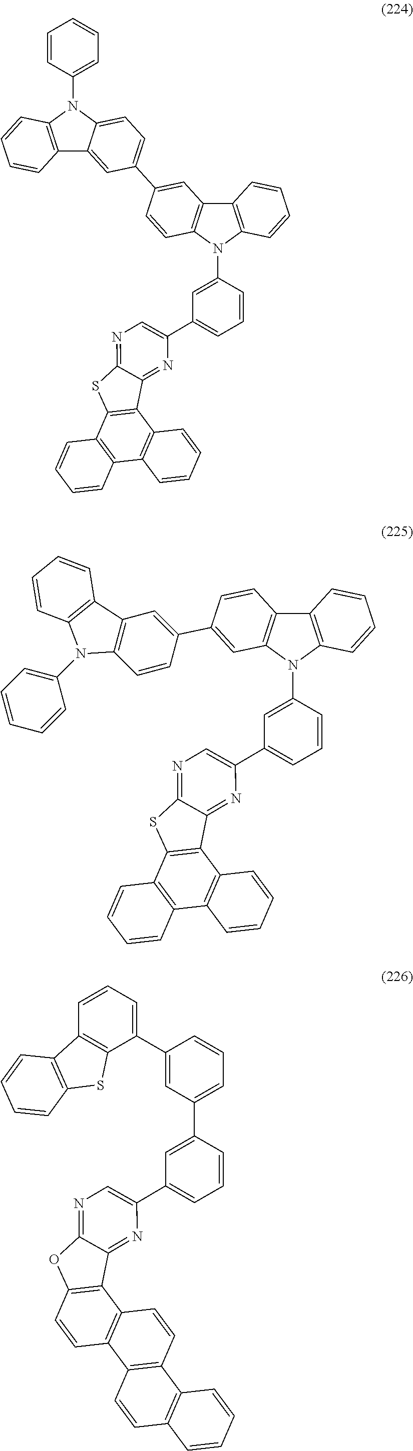

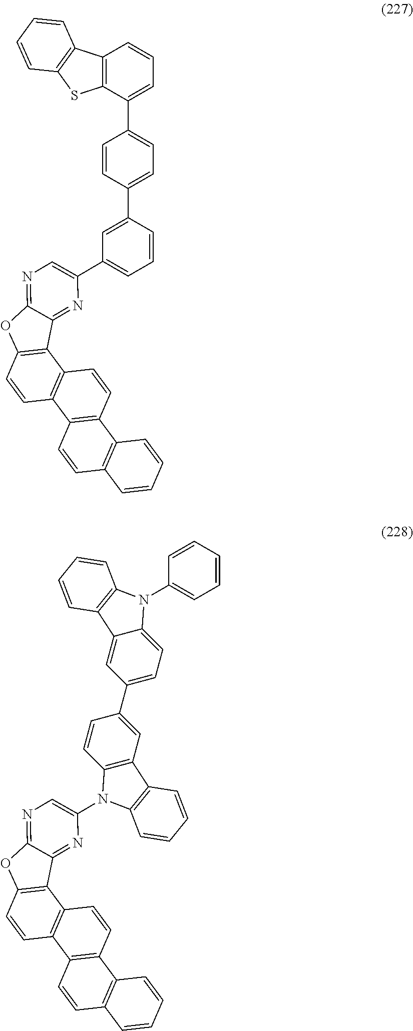 [SMILES: c1cc(-c2ccc(-c3cccc4c3sc3ccccc34)cc2)cc(-c2cnc3oc4ccc5c6ccc7ccccc7c6ccc5c4c3n2)c1 ; c1ccc(-n2c3ccccc3c3cc(-c4ccc5c(c4)c4ccccc4n5-c4cnc5oc6ccc7c8ccc9ccccc9c8ccc7c6c5n4)ccc32)cc1]

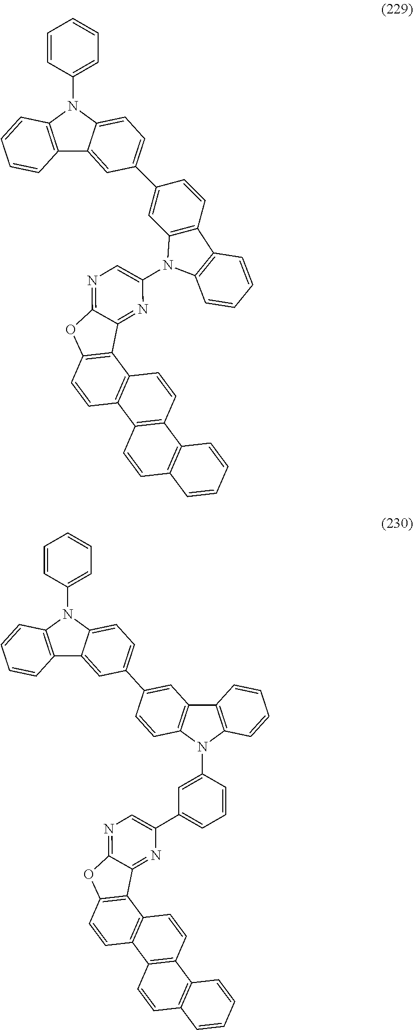 [SMILES: c1ccc(-n2c3ccccc3c3cc(-c4ccc5c6ccccc6n(-c6cnc7oc8ccc9c%10ccc%11ccccc%11c%10ccc9c8c7n6)c5c4)ccc32)cc1 ; c1ccc(-n2c3ccccc3c3cc(-c4ccc5c(c4)c4ccccc4n5-c4cccc(-c5cnc6oc7ccc8c9ccc%10ccccc%10c9ccc8c7c6n5)c4)ccc32)cc1]

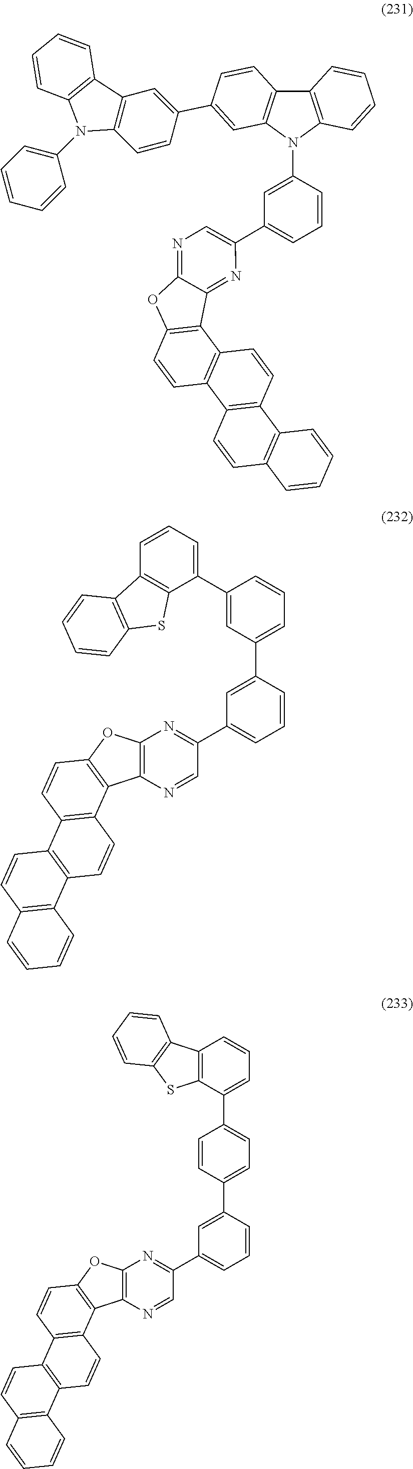 [SMILES: c1ccc(-n2c3ccccc3c3cc(-c4ccc5c6ccccc6n(-c6cccc(-c7cnc8oc9ccc%10c%11ccc%12ccccc%12c%11ccc%10c9c8n7)c6)c5c4)ccc32)cc1 ; c1cc(-c2cccc(-c3cccc4c3sc3ccccc34)c2)cc(-c2cnc3c(n2)oc2ccc4c5ccc6ccccc6c5ccc4c23)c1 ; c1cc(-c2ccc(-c3cccc4c3sc3ccccc34)cc2)cc(-c2cnc3c(n2)oc2ccc4c5ccc6ccccc6c5ccc4c23)c1]

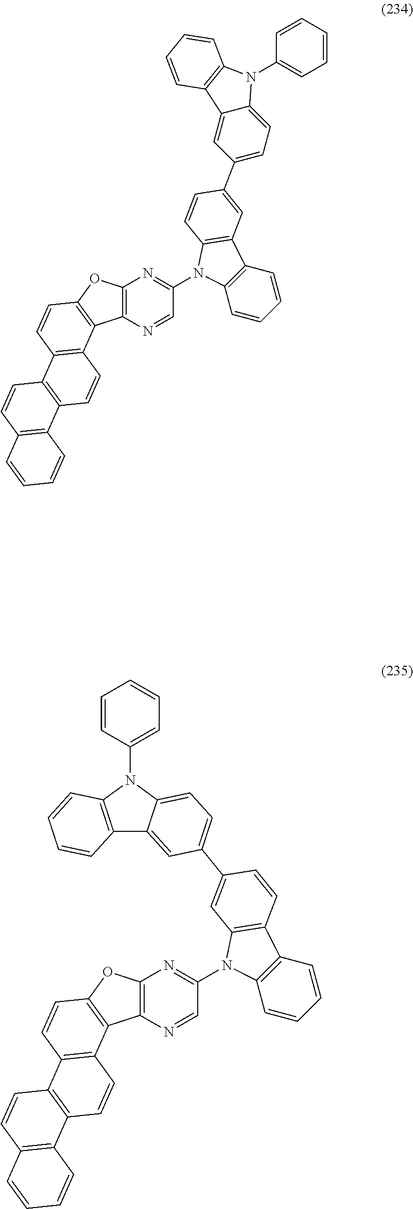 [SMILES: c1ccc(-n2c3ccccc3c3cc(-c4ccc5c(c4)c4ccccc4n5-c4cnc5c(n4)oc4ccc6c7ccc8ccccc8c7ccc6c45)ccc32)cc1 ; c1ccc(-n2c3ccccc3c3cc(-c4ccc5c6ccccc6n(-c6cnc7c(n6)oc6ccc8c9ccc%10ccccc%10c9ccc8c67)c5c4)ccc32)cc1]

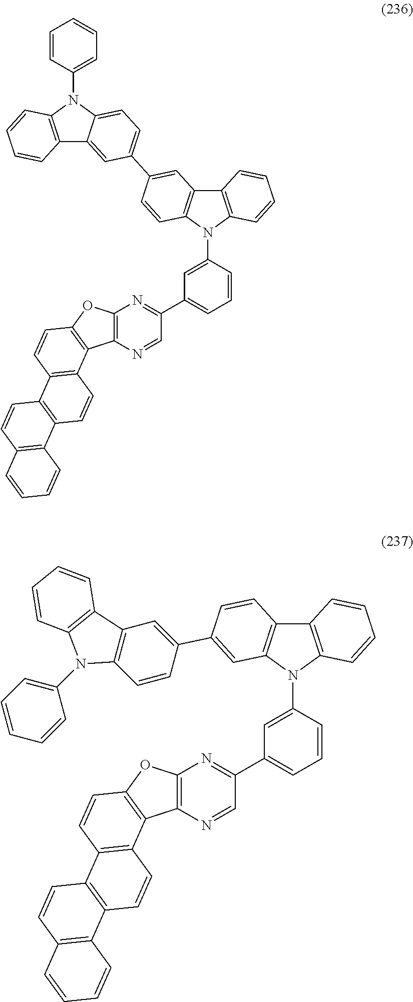 [SMILES: c1ccc(-n2c3ccccc3c3cc(-c4ccc5c(c4)c4ccccc4n5-c4cccc(-c5cnc6c(n5)oc5ccc7c8ccc9ccccc9c8ccc7c56)c4)ccc32)cc1 ; c1ccc(-n2c3ccccc3c3cc(-c4ccc5c6ccccc6n(-c6cccc(-c7cnc8c(n7)oc7ccc9c%10ccc%11ccccc%11c%10ccc9c78)c6)c5c4)ccc32)cc1]

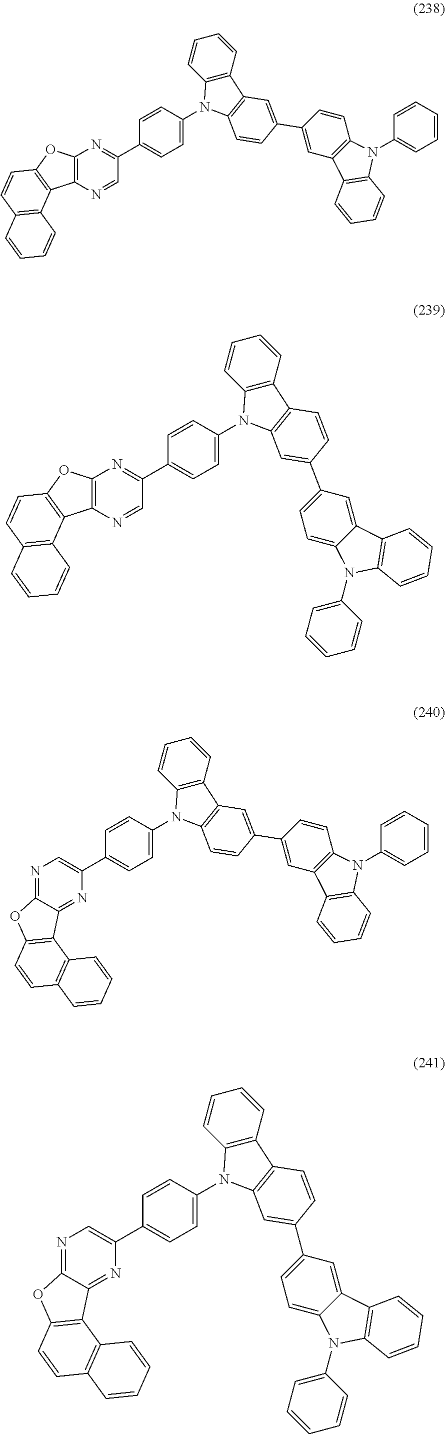 [SMILES: c1ccc(-n2c3ccccc3c3cc(-c4ccc5c(c4)c4ccccc4n5-c4ccc(-c5cnc6c(n5)oc5ccc7ccccc7c56)cc4)ccc32)cc1 ; c1ccc(-n2c3ccccc3c3cc(-c4ccc5c6ccccc6n(-c6ccc(-c7cnc8c(n7)oc7ccc9ccccc9c78)cc6)c5c4)ccc32)cc1 ; c1ccc(-n2c3ccccc3c3cc(-c4ccc5c(c4)c4ccccc4n5-c4ccc(-c5cnc6oc7ccc8ccccc8c7c6n5)cc4)ccc32)cc1 ; c1ccc(-n2c3ccccc3c3cc(-c4ccc5c6ccccc6n(-c6ccc(-c7cnc8oc9ccc%10ccccc%10c9c8n7)cc6)c5c4)ccc32)cc1]

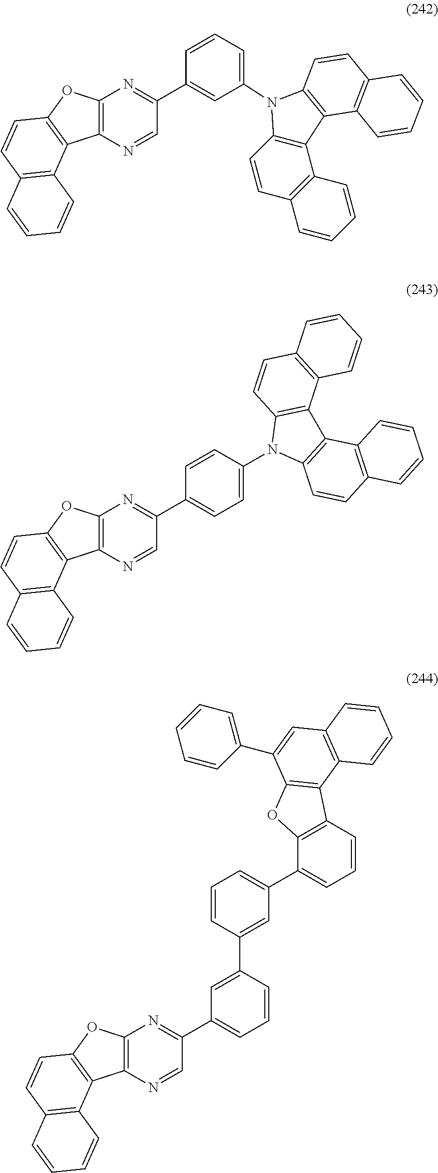 [SMILES: c1cc(-c2cnc3c(n2)oc2ccc4ccccc4c23)cc(-n2c3ccc4ccccc4c3c3c4ccccc4ccc32)c1 ; c1ccc2c(c1)ccc1oc3nc(-c4ccc(-n5c6ccc7ccccc7c6c6c7ccccc7ccc65)cc4)cnc3c12 ; c1ccc(-c2cc3ccccc3c3c2oc2c(-c4cccc(-c5cccc(-c6cnc7c(n6)oc6ccc8ccccc8c67)c5)c4)cccc23)cc1]

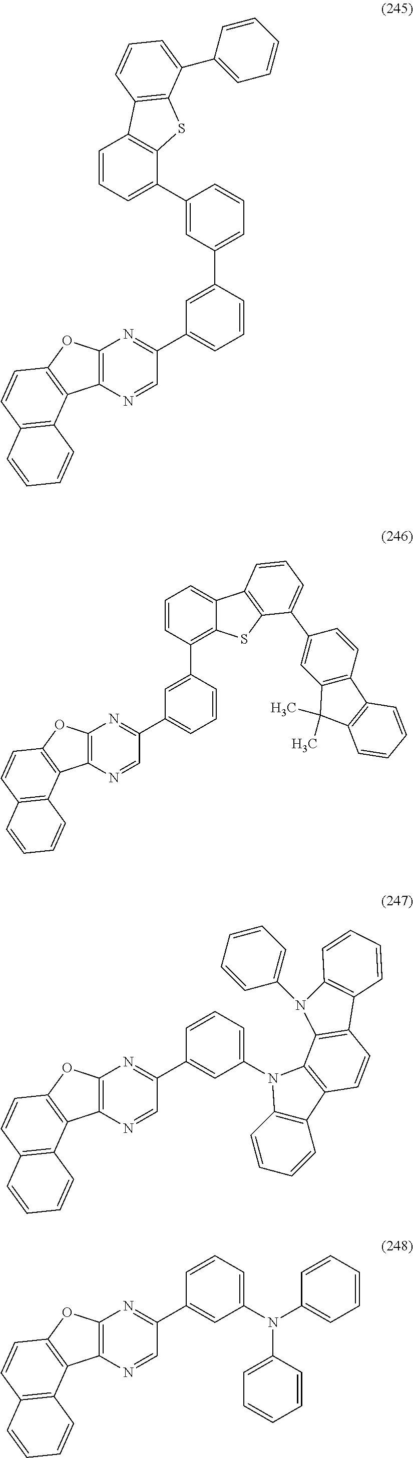 [SMILES: c1ccc(-c2cccc3c2sc2c(-c4cccc(-c5cccc(-c6cnc7c(n6)oc6ccc8ccccc8c67)c5)c4)cccc23)cc1 ; CC1(C)c2ccccc2-c2ccc(-c3cccc4c3sc3c(-c5cccc(-c6cnc7c(n6)oc6ccc8ccccc8c67)c5)cccc34)cc21 ; c1ccc(-n2c3ccccc3c3ccc4c5ccccc5n(-c5cccc(-c6cnc7c(n6)oc6ccc8ccccc8c67)c5)c4c32)cc1 ; c1ccc(N(c2ccccc2)c2cccc(-c3cnc4c(n3)oc3ccc5ccccc5c34)c2)cc1]

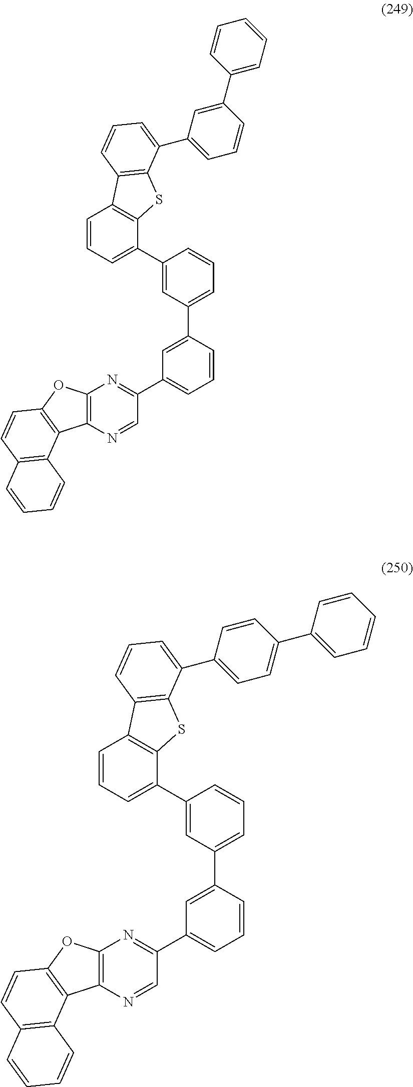 [SMILES: c1ccc(-c2cccc(-c3cccc4c3sc3c(-c5cccc(-c6cccc(-c7cnc8c(n7)oc7ccc9ccccc9c78)c6)c5)cccc34)c2)cc1 ; c1ccc(-c2ccc(-c3cccc4c3sc3c(-c5cccc(-c6cccc(-c7cnc8c(n7)oc7ccc9ccccc9c78)c6)c5)cccc34)cc2)cc1]

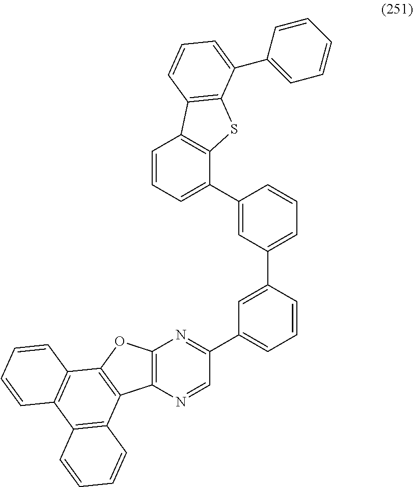 [SMILES: c1ccc(-c2cccc3c2sc2c(-c4cccc(-c5cccc(-c6cnc7c(n6)oc6c8ccccc8c8ccccc8c76)c5)c4)cccc23)cc1]

Note that the organic compounds represented by Structural Formulae (100) to (251) are examples of the organic compound represented by General Formula (G1). The organic compound of one embodiment of the present invention is not limited thereto.

Next, an example of a method for synthesizing an organic compound of one embodiment of the present invention represented by General Formula (G1′) will be described. Note that the organic compound represented by General Formula (G1′) is a furopyrazine derivative condensed with a condensed aromatic ring or a thienopyrazine derivative condensed with a condensed aromatic ring. The organic compound represented by General Formula (G1′) is one embodiment of the organic compound represented by General Formula (G1).

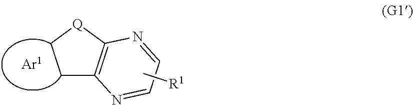

In General Formula (G1′), Q represents oxygen or sulfur, R¹ represents a group having 1 to 100 carbon atoms, R¹ represents a hole-transport skeleton, and Ar¹ represents a substituted or unsubstituted condensed aromatic ring.

### <<Method for Synthesizing Organic Compound Represented by General Formula (G1′)>>

A variety of reactions can be used for the synthesis of the organic compound represented by General Formula (G1′). The organic compound represented by General Formula (G1′) can be synthesized by a simple method shown by synthesis schemes below, for example.

First, as shown in a scheme (A-1) below, a methyloxy group-substituted or methylthio group-substituted aryl boronic acid (a1) is coupled with an amino group-and-halogen-substituted pyrazine derivative (a2) to obtain an intermediate (a3), and then the intermediate (a3) is reacted with tert-butyl nitrite and cyclized to obtain a furopyrazine derivative condensed with a condensed aromatic ring (a4) or a thienopyrazine derivative condensed with a condensed aromatic ring (a4). Note that when Y¹ in the pyrazine derivative (a4) is halogen, an intermediate (a5) obtained by coupling of the pyrazine derivative (a4) and a boronic acid of an aromatic ring containing halogen (Y³—B¹) can be used in the following reaction, similarly to the pyrazine derivative (a4).

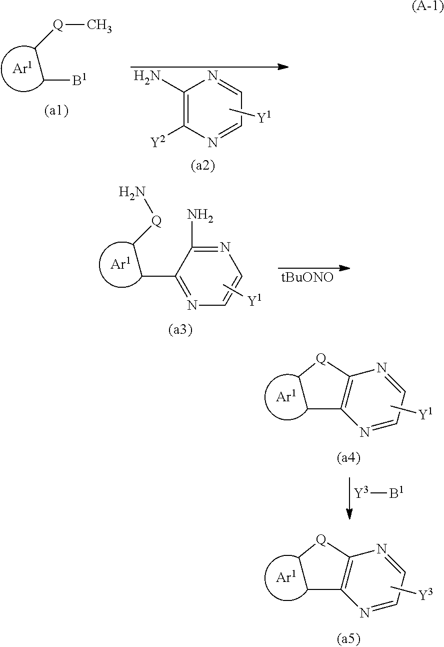

In the synthesis scheme (A-1), Q represents oxygen or sulfur, Ar¹ represents a substituted or unsubstituted condensed aromatic ring, Y¹ represents halogen or an aromatic ring containing halogen, the number of Y¹ is one or two, Y² represents halogen, Y³ represents an aromatic ring containing halogen, the number of Y³ is one or two, and B¹ represents a boronic acid, a boronic ester, a cyclic-triolborate salt, or the like. As the cyclic-triolborate salt, a lithium salt, a potassium salt, or a sodium salt may be used.

The organic compounds represented by General Formulae (a4) and (a5) in the synthesis scheme (A-1) are raw materials of the organic compound of one embodiment of the present invention as shown in a synthesis scheme (A-2) below. Note that the organic compounds represented by General Formulae (a4) and (a5) are novel organic compounds and included in one embodiment of the present invention. Specific structural formulae of the organic compounds represented by General Formulae (a4) and (a5) are shown below.

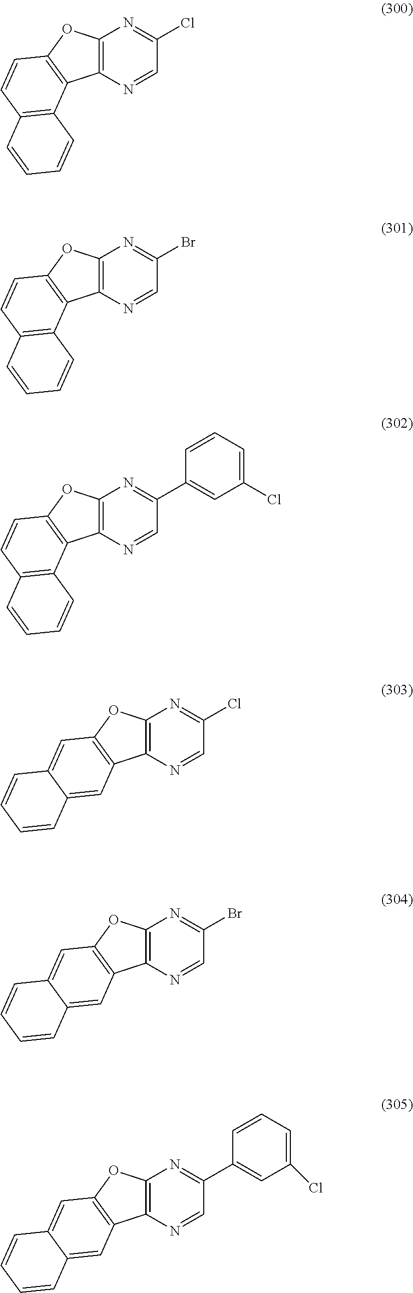

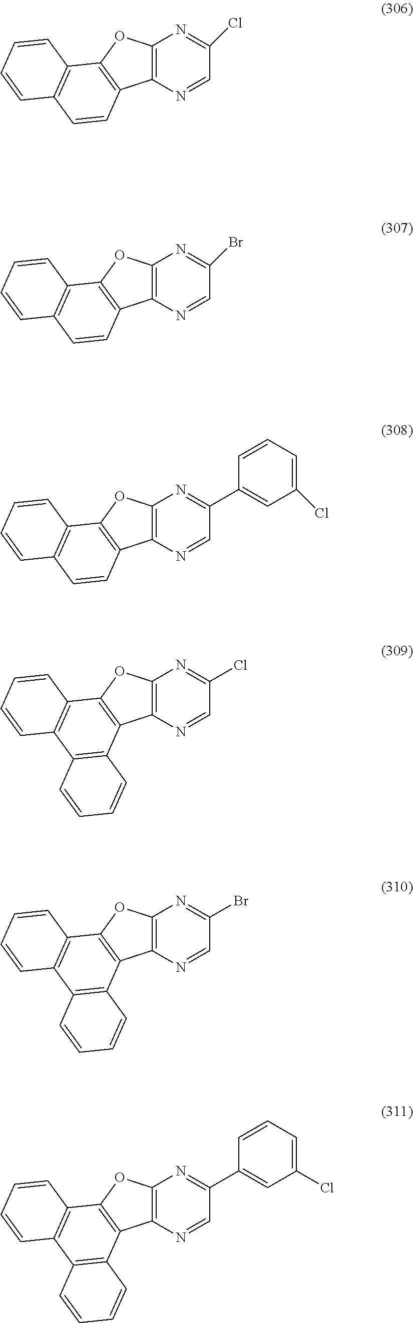

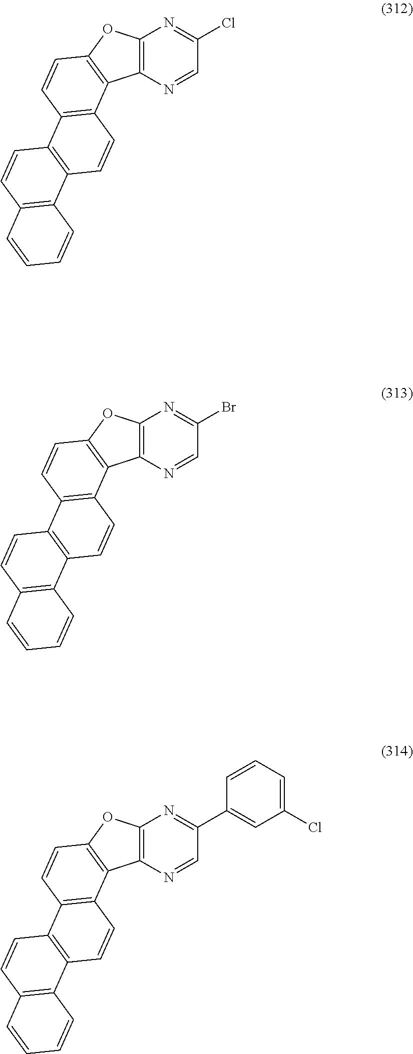

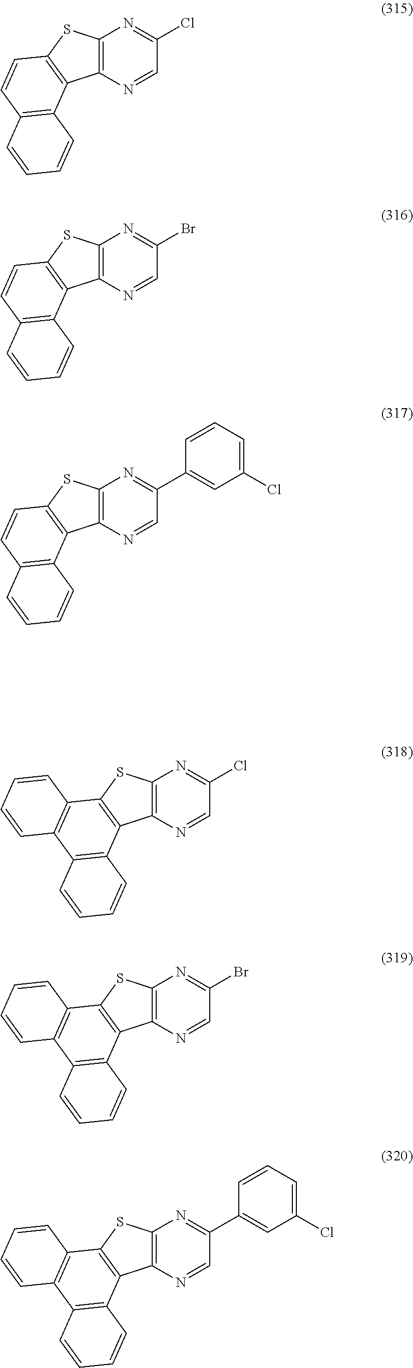

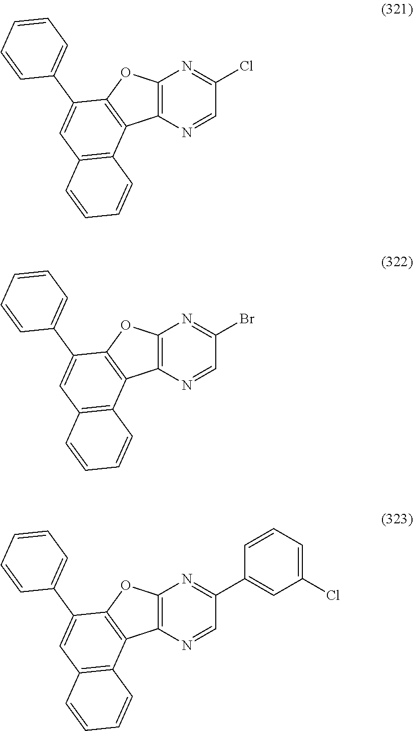

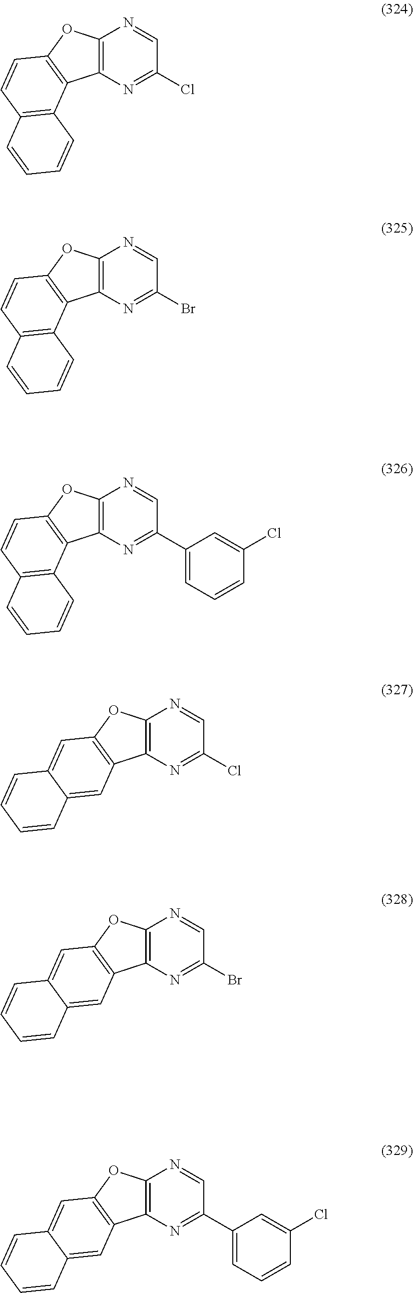

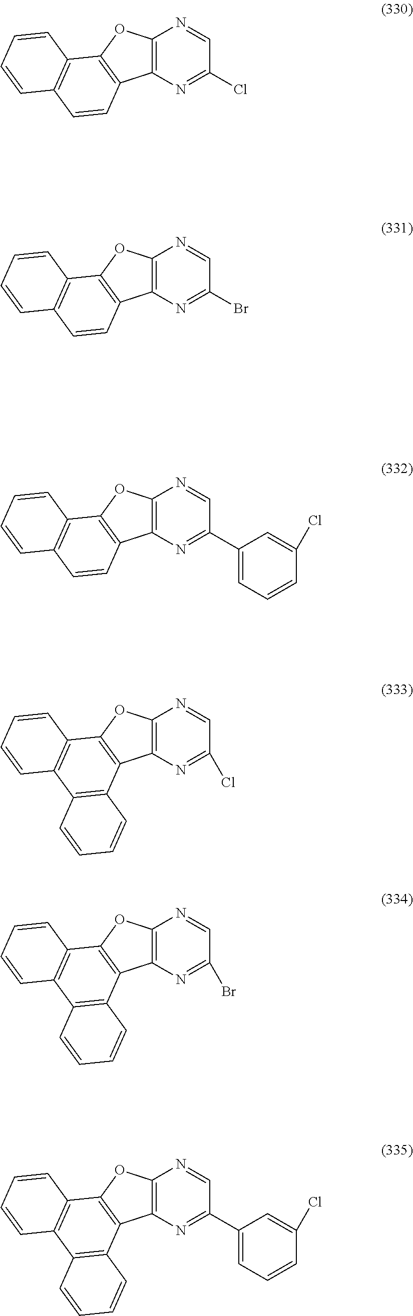

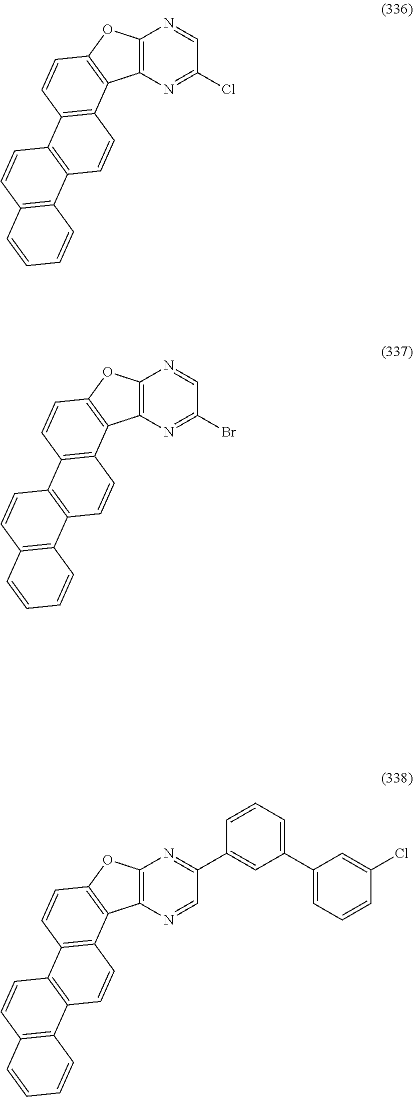

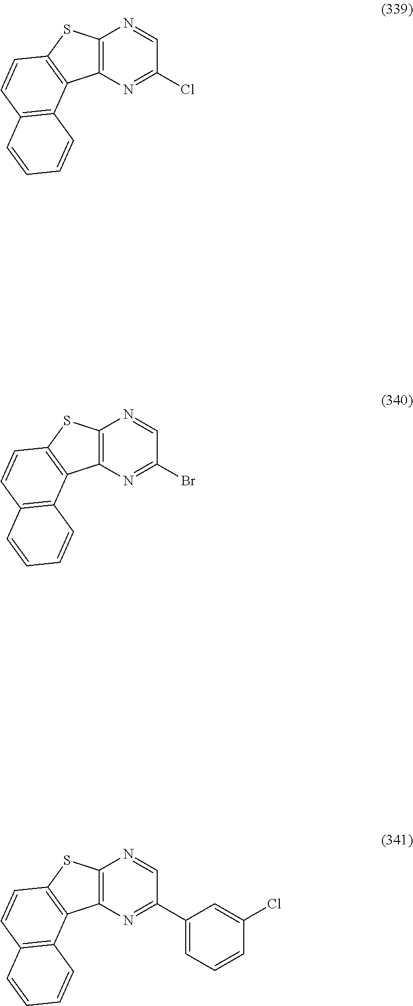

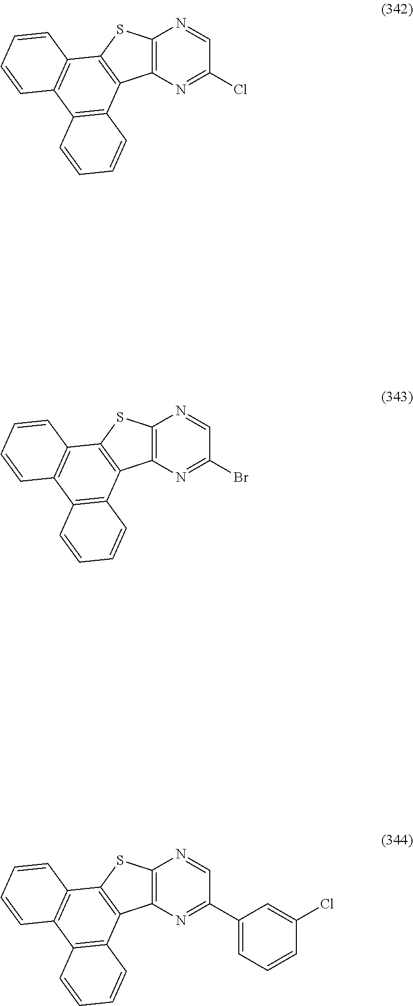

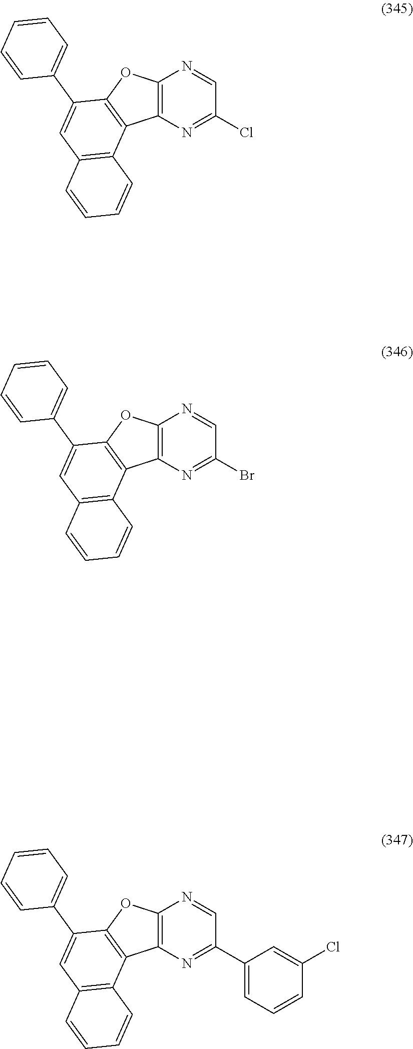

Note that the organic compounds represented by Structural Formulae (300) to (347) are examples of the organic compounds represented by General Formulae (a4) and (a5). The organic compound of one embodiment of the present invention is not limited thereto.

Next, as shown in the scheme (A-2) below, the furopyrazine derivative condensed with a condensed aromatic ring (a4) or the thienopyrazine derivative condensed with a condensed aromatic ring (a4) obtained by the scheme (A-1) is coupled with a boronic acid compound (b1) to obtain the organic compound represented by General Formula (G1′).

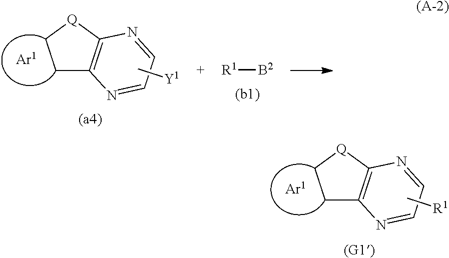

In the synthesis scheme (A-2), Q represents oxygen or sulfur, R¹ represents a group having 1 to 100 carbon atoms, R¹ has a hole-transport skeleton, Ar¹ represents a substituted or unsubstituted condensed aromatic ring, Y¹ represents one or two halogens, and B² represents a boronic acid, a boronic ester, a cyclic-triolborate salt, or the like. As the cyclic-triolborate salt, a lithium salt, a potassium salt, or a sodium salt may be used.

Since various kinds of the methyloxy group-substituted or methylthio group-substituted aryl boronic acid (a1), the amino group-and-halogen-substituted pyrazine derivative (a2), and the boronic acid compound (b1) that are used in the synthesis schemes (A-1) and (A-2) are commercially available or can be synthesized, a great variety of the furopyrazine derivative condensed with a condensed aromatic ring or the thienopyrazine derivative condensed with a condensed aromatic ring that is represented by General Formula (G1′) can be synthesized. Thus, a feature of the organic compound of one embodiment of the present invention is the abundance of variations.

Described above are the furopyrazine derivative condensed with a condensed aromatic ring or the thienopyrazine derivative condensed with a condensed aromatic ring, which is one embodiment of the present invention, and an example of the synthesis method thereof. The present invention is not limited to the one synthesized by the method, and any other synthesis methods may be employed.

In this embodiment, embodiments of the present invention have been described. Other embodiments of the present invention are described in the other embodiments. Note that embodiments of the present invention are not limited thereto. In other words, since various embodiments of the invention are described in this embodiment and the other embodiments, embodiments of the present invention are not limited to particular embodiments.

The structures described in this embodiment can be used in appropriate combination with any of the structures described in the other embodiments.

### Embodiment 2

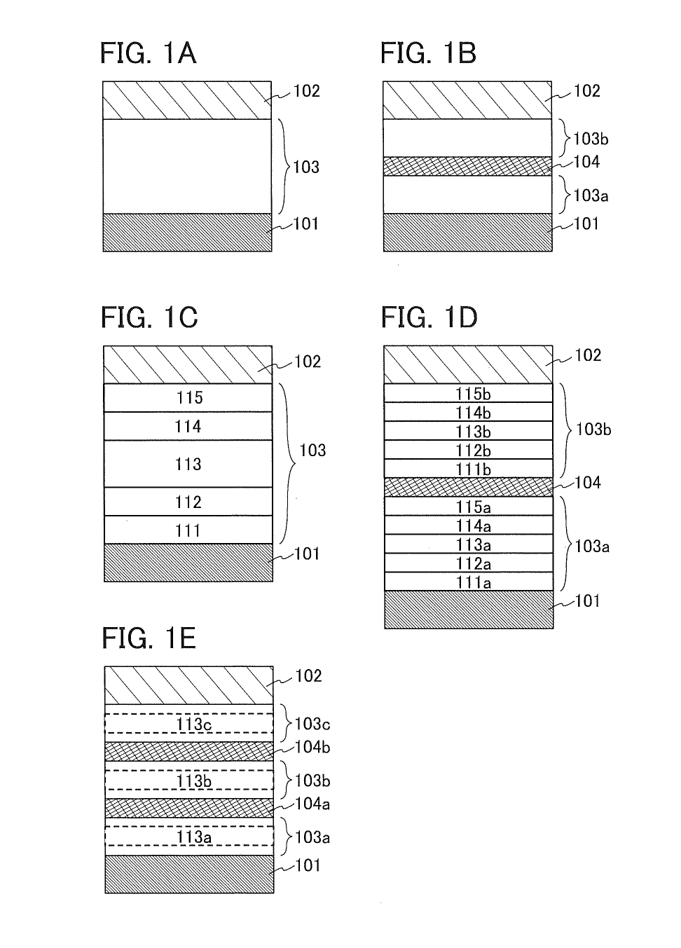

In this embodiment, a light-emitting element including any of the organic compounds described in Embodiment 1 is described with reference to FIGS. 1A to 1E.

### <<Basic Structure of Light-Emitting Element>>

First, a basic structure of a light-emitting element will be described. FIG. 1A illustrates a light-emitting element including, between a pair of electrodes, an EL layer having a light-emitting layer. Specifically, an EL layer 103 is provided between a first electrode 101 and a second electrode 102.

FIG. 1B illustrates a light-emitting element that has a stacked-layer structure (tandem structure) in which a plurality of EL layers (two EL layers 103a and 103b in FIG. 1B) are provided between a pair of electrodes and a charge-generation layer 104 is provided between the EL layers. With the use of such a tandem light-emitting element, a light-emitting device which can be driven at low voltage with low power consumption can be obtained.

The charge-generation layer 104 has a function of injecting electrons into one of the EL layers (103a or 103b) and injecting holes into the other of the EL layers (103b or 103a) when voltage is applied between the first electrode 101 and the second electrode 102. Thus, when voltage is applied in FIG. 1B such that the potential of the first electrode 101 is higher than that of the second electrode 102, the charge-generation layer 104 injects electrons into the EL layer 103a and injects holes into the EL layer 103b.

Note that in terms of light extraction efficiency, the charge-generation layer 104 preferably has a property of transmitting visible light (specifically, the charge-generation layer 104 has a visible light transmittance of 40% or more). The charge-generation layer 104 functions even when it has lower conductivity than the first electrode 101 or the second electrode 102.

FIG. 1C illustrates a stacked-layer structure of the EL layer 103 in the light-emitting element of one embodiment of the present invention. In this case, the first electrode 101 is regarded as functioning as an anode. The EL layer 103 has a structure in which a hole-injection layer 111, a hole-transport layer 112, a light-emitting layer 113, an electron-transport layer 114, and an electron-injection layer 115 are stacked in this order over the first electrode 101. Even in the case where a plurality of EL layers are provided as in the tandem structure illustrated in FIG. 1B, the layers in each EL layer are sequentially stacked from the anode side as described above. When the first electrode 101 is a cathode and the second electrode 102 is an anode, the stacking order is reversed.

The light-emitting layer 113 included in the EL layers (103, 103a, and 103b) contains an appropriate combination of a light-emitting substance and a plurality of substances, so that fluorescence or phosphorescence of a desired emission color can be obtained. The light-emitting layer 113 may have a stacked-layer structure having different emission colors. In that case, the light-emitting substance and other substances are different between the stacked light-emitting layers. Alternatively, the plurality of EL layers (103a and 103b) in FIG. 1B may exhibit their respective emission colors. Also in that case, the light-emitting substance and other substances are different between the light-emitting layers.

The light-emitting element of one embodiment of the present invention can have a micro optical resonator (microcavity) structure when, for example, the first electrode 101 is a reflective electrode and the second electrode 102 is a transflective electrode in FIG. 1C. Thus, light emission from the light-emitting layer 113 in the EL layer 103 can be resonated between the electrodes and light emission obtained through the second electrode 102 can be intensified.

Note that when the first electrode 101 of the light-emitting element is a reflective electrode in which a reflective conductive material and a light-transmitting conductive material (transparent conductive film) are stacked, optical adjustment can be performed by controlling the thickness of the transparent conductive film. Specifically, when the wavelength of light obtained from the light-emitting layer 113 is λ, the distance between the first electrode 101 and the second electrode 102 is preferably adjusted to around mλ/2 (m is a natural number).

To amplify desired light (wavelength: λ) obtained from the light-emitting layer 113, the optical path length from the first electrode 101 to a region where the desired light is obtained in the light-emitting layer 113 (light-emitting region) and the optical path length from the second electrode 102 to the region where the desired light is obtained in the light-emitting layer 113 (light-emitting region) are preferably adjusted to around (2m′+1)λ/4 (m′ is a natural number). Here, the light-emitting region means a region where holes and electrons are recombined in the light-emitting layer 113.

By such optical adjustment, the spectrum of specific monochromatic light obtained from the light-emitting layer 113 can be narrowed and light emission with high color purity can be obtained.

In that case, the optical path length between the first electrode 101 and the second electrode 102 is, to be exact, the total thickness from a reflective region in the first electrode 101 to a reflective region in the second electrode 102. However, it is difficult to precisely determine the reflective regions in the first electrode 101 and the second electrode 102; thus, it is assumed that the above effect can be sufficiently obtained wherever the reflective regions may be set in the first electrode 101 and the second electrode 102. Furthermore, the optical path length between the first electrode 101 and the light-emitting layer emitting the desired light is, to be exact, the optical path length between the reflective region in the first electrode 101 and the light-emitting region in the light-emitting layer emitting the desired light. However, it is difficult to precisely determine the reflective region in the first electrode 101 and the light-emitting region in the light-emitting layer emitting the desired light; thus, it is assumed that the above effect can be sufficiently obtained wherever the reflective region and the light-emitting region may be set in the first electrode 101 and the light-emitting layer emitting the desired light.

The light-emitting element in FIG. 1C has a microcavity structure, so that light (monochromatic light) with different wavelengths can be extracted even if the same EL layer is used. Thus, separate coloring for obtaining a plurality of emission colors (e.g., R, G, and B) is not necessary. Therefore, high resolution can be easily achieved. Note that a combination with coloring layers (color filters) is also possible. Furthermore, emission intensity of light with a specific wavelength in the front direction can be increased, whereby power consumption can be reduced.

A light-emitting element illustrated in FIG. 1E is an example of the light-emitting element with the tandem structure illustrated in FIG. 1B, and includes three EL layers (103a, 103b, and 103c) stacked with charge-generation layers (104a and 104b) positioned therebetween, as illustrated in the figure. The three EL layers (103a, 103b, and 103c) include respective light-emitting layers (113a, 113b, and 113c) and the emission colors of the light-emitting layers can be selected freely. For example, the light-emitting layer 113a can be blue, the light-emitting layer 113b can be red, green, or yellow, and the light-emitting layer 113c can be blue. For another example, the light-emitting layer 113a can be red, the light-emitting layer 113b can be blue, green, or yellow, and the light-emitting layer 113c can be red.

In the light-emitting element of one embodiment of the present invention, at least one of the first electrode 101 and the second electrode 102 is a light-transmitting electrode (e.g., a transparent electrode or a transflective electrode). In the case where the light-transmitting electrode is a transparent electrode, the transparent electrode has a visible light transmittance of higher than or equal to 40%. In the case where the light-transmitting electrode is a transflective electrode, the transflective electrode has a visible light reflectance of higher than or equal to 20% and lower than or equal to 80%, and preferably higher than or equal to 40% and lower than or equal to 70%. These electrodes preferably have a resistivity of 1×10⁻² Ωcm or less.

Furthermore, when one of the first electrode 101 and the second electrode 102 is a reflective electrode in the light-emitting element of one embodiment of the present invention, the visible light reflectance of the reflective electrode is higher than or equal to 40% and lower than or equal to 100%, and preferably higher than or equal to 70% and lower than or equal to 100%. This electrode preferably has a resistivity of 1×10⁻² Ωcm or less.

### <<Specific Structure and Fabrication Method of Light-Emitting Element>>

Specific structures and fabrication methods of light-emitting elements of embodiments of the present invention will be described with reference to FIGS. 1A to 1E. Here, a light-emitting element having the tandem structure in FIG. 1B and a microcavity structure will be described with reference to FIG. 1D. In the light-emitting element in FIG. 1D having a microcavity structure, the first electrode 101 is formed as a reflective electrode and the second electrode 102 is formed as a transflective electrode. Thus, a single-layer structure or a stacked-layer structure can be formed using one or more kinds of desired electrode materials. Note that the second electrode 102 is formed after formation of the EL layer 103b, with the use of a material selected as described above. For fabrication of these electrodes, a sputtering method or a vacuum evaporation method can be used.

### <First Electrode and Second Electrode>

As materials used for the first electrode 101 and the second electrode 102, any of the following materials can be used in an appropriate combination as long as the functions of the electrodes described above can be fulfilled. For example, a metal, an alloy, an electrically conductive compound, a mixture of these, and the like can be appropriately used. Specifically, an In—Sn oxide (also referred to as ITO), an In—Si—Sn oxide (also referred to as ITSO), an In—Zn oxide, an In—W—Zn oxide, or the like can be used. In addition, it is possible to use a metal such as aluminum (Al), titanium (Ti), chromium (Cr), manganese (Mn), iron (Fe), cobalt (Co), nickel (Ni), copper (Cu), gallium (Ga), zinc (Zn), indium (In), tin (Sn), molybdenum (Mo), tantalum (Ta), tungsten (W), palladium (Pd), gold (Au), platinum (Pt), silver (Ag), yttrium (Y), or neodymium (Nd) or an alloy containing an appropriate combination of any of these metals. It is also possible to use a Group 1 element or a Group 2 element in the periodic table, which is not described above (e.g., lithium (Li), cesium (Cs), calcium (Ca), or strontium (Sr)), a rare earth metal such as europium (Eu) or ytterbium (Yb), an alloy containing an appropriate combination of any of these elements, graphene, or the like.

In the light-emitting element in FIG. 1D, when the first electrode 101 is an anode, a hole-injection layer 111a and a hole-transport layer 112a of the EL layer 103a are sequentially stacked over the first electrode 101 by a vacuum evaporation method. After the EL layer 103a and the charge-generation layer 104 are formed, a hole-injection layer 111b and a hole-transport layer 112b of the EL layer 103b are sequentially stacked over the charge-generation layer 104 in a similar manner.

### <Hole-Injection Layer and Hole-Transport Layer>

The hole-injection layers (111, 111a, and 111b) inject holes from the first electrode 101 that is an anode and the charge-generation layer (104) to the EL layers (103, 103a, and 103b) and each contain a material with a high hole-injection property.

As examples of the material with a high hole-injection property, transition metal oxides such as molybdenum oxide, vanadium oxide, ruthenium oxide, tungsten oxide, and manganese oxide can be given. Alternatively, it is possible to use any of the following materials: phthalocyanine-based compounds such as phthalocyanine (abbreviation: H₂Pc) and copper phthalocyanine (abbreviation: CuPc); aromatic amine compounds such as 4,4′-bis[N-(4-diphenylaminophenyl)-N-phenylamino]biphenyl (abbreviation: DPAB) and N,N′-bis{4-[bis(3-methylphenyl)amino]phenyl}-N,N′-diphenyl-(1,1′-biphenyl)-4,4′-diamine (abbreviation: DNTPD); high molecular compounds such as poly(3,4-ethylenedioxythiophene)/poly(styrenesulfonic acid) (abbreviation: PEDOT/PSS); and the like.

Alternatively, as the material with a high hole-injection property, a composite material containing a hole-transport material and an acceptor material (an electron-accepting material) can also be used. In that case, the acceptor material extracts electrons from the hole-transport material, so that holes are generated in the hole-injection layers (111, 111a, and 1b) and the holes are injected into the light-emitting layers (113, 113a, and 113b) through the hole-transport layers (112, 112a, and 112b). Note that each of the hole-injection layers (111, 111a, and 111b) may be formed to have a single-layer structure using a composite material containing a hole-transport material and an acceptor material (electron-accepting material), or a stacked-layer structure in which a layer including a hole-transport material and a layer including an acceptor material (electron-accepting material) are stacked.

The hole-transport layers (112, 112a, and 112b) transport the holes, which are injected from the first electrode 101 and the charge-generation layer (104) by the hole-injection layers (111, 111a, and 111b), to the light-emitting layers (113, 113a, and 113b). Note that the hole-transport layers (112, 112a, and 112b) each contain a hole-transport material. It is particularly preferable that the HOMO level of the hole-transport material included in the hole-transport layers (112, 112a, and 112b) be the same as or close to that of the hole-injection layers (111, 111a, and 111b).

Examples of the acceptor material used for the hole-injection layers (111, 111a, and 111b) include an oxide of a metal belonging to any of Groups 4 to 8 of the periodic table. Specifically, molybdenum oxide, vanadium oxide, niobium oxide, tantalum oxide, chromium oxide, tungsten oxide, manganese oxide, and rhenium oxide can be given. Among these, molybdenum oxide is especially preferable since it is stable in the air, has a low hygroscopic property, and is easy to handle. Alternatively, organic acceptors such as a quinodimethane derivative, a chloranil derivative, and a hexaazatriphenylene derivative can be used. Specifically, 7,7,8,8-tetracyano-2,3,5,6-tetrafluoroquinodimethane (abbreviation: F₄-TCNQ), chloranil, 2,3,6,7,10,11-hexacyano-1,4,5,8,9,12-hexaazatriphenylene (abbreviation: HAT-CN), and the like can be used.

The hole-transport materials used for the hole-injection layers (111, 111a, and 111b) and the hole-transport layers (112, 112a, and 112b) are preferably substances with a hole mobility of greater than or equal to 10⁻⁶ cm²Ns. Note that other substances may be used as long as the substances have a hole-transport property higher than an electron-transport property.

Preferred hole-transport materials are π-electron rich heteroaromatic compounds (e.g., carbazole derivatives and indole derivatives) and aromatic amine compounds, examples of which include compounds having an aromatic amine skeleton, such as 4,4′-bis[N-(1-naphthyl)-N-phenylamino]biphenyl (abbreviation: NPB or α-NPD), N,N′-bis(3-methylphenyl)-N,N′-diphenyl-[1,1′-biphenyl]-4,4′-diamine (abbreviation: TPD), 4,4′-bis[N-(spiro-9,9′-bifluoren-2-yl)-N-phenylamino]biphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-(9-phenyl-9H-carbazol-3-yl)triphenylamine (abbreviation: PCBA1BP), 3-[4-(9-phenanthryl)-phenyl]-9-phenyl-9H-carbazole (abbreviation: PCPPn), N-(4-biphenyl)-N-(9,9-dimethyl-9H-fluoren-2-yl)-9-phenyl-9H-carbazol-3-amine (abbreviation: PCBiF), N-(1,1′-biphenyl-4-yl)-N-[4-(9-phenyl-9H-carbazol-3-yl)phenyl]-9,9-dimethyl-9H-fluor en-2-amine (abbreviation: PCBBiF), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 9,9-dimethyl-N-phenyl-N-[4-(9-phenyl-9H-carbazol-3-yl)phenyl]fluoren-2-amine (abbreviation: PCBAF), N-phenyl-N-[4-(9-phenyl-9H-carbazol-3-yl)phenyl]spiro-9,9′-bifluoren-2-amine (abbreviation: PCBASF), 4,4′,4″-tris(carbazol-9-yl)triphenylamine (abbreviation: TCTA), 4,4′,4″-tris(N,N-diphenylamino)triphenylamine (abbreviation: TDATA), and 4,4′,4″-tris[N-(3-methylphenyl)-N-phenylamino]triphenylamine (abbreviation: MTDATA); compounds having a carbazole skeleton, such as 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), 3,3′-bis(9-phenyl-9H-carbazole) (abbreviation: PCCP), 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), 3-[N-(1-naphthyl)-N-(9-phenylcarbazol-3-yl)amino]-9-phenylcarbazole (abbreviation: PCzPCN1), 1,3,5-tris[4-(N-carbazolyl)phenyl]benzene (abbreviation: TCPB), and 9-[4-(10-phenyl-9-anthracenyl)phenyl]-9H-carbazole (abbreviation: CzPA); compounds having a thiophene skeleton, such as 4,4′,4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), and 4-[4-(9-phenyl-9H-fluoren-9-yl)phenyl]-6-phenyldibenzothiophene (abbreviation: DBTFLP-IV); and compounds having a furan skeleton, such as 4,4′,4″-(benzene-1,3,5-triyl)tri(dibenzofuran) (abbreviation: DBF3P-II) and 4-{3-[3-(9-phenyl-9H-fluoren-9-yl)phenyl]phenyl}dibenzofuran (abbreviation: mmDBFFLBi-II).

A high molecular compound such as poly(N-vinylcarbazole) (abbreviation: PVK), poly(4-vinyltriphenylamine) (abbreviation: PVTPA), poly[N-(4-{N-[4-(4-diphenylamino)phenyl]phenyl-N-phenylamino}phenyl)methacrylamide] (abbreviation: PTPDMA), or poly[N,N′-bis(4-butylphenyl)-N,N′-bis(phenyl)benzidine] (abbreviation: Poly-TPD) can also be used.

Note that the hole-transport material is not limited to the above examples and may be one of or a combination of various known materials when used for the hole-injection layers (111, 111a, and 111b) and the hole-transport layers (112, 112a, and 112b). Note that the hole-transport layers (112, 112a, and 112b) may each be formed of a plurality of layers. That is, for example, the hole-transport layers may each have a stacked-layer structure of a first hole-transport layer and a second hole-transport layer.

In the light-emitting element in FIG. 1D, the light-emitting layer 113a is formed over the hole-transport layer 112a of the EL layer 103a by a vacuum evaporation method. After the EL layer 103a and the charge-generation layer 104 are formed, the light-emitting layer 113b is formed over the hole-transport layer 112b of the EL layer 103b by a vacuum evaporation method.

### <Light-Emitting Layer>

The light-emitting layers (113, 113a, 113b, and 113c) each contain a light-emitting substance. Note that as the light-emitting substance, a substance whose emission color is blue, violet, bluish violet, green, yellowish green, yellow, orange, red, or the like is appropriately used. When the plurality of light-emitting layers (113a, 113b, and 113c) are formed using different light-emitting substances, different emission colors can be exhibited (for example, complementary emission colors are combined to achieve white light emission). Furthermore, a stacked-layer structure in which one light-emitting layer contains two or more kinds of light-emitting substances may be employed.

The light-emitting layers (113, 113a, 113b, and 113c) may each contain one or more kinds of organic compounds (a host material and an assist material) in addition to a light-emitting substance (guest material). As the one or more kinds of organic compounds, the organic compounds of embodiments of the present invention described in Embodiment 1 or one or both of the hole-transport material and the electron-transport material described in this embodiment can be used.

As the light-emitting substance that can be used for the light-emitting layers (113, 113a, 113b, and 113c), a light-emitting substance that converts singlet excitation energy into light emission in the visible light range or a light-emitting substance that converts triplet excitation energy into light emission in the visible light range can be used.

Examples of other light-emitting substances are given below.

As an example of the light-emitting substance that converts singlet excitation energy into light emission, a substance that emits fluorescence (fluorescent material) can be given. Examples of the substance that emits fluorescence include a pyrene derivative, an anthracene derivative, a triphenylene derivative, a fluorene derivative, a carbazole derivative, a dibenzothiophene derivative, a dibenzofuran derivative, a dibenzoquinoxaline derivative, a quinoxaline derivative, a pyridine derivative, a pyrimidine derivative, a phenanthrene derivative, and a naphthalene derivative. A pyrene derivative is particularly preferable because it has a high emission quantum yield. Specific examples of the pyrene derivative include N,N′-bis(3-methylphenyl)-N,N′-bis[3-(9-phenyl-9H-fluoren-9-yl)phenyl]pyrene-1,6-diamine (abbreviation: 1,6mMemFLPAPrn), N,N′-diphenyl-N,N′-bis[4-(9-phenyl-9H-fluoren-9-yl)phenyl]pyrene-1,6-diamine (abbreviation: 1,6FLPAPrn), N,N′-bis(dibenzofuran-2-yl)-N,N′-diphenylpyrene-1,6-diamine (abbreviation: 1,6FrAPrn), N,N′-bis(dibenzothiophen-2-yl)-N,N′-diphenylpyrene-1,6-diamine (abbreviation: 1,6ThAPm), N,N′-(pyrene-1,6-diyl)bis[(N-phenylbenzo[b]naphtho[1,2-d]furan)-6-amine](abbreviation: 1,6BnfAPrn), N,N′-(pyrene-1,6-diyl)bis[(N-phenylbenzo[b]naphtho[1,2-d]furan)-8-amine](abbreviation: 1,6BnfAPm-02), and N,N′-(pyrene-1,6-diyl)bis[(6,N-diphenylbenzo[b]naphtho[1,2-d]furan)-8-amine](abbreviation: 1,6BnfAPrn-03).

In addition, it is possible to use 5,6-bis[4-(10-phenyl-9-anthryl)phenyl]-2,2′-bipyridine (abbreviation: PAP2BPy), 5,6-bis[4′-(10-phenyl-9-anthryl)biphenyl-4-yl]-2,2′-bipyridine (abbreviation: PAPP2BPy), N,N′-bis[4-(9H-carbazol-9-yl)phenyl]-N,N′-diphenylstilbene-4,4′-diamine (abbreviation: YGA2S), 4-(9H-carbazol-9-yl)-4′-(10-phenyl-9-anthryl)triphenylamine (abbreviation: YGAPA), 4-(9H-carbazol-9-yl)-4′-(9,10-diphenyl-2-anthryl)triphenylamine (abbreviation: 2YGAPPA), N,9-diphenyl-N-[4-(10-phenyl-9-anthryl)phenyl]-9H-carbazol-3-amine (abbreviation: PCAPA), 4-(10-phenyl-9-anthryl)-4′-(9-phenyl-9H-carbazol-3-yl)triphenylamine (abbreviation: PCBAPA), 4-[4-(10-phenyl-9-anthryl)phenyl]-4′-(9-phenyl-9H-carbazol-3-yl)triphenylamine (abbreviation: PCBAPBA), perylene, 2,5,8,11-tetra(tert-butyl)perylene (abbreviation: TBP), N,N′-(2-tert-butylanthracene-9,10-diyldi-4,1-phenylene)bis[N,N′,N′-triphenyl-1,4-phenylenediamine] (abbreviation: DPABPA), N,9-diphenyl-N-[4-(9,10-diphenyl-2-anthryl)phenyl]-9H-carbazol-3-amine (abbreviation: 2PCAPPA), N-[4-(9,10-diphenyl-2-anthryl)phenyl]-N,N′,N′-triphenyl-1,4-phenylenediamine (abbreviation: 2DPAPPA), or the like.

As examples of a light-emitting substance that converts triplet excitation energy into light emission, a substance that emits phosphorescence (phosphorescent material) and a thermally activated delayed fluorescence (TADF) material that exhibits thermally activated delayed fluorescence can be given.

Examples of a phosphorescent material include an organometallic complex, a metal complex (platinum complex), and a rare earth metal complex. These substances exhibit the respective emission colors (emission peaks) and thus, any of them is appropriately selected according to need.

As examples of a phosphorescent material which emits blue or green light and whose emission spectrum has a peak wavelength at greater than or equal to 450 nm and less than or equal to 570 nm, the following substances can be given.

For example, organometallic complexes having a 4H-triazole skeleton, such as tris{2-[5-(2-methylphenyl)-4-(2,6-dimethylphenyl)-4H-1,2,4-triazol-3-yl-κN²]phenyl-κC}iridium(III) (abbreviation: [Ir(mpptz-dmp)₃]), tris(5-methyl-3,4-diphenyl-4H-1,2,4-triazolato)iridium(III) (abbreviation: [Ir(Mptz)₃]), tris[4-(3-biphenyl)-5-isopropyl-3-phenyl-4H-1,2,4-triazolato]iridium(III) (abbreviation: [Ir(iPrptz-3b)₃]), and tris[3-(5-biphenyl)-5-isopropyl-4-phenyl-4H-1,2,4-triazolato]iridium(III) (abbreviation: [Ir(iPr5btz)₃]); organometallic complexes having a 1H-triazole skeleton, such as tris[3-methyl-1-(2-methylphenyl)-5-phenyl-1H-1,2,4-triazolato]iridium(III) (abbreviation: [Ir(Mptz1-mp)₃]) and tris(1-methyl-5-phenyl-3-propyl-1H-1,2,4-triazolato)iridium(III) (abbreviation: [Ir(Prptz1-Me)₃]); organometallic complexes having an imidazole skeleton, such as fac-tris[1-(2,6-diisopropylphenyl)-2-phenyl-1H-imidazole]iridium(III) (abbreviation: [Ir(iPrpmi)₃]) and tris[3-(2,6-dimethylphenyl)-7-methylimidazo[1,2-f]phenanthridinato]iridium(III) (abbreviation: [Ir(dmpimpt-Me)₃]); organometallic complexes in which a phenylpyridine derivative having an electron-withdrawing group is a ligand, such as bis[2-(4′,6′-difluorophenyl)pyridinato-N,C^{2′}]iridium(III) tetrakis(1-pyrazolyl)borate (abbreviation: FIr6), bis[2-(4′,6′-difluorophenyl)pyridinato-N,C^{2′}]iridium(III) picolinate (abbreviation: FIrpic), bis{2-[3′,5′-bis(trifluoromethyl)phenyl]pyridinato-N,C^{2′}}iridium(III) picolinate (abbreviation: [Ir(CF₃ppy)₂(pic)]), and bis[2-(4′,6′-difluorophenyl)pyridinato-N,C^{2′}]iridium(III) acetylacetonate (abbreviation: FIr(acac)); and the like can be given.

As examples of a phosphorescent material which emits green or yellow light and whose emission spectrum has a peak wavelength at greater than or equal to 495 nm and less than or equal to 590 nm, the following substances can be given.

For example, organometallic iridium complexes having a pyrimidine skeleton, such as tris(4-methyl-6-phenylpyrimidinato)iridium(III) (abbreviation: [Ir(mppm)₃]), tris(4-t-butyl-6-phenylpyrimidinato)iridium(III) (abbreviation: [Ir(tBuppm)₃]), (acetylacetonato)bis(6-methyl-4-phenylpyrimidinato)iridium(III) (abbreviation: [Ir(mppm)₂(acac)]), (acetylacetonato)bis(6-tert-butyl-4-phenylpyrimidinato)iridium(III) (abbreviation: [Ir(tBuppm)₂(acac)]), (acetylacetonato)bis[6-(2-norbornyl)-4-phenylpyrimidinato]iridium(III) (abbreviation: [Ir(nbppm)₂(acac)]), (acetylacetonato)bis[5-methyl-6-(2-methylphenyl)-4-phenylpyrimidinato]iridium(III) (abbreviation: [Ir(mpmppm)₂(acac)]), (acetylacetonato)bis{4,6-dimethyl-2-[6-(2,6-dimethylphenyl)-4-pyrimidinyl-κN³]phenyl-κC}iridium(Ill) (abbreviation: [Ir(dmppm-dmp)₂(acac)]), and (acetylacetonato)bis(4,6-diphenylpyrimidinato)iridium(III) (abbreviation: [Ir(dppm)₂(acac)]); organometallic iridium complexes having a pyrazine skeleton, such as (acetylacetonato)bis(3,5-dimethyl-2-phenylpyrazinato)iridium(III) (abbreviation: [Ir(mppr-Me)₂(acac)]) and (acetylacetonato)bis(5-isopropyl-3-methyl-2-phenylpyrazinato)iridium(III) (abbreviation: [Ir(mppr-iPr)₂(acac)]); organometallic iridium complexes having a pyridine skeleton, such as tris(2-phenylpyridinato-N,C^{2′})iridium(III) (abbreviation: [Ir(ppy)₃]), bis(2-phenylpyridinato-N,C^{2′})iridium(III) acetylacetonate (abbreviation: [Ir(ppy)₂(acac)]), bis(benzo[h]quinolinato)iridium(III) acetylacetonate (abbreviation: [Ir(bzq)₂(acac)]), tris(benzo[h]quinolinato)iridium(III) (abbreviation: [Ir(bzq)₃]), tris(2-phenylquinolinato-N,C^{2′})iridium(III) (abbreviation: [Ir(pq)₃]), bis(2-phenylquinolinato-N,C^{2′})iridium(III) acetylacetonate (abbreviation: [Ir(pq)₂(acac)]), [2-(4-phenyl-2-pyridinyl-κN)phenyl-κC]bis[2-(2-pyridinyl-κN)phenyl-κC]iridium(III) (abbreviation: Ir(ppy)₂(4dppy)), and bis[2-(2-pyridinyl-κN)phenyl-κC][2-(4-methyl-5-phenyl-2-pyridinyl-κN)phenyl-κC]; organometallic complexes such as bis(2,4-diphenyl-1,3-oxazolato-N,C^{2′})iridium(III) acetylacetonate (abbreviation: [Ir(dpo)₂(acac)]), bis{2-[4′-(perfluorophenyl)phenyl]pyridinato-N,C^{2′}}iridium(III) acetylacetonate (abbreviation: [Ir(p-PF-ph)₂(acac)]), and bis(2-phenylbenzothiazolato-N,C^{2′})iridium(III) acetylacetonate (abbreviation: [Ir(bt)₂(acac)]); and rare earth metal complexes such as tris(acetylacetonatomonophenanthroline)terbium(III) (abbreviation: [Tb(acac)₃(Phen)]) can be given.

As examples of a phosphorescent material which emits yellow or red light and whose emission spectrum has a peak wavelength at greater than or equal to 570 nm and less than or equal to 750 nm, the following substances can be given.

For example, organometallic complexes having a pyrimidine skeleton, such as (diisobutyrylmethanato)bis[4,6-bis(3-methylphenyl)pyrimidinato]iridium(III) (abbreviation: [Ir(5mdppm)₂(dibm)]), bis[4,6-bis(3-methylphenyl)pyrimidinato](dipivaloylmethanato)iridium(III) (abbreviation: [Ir(5mdppm)₂(dpm)]), bis[4,6-di(naphthalen-1-yl)pyrimidinato](dipivaloylmethanato)iridium(III) (abbreviation: [Ir(d1npm)₂(dpm)]), and tris(4-t-butyl-6-phenylpyrimidinato)iridium(III) (abbreviation: [Ir(tBuppm)₃]); organometallic complexes having a pyrazine skeleton, such as (acetylacetonato)bis(2,3,5-triphenylpyrazinato)iridium(III) (abbreviation: [Ir(tppr)₂(acac)]), bis(2,3,5-triphenylpyrazinatoXdipivaloylmethanato)iridium(III) (abbreviation: [Ir(tppr)₂(dpm)]), bis{4,6-dimethyl-2-[3-(3,5-dimethylphenyl)-5-phenyl-2-pyrazinyl-κN]phenyl-κC}(2,6-dimethyl-3,5-heptanedionato-κ²O,O′)iridium(III) (abbreviation: [Ir(dmdppr-P)₂(dibm)]), bis{4,6-dimethyl-2-[5-(4-cyano-2,6-dimethylphenyl)-3-(3,5-dimethylphenyl)-2-pyrazin yl-κN]phenyl-κC}(2,2,6,6-tetramethyl-3,5-heptanedionato-κ²O,O′)iridium(III) (abbreviation: [Ir(dmdppr-dmCP)₂(dpm)]), (acetylacetonato)bis[2-methyl-3-phenylquinoxalinato-N,C^{2′}]iridium(III) (abbreviation: [Ir(mpq)₂(acac)]), (acetylacetonato)bis(2,3-diphenylquinoxalinato-N,C^{2′})iridium(III) (abbreviation: [Ir(dpq)₂(acac)]), (acetylacetonato)bis[2,3-bis(4-fluorophenyl)quinoxalinato]iridium(III) (abbreviation: [Ir(Fdpq)₂(acac)]), and bis{4,6-dimethyl-2-[5-(5-cyano-2-methylphenyl)-3-(3,5-dimethylphenyl)-2-pyrazinyl-κN]phenyl-κC}(2,2,6,6-tetramethyl-3,5-heptanedionato-κ²O,O′)iridium(III) (abbreviation: [Ir(dmdppr-m5CP)₂(dpm)]); organometallic complexes having a pyridine skeleton, such as tris(1-phenylisoquinolinato-N,C^{2′})iridium(III) (abbreviation: [Ir(piq)₃]), bis(1-phenylisoquinolinato-N,C^{2′})iridium(III) acetylacetonate (abbreviation: [Ir(piq)₂(acac)]), and bis[4,6-dimethyl-2-(2-quinolinyl-κN)phenyl-κC](2,4-pentanedionato-κ²O,O′)iridium(III); platinum complexes such as 2,3,7,8,12,13,17,18-octaethyl-21H,23H-porphyrin platinum(II) (abbreviation: [PtOEP]); and rare earth metal complexes such as tris(1,3-diphenyl-1,3-propanedionatoXmonophenanthroline)europium(III) (abbreviation: [Eu(DBM)₃(Phen)]) and tris[1-(2-thenoyl)-3,3,3-trifluoroacetonato](monophenanthroline)europium(III) (abbreviation: [Eu(TTA)₃(Phen)]) can be given.

As the organic compounds (the host material and the assist material) used in the light-emitting layers (113, 113a, 113b, and 113c), one or more kinds of substances having a larger energy gap than the light-emitting substance (the guest material) are used. In the case where a plurality of organic compounds are used for the light-emitting layers (113, 113a, 113b, and 113c), it is preferable to use compounds that form an exciplex in combination with a phosphorescent light-emitting substance. With such a structure, light emission can be obtained by exciplex-triplet energy transfer (ExTET), which is energy transfer from an exciplex to a light-emitting substance. In that case, although any of various organic compounds can be used in an appropriate combination, in order to form an exciplex efficiently, it is particularly preferable to combine a compound that easily accepts holes (hole-transport material) and a compound that easily accepts electrons (electron-transport material). The organic compound of one embodiment of the present invention described in Embodiment 1 has a low LUMO level and thus is suitable for the compound that easily accepts electrons.

When the light-emitting substance is a fluorescent material, it is preferable to use, as the host material, an organic compound that has a high energy level in a singlet excited state and has a low energy level in a triplet excited state. For example, an anthracene derivative or a tetracene derivative is preferably used. Specific examples thereof include 9-phenyl-3-[4-(10-phenyl-9-anthryl)phenyl]-9H-carbazole (abbreviation: PCzPA), 3-[4-(1-naphthyl)-phenyl]-9-phenyl-9H-carbazole (abbreviation: PCPN), 9-[4-(10-phenyl-9-anthracenyl)phenyl]-9H-carbazole (abbreviation: CzPA), 7-[4-(10-phenyl-9-anthryl)phenyl]-7H-dibenzo[c,g]carbazole (abbreviation: cgDBCzPA), 6-[3-(9,10-diphenyl-2-anthryl)phenyl]-benzo[b]naphtho[1,2-d]furan (abbreviation: 2mBnfPPA), 9-phenyl-10-{4-(9-phenyl-9H-fluoren-9-yl)biphenyl-4′-yl}anthracene (abbreviation: FLPPA), 5,12-diphenyltetracene, and 5,12-bis(biphenyl-2-yl)tetracene.

In the case where the light-emitting substance is a phosphorescent material, an organic compound having triplet excitation energy (energy difference between a ground state and a triplet excited state) which is higher than that of the light-emitting substance is preferably selected as the host material. The organic compound of one embodiment of the present invention described in Embodiment 1 has a stable triplet excited state and thus is particularly suitable for a host material in the case where the light-emitting substance is a phosphorescent material. Owing to the triplet excitation energy level, the organic compound is particularly suitable when the phosphorescent material emits red light. Besides, a zinc- or aluminum-based metal complex, an oxadiazole derivative, a triazole derivative, a benzimidazole derivative, a quinoxaline derivative, a dibenzoquinoxaline derivative, a dibenzothiophene derivative, a dibenzofuran derivative, a pyrimidine derivative, a triazine derivative, a pyridine derivative, a bipyridine derivative, a phenanthroline derivative, an aromatic amine, a carbazole derivative, or the like can be used as the host material.

More specifically, any of the following hole-transport materials and electron-transport materials can be used as the host material, for example.

Examples of the host material having a high hole-transport property include aromatic amine compounds such as N,N′-di(p-tolyl)-N,N′-diphenyl-p-phenylenediamine (abbreviation: DTDPPA), 4,4′-bis[N-(4-diphenylaminophenyl)-N-phenylamino]biphenyl (abbreviation: DPAB), N,N′-bis{4-[bis(3-methylphenyl)amino]phenyl}-N,N′-diphenyl-(1,1′-biphenyl)-4,4′-diamine (abbreviation: DNTPD), and 1,3,5-tris[N-(4-diphenylaminophenyl)-N-phenylamino]benzene (abbreviation: DPA3B).

Carbazole derivatives such as 3-[N-(4-diphenylaminophenyl)-N-phenylamino]-9-phenylcarbazole (abbreviation: PCzDPA1), 3,6-bis[N-(4-diphenylaminophenyl)-N-phenylamino]-9-phenylcarbazole (abbreviation: PCzDPA2), 3,6-bis[N-(4-diphenylaminophenyl)-N-(1-naphthyl)amino]-9-phenylcarbazole (abbreviation: PCzTPN2), 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), and 3-[N-(1-naphthyl)-N-(9-phenylcarbazol-3-yl)amino]-9-phenylcarbazole (abbreviation: PCzPCN1) are also given. Other examples of the carbazole derivative include 4,4′-di(N-carbazolyl)biphenyl (abbreviation: CBP), 1,3,5-tris[4-(N-carbazolyl)phenyl]benzene (abbreviation: TCPB), and 1,4-bis[4-(N-carbazolyl)phenyl]-2,3,5,6-tetraphenylbenzene.

Examples of the host material having a high hole-transport property include aromatic amine compounds such as 4,4′-bis[N-(1-naphthyl)-N-phenylamino]biphenyl (abbreviation: NPB or α-NPD), N,N′-bis(3-methylphenyl)-N,N′-diphenyl-[1,1′-biphenyl]-4,4′-diamine (abbreviation: TPD), 4,4′,4″-tris(carbazol-9-yl)triphenylamine (abbreviation: TCTA), 4,4′,4″-tris[N-(1-naphthyl)-N-phenylamino]triphenylamine (abbreviation: 1-TNATA), 4,4′,4″-tris(N,N-diphenylamino)triphenylamine (abbreviation: TDATA), 4,4′,4″-tris[N-(3-methylphenyl)-N-phenylamino]triphenylamine (abbreviation: m-MTDATA), 4,4′-bis[N-(spiro-9,9′-bifluoren-2-yl)-N-phenylamino]biphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), N-(9,9-dimethyl-9H-fluoren-2-yl)-N-{9,9-dimethyl-2-[N-phenyl-N′-(9,9-dimethyl-9H-fluoren-2-yl)amino]-9H-fluoren-7-yl}phenylamine (abbreviation: DFLADFL), N-(9,9-dimethyl-2-diphenylamino-9H-fluoren-7-yl)diphenylamine (abbreviation: DPNF), 2-[N-(4-diphenylaminophenyl)-N-phenylamino]spiro-9,9′-bifluorene (abbreviation: DPASF), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(I-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 4-phenylbiphenyl-(9-phenyl-9H-carbazol-3-yl)amine (abbreviation: PCA1BP), N,N′-bis(9-phenylcarbazol-3-yl)-N,N′-diphenylbenzene-1,3-diamine (abbreviation: PCA2B), N,N′,N″-triphenyl-N,N′,N″-tris(9-phenylcarbazol-3-yl)benzene-1,3,5-triamine (abbreviation: PCA3B), N-(4-biphenyl)-N-(9,9-dimethyl-9H-fluoren-2-yl)-9-phenyl-9H-carbazol-3-amine (abbreviation: PCBBiF), N-(1,1′-biphenyl-4-yl)-N-[4-(9-phenyl-9H-carbazol-3-yl)phenyl]-9,9-dimethyl-9H-fluor en-2-amine (abbreviation: PCBBiF), 9,9-dimethyl-N-phenyl-N-[4-(9-phenyl-9H-carbazol-3-yl)phenyl]fluoren-2-amine (abbreviation: PCBAF), N-phenyl-N-[4-(9-phenyl-9H-carbazol-3-yl)phenyl]spiro-9,9′-bifluoren-2-amine (abbreviation: PCBASF), 2-[N-(9-phenylcarbazol-3-yl)-N-phenylamino]spiro-9,9′-bifluorene (abbreviation: PCASF), 2,7-bis[N-(4-diphenylaminophenyl)-N-phenylamino]spiro-9,9′-bifluorene (abbreviation: DPA2SF), N-[4-(9H-carbazol-9-yl)phenyl]-N-(4-phenyl)phenylaniline (abbreviation: YGA1BP), and N,N′-bis[4-(carbazol-9-yl)phenyl]-N,N′-diphenyl-9,9-dimethylfluorene-2,7-diamine (abbreviation: YGA2F). Other examples are carbazole compounds, thiophene compounds, furan compounds, fluorene compounds, triphenylene compounds, phenanthrene compounds, and the like such as 3-[4-(1-naphthyl)-phenyl]-9-phenyl-9H-carbazole (abbreviation: PCPN), 3-[4-(9-phenanthryl)-phenyl]-9-phenyl-9H-carbazole (abbreviation: PCPPn), 3,3′-bis(9-phenyl-9H-carbazole) (abbreviation: PCCP), 1,3-bis(N-carbazolyl)benzene (abbreviation: mCP), 3,6-bis(3,5-diphenylphenyl)-9-phenylcarbazole (abbreviation: CzTP), 4-{3-[3-(9-phenyl-9H-fluoren-9-yl)phenyl]phenyl}dibenzofuran (abbreviation: mmDBFFLBi-II), 4,4′,4″-(benzene-1,3,5-triyl)tri(dibenzofuran) (abbreviation: DBF3P-II), 1,3,5-tri(dibenzothiophen-4-yl)benzene (abbreviation: DBT3P-II), 2,8-diphenyl-4-[4-(9-phenyl-9H-fluoren-9-yl)phenyl]dibenzothiophene (abbreviation: DBTFLP-III), 4-[4-(9-phenyl-9H-fluoren-9-yl)phenyl]-6-phenyldibenzothiophene (abbreviation: DBTFLP-IV), and 4-[3-(triphenylen-2-yl)phenyl]dibenzothiophene (abbreviation: mDBTPTp-II).

Examples of the host material having a high electron-transport property include the organic compounds of embodiments of the present invention described in Embodiment 1 and a metal complex having a quinoline skeleton or a benzoquinoline skeleton, such as tris(8-quinolinolato)aluminum(III) (abbreviation: Alq), tris(4-methyl-8-quinolinolato)aluminum(III) (abbreviation: Almq₃), bis(10-hydroxybenzo[h]quinolinato)beryllium(II) (abbreviation: BeBq₂), bis(2-methyl-8-quinolinolato)(4-phenylphenolato)aluminum(III) (abbreviation: BAlq), or bis(8-quinolinolato)zinc(II) (abbreviation: Znq). Alternatively, a metal complex having an oxazole-based or thiazole-based ligand, such as bis[2-(2-benzoxazolyl)phenolato]zinc(II) (abbreviation: ZnPBO) or bis[2-(2-benzothiazolyl)phenolato]zinc(II) (abbreviation: ZnBTZ) can be used. Other than such metal complexes, any of the following can be used: oxadiazole derivatives such as 2-(4-biphenylyl)-5-(4-tert-butylphenyl)-1,3,4-oxadiazole (abbreviation: PBD), 1,3-bis[5-(p-tert-butylphenyl)-1,3,4-oxadiazol-2-yl]benzene (abbreviation: OXD-7), and 9-[4-(5-phenyl-1,3,4-oxadiazol-2-yl)phenyl]-9H-carbazole (abbreviation: CO11); a triazole derivative such as 3-(4-biphenylyl)-4-phenyl-5-(4-tert-butylphenyl)-1,2,4-triazole (abbreviation: TAZ); a compound having an imidazole skeleton (in particular, a benzimidazole derivative) such as 2,2′,2″-(1,3,5-benzenetriyl)tris(1-phenyl-1H-benzimidazole) (abbreviation: TPBI) or 2-[3-(dibenzothiophen-4-yl)phenyl]-1-phenyl-1H-benzimidazole (abbreviation: mDBTBIm-II); a compound having an oxazole skeleton (in particular, a benzoxazole derivative) such as 4,4′-bis(5-methylbenzoxazol-2-yl)stilbene (abbreviation: BzOs); a phenanthroline derivative such as bathophenanthroline (abbreviation: Bphen), bathocuproine (abbreviation: BCP), and 2,9-bis(naphthalen-2-yl)-4,7-diphenyl-1,10-phenanthroline (abbreviation: NBphen); heterocyclic compounds having a diazine skeleton such as 2-[3-(dibenzothiophen-4-yl)phenyl]dibenzo[f,h]quinoxaline (abbreviation: 2mDBTPDBq-II), 2-[3′-(dibenzothiophen-4-yl)biphenyl-3-yl]dibenzo[f,h]quinoxaline (abbreviation: 2mDBTBPDBq-II), 2-[3′-(9H-carbazol-9-yl)biphenyl-3-yl]dibenzo[f,h]quinoxaline (abbreviation: 2mCzBPDBq), 2-[4-(3,6-diphenyl-9H-carbazol-9-yl)phenyl]dibenzo[f,h]quinoxaline (abbreviation: 2CzPDBq-III), 7-[3-(dibenzothiophen-4-yl)phenyl]dibenzo[f,h]quinoxaline (abbreviation: 7mDBTPDBq-II), 6-[3-(dibenzothiophen-4-yl)phenyl]dibenzo[f,h]quinoxaline (abbreviation: 6mDBTPDBq-II), 4,6-bis[3-(phenanthren-9-yl)phenyl]pyrimidine (abbreviation: 4,6mPnP2Pm), 4,6-bis[3-(4-dibenzothienyl)phenyl]pyrimidine (abbreviation: 4,6mDBTP2Pm-II), and 4,6-bis[3-(9H-carbazol-9-yl)phenyl]pyrimidine (abbreviation: 4,6mCzP2Pm); heterocyclic compounds having a triazine skeleton such as 2-{4-[3-(N-phenyl-9H-carbazol-3-yl)-9H-carbazol-9-yl]phenyl}-4,6-diphenyl-1,3,5-triazine (abbreviation: PCCzPTzn) and 9-[3-(4,6-diphenyl-1,3,5-triazin-2-yl)phenyl]-9′-phenyl-2,3′-bi-9H-carbazole (abbreviation: mPCCzPTzn-02); and heterocyclic compounds having a pyridine skeleton such as 3,5-bis[3-(9H-carbazol-9-yl)phenyl]pyridine (abbreviation: 35DCzPPy) and 1,3,5-tri[3-(3-pyridyl)phenyl]benzene (abbreviation: TmPyPB). Further alternatively, a high molecular compound such as poly(2,5-pyridinediyl) (abbreviation: PPy), poly[(9,9-dihexylfluorene-2,7-diyl)-co-(pyridine-3,5-diyl)](abbreviation: PF-Py), or poly[(9,9-dioctylfluorene-2,7-diyl)-co-(2,2′-bipyridine-6,6′-diyl)] (abbreviation: PF-BPy) can be used.

Examples of the host material include condensed polycyclic aromatic compounds such as anthracene derivatives, phenanthrene derivatives, pyrene derivatives, chrysene derivatives, and dibenzo[g,p]chrysene derivatives. Specific examples of the condensed polycyclic aromatic compound include 9,10-diphenylanthracene (abbreviation: DPAnth), N,N-diphenyl-9-[4-(10-phenyl-9-anthryl)phenyl]-9H-carbazol-3-amine (abbreviation: CzA1PA), 4-(10-phenyl-9-anthryl)triphenylamine (abbreviation: DPhPA), YGAPA, PCAPA, N,9-diphenyl-N-{4-[4-(10-phenyl-9-anthryl)phenyl]phenyl}-9H-carbazol-3-amine (abbreviation: PCAPBA), 2PCAPA, 6,12-dimethoxy-5,11-diphenylchrysene, DBC1,9-[4-(10-phenyl-9-anthracenyl)phenyl]-9H-carbazole (abbreviation: CzPA), 3,6-diphenyl-9-[4-(10-phenyl-9-anthryl)phenyl]-9H-carbazole (abbreviation: DPCzPA), 9,10-bis(3,5-diphenylphenyl)anthracene (abbreviation: DPPA), 9,10-di(2-naphthyl)anthracene (abbreviation: DNA), 2-tert-butyl-9,10-di(2-naphthyl)anthracene (abbreviation: t-BuDNA), 9,9′-bianthryl (abbreviation: BANT), 9,9′-(stilbene-3,3′-diyl)diphenanthrene (abbreviation: DPNS), 9,9′-(stilbene-4,4′-diyl)diphenanthrene (abbreviation: DPNS2), and 1,3,5-tri(1-pyrenyl)benzene (abbreviation: TPB3).

In the case where a plurality of organic compounds are used for the light-emitting layers (113, 113a, 113b, and 113c), it is possible to use two compounds that form an exciplex (a first compound and a second compound) combined with an organometallic complex. In that case, although any of various organic compounds can be used in an appropriate combination, in order to form an exciplex efficiently, it is particularly preferable to combine a compound that easily accepts holes (a hole-transport material) and a compound that easily accepts electrons (an electron-transport material). As the hole-transport material and the electron-transport material, specifically, any of the materials described in this embodiment can be used. With the above structure, high efficiency, low voltage, and a long lifetime can be achieved at the same time.

The TADF material is a material that can up-convert a triplet excited state into a singlet excited state (i.e., reverse intersystem crossing is possible) using a little thermal energy and efficiently exhibits light emission (fluorescence) from the singlet excited state. The TADF is efficiently obtained under the condition where the difference in energy between the triplet excited level and the singlet excited level is greater than or equal to 0 eV and less than or equal to 0.2 eV, preferably greater than or equal to 0 eV and less than or equal to 0.1 eV. Note that “delayed fluorescence” exhibited by the TADF material refers to light emission having the same spectrum as normal fluorescence and an extremely long lifetime. The lifetime is 10-seconds or longer, preferably 10⁻³ seconds or longer.

Examples of the TADF material include fullerene, a derivative thereof, an acridine derivative such as proflavine, and eosin. Other examples include a metal-containing porphyrin, such as a porphyrin containing magnesium (Mg), zinc (Zn), cadmium (Cd), tin (Sn), platinum (Pt), indium (In), or palladium (Pd). Examples of the metal-containing porphyrin include a protoporphyrin-tin fluoride complex (abbreviation: SnF₂(Proto IX)), a mesoporphyrin-tin fluoride complex (abbreviation: SnF₂(Meso IX)), a hematoporphyrin-tin fluoride complex (abbreviation: SnF₂(Hemato IX)), a coproporphyrin tetramethyl ester-tin fluoride complex (abbreviation: SnF₂(Copro III-4Me)), an octaethylporphyrin-tin fluoride complex (abbreviation: SnF₂(OEP)), an etioporphyrin-tin fluoride complex (abbreviation: SnF₂(Etio I)), and an octaethylporphyrin-platinum chloride complex (abbreviation: PtCl₂OEP).

Alternatively, a heterocyclic compound having a π-electron rich heteroaromatic ring and a π-electron deficient heteroaromatic ring, such as 2-(biphenyl-4-yl)-4,6-bis(12-phenylindolo[2,3-a]carbazol-11-yl)-1,3,5-triazine (abbreviation: PIC-TRZ), 2-{4-[3-(N-phenyl-9H-carbazol-3-yl)-9H-carbazol-9-yl]phenyl}-4,6-diphenyl-1,3,5-triazine (abbreviation: PCCzPTzn), 2-[4-(10 OH-phenoxazin-10-yl)phenyl]-4,6-diphenyl-1,3,5-triazine (abbreviation: PXZ-TRZ), 3-[4-(5-phenyl-5,10-dihydrophenazin-10-yl)phenyl]-4,5-diphenyl-1,2,4-triazole (abbreviation: PPZ-3TPT), 3-(9,9-dimethyl-9H-acridin-10-yl)-9H-xanthen-9-one (abbreviation: ACRXTN), bis[4-(9,9-dimethyl-9,10-dihydroacridine)phenyl]sulfone (abbreviation: DMAC-DPS), or 10-phenyl-10H,10′H-spiro[acridin-9,9′-anthracen]-10′-one (abbreviation: ACRSA) can be used. Note that a substance in which the π-electron rich heteroaromatic ring is directly bonded to the π-electron deficient heteroaromatic ring is particularly preferable because both the donor property of the π-electron rich heteroaromatic ring and the acceptor property of the π-electron deficient heteroaromatic ring are increased and the energy difference between the singlet excited state and the triplet excited state becomes small.

Note that when a TADF material is used, the TADF material can be combined with another organic compound. In particular, the TADF material can be combined with the host materials, the hole-transport materials, and the electron-transport materials described above. The organic compound of one embodiment of the present invention described in Embodiment 1 is preferably used as a host material combined with the TADF material.

In the light-emitting element in FIG. 1D, an electron-transport layer 114a is formed over the light-emitting layer 113a of the EL layer 103a by a vacuum evaporation method. After the EL layer 103a and the charge-generation layer 104 are formed, an electron-transport layer 114b is formed over the light-emitting layer 113b of the EL layer 103b by a vacuum evaporation method.

### <Electron-Transport Layer>

The electron-transport layers (114, 114a, and 114b) transport the electrons, which are injected from the second electrode 102 and the charge-generation layer (104) by the electron-injection layers (115, 115a, and 115b), to the light-emitting layers (113, 113a, and 113b). Note that the electron-transport layers (114, 114a, and 114b) each contain an electron-transport material. It is preferable that the electron-transport materials included in the electron-transport layers (114, 114a, and 114b) be substances with an electron mobility of higher than or equal to 1×10⁻⁶ cm²NVs. Note that other substances may also be used as long as the substances have an electron-transport property higher than a hole-transport property. The organic compound of one embodiment of the present invention described in Embodiment 1 has an excellent electron-transport property and thus can also be used for an electron-transport layer.

Examples of the electron-transport material include metal complexes having a quinoline ligand, a benzoquinoline ligand, an oxazole ligand, and a thiazole ligand; an oxadiazole derivative; a triazole derivative; a phenanthroline derivative; a pyridine derivative; and a bipyridine derivative. In addition, a π-electron deficient heteroaromatic compound such as a nitrogen-containing heteroaromatic compound can also be used.

Specifically, it is possible to use metal complexes such as Alq₃, tris(4-methyl-8-quinolinolato)aluminum(III) (abbreviation: Almq₃), bis(10-hydroxybenzo[h]quinolinato)beryllium(II) (abbreviation: BeBq₂), BAlq, bis[2-(2-hydroxyphenyl)benzoxazolato]zinc(II) (abbreviation: Zn(BOX)₂), and bis[2-(2-hydroxyphenyl)benzothiazolato]zinc(II) (abbreviation: Zn(BTZ)₂), heteroaromatic compounds such as 2-(4-biphenylyl)-5-(4-tert-butylphenyl)-1,3,4-oxadiazole (abbreviation: PBD), OXD-7,3-(4′-tert-butylphenyl)-4-phenyl-5-(4″-biphenyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), bathophenanthroline (abbreviation: Bphen), bathocuproine (abbreviation: BCP), and 4,4′-bis(5-methylbenzoxazol-2-yl)stilbene (abbreviation: BzOs), and quinoxaline derivatives and dibenzoquinoxaline derivatives such as 2-[3-(dibenzothiophen-4-yl)phenyl]dibenzo[f,h]quinoxaline (abbreviation: 2mDBTPDBq-II), 2-[3′-(dibenzothiophen-4-yl)biphenyl-3-yl]dibenzo[f,h]quinoxaline (abbreviation: 2mDBTBPDBq-II), 2-[4-(3,6-diphenyl-9H-carbazol-9-yl)phenyl]dibenzo[f,h]quinoxaline (abbreviation: 2CzPDBq-III), 7-[3-(dibenzothiophen-4-yl)phenyl]dibenzo[f,h]quinoxaline (abbreviation: 7mDBTPDBq-II), and 6-[3-(dibenzothiophen-4-yl)phenyl]dibenzo[f,h]quinoxaline (abbreviation: 6mDBTPDBq-II).

Alternatively, a high molecular compound such as poly(2,5-pyridinediyl) (abbreviation: PPy), poly[(9,9-dihexylfluorene-2,7-diyl)-co-(pyridine-3,5-diyl)](abbreviation: PF-Py), or poly[(9,9-dioctylfluorene-2,7-diyl)-co-(2,2′-bipyridine-6,6′-diyl)] (abbreviation: PF-BPy) can be used.

Each of the electron-transport layers (114, 114a, and 114b) is not limited to a single layer, and may be a stack of two or more layers each containing any of the above substances.

In the light-emitting element in FIG. 1D, the electron-injection layer 115a is formed over the electron-transport layer 114a of the EL layer 103a by a vacuum evaporation method. Subsequently, the EL layer 103a and the charge-generation layer 104 are formed, the components up to the electron-transport layer 114b of the EL layer 103b are formed, and then the electron-injection layer 115b is formed thereover by a vacuum evaporation method.

### <Electron-Injection Layer>

The electron-injection layers (115, 115a, and 115b) each contain a substance having a high electron-injection property. The electron-injection layers (115, 115a, and 115b) can each be formed using an alkali metal, an alkaline earth metal, or a compound thereof, such as lithium fluoride (LiF), cesium fluoride (CsF), calcium fluoride (CaF₂), or lithium oxide (LiOₓ). A rare earth metal compound like erbium fluoride (ErF₃) can also be used. Electride may also be used for the electron-injection layers (115, 115a, and 115b). Examples of the electride include a substance in which electrons are added at high concentration to calcium oxide-aluminum oxide. Any of the substances for forming the electron-transport layers (114, 114a, and 114b), which are given above, can also be used.

A composite material in which an organic compound and an electron donor (donor) are mixed may also be used for the electron-injection layers (115, 115a, and 115b). Such a composite material is excellent in an electron-injection property and an electron-transport property because electrons are generated in the organic compound by the electron donor. The organic compound here is preferably a material excellent in transporting the generated electrons; specifically, for example, the electron-transport materials for forming the electron-transport layers (114, 114a, and 114b) (e.g., a metal complex or a heteroaromatic compound) can be used. As the electron donor, a substance showing an electron-donating property with respect to the organic compound may be used. Preferable examples are an alkali metal, an alkaline earth metal, and a rare earth metal. Specifically, lithium, cesium, magnesium, calcium, erbium, ytterbium, and the like can be given. Furthermore, an alkali metal oxide and an alkaline earth metal oxide are preferable, and a lithium oxide, a calcium oxide, a barium oxide, and the like can be given. Alternatively, a Lewis base such as magnesium oxide can be used. Further alternatively, an organic compound such as tetrathiafulvalene (abbreviation: TTF) can be used.

In the case where light obtained from the light-emitting layer 113b is amplified, for example, the optical path length between the second electrode 102 and the light-emitting layer 113b is preferably less than one fourth of the wavelength λ of light emitted from the light-emitting layer 113b. In that case, the optical path length can be adjusted by changing the thickness of the electron-transport layer 114b or the electron-injection layer 115b.

### <Charge-Generation Layer>

The charge-generation layer 104 has a function of injecting electrons into the EL layer 103a and injecting holes into the EL layer 103b when voltage is applied between the first electrode (anode) 101 and the second electrode (cathode) 102. The charge-generation layer 104 may have either a structure in which an electron acceptor (acceptor) is added to a hole-transport material or a structure in which an electron donor (donor) is added to an electron-transport material. Alternatively, both of these structures may be stacked. Note that forming the charge-generation layer 104 by using any of the above materials can suppress an increase in drive voltage caused by the stack of the EL layers.

In the case where the charge-generation layer 104 has a structure in which an electron acceptor is added to a hole-transport material, any of the materials described in this embodiment can be used as the hole-transport material. As the electron acceptor, it is possible to use 7,7,8,8-tetracyano-2,3,5,6-tetrafluoroquinodimethane (abbreviation: F₄-TCNQ), chloranil, and the like. In addition, oxides of metals that belong to Group 4 to Group 8 of the periodic table can be given. Specifically, vanadium oxide, niobium oxide, tantalum oxide, chromium oxide, molybdenum oxide, tungsten oxide, manganese oxide, rhenium oxide, or the like is used.

In the case where the charge-generation layer 104 has a structure in which an electron donor is added to an electron-transport material, any of the materials described in this embodiment can be used as the electron-transport material. As the electron donor, it is possible to use an alkali metal, an alkaline earth metal, a rare earth metal, metals that belong to Groups 2 and 13 of the periodic table, or an oxide or carbonate thereof. Specifically, lithium (Li), cesium (Cs), magnesium (Mg), calcium (Ca), ytterbium (Yb), indium (In), lithium oxide, cesium carbonate, or the like is preferably used. Alternatively, an organic compound such as tetrathianaphthacene may be used as the electron donor.

Note that the EL layer 103c in FIG. 1E has a structure similar to those of the above-described EL layers (103, 103a, and 103b). In addition, the charge-generation layers 104a and 104b each have a structure similar to that of the above-described charge-generation layer 104.

### <Substrate>

The light-emitting element described in this embodiment can be formed over any of a variety of substrates. Note that the type of the substrate is not limited to a certain type. Examples of the substrate include a semiconductor substrate (e.g., a single crystal substrate or a silicon substrate), an SOI substrate, a glass substrate, a quartz substrate, a plastic substrate, a metal substrate, a stainless steel substrate, a substrate including stainless steel foil, a tungsten substrate, a substrate including tungsten foil, a flexible substrate, an attachment film, paper including a fibrous material, and a base material film.

Examples of the glass substrate include a barium borosilicate glass substrate, an aluminoborosilicate glass substrate, and a soda lime glass substrate. Examples of the flexible substrate, the attachment film, and the base material film include plastics typified by polyethylene terephthalate (PET), polyethylene naphthalate (PEN), and polyether sulfone (PES); a synthetic resin such as acrylic; polypropylene; polyester, polyvinyl fluoride; polyvinyl chloride; polyamide; polyimide; aramid; epoxy; an inorganic vapor deposition film; and paper.

For fabrication of the light-emitting element in this embodiment, a vacuum process such as an evaporation method or a solution process such as a spin coating method or an ink-jet method can be used. When an evaporation method is used, a physical vapor deposition method (PVD method) such as a sputtering method, an ion plating method, an ion beam evaporation method, a molecular beam evaporation method, or a vacuum evaporation method, a chemical vapor deposition method (CVD method), or the like can be used. Specifically, the functional layers (the hole-injection layers (111, 111a, and 111b), the hole-transport layers (112, 112a, and 112b), the light-emitting layers (113, 113a, 113b, and 113c), the electron-transport layers (114, 114a, and 114b), the electron-injection layers (115, 115a, and 115b)) included in the EL layers and the charge-generation layers (104, 104a, and 104b) of the light-emitting element can be formed by an evaporation method (e.g., a vacuum evaporation method), a coating method (e.g., a dip coating method, a die coating method, a bar coating method, a spin coating method, or a spray coating method), a printing method (e.g., an ink-jet method, screen printing (stencil), offset printing (planography), flexography (relief printing), gravure printing, or micro-contact printing), or the like.

Note that materials that can be used for the functional layers (the hole-injection layers (111, 111a, and 111b), the hole-transport layers (112, 112a, and 112b), the light-emitting layers (113, 113a, 113b, and 113c), the electron-transport layers (114, 114a, and 114b), and the electron-injection layers (115, 115a, and 115b)) that are included in the EL layers (103, 103a, and 103b) and the charge-generation layers (104, 104a, and 104b) in the light-emitting element described in this embodiment are not limited to the above materials, and other materials can be used in combination as long as the functions of the layers are fulfilled. For example, a high molecular compound (e.g., an oligomer, a dendrimer, or a polymer), a middle molecular compound (a compound between a low molecular compound and a high molecular compound with a molecular weight of 400 to 4000), an inorganic compound (e.g., a quantum dot material), or the like can be used. The quantum dot may be a colloidal quantum dot, an alloyed quantum dot, a core-shell quantum dot, a core quantum dot, or the like.

The structures described in this embodiment can be combined with any of the structures described in the other embodiments as appropriate.

### Embodiment 3

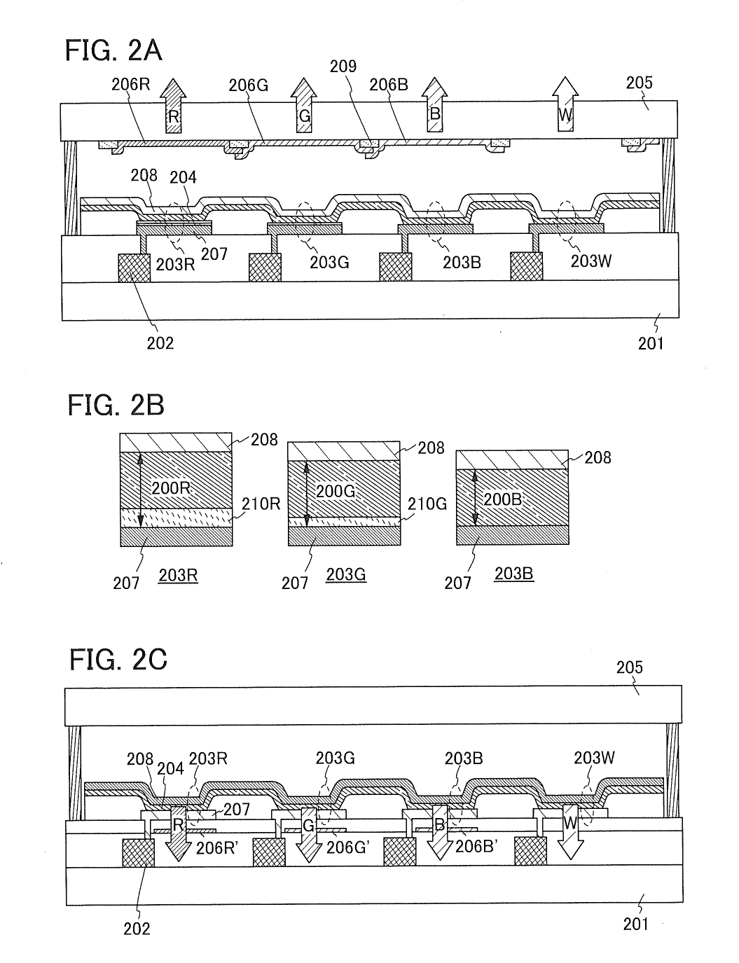

In this embodiment, a light-emitting device of one embodiment of the present invention is described. Note that a light-emitting device illustrated in FIG. 2A is an active-matrix light-emitting device in which transistors (FETs) 202 are electrically connected to light-emitting elements (203R, 203G, 203B, and 203W) over a first substrate 201. The light-emitting elements (203R, 203G, 203B, and 203W) include a common EL layer 204 and each have a microcavity structure in which the optical path length between electrodes is adjusted depending on the emission color of the light-emitting element. The light-emitting device is a top-emission light-emitting device in which light is emitted from the EL layer 204 through color filters (206R, 206G, and 206B) formed on a second substrate 205.

The light-emitting device illustrated in FIG. 2A is fabricated such that a first electrode 207 functions as a reflective electrode and a second electrode 208 functions as a transflective electrode. Note that description in any of the other embodiments can be referred to as appropriate for electrode materials for the first electrode 207 and the second electrode 208.

In the case where the light-emitting element 203R functions as a red light-emitting element, the light-emitting element 203G functions as a green light-emitting element, the light-emitting element 203B functions as a blue light-emitting element, and the light-emitting element 203W functions as a white light-emitting element in FIG. 2A, for example, a gap between the first electrode 207 and the second electrode 208 in the light-emitting element 203R is adjusted to have an optical path length 200R, a gap between the first electrode 207 and the second electrode 208 in the light-emitting element 203G is adjusted to have an optical path length 200G, and a gap between the first electrode 207 and the second electrode 208 in the light-emitting element 203B is adjusted to have an optical path length 200B as illustrated in FIG. 2B. Note that optical adjustment can be performed in such a manner that a conductive layer 210R is stacked over the first electrode 207 in the light-emitting element 203R and a conductive layer 210G is stacked over the first electrode 207 in the light-emitting element 203G as illustrated in FIG. 2B.

The second substrate 205 is provided with the color filters (206R, 206G, and 206B). Note that the color filters each transmit visible light in a specific wavelength range and blocks visible light in a specific wavelength range. Thus, as illustrated in FIG. 2A, the color filter 206R that transmits only light in the red wavelength range is provided in a position overlapping with the light-emitting element 203R, whereby red light emission can be obtained from the light-emitting element 203R. Furthermore, the color filter 206G that transmits only light in the green wavelength range is provided in a position overlapping with the light-emitting element 203G, whereby green light emission can be obtained from the light-emitting element 203G. Moreover, the color filter 206B that transmits only light in the blue wavelength range is provided in a position overlapping with the light-emitting element 203B, whereby blue light emission can be obtained from the light-emitting element 203B. Note that the light-emitting element 203W can emit white light without a color filter. Note that a black layer (black matrix) 209 may be provided at an end portion of each color filter. The color filters (206R, 206G, and 206B) and the black layer 209 may be covered with an overcoat layer formed using a transparent material.

Although the light-emitting device in FIG. 2A has a structure in which light is extracted from the second substrate 205 side (top emission structure), a structure in which light is extracted from the first substrate 201 side where the FETs 202 are formed (bottom emission structure) may be employed as illustrated in FIG. 2C. In the case of a bottom-emission light-emitting device, the first electrode 207 is formed as a transflective electrode and the second electrode 208 is formed as a reflective electrode. As the first substrate 201, a substrate having at least a light-transmitting property is used. As illustrated in FIG. 2C, color filters (206R′, 206G′, and 206B′) are provided so as to be closer to the first substrate 201 than the light-emitting elements (203R, 203G, and 203B) are.

In FIG. 2A, the light-emitting elements are the red light-emitting element, the green light-emitting element, the blue light-emitting element, and the white light-emitting element; however, the light-emitting elements of one embodiment of the present invention are not limited to the above, and a yellow light-emitting element or an orange light-emitting element may be used. Note that description in any of the other embodiments can be referred to as appropriate for materials that are used for the EL layers (a light-emitting layer, a hole-injection layer, a hole-transport layer, an electron-transport layer, an electron-injection layer, a charge-generation layer, and the like) to fabricate each of the light-emitting elements. In that case, a color filter needs to be appropriately selected depending on the emission color of the light-emitting element.

With the above structure, a light-emitting device including light-emitting elements that exhibit a plurality of emission colors can be fabricated.

Note that the structures described in this embodiment can be combined with any of the structures described in the other embodiments as appropriate.

### Embodiment 4

In this embodiment, a light-emitting device of one embodiment of the present invention is described.

The use of the element structure of the light-emitting element of one embodiment of the present invention allows fabrication of an active-matrix light-emitting device or a passive-matrix light-emitting device. Note that an active-matrix light-emitting device has a structure including a combination of a light-emitting element and a transistor (FET). Thus, each of a passive-matrix light-emitting device and an active-matrix light-emitting device is one embodiment of the present invention. Note that any of the light-emitting elements described in other embodiments can be used in the light-emitting device described in this embodiment.

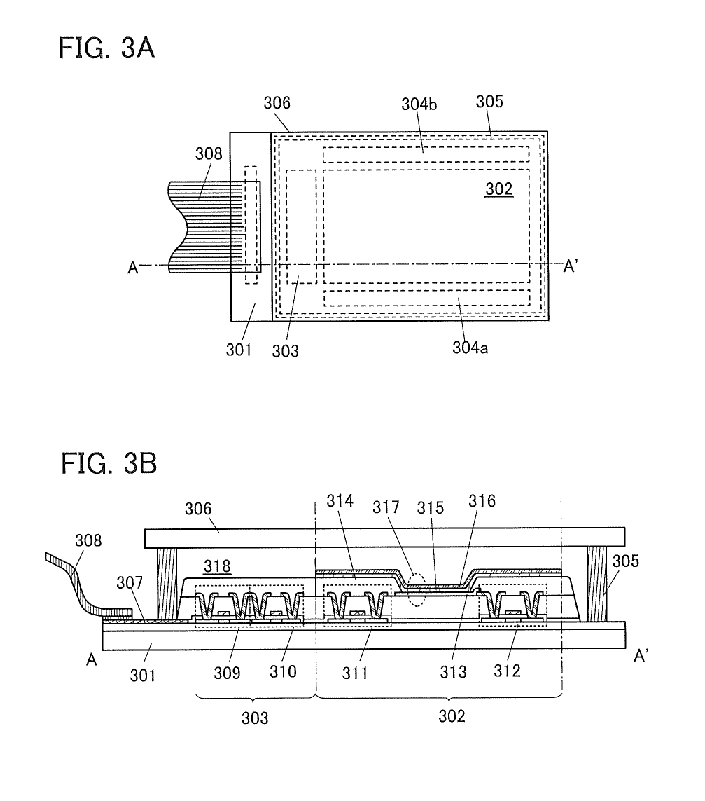

In this embodiment, an active-matrix light-emitting device will be described with reference to FIGS. 3A and 3B.

FIG. 3A is a top view illustrating the light-emitting device, and FIG. 3B is a cross-sectional view taken along chain line A-A′ in FIG. 3A. The active-matrix light-emitting device includes a pixel portion 302, a driver circuit portion (source line driver circuit) 303, and driver circuit portions (gate line driver circuits) (304a and 304b) that are provided over a first substrate 301. The pixel portion 302 and the driver circuit portions (303, 304a, and 304b) are sealed between the first substrate 301 and a second substrate 306 with a sealant 305.

A lead wiring 307 is provided over the first substrate 301. The lead wiring 307 is connected to an FPC 308 that is an external input terminal. Note that the FPC 308 transmits a signal (e.g., a video signal, a clock signal, a start signal, or a reset signal) or a potential from the outside to the driver circuit portions (303, 304a, and 304b). The FPC 308 may be provided with a printed wiring board (PWB). Note that the light-emitting device provided with an FPC or a PWB is included in the category of a light-emitting device.

FIG. 3B illustrates a cross-sectional structure of the light-emitting device.

The pixel portion 302 includes a plurality of pixels each of which includes an FET (switching FET) 311, an FET (current control FET) 312, and a first electrode 313 electrically connected to the FET 312. Note that the number of FETs included in each pixel is not particularly limited and can be set appropriately.

As FETs 309, 310, 311, and 312, for example, a staggered transistor or an inverted staggered transistor can be used without particular limitation. A top-gate transistor, a bottom-gate transistor, or the like may be used.

Note that there is no particular limitation on the crystallinity of a semiconductor that can be used for the FETs 309, 310, 311, and 312, and an amorphous semiconductor or a semiconductor having crystallinity (a microcrystalline semiconductor, a polycrystalline semiconductor, a single crystal semiconductor, or a semiconductor partly including crystal regions) may be used. A semiconductor having crystallinity is preferably used, in which case deterioration of the transistor characteristics can be suppressed.

For the semiconductor, a Group 14 element, a compound semiconductor, an oxide semiconductor, an organic semiconductor, or the like can be used, for example. As a typical example, a semiconductor containing silicon, a semiconductor containing gallium arsenide, or an oxide semiconductor containing indium can be used.

The driver circuit portion 303 includes the FET 309 and the FET 310. The FET 309 and the FET 310 may be formed with a circuit including transistors having the same conductivity type (either n-channel transistors or p-channel transistors) or a CMOS circuit including an n-channel transistor and a p-channel transistor. Furthermore, a driver circuit may be provided outside.

An end portion of the first electrode 313 is covered with an insulator 314. The insulator 314 can be formed using an organic compound such as a negative photosensitive resin or a positive photosensitive resin (acrylic resin), or an inorganic compound such as silicon oxide, silicon oxynitride, or silicon nitride. The insulator 314 preferably has a curved surface with curvature at an upper end portion or a lower end portion thereof. In that case, favorable coverage with a film formed over the insulator 314 can be obtained.

An EL layer 315 and a second electrode 316 are stacked over the first electrode 313. The EL layer 315 includes a light-emitting layer, a hole-injection layer, a hole-transport layer, an electron-transport layer, an electron-injection layer, a charge-generation layer, and the like.

The structure and materials described in any of the other embodiments can be used for the components of a light-emitting element 317 described in this embodiment. Although not illustrated, the second electrode 316 is electrically connected to the FPC 308 that is an external input terminal.

Although the cross-sectional view in FIG. 3B illustrates only one light-emitting element 317, a plurality of light-emitting elements are arranged in a matrix in the pixel portion 302. Light-emitting elements that emit light of three kinds of colors (R, G, and B) are selectively formed in the pixel portion 302, whereby a light-emitting device capable of displaying a full-color image can be obtained. In addition to the light-emitting elements that emit light of three kinds of colors (R, G, and B), for example, light-emitting elements that emit light of white (W), yellow (Y), magenta (M), cyan (C), and the like may be formed. For example, the light-emitting elements that emit light of some of the above colors are used in combination with the light-emitting elements that emit light of three kinds of colors (R, G, and B), whereby effects such as an improvement in color purity and a reduction in power consumption can be achieved. Alternatively, a light-emitting device which is capable of displaying a full-color image may be fabricated by a combination with color filters. As color filters, red (R), green (G), blue (B), cyan (C), magenta (M), and yellow (Y) color filters and the like can be used.

When the second substrate 306 and the first substrate 301 are bonded to each other with the sealant 305, the FETs (309, 310, 311, and 312) and the light-emitting element 317 over the first substrate 301 are provided in a space 318 surrounded by the first substrate 301, the second substrate 306, and the sealant 305. Note that the space 318 may be filled with an inert gas (e.g., nitrogen or argon) or an organic substance (including the sealant 305).

An epoxy-based resin, glass frit, or the like can be used for the sealant 305. It is preferable to use a material that is permeable to as little moisture and oxygen as possible for the sealant 305. As the second substrate 306, a substrate that can be used as the first substrate 301 can be similarly used. Thus, any of the various substrates described in the other embodiments can be appropriately used. As the substrate, a glass substrate, a quartz substrate, or a plastic substrate made of fiber-reinforced plastic (FRP), polyvinyl fluoride (PVF), polyester, acrylic, or the like can be used. In the case where glass frit is used for the sealant, the first substrate 301 and the second substrate 306 are preferably glass substrates in terms of adhesion.

Accordingly, the active-matrix light-emitting device can be obtained.

In the case where the active-matrix light-emitting device is provided over a flexible substrate, the FETs and the light-emitting element may be directly formed over the flexible substrate; alternatively, the FETs and the light-emitting element may be formed over a substrate provided with a separation layer and then separated at the separation layer by application of heat, force, laser, or the like to be transferred to a flexible substrate. For the separation layer, a stack including inorganic films such as a tungsten film and a silicon oxide film, or an organic resin film of polyimide or the like can be used, for example. Examples of the flexible substrate include, in addition to a substrate over which a transistor can be formed, a paper substrate, a cellophane substrate, an aramid film substrate, a polyimide film substrate, a cloth substrate (including a natural fiber (e.g., silk, cotton, or hemp), a synthetic fiber (e.g., nylon, polyurethane, or polyester), a regenerated fiber (e.g., acetate, cupra, rayon, or regenerated polyester), or the like), a leather substrate, and a rubber substrate. With the use of any of these substrates, an increase in durability, an increase in heat resistance, a reduction in weight, and a reduction in thickness can be achieved.

Note that the structures described in this embodiment can be combined with any of the structures described in the other embodiments as appropriate.

### Embodiment 5

In this embodiment, examples of a variety of electronic devices and an automobile manufactured using the light-emitting device of one embodiment of the present invention or a display device including the light-emitting element of one embodiment of the present invention are described.

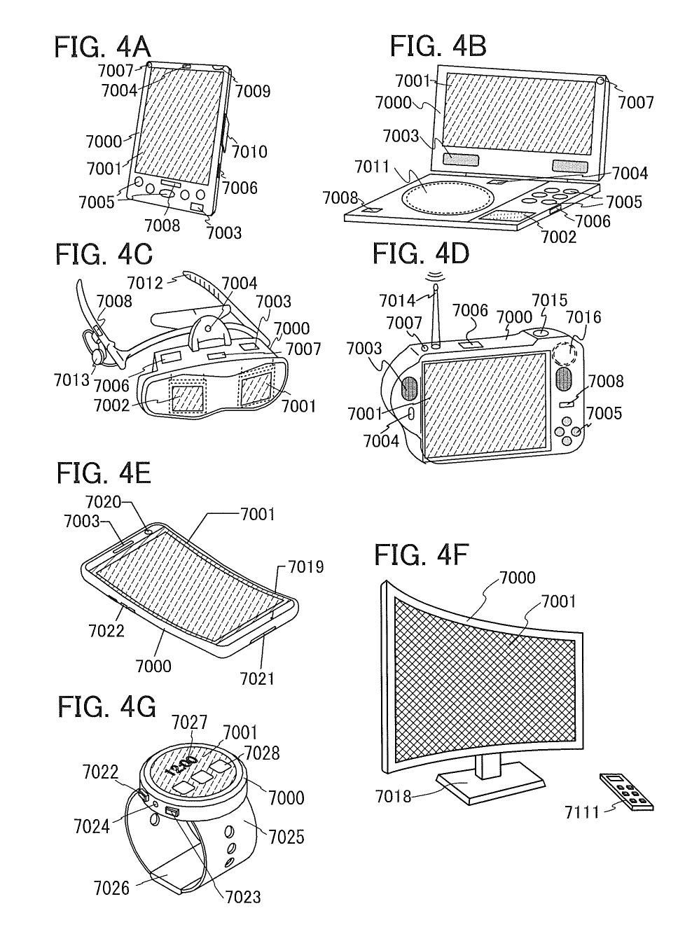

Electronic devices illustrated in FIGS. 4A to 4E can include a housing 7000, a display portion 7001, a speaker 7003, an LED lamp 7004, operation keys 7005 (including a power switch or an operation switch), a connection terminal 7006, a sensor 7007 (a sensor having a function of measuring or sensing force, displacement, position, speed, acceleration, angular velocity, rotational frequency, distance, light, liquid, magnetism, temperature, chemical substance, sound, time, hardness, electric field, current, voltage, electric power, radiation, flow rate, humidity, gradient, oscillation, odor, or infrared ray), a microphone 7008, and the like.

FIG. 4A illustrates a mobile computer that can include a switch 7009, an infrared port 7010, and the like in addition to the above components.

FIG. 4B illustrates a portable image reproducing device (e.g., a DVD player) that is provided with a recording medium and can include a second display portion 7002, a recording medium reading portion 7011, and the like in addition to the above components.

FIG. 4C illustrates a goggle-type display that can include the second display portion 7002, a support 7012, an earphone 7013, and the like in addition to the above components.

FIG. 4D illustrates a digital camera that has a television reception function and can include an antenna 7014, a shutter button 7015, an image receiving portion 7016, and the like in addition to the above components.

FIG. 4E illustrates a cellular phone (including a smartphone) that can include the display portion 7001, a microphone 7019, the speaker 7003, a camera 7020, an external connection portion 7021, an operation button 7022, and the like in the housing 7000.

FIG. 4F illustrates a large-size television set (also referred to as TV or a television receiver) that can include the housing 7000, the display portion 7001, and the like. In addition, here, the housing 7000 is supported by a stand 7018. The television set can be operated with a separate remote controller 7111 or the like. The display portion 7001 may include a touch sensor. The television set can be operated by touching the display portion 7001 with a finger or the like. The remote controller 7111 may be provided with a display portion for displaying information output from the remote controller 7111. With operation keys or a touch panel of the remote controller 7111, channels and volume can be controlled and images displayed on the display portion 7001 can be controlled.

The electronic devices illustrated in FIGS. 4A to 4F can have a variety of functions, such as a function of displaying a variety of information (a still image, a moving image, a text image, and the like) on the display portion, a touch panel function, a function of displaying a calendar, date, time, and the like, a function of controlling processing with a variety of types of software (programs), a wireless communication function, a function of connecting to a variety of computer networks with a wireless communication function, a function of transmitting and receiving a variety of data with a wireless communication function, a function of reading a program or data stored in a recording medium and displaying the program or data on the display portion, and the like. Furthermore, the electronic device including a plurality of display portions can have a function of displaying image data mainly on one display portion while displaying text data mainly on another display portion, a function of displaying a three-dimensional image by displaying images on a plurality of display portions with a parallax taken into account, or the like. Furthermore, the electronic device including an image receiving portion can have a function of taking a still image, a function of taking a moving image, a function of automatically or manually correcting a taken image, a function of storing a taken image in a recording medium (an external recording medium or a recording medium incorporated in the camera), a function of displaying a taken image on the display portion, or the like. Note that functions that can be provided for the electronic devices illustrated in FIGS. 4A to 4F are not limited to those described above, and the electronic devices can have a variety of functions.

FIG. 4G illustrates a smart watch, which includes the housing 7000, the display portion 7001, operation buttons 7022 and 7023, a connection terminal 7024, a band 7025, a clasp 7026, and the like.

The display portion 7001 mounted in the housing 7000 serving as a bezel includes a non-rectangular display region. The display portion 7001 can display an icon 7027 indicating time, another icon 7028, and the like. The display portion 7001 may be a touch panel (an input/output device) including a touch sensor (an input device).

The smart watch illustrated in FIG. 4G can have a variety of functions, such as a function of displaying a variety of information (e.g., a still image, a moving image, and a text image) on the display portion, a touch panel function, a function of displaying a calendar, date, time, and the like, a function of controlling processing with a variety of types of software (programs), a wireless communication function, a function of connecting to a variety of computer networks with a wireless communication function, a function of transmitting and receiving a variety of data with a wireless communication function, a function of reading a program or data stored in a recording medium and displaying the program or data on the display portion, and the like.

The housing 7000 can include a speaker, a sensor (a sensor having a function of measuring or sensing force, displacement, position, speed, acceleration, angular velocity, rotational frequency, distance, light, liquid, magnetism, temperature, chemical substance, sound, time, hardness, electric field, current, voltage, electric power, radiation, flow rate, humidity, gradient, oscillation, odor, or infrared rays), a microphone, and the like.

Note that the light-emitting device of one embodiment of the present invention or the display device including the light-emitting element of one embodiment of the present invention can be used in the display portion of each electronic device described in this embodiment, so that a long lifetime electronic device can be obtained.

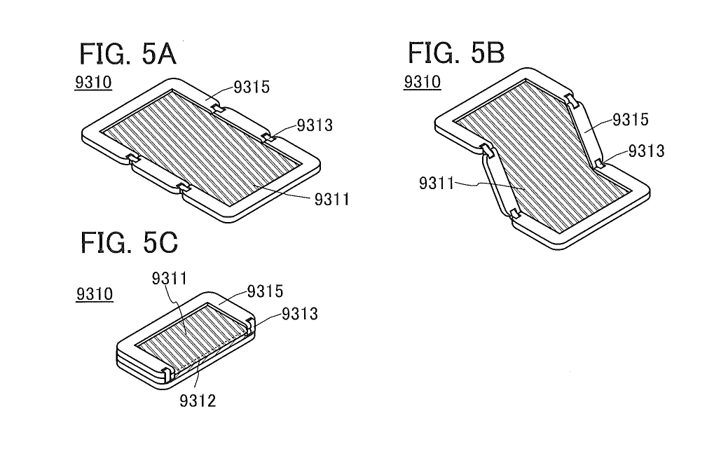

Another electronic device including the light-emitting device is a foldable portable information terminal illustrated in FIGS. 5A to 5C. FIG. 5A illustrates a portable information terminal 9310 which is opened. FIG. 5B illustrates the portable information terminal 9310 which is being opened or being folded. FIG. 5C illustrates the portable information terminal 9310 which is folded. The portable information terminal 9310 is highly portable when folded. The portable information terminal 9310 is highly browsable when opened because of a seamless large display region.

A display portion 9311 is supported by three housings 9315 joined together by hinges 9313. Note that the display portion 9311 may be a touch panel (an input/output device) including a touch sensor (an input device). By bending the display portion 9311 at a connection portion between two housings 9315 with the use of the hinges 9313, the portable information terminal 9310 can be reversibly changed in shape from an opened state to a folded state. The light-emitting device of one embodiment of the present invention can be used for the display portion 9311. In addition, a long lifetime electronic device can be obtained. A display region 9312 in the display portion 9311 is a display region that is positioned at a side surface of the portable information terminal 9310 which is folded. On the display region 9312, information icons, file shortcuts of frequently used applications or programs, and the like can be displayed, and confirmation of information and start of application and the like can be smoothly performed.

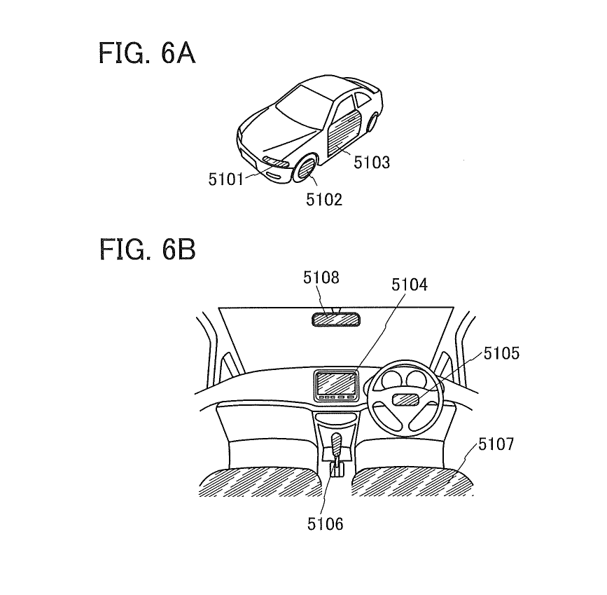

FIGS. 6A and 6B illustrate an automobile including the light-emitting device. The light-emitting device can be incorporated in the automobile, and specifically, can be included in lights 5101 (including lights of the rear part of the car), a wheel cover 5102, a part or whole of a door 5103, or the like on the outer side of the automobile which is illustrated in FIG. 6A. The light-emitting device can also be included in a display portion 5104, a steering wheel 5105, a gear lever 5106, a seat 5107, an inner rearview mirror 5108, or the like on the inner side of the automobile which is illustrated in FIG. 6B, or in a part of a glass window.

In the above manner, the electronic devices and automobiles can be obtained using the light-emitting device or the display device of one embodiment of the present invention. In that case, a long lifetime electronic device can be obtained. Note that the light-emitting device or the display device can be used for electronic devices and automobiles in a variety of fields without being limited to those described in this embodiment.

Note that the structures described in this embodiment can be combined with any of the structures described in the other embodiments as appropriate.

### Embodiment 6

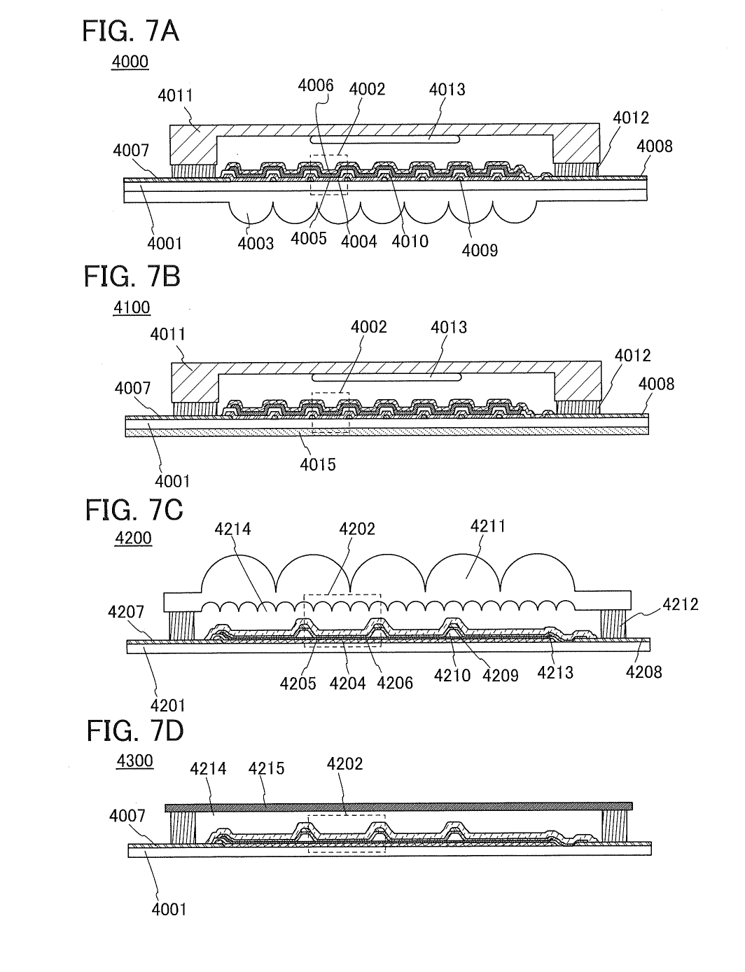

In this embodiment, a structure of a lighting device fabricated using the light-emitting device of one embodiment of the present invention or the light-emitting element which is a part of the light-emitting device is described with reference to FIGS. 7A to 7D.

FIGS. 7A to 7D are examples of cross-sectional views of lighting devices. FIGS. 7A and 7B illustrate bottom-emission lighting devices in which light is extracted from the substrate side, and FIGS. 7C and 7D illustrate top-emission lighting devices in which light is extracted from the sealing substrate side.

A lighting device 4000 illustrated in FIG. 7A includes a light-emitting element 4002 over a substrate 4001. In addition, the lighting device 4000 includes a substrate 4003 with unevenness on the outside of the substrate 4001. The light-emitting element 4002 includes a first electrode 4004, an EL layer 4005, and a second electrode 4006.

The first electrode 4004 is electrically connected to an electrode 4007, and the second electrode 4006 is electrically connected to an electrode 4008. In addition, an auxiliary wiring 4009 electrically connected to the first electrode 4004 may be provided. Note that an insulating layer 4010 is formed over the auxiliary wiring 4009.

The substrate 4001 and a sealing substrate 4011 are bonded to each other with a sealant 4012. A desiccant 4013 is preferably provided between the sealing substrate 4011 and the light-emitting element 4002. The substrate 4003 has the unevenness illustrated in FIG. 7A, whereby the extraction efficiency of light emitted from the light-emitting element 4002 can be increased.

Instead of the substrate 4003, a diffusion plate 4015 may be provided on the outside of the substrate 4001 as in a lighting device 4100 illustrated in FIG. 7B.

A lighting device 4200 illustrated in FIG. 7C includes a light-emitting element 4202 over a substrate 4201. The light-emitting element 4202 includes a first electrode 4204, an EL layer 4205, and a second electrode 4206.

The first electrode 4204 is electrically connected to an electrode 4207, and the second electrode 4206 is electrically connected to an electrode 4208. An auxiliary wiring 4209 electrically connected to the second electrode 4206 may be provided. An insulating layer 4210 may be provided under the auxiliary wiring 4209.

The substrate 4201 and a sealing substrate 4211 with unevenness are bonded to each other with a sealant 4212. A barrier film 4213 and a planarization film 4214 may be provided between the sealing substrate 4211 and the light-emitting element 4202. The sealing substrate 4211 has the unevenness illustrated in FIG. 7C, whereby the extraction efficiency of light emitted from the light-emitting element 4202 can be increased.

Instead of the sealing substrate 4211, a diffusion plate 4215 may be provided over the light-emitting element 4202 as in a lighting device 4300 illustrated in FIG. 7D.

Note that with the use of the light-emitting device of one embodiment of the present invention or the light-emitting element which is a part of the light-emitting device as described in this embodiment, a lighting device having desired chromaticity can be provided.

Note that the structures described in this embodiment can be combined with any of the structures described in the other embodiments as appropriate.

### Embodiment 7

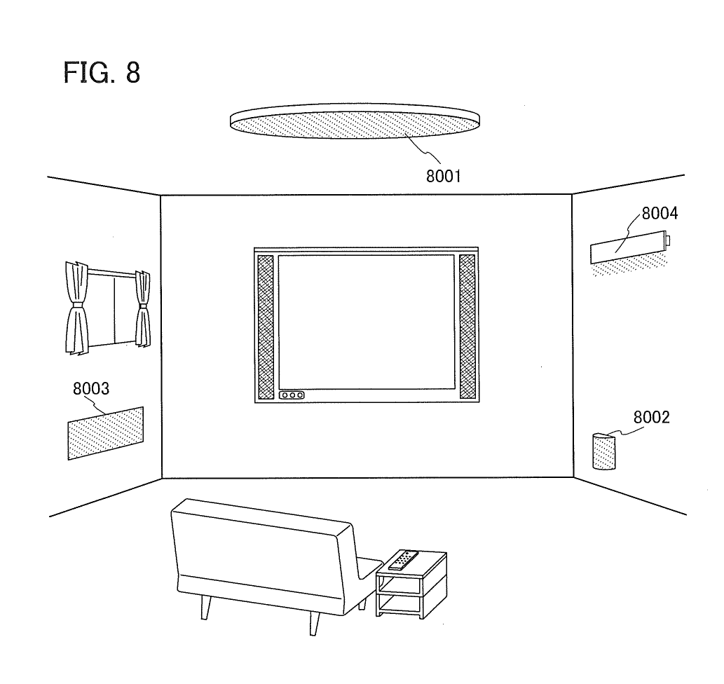

In this embodiment, application examples of lighting devices fabricated using the light-emitting device of one embodiment of the present invention or the light-emitting element which is a part of the light-emitting device will be described with reference to FIG. 8.

A ceiling light 8001 can be used as an indoor lighting device. Examples of the ceiling light 8001 include a direct-mount light and an embedded light. Such a lighting device is fabricated using the light-emitting device and a housing or a cover in combination. Besides, application to a cord pendant light (light that is suspended from a ceiling by a cord) is also possible.

A foot light 8002 lights a floor so that safety on the floor can be improved. For example, it can be effectively used in a bedroom, on a staircase, or on a passage. In that case, the size or shape of the foot light can be changed depending on the area or structure of a room. The foot light 8002 can be a stationary lighting device fabricated using the light-emitting device and a support in combination.

A sheet-like lighting 8003 is a thin sheet-like lighting device. The sheet-like lighting, which is attached to a wall when used, is space-saving and thus can be used for a wide variety of uses. Furthermore, the area of the sheet-like lighting can be easily increased. The sheet-like lighting can also be used on a wall or housing having a curved surface.

In addition, a lighting device 8004 in which the direction of light from a light source is controlled to be only a desired direction can be used.

Besides the above examples, when the light-emitting device of one embodiment of the present invention or the light-emitting element which is a part of the light-emitting device is used as part of furniture in a room, a lighting device that functions as the furniture can be obtained.

As described above, a variety of lighting devices that include the light-emitting device can be obtained. Note that these lighting devices are also embodiments of the present invention.

The structures described in this embodiment can be combined with any of the structures described in the other embodiments as appropriate.

### Example 1

### Synthesis Example 1

This example describes a method for synthesizing 9-[(3′-dibenzothiophen-4-yl)biphenyl-3-yl]naphtho[1′,2′:4,5]furo[2,3-b]pyrazine (abbreviation: 9mDBtBPNfpr), which is the organic compound of one embodiment of the present invention represented by Structural Formula (100) in Embodiment 1. The structure of 9mDBtBPNfpr is shown below.

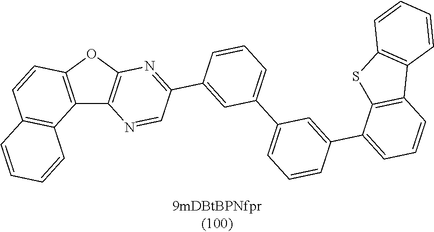 [SMILES: c1cc(-c2cccc(-c3cccc4c3sc3ccccc34)c2)cc(-c2cnc3c(n2)oc2ccc4ccccc4c23)c1]

### Step 1: Synthesis of 6-chloro-3-(2-methoxynaphthalen-1-yl)pyrazin-2-amine

First, into a three-neck flask equipped with a reflux pipe were put 4.37 g of 3-bromo-6-chloropyrazin-2-amine, 4.23 g of 2-methoxynaphthalene-1-boronic acid, 4.14 g of potassium fluoride, and 75 mL of dehydrated tetrahydrofuran, and the air in the flask was replaced with nitrogen. The mixture in the flask was degassed by being stirred under reduced pressure, and then 0.57 g of tris(dibenzylideneacetone)dipalladium(0) (abbreviation: Pd₂(dba)₃) and 4.5 mL of tri-tert-butylphosphine (abbreviation: P(tBu)₃) were added thereto. The mixture was stirred at 80° C. for 54 hours to be reacted.

After a predetermined time elapsed, the obtained mixture was subjected to suction filtration and the filtrate was concentrated. Then, purification by silica gel column chromatography using a developing solvent (toluene:ethyl acetate=9:1) was performed, so that 2.19 g of a target pyrazine derivative (yellowish white powder) was obtained in a yield of 36%. A synthesis scheme of Step 1 is shown in (a-1) below.

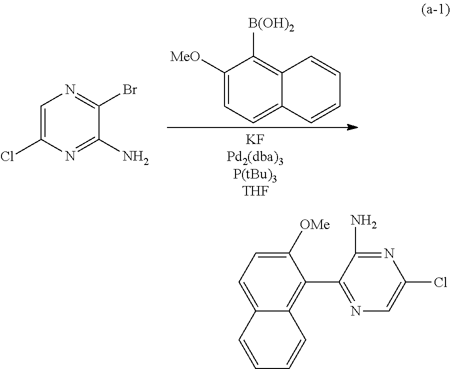 [SMILES: Nc1nc(Cl)cnc1Br ; COc1ccc2ccccc2c1B(O)O ; COc1ccc2ccccc2c1-c1ncc(Cl)nc1N]

### Step 2: Synthesis of 9-chloronaphtho[1′,2′:4,5]furo[2,3-b]pyrazine

Next, into a three-neck flask were put 2.18 g of 6-chloro-3-(2-methoxynaphthalen-1-yl)pyrazin-2-amine obtained in Step 1, 63 mL of dehydrated tetrahydrofuran, and 84 mL of a glacial acetic acid, and the air in the flask was replaced with nitrogen. After the flask was cooled down to −10° C., 2.8 mL of tert-butyl nitrite was dripped, and the mixture was stirred at −10° C. for 30 minutes and at 0° C. for 3 hours. After a predetermined time elapsed, 250 mL of water was added to the obtained suspension and suction filtration was performed, so that 1.48 g of a target pyrazine derivative (yellowish white powder) was obtained in a yield of 77%. A synthesis scheme of Step 2 is shown in (a-2) below.

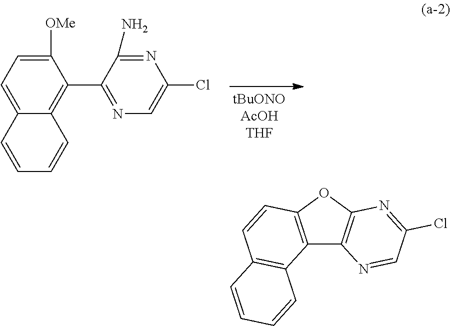 [SMILES: COc1ccc2ccccc2c1-c1ncc(Cl)nc1N ; Clc1cnc2c(n1)oc1ccc3ccccc3c12]

### Step 3: Synthesis of 9-[(3′-dibenzothiophen-4-yl)biphenyl-3-yl]naphtho[1′,2′:4,5]furo[2,3-b]pyrazine (Abbreviation: 9mDBtBPNfpr)

Into a three-neck flask were put 1.48 g of 9-chloronaphtho[1′,2′:4,5]furo[2,3-b]pyrazine obtained in Step 2, 3.41 g of 3′-(4-dibenzothiophene)-1,1′-biphenyl-3-boronic acid, 8.8 mL of a 2M potassium carbonate aqueous solution, 100 mL of toluene, and 10 mL of ethanol, and the air in the flask was replaced with nitrogen. The mixture in the flask was degassed by being stirred under reduced pressure, and then 0.84 g of bis(triphenylphosphine)palladium(II) dichloride (abbreviation: Pd(PPh₃)₂Cl₂) was added thereto. The mixture was stirred at 80° C. for 18 hours to be reacted.

After a predetermined time elapsed, the obtained suspension was subjected to suction filtration and was washed with water and ethanol. The obtained solid was dissolved in toluene, and the mixture was filtered through a filter aid in which Celite, alumina, and Celite were stacked in this order and was recrystallized with a mixed solvent of toluene and hexane, so that 2.66 g of a target pale yellow solid was obtained in a yield of 82%.

By a train sublimation method, 2.64 g of the obtained pale yellow solid was purified by sublimation. In the purification by sublimation, the solid was heated at 315° C. under a pressure of 2.6 Pa with an argon gas flow rate of 15 m/min. After the purification by sublimation, 2.34 g of a target pale yellow solid was obtained in a yield of 89%. A synthesis scheme of Step 3 is shown in (a-3) below.

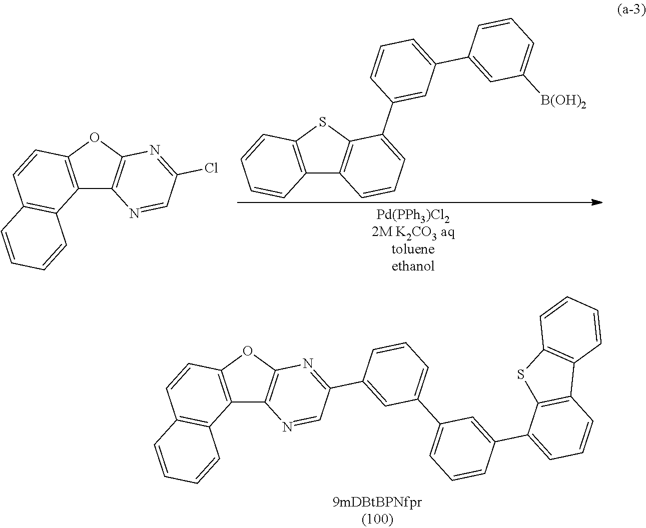

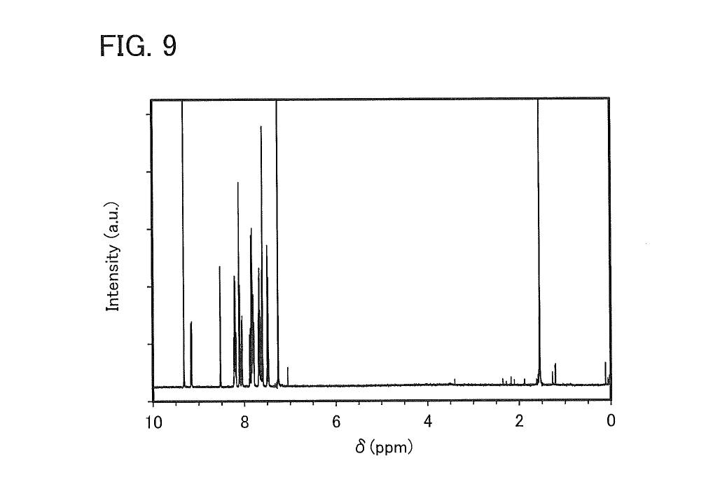

Analysis results by nuclear magnetic resonance (¹H-NMR) spectroscopy of the pale yellow solid obtained in Step 3 are shown below. FIG. 9 is the ¹H-NMR chart. The results revealed that 9mDBtBPNfpr, the organic compound represented by Structural Formula (100), was obtained in this example.

¹H-NMR. δ (CD₂Cl₂): 7.47-7.51 (m, 2H), 7.60-7.69 (m, 5H), 7.79-7.89 (m, 6H), 8.05 (d, 1H), 8.10-8.11 (m, 2H), 8.18-8.23 (m, 3H), 8.53 (s, 1H), 9.16 (d, 1H), 9.32 (s, 1H).

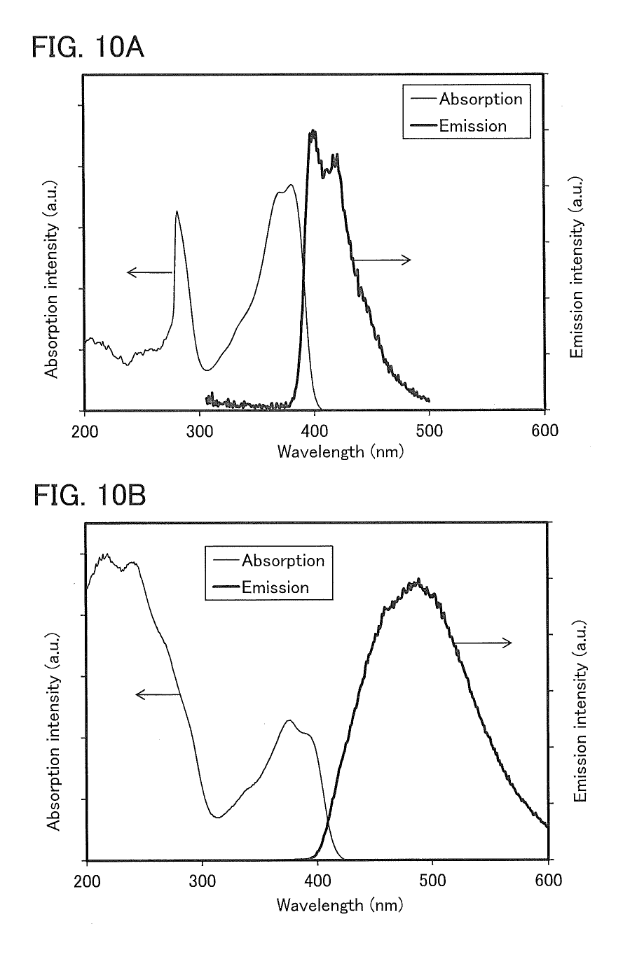

FIG. 10A shows an ultraviolet-visible absorption spectrum (hereinafter, simply referred to as “absorption spectrum”) and an emission spectrum of 9mDBtBPNfpr in a toluene solution. The horizontal axis represents wavelength and the vertical axes represent absorption intensity and emission intensity.

The absorption spectrum was measured with an ultraviolet-visible spectrophotometer (V-550, produced by JASCO Corporation). To calculate the absorption spectrum of 9mDBtBPNfpr in a toluene solution, the absorption spectrum of toluene put in a quartz cell was measured and then subtracted from the absorption spectrum of a toluene solution of 9mDBtBPNfpr put in a quartz cell. The emission spectrum was measured with a fluorescence spectrophotometer (FS920 produced by Hamamatsu Photonics K.K.). The emission spectrum of 9mDBtBPNfpr in the toluene solution was measured with the toluene solution of 9mDBtBPNfpr put in a quartz cell.

FIG. 10A shows that 9mDBtBPNfpr in the toluene solution has absorption peaks at around 370 nm and 380 nm and emission wavelength peaks at around 400 nm and 421 nm (the excitation wavelength: 291 nm).

Next, the absorption spectrum and the emission spectrum of a solid thin film of 9mDBtBPNfpr were measured. The solid thin film was fabricated over a quartz substrate by a vacuum evaporation method. The absorption spectrum of the thin film was calculated using an absorbance (−log₁₀ [% T/(100−% R)]) obtained from the transmittance and reflectance of the thin film including the substrate. Note that % T represents transmittance and % R represents reflectance. The absorption spectrum was measured with a UV-visible spectrophotometer (U-4100 produced by Hitachi High-Technologies Corporation). The emission spectrum was measured with a fluorescence spectrophotometer (FS920 produced by Hamamatsu Photonics K.K.). The obtained absorption and emission spectra of the solid thin film are shown in FIG. 10B. The horizontal axis represents wavelength and the vertical axes represent absorption intensity and emission intensity.

FIG. 10B shows that the solid thin film of 9mDBtBPNfpr has absorption peaks at around 377 nm and 395 nm and an emission wavelength peak at around 489 nm (the excitation wavelength: 370 nm).

Accordingly, 9mDBtBPNfpr, the organic compound of one embodiment of the present invention, is a host material that is suitably used with a phosphorescent material that emits light with energy at a wavelength longer than or equal to that of red light. Note that 9mDBtBPNfpr, the organic compound of one embodiment of the present invention, can also be used as a host material for a substance that emits phosphorescence in the visible region or a light-emitting substance.

Next, the LUMO level of 9mDBtBPNfpr is described. The LUMO level was estimated from the values of a reduction potential and potential energy (approximately −4.94 eV with respect to the vacuum level) of a reference electrode (Ag/Ag⁺), which were obtained by cyclic voltammetry (CV) measurement in a dimethylformamide solvent. Specifically, −4.94 [eV]−(the value of the reduction potential)=the LUMO level. The measured LUMO level calculated using the above formula was −3.05 eV. This indicates that 9mDBtBPNfpr accepts electrons easily and has high electron stability.

### Example 2

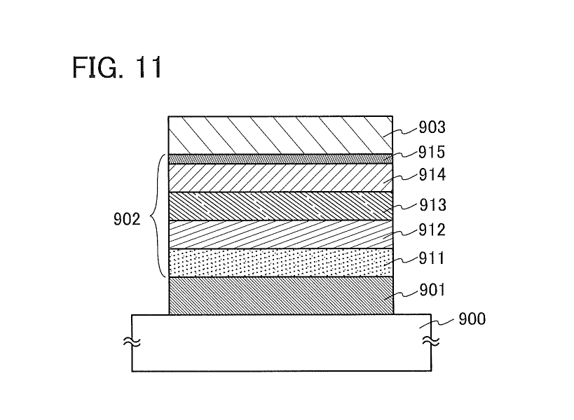

This example describes element structures, fabrication methods, and characteristics of a light-emitting element 1 (light-emitting element of one embodiment of the present invention) in which 9-[(3′-dibenzothiophen-4-yl)biphenyl-3-yl]naphtho[1′,2′:4,5]furo[2,3-b]pyrazine (abbreviation: 9mDBtBPNfpr) (Structural Formula (100)) described in Example 1 is used in a light-emitting layer and a comparative light-emitting element 2 in which 2-[3′-(dibenzothiophen-4-yl)biphenyl-3-yl]dibenzo[f,h]quinoxaline (abbreviation: 2mDBTBPDBq-II) is used in a light-emitting layer. Note that FIG. 11 illustrates an element structure of a light-emitting element used in this example, and Table 1 shows specific structures. Chemical formulae of materials used in this example are shown below.

TABLE 1

| | | | Hole- | Light- | | | Electron- | |
|---|---|---|---|---|---|---|---|---|
| | First | Hole-injection | transport | emitting | | | injection | Second |
| | electrode | layer | layer | layer | Electron-transport layer | | layer | electrode |
| | 901 | 911 | 912 | 913 | 914 | | 915 | 903 |
| Light- | ITSO | DBT3P-II:MoOx | BPAFLP | * | 9mDBtBPNfpr | NBphen | LiF | Al |
| emitting | (70 nm) | (2:1, 75 nm) | (20 nm) | | (30 nm) | (15 nm) | (1 nm) | (200 nm) |
| element 1 | | | | | | | | |
| Comparative | ITSO | DBT3P-II:MoOx | BPAFLP | ** | 2mDBTBPDBq-II | NBphen | LiF | Al |
| light-emitting | (70 nm) | (2:1, 75 nm) | (20 nm) | | (30 nm) | (15 nm) | (1 nm) | (200 nm) |
| element 2 | | | | | | | | |

* 9mDBtBPNfpr:PCBBiF:[Ir(dmdppr-P)₂(dibm)] (0.75:0.25:0.1, 40 nm)
** 2mDBTBPDBq-II:PCBBiF:[Ir(dmdppr-P)₂(dibm)] (0.75:0.25:0.1, 40 nm)

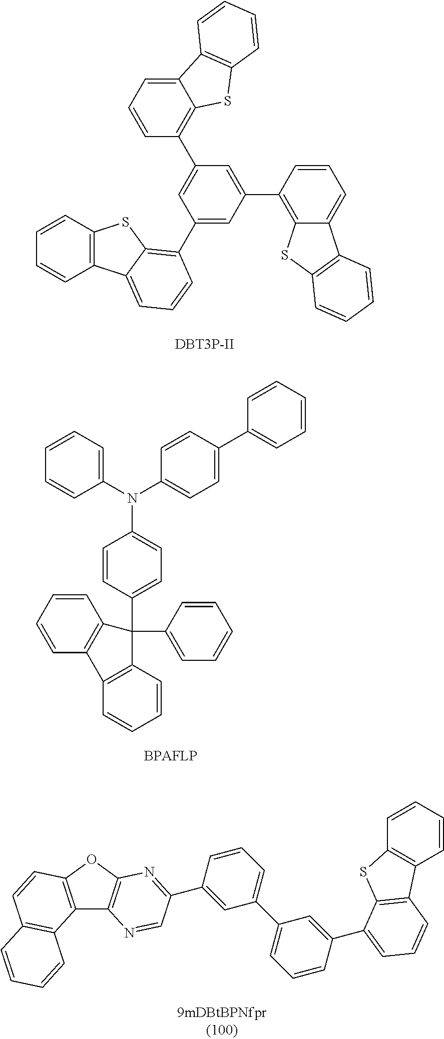

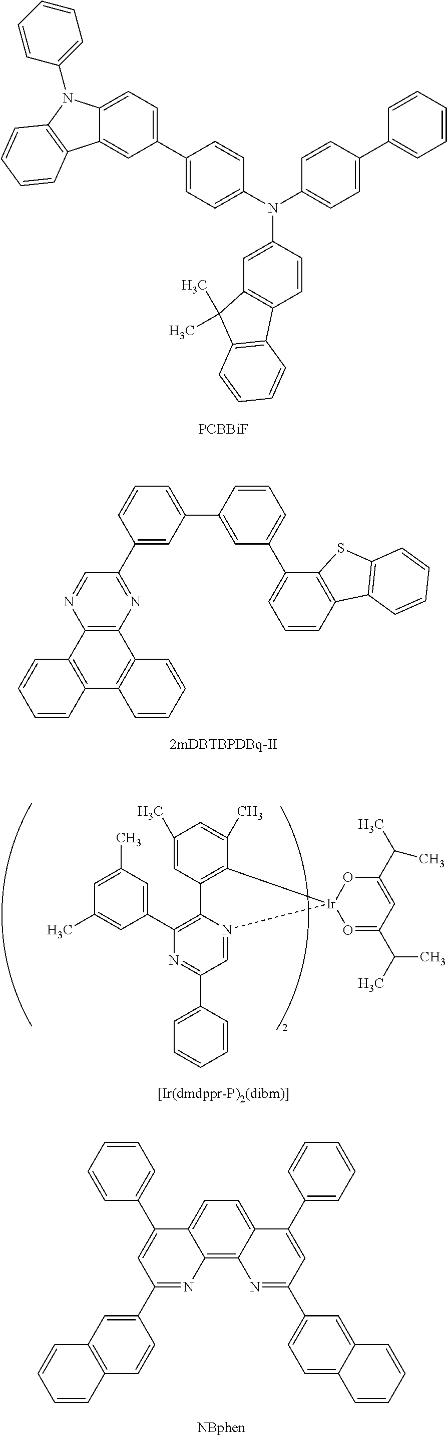

### <<Fabrication of Light-Emitting Elements>>

In each of the light-emitting elements described in this example, as illustrated in FIG. 11, a hole-injection layer 911, a hole-transport layer 912, a light-emitting layer 913, an electron-transport layer 914, and an electron-injection layer 915 are stacked in this order over a first electrode 901 formed over a substrate 900, and a second electrode 903 is stacked over the electron-injection layer 915.

First, the first electrode 901 was formed over the substrate 900. The electrode area was set to 4 mm² (2 mm×2 mm). A glass substrate was used as the substrate 900. The first electrode 901 was formed to a thickness of 70 nm using indium tin oxide containing silicon oxide (ITSO) by a sputtering method.

As pretreatment, a surface of the substrate was washed with water, baking was performed at 200° C. for 1 hour, and then UV ozone treatment was performed for 370 seconds. After that, the substrate was transferred into a vacuum evaporation apparatus where the pressure was reduced to approximately 10⁻⁴ Pa, vacuum baking was performed at 170° C. for 30 minutes in a heating chamber of the vacuum evaporation apparatus, and then the substrate was cooled down for approximately 30 minutes.

Next, the hole-injection layer 911 was formed over the first electrode 901. After the pressure in the vacuum evaporation apparatus was reduced to 10⁻⁴ Pa, the hole-injection layer 911 was formed by co-evaporation to have a mass ratio of 1,3,5-tri(dibenzothiophen-4-yl)benzene (abbreviation: DBT3P-II) to molybdenum oxide of 2:1 and a thickness of 75 nm.

Then, the hole-transport layer 912 was formed over the hole-injection layer 911. The hole-transport layer 912 was formed to a thickness of 20 nm by evaporation of 4-phenyl-4′-(9-phenylfluoren-9-yl)triphenylamine (abbreviation: BPAFLP).

Next, the light-emitting layer 913 was formed over the hole-transport layer 912.

The light-emitting layer 913 in the light-emitting element 1 was formed in the following manner: 9mDBtBPNfpr, N-(1,1′-biphenyl-4-yl)-N-[4-(9-phenyl-9H-carbazol-3-yl)phenyl]-9,9-dimethyl-9H-fluor en-2-amine (abbreviation: PCBBiF), and bis{4,6-dimethyl-2-[3-(3,5-dimethylphenyl)-5-phenyl-2-pyrazinyl-N]phenyl-κC}(2,6-dimethyl-3,5-heptanedionato-κ²O,O′)iridium(III) (abbreviation: [Ir(dmdppr-P)₂(dibm)]), which was used as a guest material (phosphorescent light-emitting material), were deposited by co-evaporation to have a weight ratio of 9mDBtBPNfpr to PCBBiF and [Ir(dmdppr-P)₂(dibm)] of 0.75:0.25:0.1. The thickness was set to 40 nm. The light-emitting layer 913 in the comparative light-emitting element 2 was formed in the following manner: 2mDBTBPDBq-II, PCBBiF, and [Ir(dmdppr-P)₂(dibm)], which was used as a guest material (phosphorescent light-emitting material), were deposited by co-evaporation to have a weight ratio of 2mDBTBPDBq-II to PCBBiF and [Ir(dmdppr-P)₂(dibm)] of 0.75:0.25:0.1. The thickness was set to 40 nm.

Next, the electron-transport layer 914 was formed over the light-emitting layer 913. The electron-transport layer 914 in the light-emitting element 1 was formed in the following manner: 9mDBtBPNfpr and 2,9-bis(naphthalen-2-yl)-4,7-diphenyl-1,10-phenanthroline (abbreviation: NBphen) were sequentially deposited by evaporation to thicknesses of 30 nm and 15 nm, respectively. The electron-transport layer 914 in the comparative light-emitting element 2 was formed in the following manner: 2mDBTBPDBq-II and NBphen were sequentially deposited by evaporation to thicknesses of 30 nm and 15 nm, respectively.

Then, the electron-injection layer 915 was formed over the electron-transport layer 914. The electron-injection layer 915 was formed to a thickness of 1 nm by evaporation of lithium fluoride (LiF).

After that, the second electrode 903 was formed over the electron-injection layer 915. The second electrode 903 was formed using aluminum to a thickness of 200 nm by an evaporation method. In this example, the second electrode 903 functioned as a cathode.

Through the above steps, the light-emitting elements each including an EL layer between a pair of electrodes were formed over the substrate 900. The hole-injection layer 911, the hole-transport layer 912, the light-emitting layer 913, the electron-transport layer 914, and the electron-injection layer 915 described above were functional layers forming the EL layer of one embodiment of the present invention. Furthermore, in all the evaporation steps in the above fabrication method, evaporation was performed by a resistance-heating method.

Each of the light-emitting elements fabricated as described above was sealed using another substrate (not illustrated) in such a manner that the substrate (not illustrated) with an ultraviolet curable sealant was fixed to the substrate 900 in a glove box containing a nitrogen atmosphere, and the substrates were bonded to each other with the sealant attached to the periphery of the light-emitting element formed over the substrate 900. At the time of the sealing, the sealant was irradiated with 365-nm ultraviolet light at 6 J/cm² to be solidified, and the sealant was heated at 80° C. for 1 hour to be stabilized.

### <<Operation Characteristics of Light-Emitting Elements>>

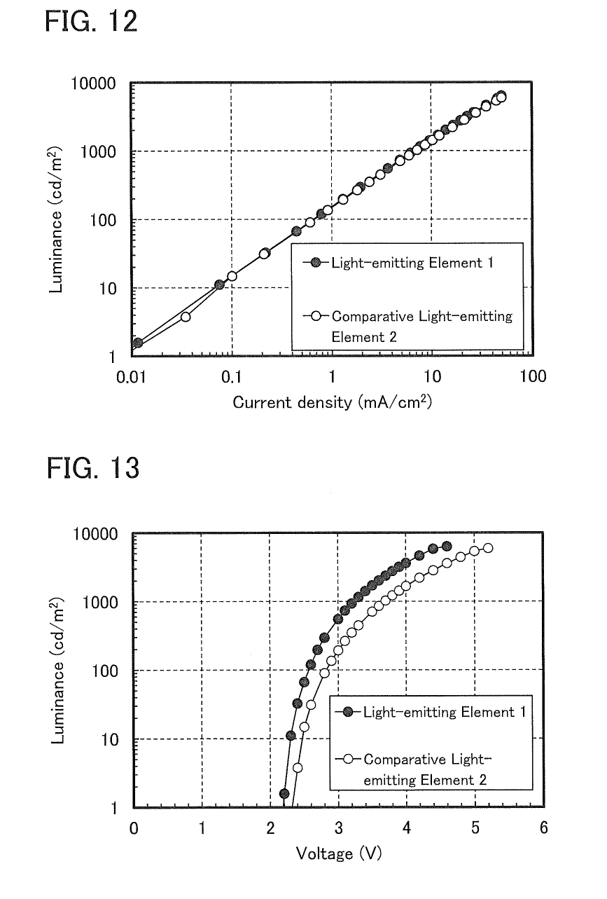

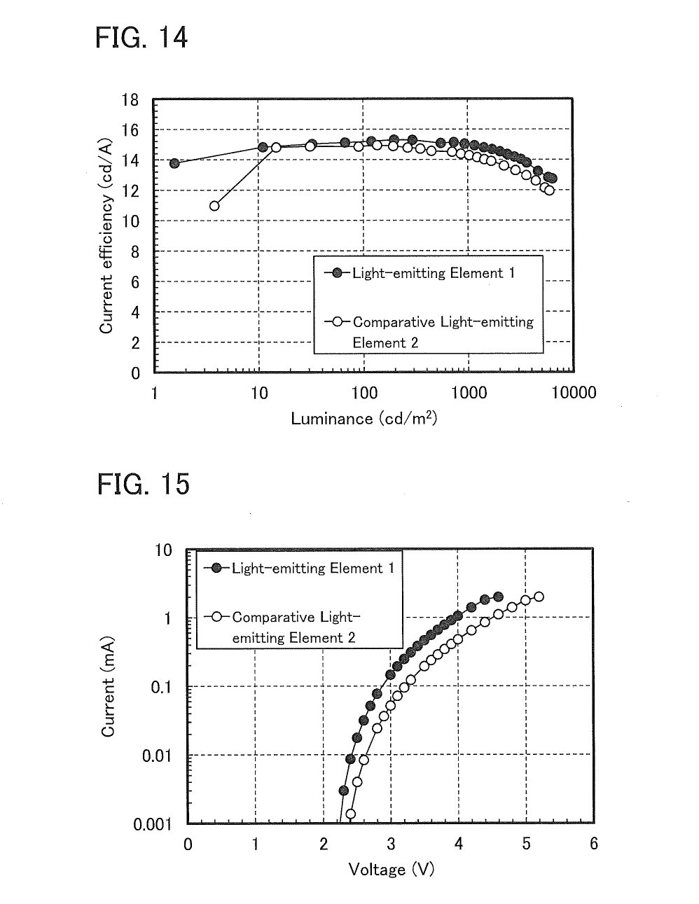

Operation characteristics of the fabricated light-emitting elements were measured. Note that the measurement was performed at room temperature (in an atmosphere kept at 25° C.). As the results of the operation characteristics of the light-emitting elements, the current density-luminance characteristics are shown in FIG. 12, the voltage-luminance characteristics are shown in FIG. 13, the luminance-current efficiency characteristics are shown in FIG. 14, and the voltage-current characteristics are shown in FIG. 15.

Table 2 shows initial values of main characteristics of the light-emitting elements at around 1000 cd/m².

TABLE 2

| | | | | | | | | External |
|---|---|---|---|---|---|---|---|---|
| | | | Current | | | Current | Power | quantum |
| | Voltage | Current | density | Chromaticity | Luminance | efficiency | efficiency | efficiency |
| | (V) | (mA) | (mA/cm²) | (x, y) | (cd/m²) | (cd/A) | (lm/W) | (%) |
| Light-emitting | 3.2 | 0.25 | 6.2 | (0.71, 0.29) | 930 | 15 | 15 | 26 |
| element 1 | | | | | | | | |
| Comparative | 3.7 | 0.29 | 7.2 | (0.71, 0.29) | 1000 | 14 | 12 | 25 |
| light-emitting | | | | | | | | |
| element 2 | | | | | | | | |

The above results show that the light-emitting element 1 fabricated in this example has high efficiency.

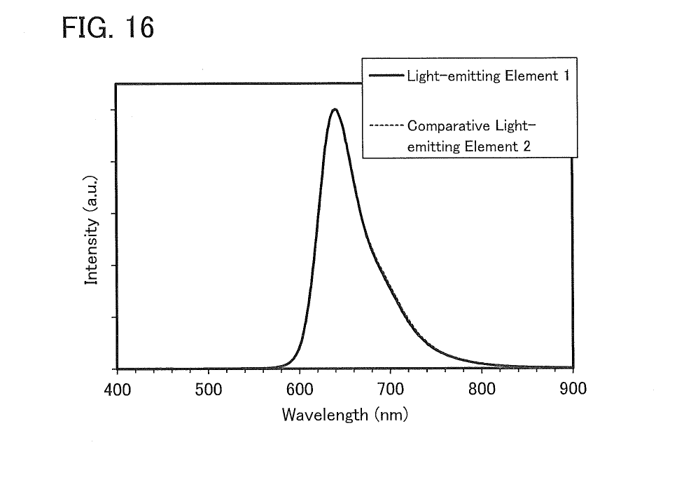

FIG. 16 shows emission spectra when current at a current density of 2.5 mA/cm² was applied to the light-emitting element 1 and the comparative light-emitting element 2. As shown in FIG. 16, the emission spectrum of each of the light-emitting element 1 and the comparative light-emitting element 2 has a peak at around 640 nm that is probably derived from light emission of [Ir(dmdppr-P)₂(dibm)] contained in the light-emitting layer 913.

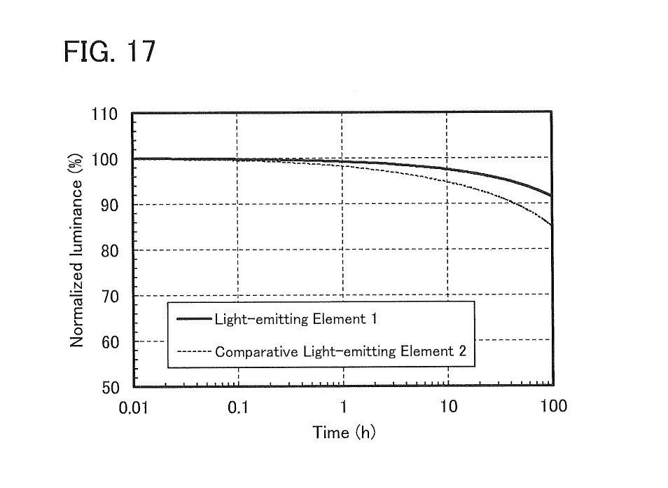

Next, reliability tests were performed on the light-emitting element 1 and the comparative light-emitting element 2. FIG. 17 shows results of the reliability tests. In FIG. 17, the vertical axis represents normalized luminance (%) with an initial luminance of 100%, and the horizontal axis represents driving time (h) of the elements. As the reliability tests, constant current driving tests at a constant current density of 50 mA/cm² were performed.

The results of the reliability tests show that the light-emitting element 1 has higher reliability than the comparative light-emitting element 2. This is probably derived from a difference in molecular structures between 9mDBtBPNfpr and 2mDBTBPDBq-II, that is, a difference between a naphthofuropyrazine skeleton and a dibenzoquinoxaline skeleton, thus showing robustness of a furopyrazine derivative of one embodiment of the present invention. Accordingly, it is indicated that the use of 9mDBtBPNfpr (Structural Formula (100)), which is the organic compound of one embodiment of the present invention, is effective in improving the element characteristics of a light-emitting element.

### Example 3

In this example, a light-emitting element 3 using 9mDBtBPNfpr (Structural Formula (100), Example 1) in its light-emitting layer was fabricated as a light-emitting element of one embodiment of the present invention. The measured characteristic results of the light-emitting element 3 will be described below.

Note that the first electrode 901 and the hole-injection layer 911 of the light-emitting element 3 were formed in the same manner as those of the light-emitting element 1 in Example 2.

The hole-transport layer 912 was formed over the hole-injection layer 911 to a thickness of 20 nm by evaporation of 4,4′-diphenyl-4″-(9-phenyl-9H-carbazol-3-yl)triphenylamine (abbreviation: PCBBi1BP).

The light-emitting layer 913 was formed over the hole-transport layer 912 in the following manner: 9mDBtBPNfpr, PCBBiF, and bis[4,6-dimethyl-2-(2-quinolinyl-κN)phenyl-κC](2,4-pentanedionato-κ²O,O′)iridium(III) (abbreviation: [Ir(dmpqn)₂(acac)]), which was used as a guest material (phosphorescent light-emitting material), were deposited by co-evaporation to have a weight ratio of 9mDBtBPNfpr to PCBBiF and [Ir(dmpqn)₂(acac)] of 0.8:0.2:0.1. The thickness was set to 40 nm.

The electron-transport layer 914 was formed over the light-emitting layer 913 in the following manner: 9mDBtBPNfpr and NBphen were sequentially deposited by evaporation to thicknesses of 30 nm and 15 nm, respectively.

The electron-injection layer 915 and the second electrode 903 were formed in the same manner as those of the light-emitting element 1 in Example 2; thus, the description thereof is omitted. Table 3 shows a specific element structure of the light-emitting element 3. Chemical formulae of materials used in this example are shown below.

TABLE 3

| | | | Hole- | Light- | | | Electron- | |
|---|---|---|---|---|---|---|---|---|
| | First | Hole-injection | transport | emitting | | | injection | Second |
| | electrode | layer | layer | layer | Electron-transport layer | | layer | electrode |
| | 901 | 911 | 912 | 913 | 914 | | 915 | 903 |
| Light- | ITSO | DBT3P-II:MoOx | PCBBi1BP | * | 9mDBtBPNfpr | NBphen | LiF | Al |
| emitting | (70 nm) | (2:1, 70 nm) | (20 nm) | | (30 nm) | (15 nm) | (1 nm) | (200 nm) |
| element 3 | | | | | | | | |

* 9mDBtBPNfpr:PCBBiF:[Ir(dmpqn)₂(acac)] (0.8:0.2:0.1, 40 nm)

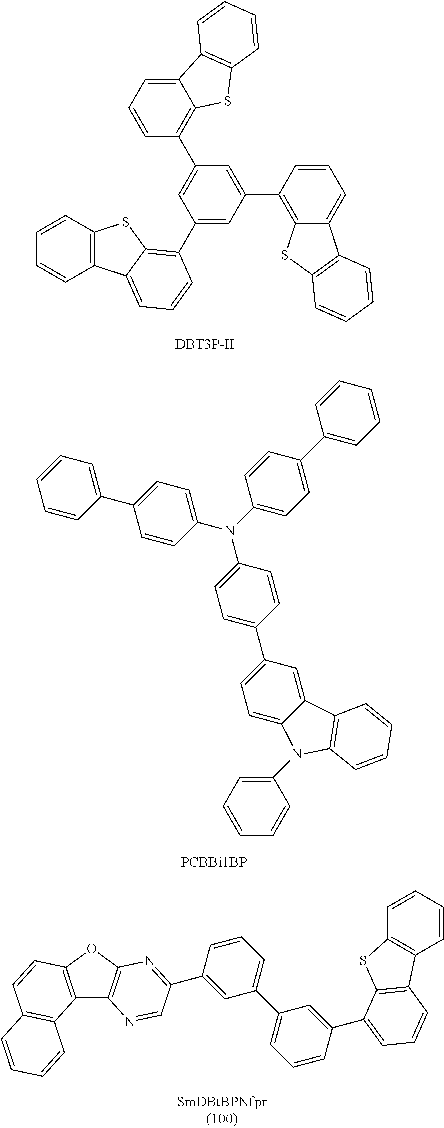

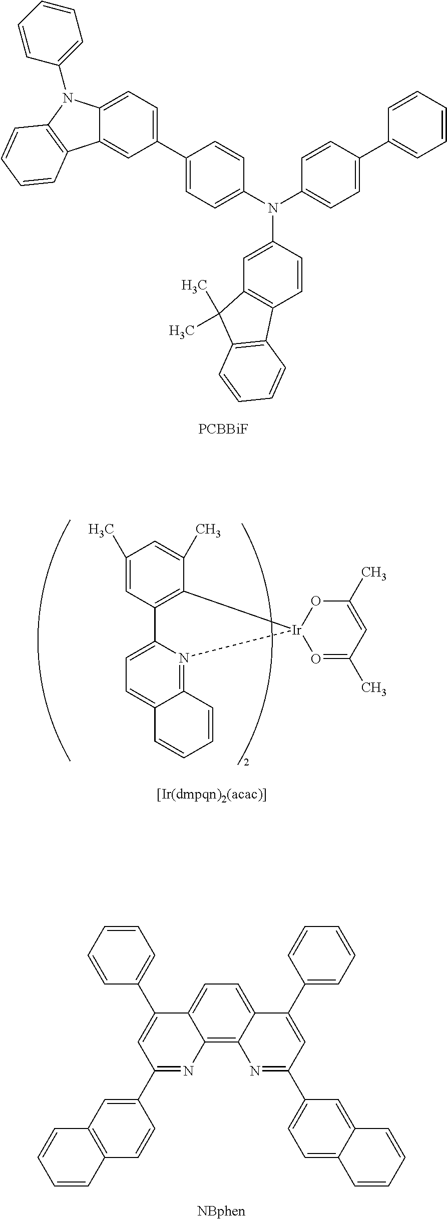

### <<Operation Characteristics of Light-Emitting Element 3>>

Operation characteristics of the fabricated light-emitting element 3 were measured. Note that the measurement was performed at room temperature (in an atmosphere kept at 25° C.).

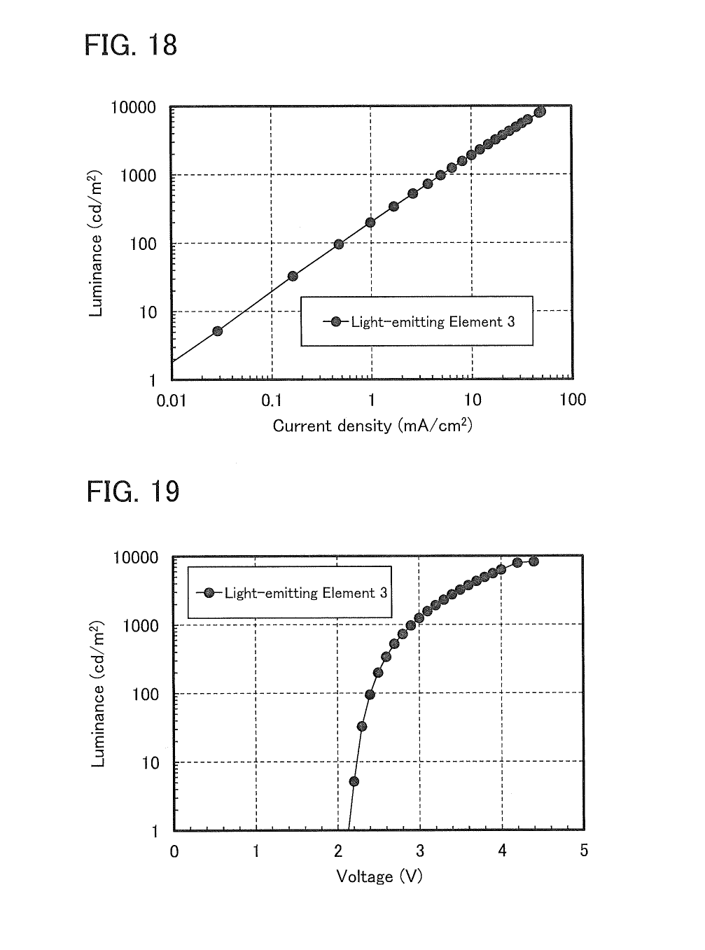

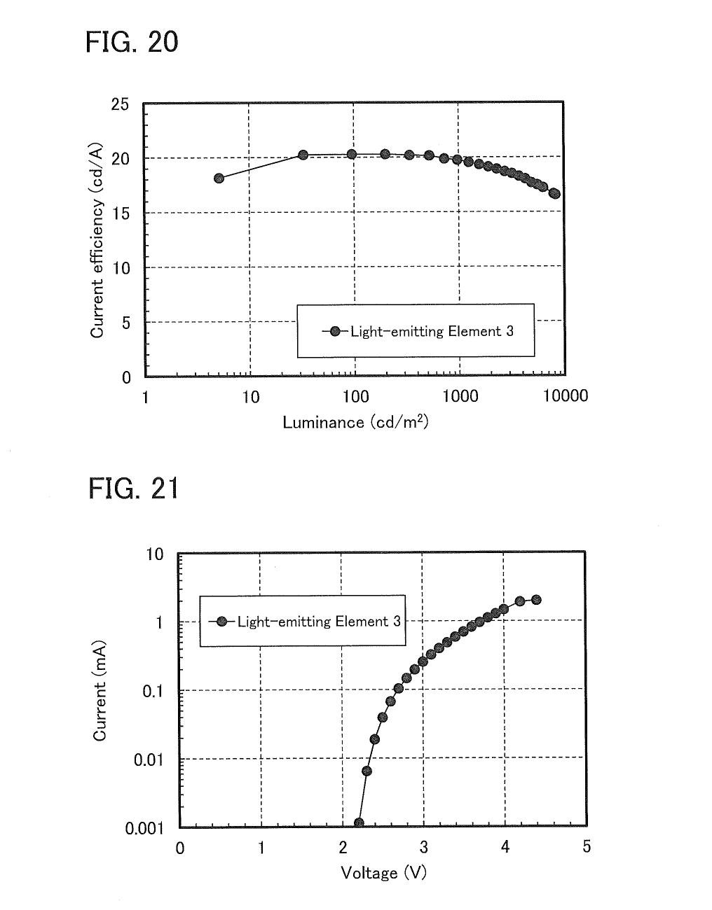

FIG. 18, FIG. 19, FIG. 20, and FIG. 21 show the current density-luminance characteristics, the voltage-luminance characteristics, the luminance-current efficiency characteristics, and the voltage-current characteristics, respectively, of the light-emitting element 3.

Table 4 shows initial values of main characteristics of the light-emitting element 3 at around 1000 cd/m².

TABLE 4

| | | | | | | | | External |
|---|---|---|---|---|---|---|---|---|
| | | | Current | | | Current | Power | quantum |
| | Voltage | Current | density | Chromaticity | Luminance | efficiency | efficiency | efficiency |
| | (V) | (mA) | (mA/cm²) | (x, y) | (cd/m²) | (cd/A) | (lm/W) | (%) |
| Light- | 2.9 | 0.20 | 4.9 | (0.68, 0.32) | 970 | 20 | 21 | 21 |
| emitting | | | | | | | | |
| element 3 | | | | | | | | |

The above results show that the light-emitting element 3 fabricated in this example has high efficiency.

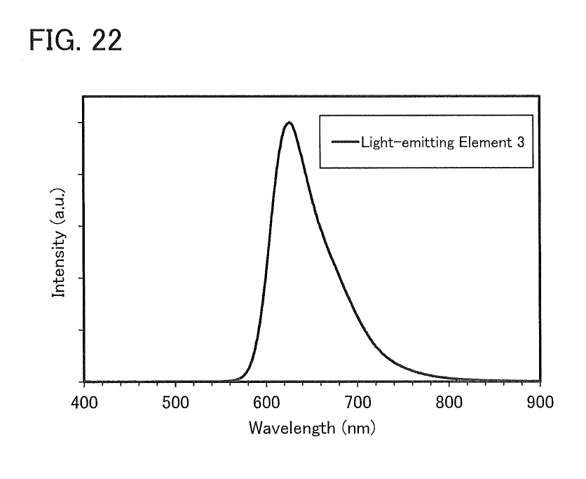

FIG. 22 shows an emission spectrum when current at a current density of 2.5 mA/cm² was applied to the light-emitting element 3. As shown in FIG. 22, the emission spectrum of the light-emitting element has a peak at around 626 nm that is probably derived from light emission of [Ir(dmpqn)₂(acac)] contained in the light-emitting layer 913.

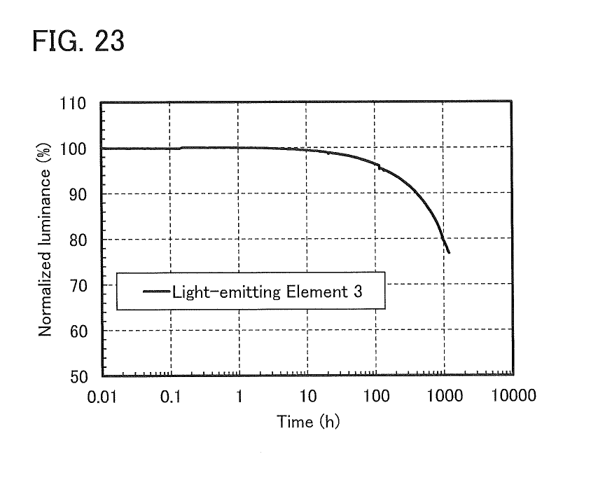

Next, a reliability test was performed on the light-emitting element 3. FIG. 23 shows results of the reliability test. In FIG. 23, the vertical axis represents normalized luminance (%) with an initial luminance of 100%, and the horizontal axis represents driving time (h) of the element. As the reliability test, a constant current driving test at a constant current density of 75 mA/cm² was performed.

The results of the reliability test show that the light-emitting element 3 has high reliability. This indicates that the use of 9mDBtBPNfpr (Structural Formula (100)), which is the organic compound of one embodiment of the present invention, is effective in improving the element characteristics of a light-emitting element.

### Example 4

In this example, a light-emitting element 4 using 9mDBtBPNfpr (Structural Formula (100), Example 1) in its light-emitting layer was fabricated as a light-emitting element of one embodiment of the present invention. The measured characteristic results of the light-emitting element 4 will be described below.

Note that the first electrode 901 and the hole-injection layer 911 of the light-emitting element 4 were formed in the same manner as those of the light-emitting element 1 in Example 2.

The hole-transport layer 912 was formed over the hole-injection layer 911 to a thickness of 20 nm by evaporation of PCBBiF.

The light-emitting layer 913 was formed over the hole-transport layer 912 in the following manner: 9mDBtBPNfpr, PCBBiF, and bis{4,6-dimethyl-2-[5-(5-cyano-2-methylphenyl)-3-(3,5-dimethylphenyl)-2-pyrazinyl-κN]phenyl-κC}(2,2,6,6-tetramethyl-3,5-heptanedionato-κ²O,O′)iridium(III) (abbreviation: [Ir(dmdppr-m5CP)₂(dpm)]), which was used as a guest material (phosphorescent light-emitting material), were deposited by co-evaporation to have a weight ratio of 9mDBtBPNfpr to PCBBiF and [Ir(dmdppr-m5CP)₂(dpm)] of 0.8:0.2:0.1. The thickness was set to 40 nm.

The electron-transport layer 914 was formed over the light-emitting layer 913 in the following manner 9mDBtBPNfpr and NBphen were sequentially deposited by evaporation to thicknesses of 30 nm and 15 nm, respectively.

The electron-injection layer 915 and the second electrode 903 were formed in the same manner as those of the light-emitting element 1 in Example 2; thus, the description thereof is omitted. Table 5 shows a specific element structure of the light-emitting element 4. Chemical formulae of materials used in this example are shown below.

TABLE 5

| | | | Hole- | Light- | | | Electron- | |
|---|---|---|---|---|---|---|---|---|
| | First | Hole-injection | transport | emitting | | | injection | Second |
| | electrode | layer | layer | layer | Electron-transport layer | | layer | electrode |
| | 901 | 911 | 912 | 913 | 914 | | 915 | 903 |
| Light- | ITSO | DBT3P-II:MoOx | PCBBiF | * | 9mDBtBPNfpr | NBphen | LiF | Al |
| emitting | (70 nm) | (2:1, 75 nm) | (20 nm) | | (30 nm) | (15 nm) | (1 nm) | (200 nm) |
| element 4 | | | | | | | | |

* 9mDBtBPNfpr:PCBBiF:[Ir(dmdppr-m5CP)₂(dpm)] (0.8:0.2:0.1, 40 nm)

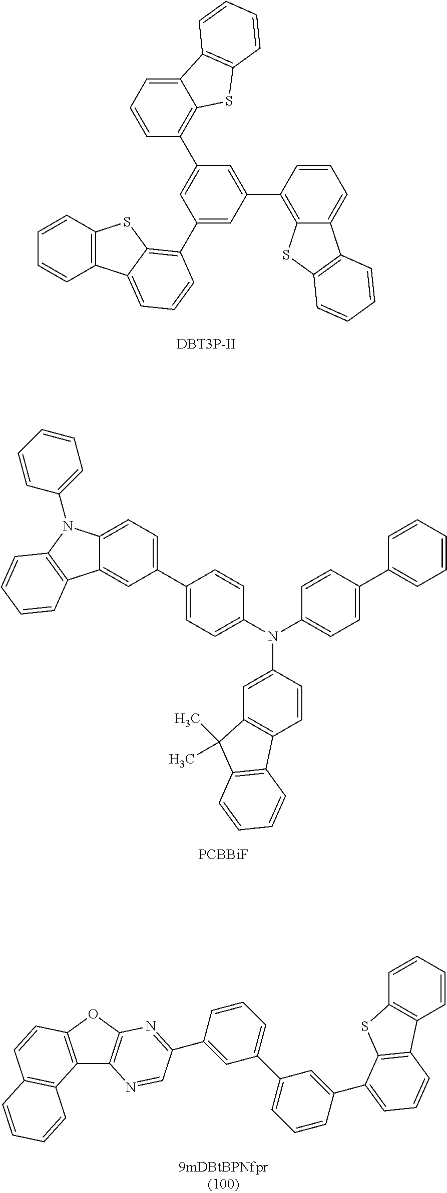

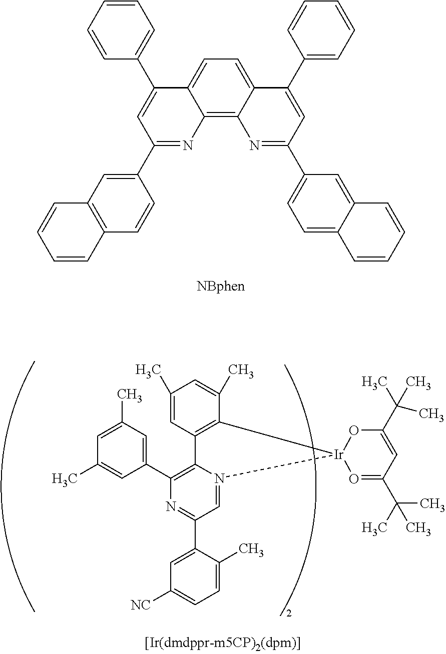

### <<Operation Characteristics of Light-Emitting Element 4>>

Operation characteristics of the fabricated light-emitting element 4 were measured. Note that the measurement was performed at room temperature (in an atmosphere kept at 25° C.).

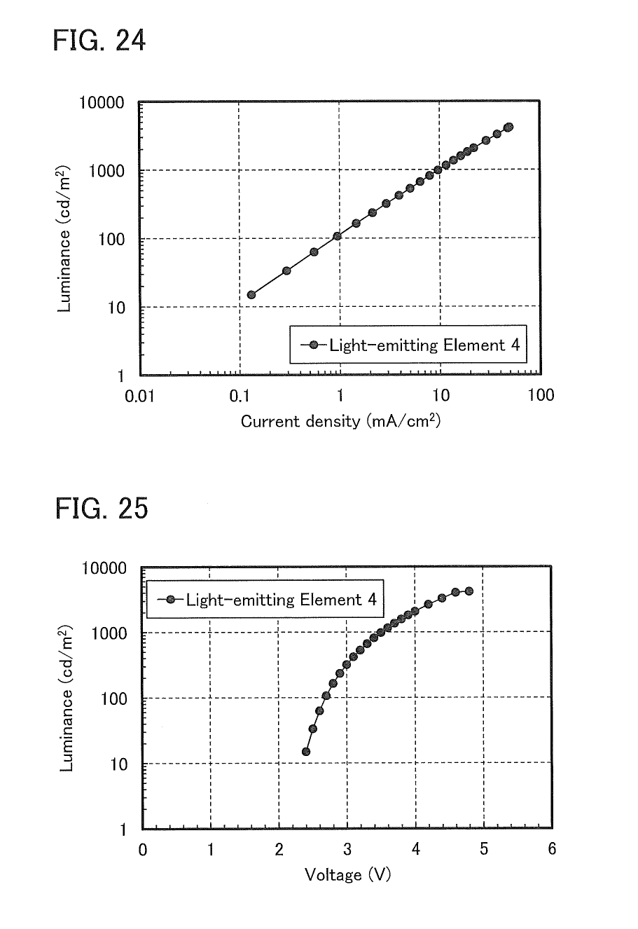

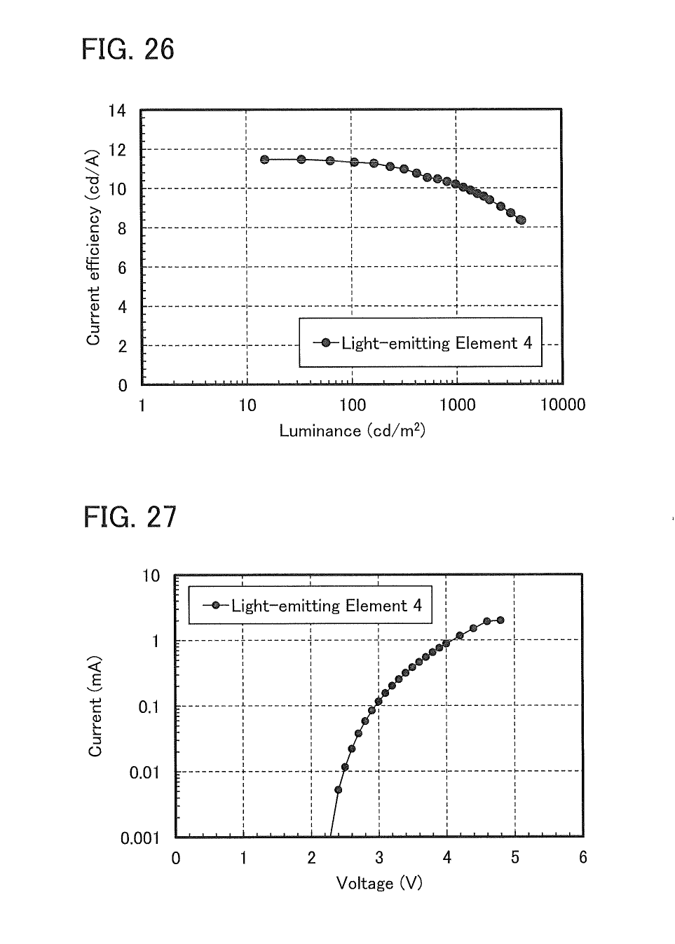

FIG. 24, FIG. 25, FIG. 26, and FIG. 27 show the current density-luminance characteristics, the voltage-luminance characteristics, the luminance-current efficiency characteristics, and the voltage-current characteristics, respectively, of the light-emitting element 4.

Table 6 shows initial values of main characteristics of the light-emitting element 4 at around 1000 cd/m².

TABLE 6

| | | | | | | | | External |
|---|---|---|---|---|---|---|---|---|
| | | | Current | | | Current | Power | quantum |
| | Voltage | Current | density | Chromaticity | Luminance | efficiency | efficiency | efficiency |
| | (V) | (mA) | (mA/cm²) | (x, y) | (cd/m²) | (cd/A) | (lm/W) | (%) |
| Light- | 3.5 | 0.39 | 9.7 | (0.71, 0.29) | 980 | 10 | 9.2 | 23 |
| emitting | | | | | | | | |
| element 4 | | | | | | | | |

The above results show that the light-emitting element 4 fabricated in this example has high efficiency.

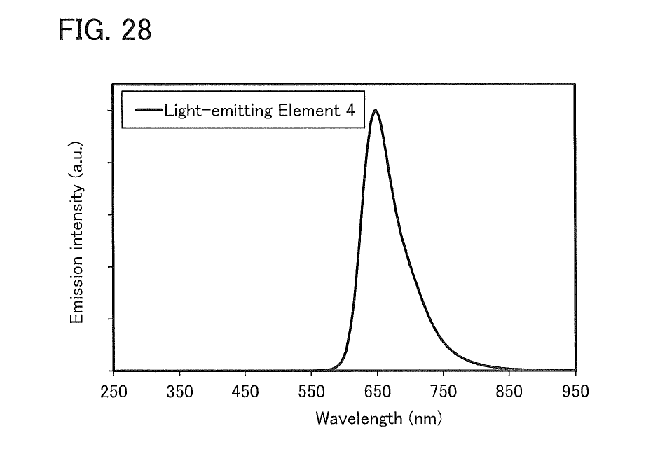

FIG. 28 shows an emission spectrum when current at a current density of 2.5 mA/cm² was applied to the light-emitting element 4. As shown in FIG. 28, the emission spectrum of the light-emitting element has a peak at around 648 nm that is probably derived from light emission of [Ir(dmdppr-m5CP)₂(dpm)] contained in the light-emitting layer 913.

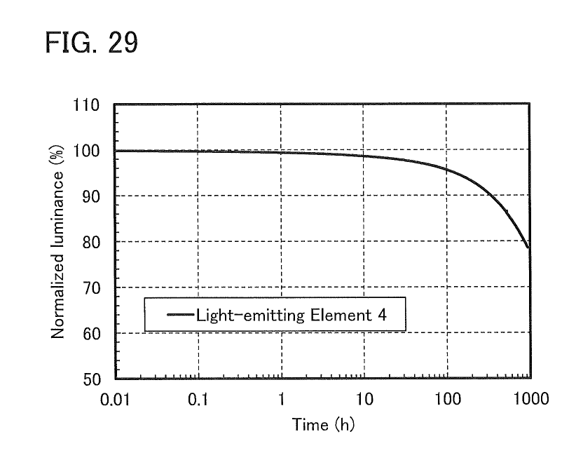

Next, a reliability test was performed on the light-emitting element 4. FIG. 29 shows results of the reliability test. In FIG. 29, the vertical axis represents normalized luminance (%) with an initial luminance of 100%, and the horizontal axis represents driving time (h) of the element. As the reliability test, a constant current driving test at a constant current density of 75 mA/cm² was performed.

The results of the reliability test show that the light-emitting element 4 has high reliability. This indicates that the use of 9mDBtBPNfpr (Structural Formula (100)), which is the organic compound of one embodiment of the present invention, is effective in improving the element characteristics of a light-emitting element.

### Example 5

In this example, a light-emitting element 5 using 9mDBtBPNfpr (Structural Formula (100), Example 1) in its light-emitting layer was fabricated as a light-emitting element of one embodiment of the present invention. The measured characteristic results of the light-emitting element 5 will be described below.

Table 7 shows a specific element structure of the light-emitting element 5. In the table, APC represents an alloy of silver, palladium, and copper (Ag—Pd—Cu). Refer to FIG. 11 for the stacked-layer structure of the light-emitting element. Note that the light-emitting element 5 also included a cap layer in contact with the second electrode 903. Chemical formulae of materials used in this example are shown below.

TABLE 7

| | | | Hole- | Light- | | | Electron- | | |
|---|---|---|---|---|---|---|---|---|---|
| | First | Hole-injection | transport | emitting | | | injection | Second | |
| | electrode | layer | layer | layer | Electron-transport layer | | layer | electrode | Cap layer |
| | 901 | 911 | 912 | 913 | 914 | | 915 | 903 | — |
| Light- | APC\ITSO | DBT3P-II:MoOx | PCBBiF | * | 9mDBtBPNfpr | NBphen | LiF | Ag:Mg | DBT3P-II |
| emitting | (110 nm) | (2:1, 70 nm) | (15 nm) | | (30 nm) | (20 nm) | (1 nm) | (25 nm) | (70 nm) |
| element 5 | | | | | | | | | |

* 9mDBtBPNfpr:PCBBiF:[Ir(dmdppr-m5CP)₂(dpm)] (0.8:0.2:0.04, 40 nm)

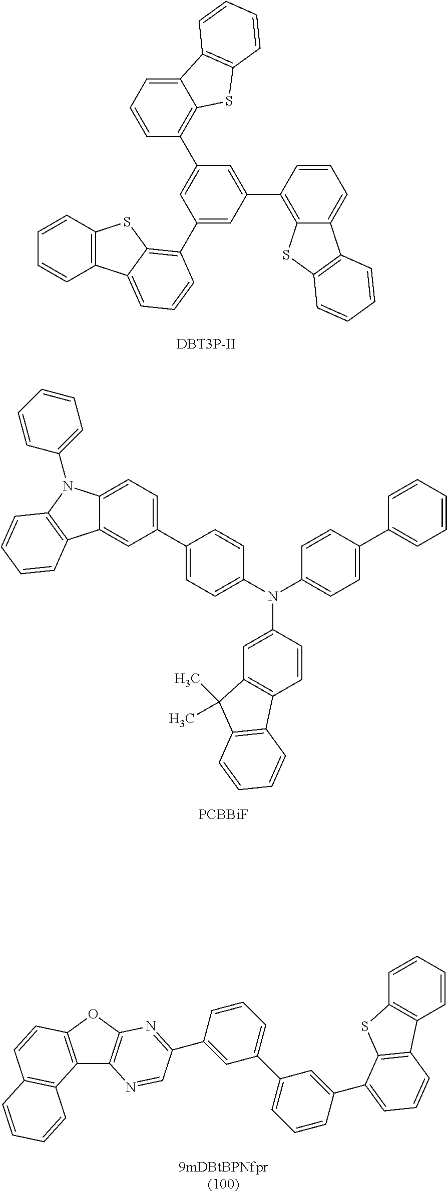

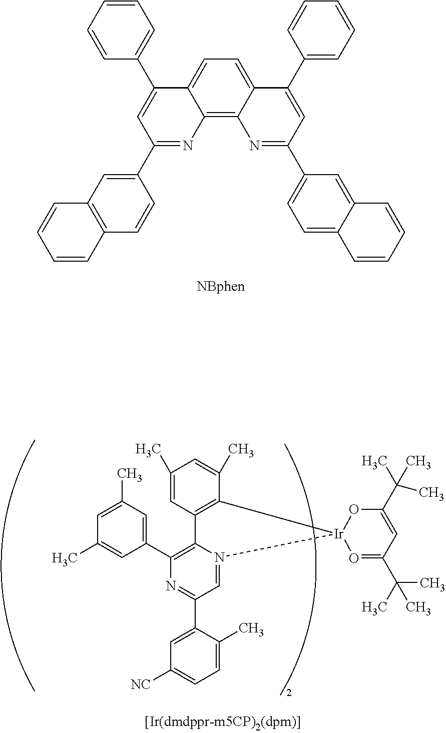

### <<Operation Characteristics of Light-Emitting Element 5>>

Operation characteristics of the fabricated light-emitting element 5 were measured. Note that the measurement was performed at room temperature (in an atmosphere kept at 25° C.).

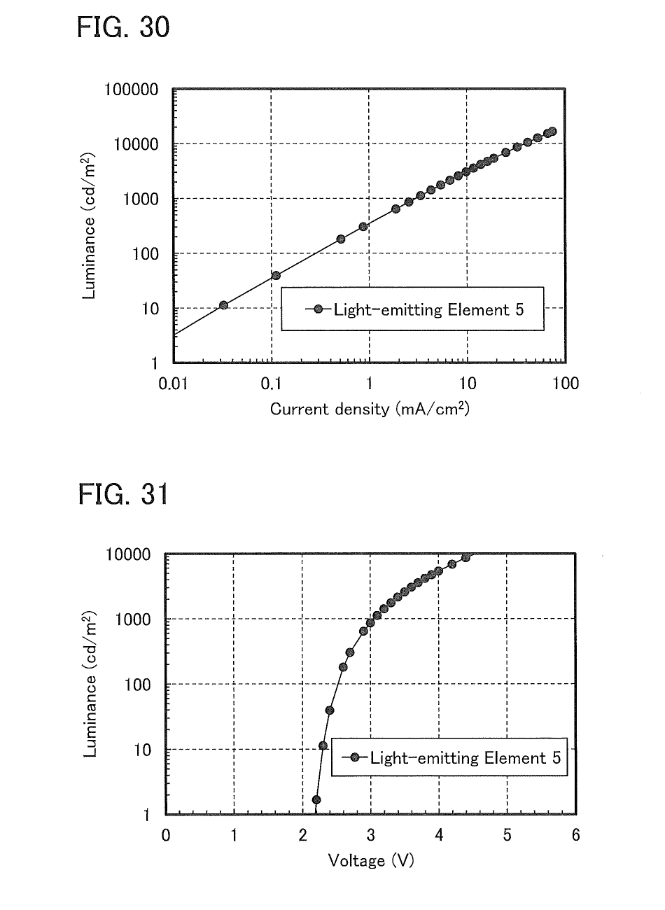

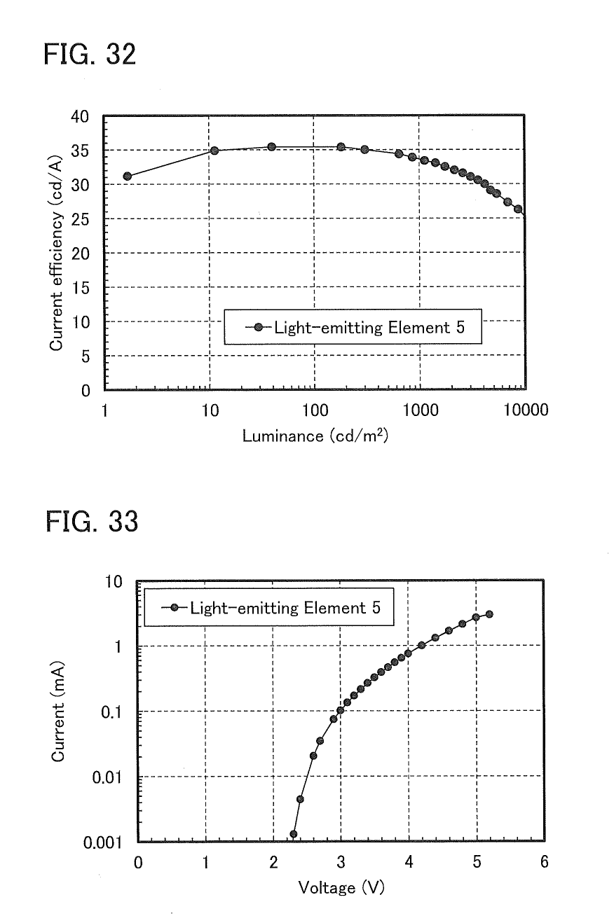

FIG. 30, FIG. 31, FIG. 32, and FIG. 33 show the current density-luminance characteristics, the voltage-luminance characteristics, the luminance-current efficiency characteristics, and the voltage-current characteristics, respectively, of the light-emitting element 5.

Table 8 shows initial values of main characteristics of the light-emitting element 5 at around 1000 cd/m².

TABLE 8

| | | | | | | | | External |
|---|---|---|---|---|---|---|---|---|
| | | | Current | | | Current | Power | quantum |
| | Voltage | Current | density | Chromaticity | Luminance | efficiency | efficiency | efficiency |
| | (V) | (mA) | (mA/cm²) | (x, y) | (cd/m²) | (cd/A) | (lm/W) | (%) |
| Light- | 3.1 | 0.13 | 3.4 | (0.70, 0.30) | 1100 | 33 | 34 | 37 |
| emitting | | | | | | | | |
| element 5 | | | | | | | | |

The above results show that the light-emitting element 5 fabricated in this example has high efficiency.

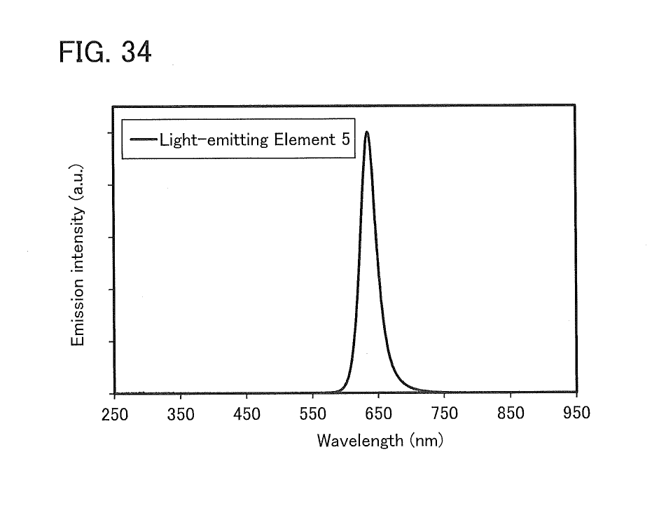

FIG. 34 shows an emission spectrum when current at a current density of 2.5 mA/cm² was applied to the light-emitting element 5. As shown in FIG. 34, the emission spectrum of the light-emitting element has a peak at around 635 nm that is probably derived from light emission of [Ir(dmdppr-m5CP)₂(dpm)] contained in the light-emitting layer 913. Accordingly, 9mDBtBPNfpr, the organic compound of one embodiment of the present invention, is a host material that is suitably used with a phosphorescent material that emits light with energy at a wavelength longer than or equal to that of red light.

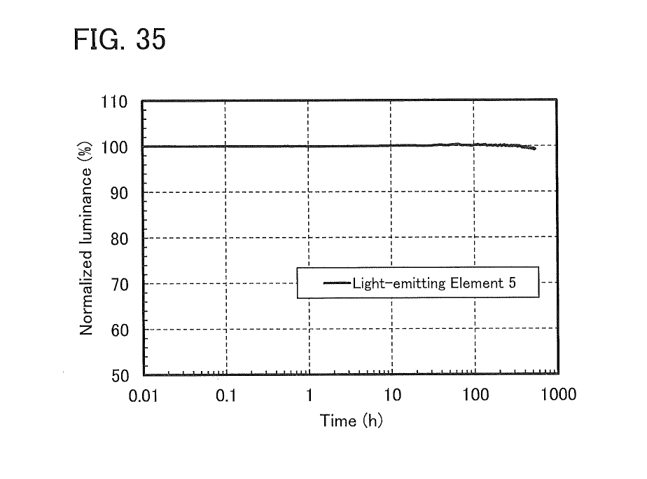

Next, a reliability test was performed on the light-emitting element 5. FIG. 35 shows results of the reliability test. In FIG. 35, the vertical axis represents normalized luminance (%) with an initial luminance of 100%, and the horizontal axis represents driving time (h) of the element. As the reliability test, a constant current driving test at a constant current density of 12.5 mA/cm² was performed.

The results of the reliability test show that the light-emitting element 5 has high reliability. This indicates that the use of 9mDBtBPNfpr (Structural Formula (100)), which is the organic compound of one embodiment of the present invention, is effective in improving the element characteristics of a light-emitting element.

Here, a top-emission panel formed by combination of the light-emitting element 5 and light-emitting elements 6 and 7 having element structures in Table 9 and operation characteristics in Table 10 was assumed. Then, simulation was performed under the following conditions: an aperture ratio was 15% (5% for each of R, G, and B pixels), attenuation of light by a circularly polarizing plate or the like was 60%, and a white color at D65 and 300 cd/m² was displayed entirely.

TABLE 9

| | | | Hole- | Light- | | | Electron- | | |
|---|---|---|---|---|---|---|---|---|---|
| | First | Hole-injection | transport | emitting | | | injection | | |
| | electrode | layer | layer | layer | Electron-transport layer | | layer | Second electrode | |
| Light- | APC\ITO | DBT3P-II:MoOx | BPAFLP | ** | 2mDBTBPDBcp-II | Bphen | LiF | Ag:Mg | ITO |
| emitting | (110 nm) | (1:0.5) | (15 nm) | | (15 nm) | (15 nm) | (1 nm) | (1:0.1) | (70 nm) |
| element | | (25 nm) | | | | | | (25 nm) | |
| 6(G) | | | | | | | | | |
| Light- | APC\ITO | PCPPn:MoOx | PCPPn | *** | cgDBCzPA | NBphen | LiF | Ag:Mg | ITO |
| emitting | (85 nm) | (1:0.5) | (15 nm) | | (5 nm) | (15 nm) | (1 nm) | (1:0.1) | (70 nm) |
| element | | (37.5 nm) | | | | | | (25 nm) | |
| 7(B) | | | | | | | | | |

** 2mDBTBPDBq-II:PCBBiF:[Ir(tBuppm)₃] (0.7:0.3:0.06 (20 nm)\0.8:0.2:0.06 (20 nm))
*** cgDBCzPA:1,6BnfAPrn-03 (1:0.03 (25 nm))

The chemical formulae of some of the materials used in the light-emitting elements in Table 9 are shown below.

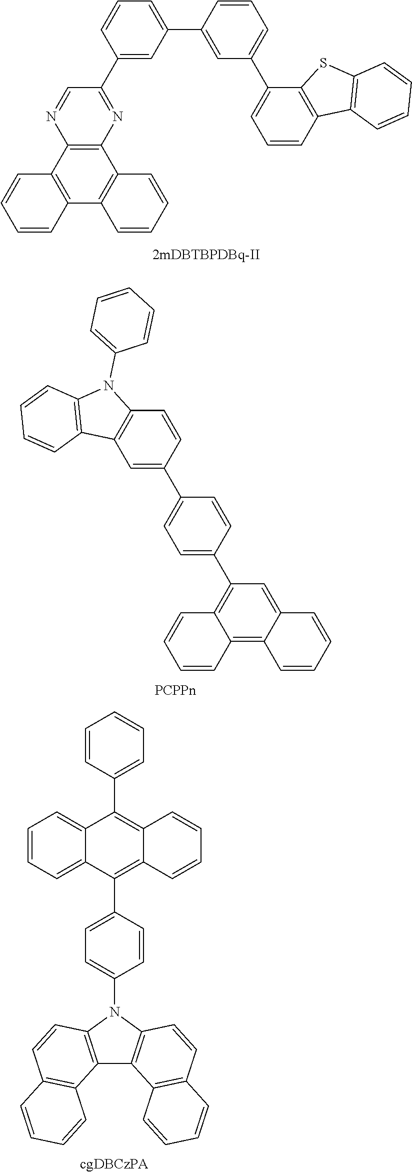 [SMILES: c1cc(-c2cccc(-c3cccc4c3sc3ccccc34)c2)cc(-c2cnc3c4ccccc4c4ccccc4c3n2)c1 ; c1ccc(-n2c3ccccc3c3cc(-c4ccc(-c5cc6ccccc6c6ccccc56)cc4)ccc32)cc1 ; c1ccc(-c2c3ccccc3c(-c3ccc(-n4c5ccc6ccccc6c5c5c6ccccc6ccc54)cc3)c3ccccc23)cc1]

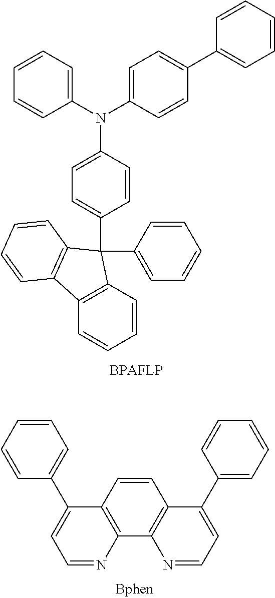 [SMILES: c1ccc(-c2ccc(N(c3ccccc3)c3ccc(C4(c5ccccc5)c5ccccc5-c5ccccc54)cc3)cc2)cc1 ; c1ccc(-c2ccnc3c2ccc2c(-c4ccccc4)ccnc23)cc1]

TABLE 10

| | | | | | | | | External |
|---|---|---|---|---|---|---|---|---|
| | | | Current | | | Current | Power | quantum |
| | Voltage | Current | density | Chromaticity | Luminance | efficiency | efficiency | efficiency |
| | (V) | (mA) | (mA/cm²) | (x, y) | (cd/m²) | (cd/A) | (lm/W) | (%) |
| Light- | 2.7 | 0.04 | 1.1 | (0.183, 0.786) | 1100 | 99 | 110 | 24 |
| emitting | | | | | | | | |
| element 6(G) | | | | | | | | |
| Light- | 3.3 | 1.20 | 29 | (0.141, 0.044) | 1100 | 3.6 | 3.5 | 6.9 |
| emitting | | | | | | | | |
| element 7(B) | | | | | | | | |

Table 11 shows some measurement results of the light-emitting elements used in the simulation.

TABLE 11

| Light- | | | Panel | Pixel | Current | Current | | Power |
|---|---|---|---|---|---|---|---|---|
| emitting | CIE | | luminance | luminance | efficiency | density | Voltage | consumption |
| element | x | y | (cd/m²) | (cd/m²) | (cd/A) | (mA/cm²) | (V) | (mW/cm²) |
| Light- | 0.703 | 0.297 | 77 | 3864 | 30.3 | 12.8 | 3.75 | 2.39 |
| emitting | | | | | | | | |
| element | | | | | | | | |
| 5(R) | | | | | | | | |
| Light- | 0.182 | 0.786 | 205 | 10257 | 95.4 | 10.8 | 3.40 | 1.83 |
| emitting | | | | | | | | |
| element | | | | | | | | |
| 6(G) | | | | | | | | |
| Light- | 0.141 | 0.045 | 18 | 879 | 3.7 | 24.1 | 3.20 | 3.85 |
| emitting | | | | | | | | |
| element | | | | | | | | |
| 7(B) | | | | | | | | |

According to the simulation using the data in Table 11, the ratio of the area of the panel formed by the combination of the light-emitting elements 5(R), 6(G), and 7(B) to the BT.2020 color gamut was 97% when being calculated from the chromaticities (x,y) of the light-emitting elements on the CIE1976 chromaticity coordinates (u′,v′ chromaticity coordinates).

### Example 6

### Synthesis Example 2

This example describes a method for synthesizing 9-(9′-phenyl-3,3′-bi-9H-carbazol-9-yl)naphtho[1′,2′:4,5]furo[2,3-b]pyrazine (abbreviation: 9PCCzNfpr), which is the organic compound of one embodiment of the present invention represented by Structural Formula (123) in Embodiment 1. The structure of 9PCCzNfpr is shown below.

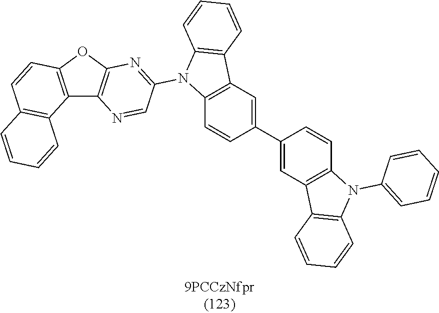 [SMILES: c1ccc(-n2c3ccccc3c3cc(-c4ccc5c(c4)c4ccccc4n5-c4cnc5c(n4)oc4ccc6ccccc6c45)ccc32)cc1]

Into a three-neck flask were put 0.94 g of 9-chloronaphtho[1′,2′:4,5]furo[2,3-b]pyrazine whose synthesis method is described in Step 2 in Example 1, 1.69 g of 9′-phenyl-3,3′-bi-9H-carbazole, and 37 mL of mesitylene, and the air in the flask was replaced with nitrogen. The mixture in the flask was degassed by being stirred under reduced pressure, and then 1.23 g of sodium tert-butoxide, 0.021 g of tris(dibenzylideneacetone)dipalladium(0) (abbreviation: Pd₂(dba)₃), and 0.030 g of 2-dicyclohexylphosphino-2′,6′-dimethoxybiphenyl (abbreviation: S-Phos) were added thereto. The mixture was stirred at 120° C. for 8 hours to be reacted.

After a predetermined time elapsed, the obtained suspension was subjected to suction filtration and was washed with water and ethanol. The obtained solid was dissolved in toluene, and the mixture was filtered through a filter aid in which Celite, alumina, and Celite were stacked in this order and was recrystallized with a mixed solvent of toluene and hexane, so that 0.85 g of a target yellow solid was obtained in a yield of 36%.

By a train sublimation method, 0.84 g of the obtained yellow solid was purified by sublimation. In the purification by sublimation, the solid was heated at 350° C. under a pressure of 2.5 Pa with an argon gas flow rate of 10 mL/min. After the purification by sublimation, 0.64 g of a target yellow solid was obtained in a yield of 76%. A synthesis scheme of the above synthesis method is shown in (b-1) below.

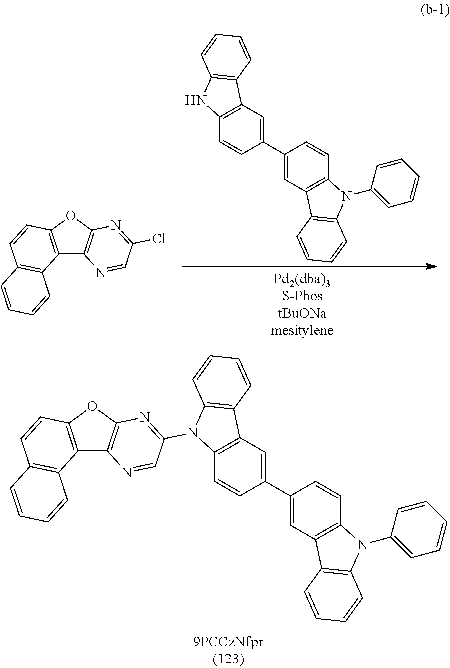 [SMILES: Clc1cnc2c(n1)oc1ccc3ccccc3c12 ; c1ccc(-n2c3ccccc3c3cc(-c4ccc5[nH]c6ccccc6c5c4)ccc32)cc1 ; c1ccc(-n2c3ccccc3c3cc(-c4ccc5c(c4)c4ccccc4n5-c4cnc5c(n4)oc4ccc6ccccc6c45)ccc32)cc1]

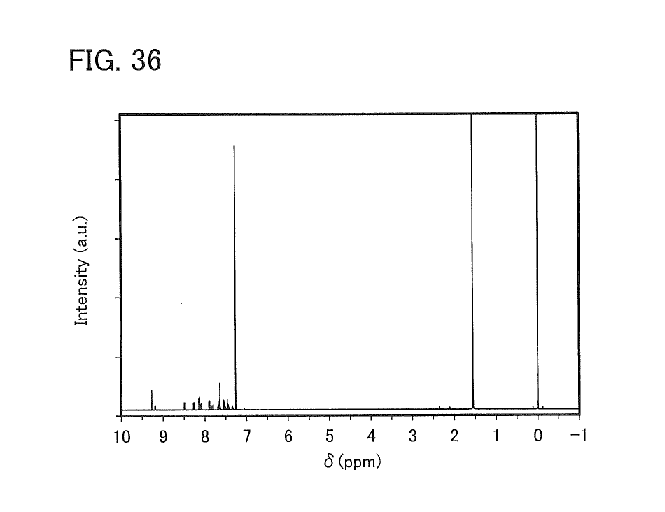

Analysis results by nuclear magnetic resonance (¹H-NMR) spectroscopy of the yellow solid obtained by the above synthesis method are shown below. FIG. 36 is the ¹H-NMR chart. The results revealed that 9PCCzNfpr, the organic compound represented by Structural Formula (123), was obtained in this example.

¹H-NMR. δ (CDCl₃): 7.32-7.35 (m, 1H), 7.42-7.57 (m, 6H), 7.63-7.70 (m, 5H), 7.80-7.90 (m, 4H), 8.09 (d, 2H), 8.14 (d, 2H), 8.27 (d, 2H), 8.49 (d, 2H), 9.20 (d, 1H), 9.27 (s, 1H).

### Example 7

### Synthesis Example 3

This example describes a method for synthesizing 9-[3-(9′-phenyl-3,3′-bi-9H-carbazol-9-yl)phenyl]naphtho[1′,2′:4,5]furo[2,3-b]pyrazine (abbreviation: 9mPCCzPNfpr), which is the organic compound of one embodiment of the present invention represented by Structural Formula (125) in Embodiment 1. The structure of 9mPCCzPNfpr is shown below.

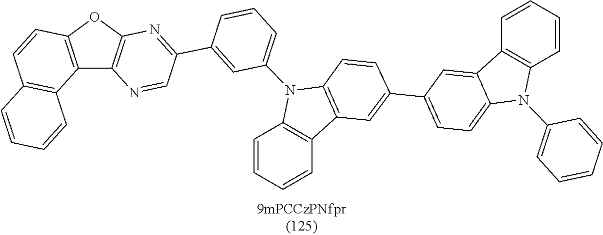 [SMILES: c1ccc(-n2c3ccccc3c3cc(-c4ccc5c(c4)c4ccccc4n5-c4cccc(-c5cnc6c(n5)oc5ccc7ccccc7c56)c4)ccc32)cc1]

### Step 1: Synthesis of 9-(3-chlorophenyl)naphtho[1′,2′:4,5]furo[2,3-b]pyrazine

Into a three-neck flask were put 2.12 g of 9-chloronaphtho[1′,2′:4,5]furo[2,3-b]pyrazine whose synthesis method is described in Step 2 in Example 1, 1.41 g of 3-chlorophenylboronic acid, 14 mL of a 2M potassium carbonate aqueous solution, 83 mL of toluene, and 8.3 mL of ethanol, and the air in the flask was replaced with nitrogen. The mixture in the flask was degassed by being stirred under reduced pressure, and then 0.19 g of palladium(II) acetate (abbreviation: Pd(OAc)₂) and 1.12 g of tris(2,6-dimethoxyphenyl)phosphine (abbreviation: P(2,6-MeOPh)₃) were added thereto. The mixture was stirred at 90° C. for 7.5 hours to be reacted.

After a predetermined time elapsed, the obtained mixture was subjected to suction filtration and was washed with ethanol. Then, purification by silica gel column chromatography using toluene as a developing solvent was performed, so that 1.97 g of a target pyrazine derivative (yellowish white powder) was obtained in a yield of 73%. A synthesis scheme of Step 1 is shown in (c-1) below.

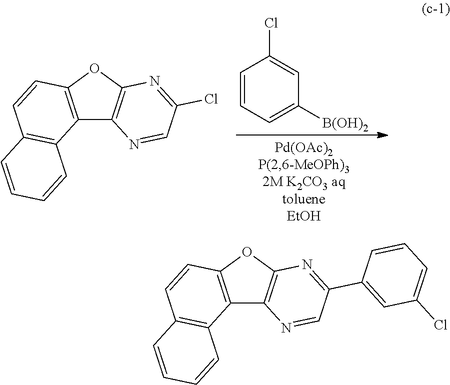

### Step 2: Synthesis of 9mPCCzPNfpr

Next, into a three-neck flask were put 1.45 g of 9-(3-chlorophenyl)naphtho[1′,2′:4,5]furo[2,3-b]pyrazine obtained in Step 1, 1.82 g of 9′-phenyl-3,3′-bi-9H-carbazole, and 22 mL of mesitylene, and the air in the flask was replaced with nitrogen. The mixture in the flask was degassed by being stirred under reduced pressure, and then 0.85 g of sodium tert-butoxide, 0.025 g of tris(dibenzylideneacetone)dipalladium(0) (abbreviation: Pd₂(dba)₃), and 0.036 g of 2-dicyclohexylphosphino-2′,6′-dimethoxybiphenyl (abbreviation: S-Phos) were added thereto. The mixture was stirred at 150° C. for 7 hours to be reacted.

After a predetermined time elapsed, the obtained suspension was subjected to suction filtration and was washed with water and ethanol. The obtained solid was dissolved in toluene, and the mixture was filtered through a filter aid in which Celite, alumina, and Celite were stacked in this order and was recrystallized with a mixed solvent of toluene and hexane, so that 2.22 g of a target yellow solid was obtained in a yield of 71%.

By a train sublimation method, 2.16 g of the obtained yellow solid was purified by sublimation. In the purification by sublimation, the solid was heated at 385° C. under a pressure of 2.6 Pa with an argon gas flow rate of 18 mL/min. After the purification by sublimation, 1.67 g of a target yellow solid was obtained in a yield of 77%. A synthesis scheme of Step 2 is shown in (c-2) below.

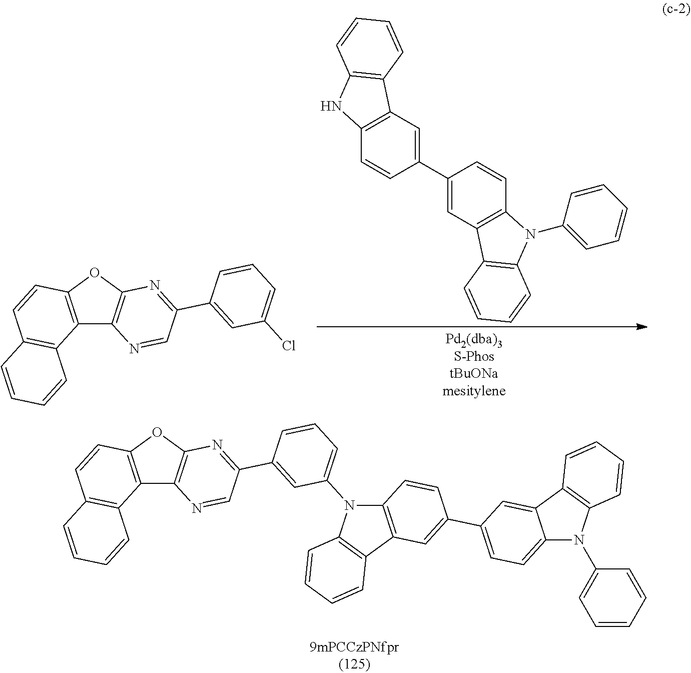 [SMILES: Clc1cccc(-c2cnc3c(n2)oc2ccc4ccccc4c23)c1 ; c1ccc(-n2c3ccccc3c3cc(-c4ccc5[nH]c6ccccc6c5c4)ccc32)cc1 ; c1ccc(-n2c3ccccc3c3cc(-c4ccc5c(c4)c4ccccc4n5-c4cccc(-c5cnc6c(n5)oc5ccc7ccccc7c56)c4)ccc32)cc1]

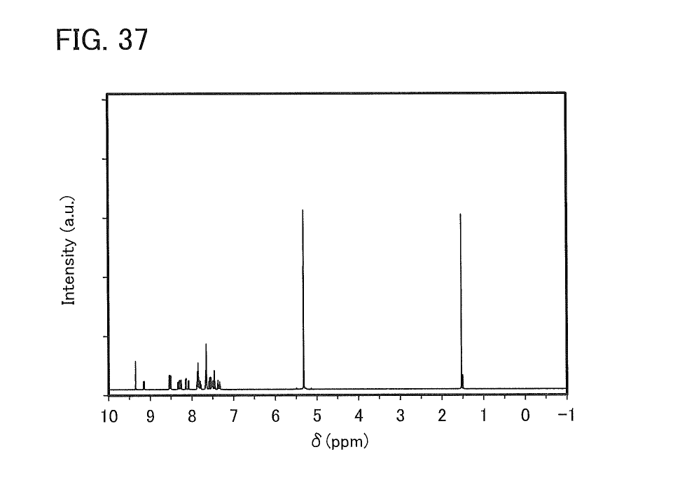

Analysis results by nuclear magnetic resonance (¹H-NMR) spectroscopy of the yellow solid obtained in Step 2 are shown below. FIG. 37 is the ¹H-NMR chart. The results revealed that 9mPCCzPNfpr, the organic compound represented by Structural Formula (125), was obtained in this example.

¹H-NMR. δ (CD₂Cl₂): 7.31-7.39 (m, 2H), 7.43-7.59 (m, 6H), 7.64-7.69 (m, 6H), 7.78-7.88 (m, 6H), 8.09 (d, 1H), 8.15 (d, 1H), 8.26 (d, 1H), 8.30 (d, 1H), 8.34 (d, 1H), 8.51-8.55 (m, 3H), 9.15 (d, 1H), 9.35 (s, 1H).

### Example 8

### Synthesis Example 4

This example describes a method for synthesizing 9-[3-(9′-phenyl-2,3′-bi-9H-carbazol-9-yl)phenyl]naphtho[1′,2′:4,5]furo[2,3-b]pyrazine (abbreviation: 9mPCCzPNfpr-02), which is the organic compound of one embodiment of the present invention represented by Structural Formula (126) in Embodiment 1. The structure of 9mPCCzPNfpr-02 is shown below.

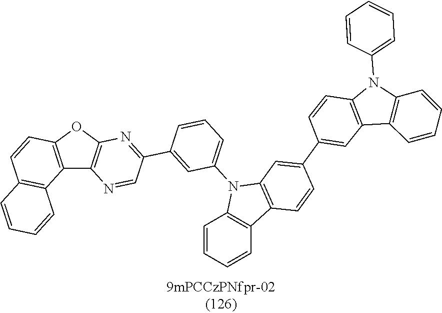 [SMILES: c1ccc(-n2c3ccccc3c3cc(-c4ccc5c6ccccc6n(-c6cccc(-c7cnc8c(n7)oc7ccc9ccccc9c78)c6)c5c4)ccc32)cc1]

Into a three-neck flask were put 1.19 g of 9-chloronaphtho[1′,2′:4,5]furo[2,3-b]pyrazine whose synthesis method is described in Step 2 in Example 1, 3.51 g of 3-(9′-phenyl-2,3′-bi-9H-carbazol-9-yl)phenylboronic acid pinacol ester, 6.0 mL of a 2M potassium carbonate aqueous solution, 60 mL of toluene, and 6 mL of ethanol, and the air in the flask was replaced with nitrogen. The mixture in the flask was degassed by being stirred under reduced pressure, and then 0.33 g of bis(triphenylphosphine)palladium(II) dichloride (abbreviation: Pd(PPh₃)₂Cl₂) was added thereto. The mixture was stirred at 90° C. for 16 hours to be reacted.

After a predetermined time elapsed, the obtained suspension was subjected to suction filtration and was washed with water and ethanol. The obtained solid was dissolved in toluene, and the mixture was filtered through a filter aid in which Celite, alumina, and Celite were stacked in this order and was recrystallized with a mixed solvent of toluene and hexane, so that 3.01 g of a target yellow solid was obtained in a yield of 90%.

By a train sublimation method, 3.00 g of the obtained yellow solid was purified by sublimation. In the purification by sublimation, the solid was heated at 380° C. under a pressure of 2.7 Pa with an argon gas flow rate of 16 mL/min. After the purification by sublimation, 2.47 g of a target yellow solid was obtained in a yield of 82%. A synthesis scheme is shown in (d-1) below.

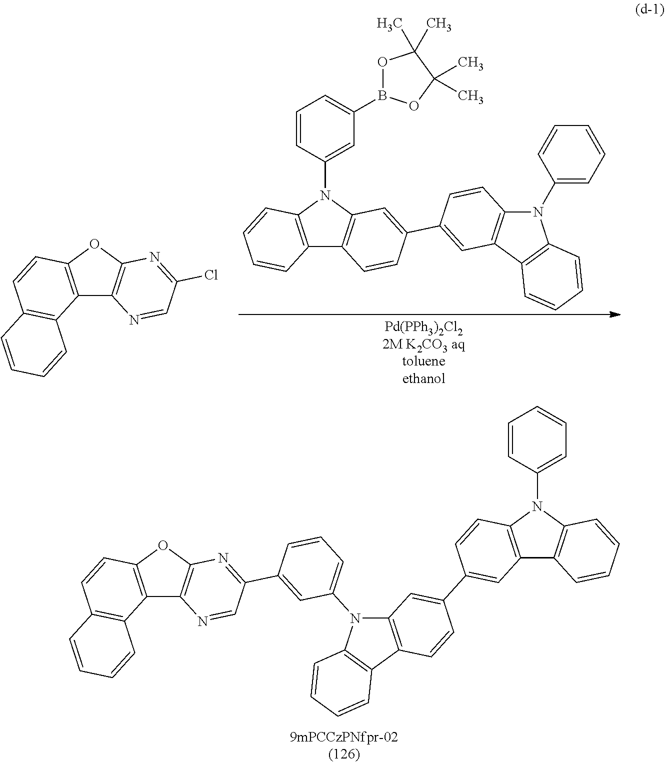

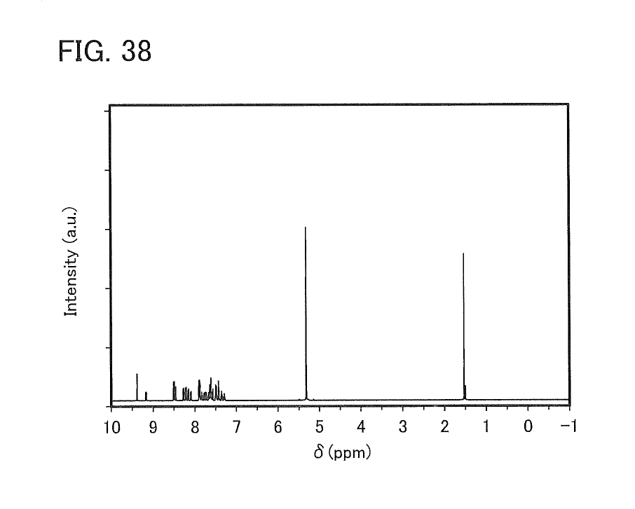

Analysis results by nuclear magnetic resonance (¹H-NMR) spectroscopy of the yellow solid obtained above are shown below. FIG. 38 is the ¹H-NMR chart. The results revealed that 9mPCCzPNfpr-02, the organic compound represented by Structural Formula (126), was obtained in this example.

¹H-NMR. δ (CD₂Cl₂): 7.22-7.25 (m, 1H), 7.34-7.42 (m, 3H), 7.46-7.49 (m, 3H), 7.55-7.66 (m, 6H), 7.72-7.88 (m, 7H), 8.07 (d, 1H), 8.13 (d, 1H), 8.19-8.22 (m, 2H), 8.28 (d, 1H), 8.33 (d, 1H), 8.46 (s, 1H), 8.54 (s, 1H), 9.14 (d, 1H), 9.34 (s, 1H).

### Example 9

### Synthesis Example 5

This example describes a method for synthesizing 10-[(3′-dibenzothiophen-4-yl)biphenyl-3-yl]naphtho[1′,2′:4,5]furo[2,3-b]pyrazine (abbreviation: 10mDBtBPNfpr), which is the organic compound of one embodiment of the present invention represented by Structural Formula (133) in Embodiment 1. The structure of 10mDBtBPNfpr is shown below.

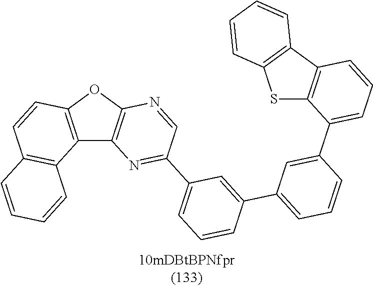 [SMILES: c1cc(-c2cccc(-c3cccc4c3sc3ccccc34)c2)cc(-c2cnc3oc4ccc5ccccc5c4c3n2)c1]

### Step 1: Synthesis of 5-chloro-3-(2-methoxynaphthalen-1-yl)pyrazin-2-amine

First, into a three-neck flask equipped with a reflux pipe were put 5.01 g of 3-bromo-5-chloropyrazin-2-amine, 6.04 g of 2-methoxynaphthalene-1-boronic acid, 5.32 g of potassium fluoride, and 86 mL of dehydrated tetrahydrofuran, and the air in the flask was replaced with nitrogen. The mixture in the flask was degassed by being stirred under reduced pressure, and then 0.44 g of tris(dibenzylideneacetone)dipalladium(0) (abbreviation: Pd₂(dba)₃) and 3.4 mL of tri-tert-butylphosphine (abbreviation: P(tBu)₃) were added thereto. The mixture was stirred at 80° C. for 22 hours to be reacted.

After a predetermined time elapsed, the obtained mixture was subjected to suction filtration and the filtrate was concentrated. Then, purification by silica gel column chromatography using a developing solvent (toluene:ethyl acetate=10:1) was performed, so that 5.69 g of a target pyrazine derivative (yellowish white powder) was obtained in a yield of 83%. A synthesis scheme of Step 1 is shown in (e-1) below.

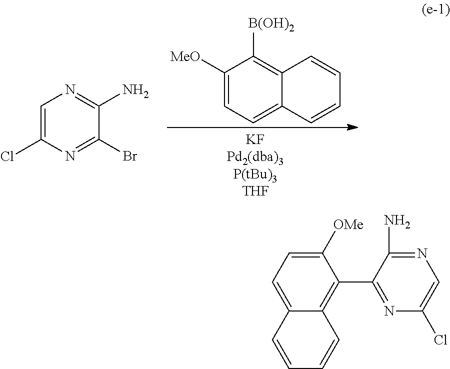 [SMILES: Nc1ncc(Cl)nc1Br ; COc1ccc2ccccc2c1B(O)O ; COc1ccc2ccccc2c1-c1nc(Cl)cnc1N]

### Step 2: Synthesis of 10-chloronaphtho[1′,2′:4,5]furo[2,3-b]pyrazine

Next, into a three-neck flask were put 5.69 g of 5-chloro-3-(2-methoxynaphthalen-1-yl)pyrazin-2-amine obtained in Step 1, 150 mL of dehydrated tetrahydrofuran, and 150 mL of a glacial acetic acid, and the air in the flask was replaced with nitrogen. After the flask was cooled down to −10° C., 7.1 mL of tert-butyl nitrite was dripped, and the mixture was stirred at −10° C. for 1 hour and at 0° C. for 3.5 hours. After a predetermined time elapsed, 1 L of water was added to the obtained suspension and suction filtration was performed, so that 4.06 g of a target pyrazine derivative (yellowish white powder) was obtained in a yield of 81%. A synthesis scheme of Step 2 is shown in (e-2) below.

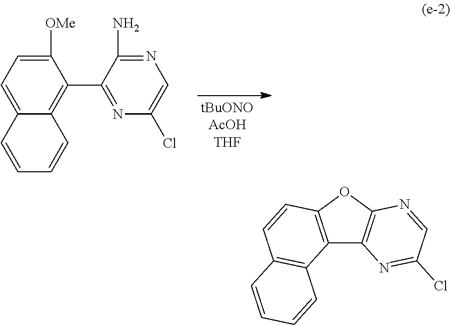 [SMILES: COc1ccc2ccccc2c1-c1nc(Cl)cnc1N ; Clc1cnc2oc3ccc4ccccc4c3c2n1]

### Step 3: Synthesis of 10mDBtBPNfpr

Into a three-neck flask were put 1.18 g of 10-chloronaphtho[1′,2′:4,5]furo[2,3-b]pyrazine obtained in Step 2, 2.75 g of 3′-(4-dibenzothiophene)-1,1′-biphenyl-3-boronic acid, 7.5 mL of a 2M potassium carbonate aqueous solution, 60 mL of toluene, and 6 mL of ethanol, and the air in the flask was replaced with nitrogen. The mixture in the flask was degassed by being stirred under reduced pressure, and then 0.66 g of bis(triphenylphosphine)palladium(II) dichloride (abbreviation: Pd(PPh₃)₂Cl₂) was added thereto. The mixture was stirred at 90° C. for 22.5 hours to be reacted.

After a predetermined time elapsed, the obtained suspension was subjected to suction filtration and was washed with water and ethanol. The obtained solid was dissolved in toluene, and the mixture was filtered through a filter aid in which Celite, alumina, and Celite were stacked in this order and was recrystallized with a mixed solvent of toluene and hexane, so that 2.27 g of a target white solid was obtained in a yield of 87%.

By a train sublimation method, 2.24 g of the obtained white solid was purified by sublimation. In the purification by sublimation, the solid was heated at 310° C. under a pressure of 2.3 Pa with an argon gas flow rate of 16 mL/min. After the purification by sublimation, 1.69 g of a target white solid was obtained in a yield of 75%. A synthesis scheme of Step 3 is shown in (e-3) below.

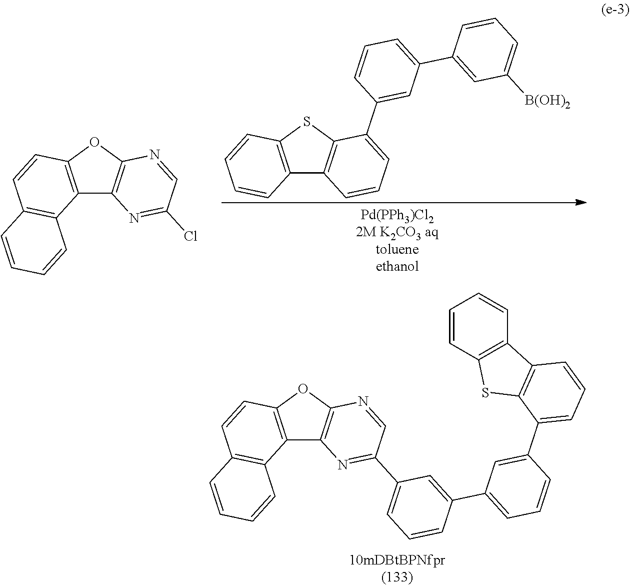 [SMILES: Clc1cnc2oc3ccc4ccccc4c3c2n1 ; OB(O)c1cccc(-c2cccc(-c3cccc4c3sc3ccccc34)c2)c1 ; c1cc(-c2cccc(-c3cccc4c3sc3ccccc34)c2)cc(-c2cnc3oc4ccc5ccccc5c4c3n2)c1]

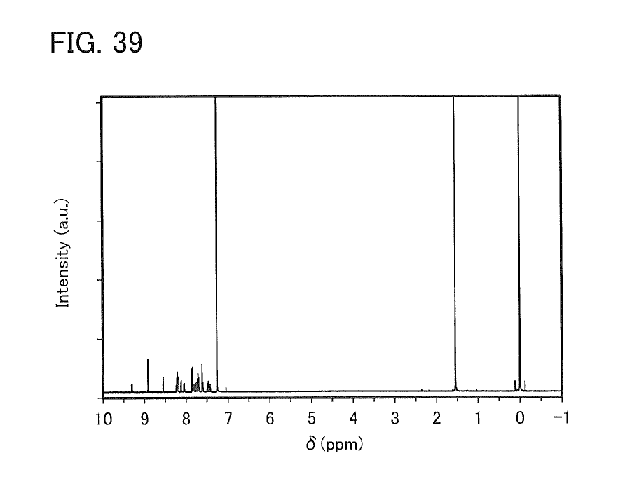

Analysis results by nuclear magnetic resonance (¹H-NMR) spectroscopy of the white solid obtained in Step 3 are shown below. FIG. 39 is the ¹H-NMR chart. The results revealed that 10mDBtBPNfpr, the organic compound represented by Structural Formula (133), was obtained in this example.

¹H-NMR. δ (CDCl₃): 7.43 (t, 1H), 7.48 (t, 1H), 7.59-7.62 (m, 3H), 7.68-7.86 (m, 8H), 8.05 (d, 1H), 8.12 (d, 1H), 8.18 (s, 1H), 8.20-8.24 (m, 3H), 8.55 (s, 1H), 8.92 (s, 1H), 9.31 (d, 1H).

### Example 10

### Synthesis Example 6

This example describes a method for synthesizing 10-(9′-phenyl-3,3′-bi-9H-carbazol-9-yl)naphtho[1′,2′:4,5]furo[2,3-b]pyrazine (abbreviation: 10PCCzNfpr), which is the organic compound of one embodiment of the present invention represented by Structural Formula (156) in Embodiment 1. The structure of 10PCCzNfpr is shown below.

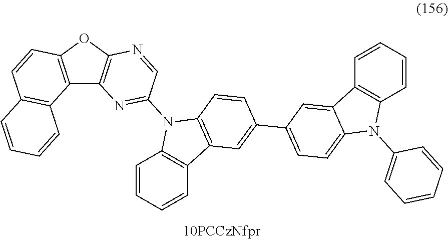 [SMILES: c1ccc(-n2c3ccccc3c3cc(-c4ccc5c(c4)c4ccccc4n5-c4cnc5oc6ccc7ccccc7c6c5n4)ccc32)cc1]

Into a three-neck flask were put 1.80 g of 10-chloronaphtho[1′,2′:4,5]furo[2,3-b]pyrazine whose synthesis method is described in Step 2 in Example 9, 3.10 g of 9′-phenyl-3,3′-bi-9H-carbazole, and 71 mL of mesitylene, and the air in the flask was replaced with nitrogen. The mixture in the flask was degassed by being stirred under reduced pressure, and then 2.21 g of sodium tert-butoxide, 0.041 g of tris(dibenzylideneacetone)dipalladium(0) (abbreviation: Pd₂(dba)₃), and 0.061 g of 2-dicyclohexylphosphino-2′,6′-dimethoxybiphenyl (abbreviation: S-Phos) were added thereto. The mixture was stirred at 120° C. for 2 hours to be reacted.

After a predetermined time elapsed, the obtained suspension was subjected to suction filtration and was washed with water and ethanol. The obtained solid was dissolved in toluene, and the mixture was filtered through a filter aid in which Celite, alumina, and Celite were stacked in this order and was recrystallized with a mixed solvent of toluene and hexane, so that 3.47 g of a target orange solid was obtained in a yield of 78%.

By a train sublimation method, 3.42 g of the obtained orange solid was purified by sublimation. In the purification by sublimation, the solid was heated at 350° C. under a pressure of 2.4 Pa with an argon gas flow rate of 16 mL/min. After the purification by sublimation, 2.86 g of a target orange solid was obtained in a yield of 84%. A synthesis scheme of Step 3 is shown in (f-1) below.

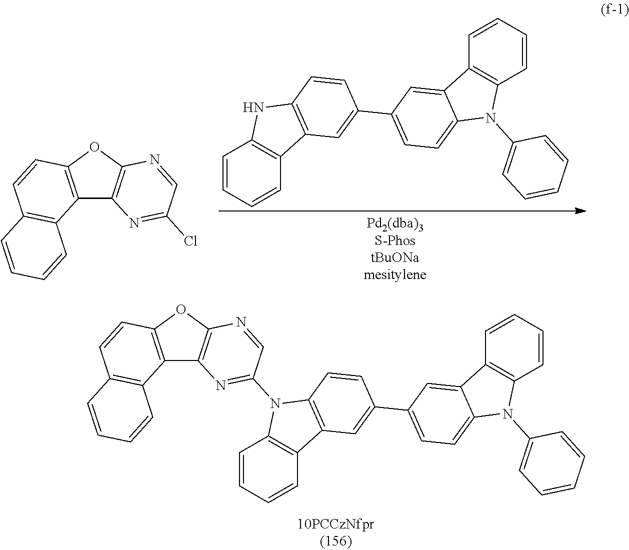

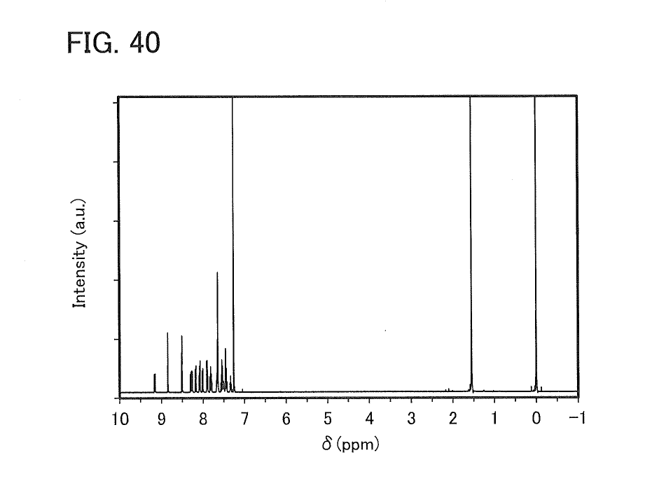

Analysis results by nuclear magnetic resonance (¹H-NMR) spectroscopy of the orange solid obtained by the above synthesis method are shown below. FIG. 40 is the ¹H-NMR chart. The results revealed that 10PCCzNfpr, the organic compound represented by Structural Formula (156), was obtained in this example.

¹H-NMR. δ (CDCl₃): 7.32-7.35 (m, 1H), 7.43-7.57 (m, 6H), 7.63-7.68 (m, 5H), 7.79-7.84 (m, 2H), 7.89-7.91 (m, 2H), 8.01 (d, 1H), 8.07-8.09 (m, 2H), 8.18 (d, 1H), 8.27 (d, 1H), 8.30 (d, 1H), 8.51 (s, 2H), 8.85 (s, 1H), 9.16 (d, 1H).

### Example 11

### Synthesis Example 7

This example describes a method for synthesizing 12-[(3′-dibenzothiophen-4-yl)biphenyl-3-yl]phenanthro[9′,10′:4,5]furo[2,3-b]pyrazine (abbreviation: 12mDBtBPPnfpr), which is the organic compound of one embodiment of the present invention represented by Structural Formula (208) in Embodiment 1. The structure of 12mDBtBPPnfpr is shown below.

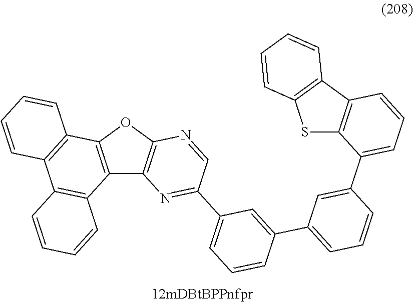 [SMILES: c1cc(-c2cccc(-c3cccc4c3sc3ccccc34)c2)cc(-c2cnc3oc4c5ccccc5c5ccccc5c4c3n2)c1]

### Step 1: Synthesis of 9-methoxyphenanthrene

First, into a three-neck flask equipped with a reflux pipe were put 4.02 g of 9-bromo-phenanthrene, 7.80 g of cesium carbonate, 16 mL of toluene, and 16 mL of methanol, and the air in the flask was replaced with nitrogen. The mixture in the flask was degassed by being stirred under reduced pressure, and then 0.11 g of palladium(II) acetate (abbreviation: Pd(OAc)₂) and 0.41 g of 2-di-tert-butylphosphino-2′,4′,6′-triisopropylbiphenyl (abbreviation: tBuXPhos) were added thereto. The mixture was stirred at 80° C. for 17 hours to be reacted.

After a predetermined time elapsed, the obtained mixture was subjected to suction filtration and the filtrate was concentrated. Then, purification by silica gel column chromatography using a developing solvent (toluene:hexane=1:3) was performed, so that 2.41 g of a target white powder was obtained in a yield of 74%. A synthesis scheme of Step 1 is shown in (g-1) below.

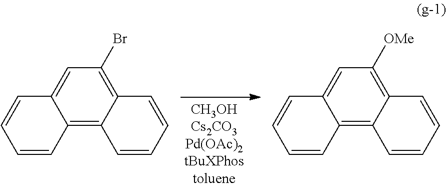 [SMILES: Brc1cc2ccccc2c2ccccc12 ; COc1cc2ccccc2c2ccccc12]

### Step 2: Synthesis of 9-bromo-10-methoxyphenanthrene

Next, into a conical flask were put 2.75 g of 9-methoxyphenanthrene obtained in Step 1, 0.18 mL of diisopropylamine, 150 mL of dehydrated dichloromethane, and 2.52 g of N-bromosuccinimide (abbreviation: NBS), and the mixture was stirred at room temperature for 18 hours. After a predetermined time elapsed, the mixture was washed with water and an aqueous solution of sodium thiosulfate, and then concentrated. Then, purification by silica gel column chromatography using a developing solvent (hexane:ethyl acetate=5:1) was performed, so that 2.46 g of a target yellowish white powder was obtained in a yield of 65%. A synthesis scheme of Step 2 is shown in (g-2) below.

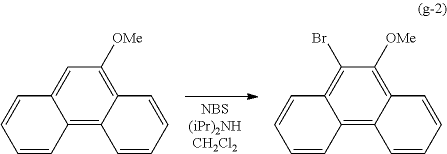 [SMILES: COc1cc2ccccc2c2ccccc12 ; COc1c(Br)c2ccccc2c2ccccc12]

### Step 3: Synthesis of 10-methoxyphenanthrene-9-boronic acid

Next, into a three-neck flask were put 8.49 g of 9-bromo-10-methoxyphenanthrene obtained in Step 2 and 250 mL of dehydrated THF, and the air in the flask was replaced with nitrogen. After the flask was cooled down to −78° C., 22 mL of a 1.6M hexane solution of n-butyllithium was added, and the mixture was stirred at −78° C. for 3 hours. Then, 5.7 mL of tetramethylethylenediamine and 4.3 mL of trimethyl borate were added, and the mixture was stirred at room temperature for 18 hours to be reacted.

After a predetermined time elapsed, 50 mL of 1M hydrochloric acid was added, and the mixture was stirred at room temperature for 1 hour. Then, extraction with toluene was performed, so that 2.87 g of a target pale orange powder was obtained in a yield of 39%. A synthesis scheme of Step 3 is shown in (g-3) below.

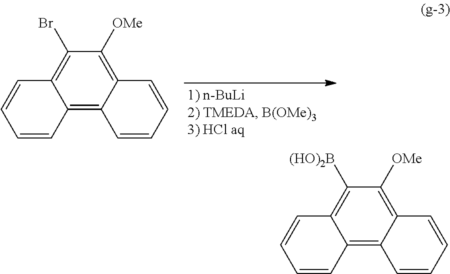 [SMILES: COc1c(Br)c2ccccc2c2ccccc12 ; COc1c(B(O)O)c2ccccc2c2ccccc12]

### Step 4: Synthesis of 5-chloro-3-(10-methoxyphenanthren-9-yl)pyrazin-2-amine

Next, into a three-neck flask equipped with a reflux pipe were put 3.69 g of 10-methoxyphenanthrene-9-boronic acid obtained in Step 3, 3.02 g of 3-bromo-5-chloropyrazin-2-amine, 70 mL of toluene, and 35 mL of a 2M sodium carbonate aqueous solution, and the air in the flask was replaced with nitrogen. The mixture in the flask was degassed by being stirred under reduced pressure, and then 0.16 g of tetrakis(triphenylphosphine)palladium(0) (abbreviation: Pd(PPh₃)₄) was added thereto. The mixture was stirred at 110° C. for 7.5 hours to be reacted.

After a predetermined time elapsed, extraction with toluene was performed. Then, purification by flash column chromatography using a developing solvent (dichloromethane:ethyl acetate=50:1) was performed, so that 3.00 g of a target pyrazine derivative (yellowish white powder) was obtained in a yield of 62%. A synthesis scheme of Step 4 is shown in (g-4) below.

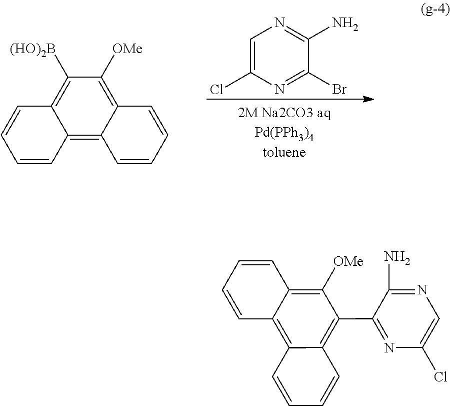 [SMILES: COc1c(B(O)O)c2ccccc2c2ccccc12 ; Nc1ncc(Cl)nc1Br ; COc1c(-c2nc(Cl)cnc2N)c2ccccc2c2ccccc12]

### Step 5: Synthesis of 12-chlorophenanthro[9′,10′:4,5]furo[2,3-b]pyrazine

Next, into a three-neck flask were put 2.92 g of 5-chloro-3-(10-methoxyphenanthren-9-yl)pyrazin-2-amine obtained in Step 4, 60 mL of dehydrated tetrahydrofuran, and 60 mL of a glacial acetic acid, and the air in the flask was replaced with nitrogen. After the flask was cooled down to −10° C., 3.1 mL of tert-butyl nitrite was dripped, and the mixture was stirred at −10° C. for 1 hour and at 0° C. for 22 hours.

After a predetermined time elapsed, 200 mL of water was added to the obtained suspension and suction filtration was performed, so that 2.06 g of a target pyrazine derivative (yellowish white powder) was obtained in a yield of 80%. A synthesis scheme of Step 5 is shown in (g-5) below.

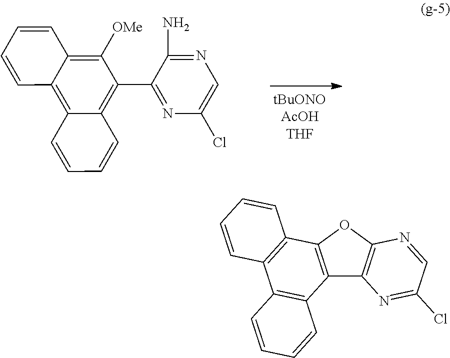 [SMILES: COc1c(-c2nc(Cl)cnc2N)c2ccccc2c2ccccc12 ; Clc1cnc2oc3c4ccccc4c4ccccc4c3c2n1]

### Step 6: Synthesis of 12-(3-chlorophenyl)phenanthro[9′,10′:4,5]furo[2,3-b]pyrazine

Next, into a three-neck flask were put 1.02 g of 12-chlorophenanthro[9′,10′:4,5]furo[2,3-b]pyrazine obtained in Step 5, 0.56 g of 3-chlorophenylboronic acid, 5 mL of a 2M potassium carbonate aqueous solution, 33 mL of toluene, and 3.3 mL of ethanol, and the air in the flask was replaced with nitrogen. The mixture in the flask was degassed by being stirred under reduced pressure, and then 0.074 g of palladium(II) acetate (abbreviation: Pd(OAc)₂) and 0.44 g of tris(2,6-dimethoxyphenyl)phosphine (abbreviation: P(2,6-MeOPh)₃) were added thereto. The mixture was stirred at 90° C. for 5.5 hours to be reacted.

After a predetermined time elapsed, the obtained mixture was subjected to suction filtration and the filtrate was concentrated. Then, purification by silica gel column chromatography using toluene as a developing solvent was performed, so that 0.87 g of a target pyrazine derivative (white powder) was obtained in a yield of 70%. A synthesis scheme of Step 6 is shown in (g-6) below.

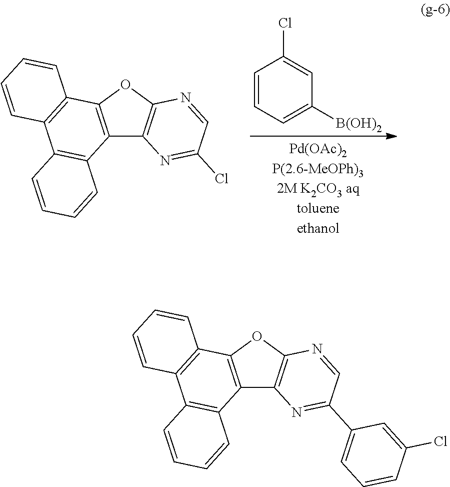 [SMILES: Clc1cnc2oc3c4ccccc4c4ccccc4c3c2n1 ; OB(O)c1cccc(Cl)c1 ; Clc1cccc(-c2cnc3oc4c5ccccc5c5ccccc5c4c3n2)c1]

### Step 7: Synthesis of 12mDBtBPPnfpr

Next, into a three-neck flask were put 0.85 g of 12-(3-chlorophenyl)phenanthro[9′,10′:4,5]furo[2,3-b]pyrazine obtained in Step 6, 0.73 g of 3-(4-dibenzothiophene)phenylboronic acid, 1.41 g of tripotassium phosphate, 0.49 g of tert-butyl alcohol, and 18 mL of diethylene glycol dimethyl ether (abbreviation: diglyme), and the air in the flask was replaced with nitrogen. The mixture in the flask was degassed by being stirred under reduced pressure, and then 9.8 mg of palladium(II) acetate (abbreviation: Pd(OAc)₂) and 32 mg of di(1-adamantyl)-n-butylphosphine (abbreviation: CataCXium A) were added thereto. The mixture was stirred at 140° C. for 11.5 hours to be reacted.

After a predetermined time elapsed, the obtained suspension was subjected to suction filtration and was washed with water and ethanol. The obtained solid was dissolved in toluene, and the mixture was filtered through a filter aid in which Celite, alumina, and Celite were stacked in this order and was recrystallized with toluene, so that 0.74 g of a target white solid was obtained in a yield of 55%.

By a train sublimation method, 0.73 g of the obtained white solid was purified by sublimation. In the purification by sublimation, the solid was heated at 330° C. under a pressure of 2.6 Pa with an argon gas flow rate of 11 mL/min. After the purification by sublimation, 0.49 g of a target white solid was obtained in a yield of 67%. A synthesis scheme of Step 7 is shown in (g-7) below.

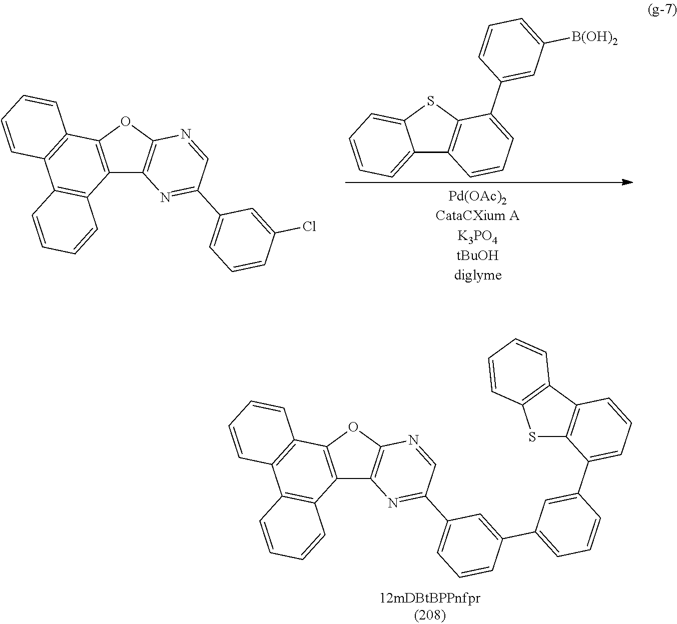 [SMILES: Clc1cccc(-c2cnc3oc4c5ccccc5c5ccccc5c4c3n2)c1 ; OB(O)c1cccc(-c2cccc3c2sc2ccccc23)c1 ; c1cc(-c2cccc(-c3cccc4c3sc3ccccc34)c2)cc(-c2cnc3oc4c5ccccc5c5ccccc5c4c3n2)c1]

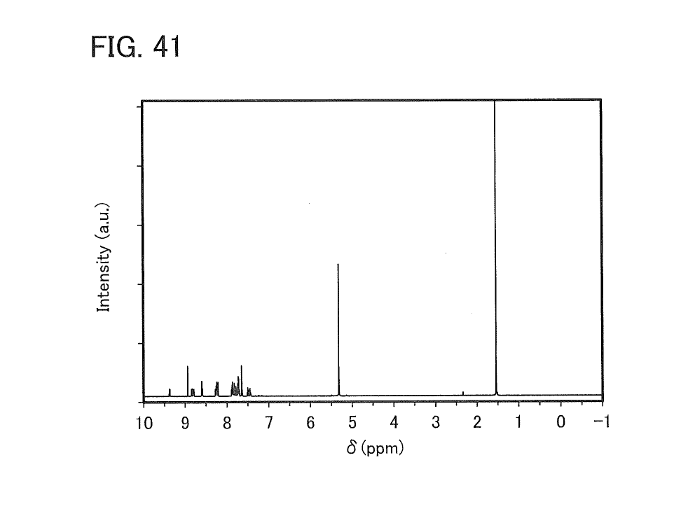

Analysis results by nuclear magnetic resonance (¹H-NMR) spectroscopy of the white solid obtained in Step 7 are shown below. FIG. 41 is the ¹H-NMR chart. The results revealed that 12mDBtBPPnfpr, the organic compound represented by Structural Formula (208), was obtained in this example.

¹H-NMR. δ (CD₂Cl₂): 7.45 (t, 1H), 7.50 (t, 1H), 7.62-7.66 (m, 2H), 7.70-7.89 (m, 10H), 8.21-8.28 (m, 4H), 8.58-8.61 (m, 2H), 8.80 (d, 1H), 8.84 (d, 1H), 8.94 (s, 1H), 9.37 (d, 1H).

### Example 12

### Synthesis Example 8

This example describes a method for synthesizing 9-[4-(9′-phenyl-3,3′-bi-9H-carbazol-9-yl)phenyl]naphtho[1′,2′:4,5]furo[2,3-b]pyrazine (abbreviation: 9pPCCzPNfpr), which is the organic compound of one embodiment of the present invention represented by Structural Formula (238) in Embodiment 1. The structure of 9pPCCzPNfpr is shown below.

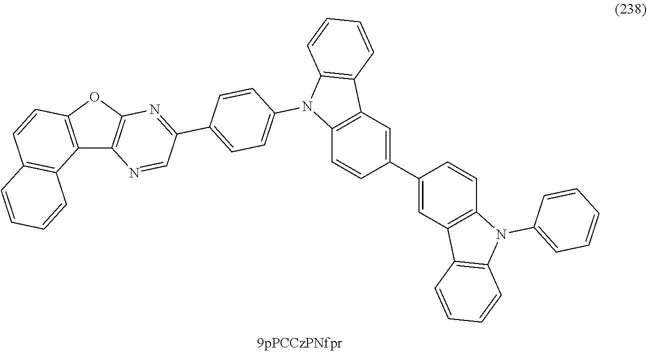 [SMILES: c1ccc(-n2c3ccccc3c3cc(-c4ccc5c(c4)c4ccccc4n5-c4ccc(-c5cnc6c(n5)oc5ccc7ccccc7c56)cc4)ccc32)cc1]

### Step 1: Synthesis of 9-(4-chlorophenyl)naphtho[1′,2′:4,5]furo[2,3-b]pyrazine

Into a three-neck flask were put 4.10 g of 9-chloronaphtho[1′,2′:4,5]furo[2,3-b]pyrazine whose synthesis method is described in Step 2 in Example 1, 2.80 g of 4-chlorophenylboronic acid, 27 mL of a 2M potassium carbonate aqueous solution, 160 mL of toluene, and 16 mL of ethanol, and the air in the flask was replaced with nitrogen. The mixture in the flask was degassed by being stirred under reduced pressure, and then 0.36 g of palladium(II) acetate (abbreviation: Pd(OAc)₂) and 2.08 g of tris(2,6-dimethoxyphenyl)phosphine (abbreviation: P(2,6-MeOPh)₃) were added thereto. The mixture was stirred at 90° C. for 7 hours to be reacted.

After a predetermined time elapsed, the obtained mixture was subjected to suction filtration and was washed with ethanol. Then, purification by silica gel column chromatography using toluene as a developing solvent was performed, so that 2.81 g of a target pyrazine derivative (yellowish white powder) was obtained in a yield of 52%. A synthesis scheme of Step 1 is shown in (h-1) below.

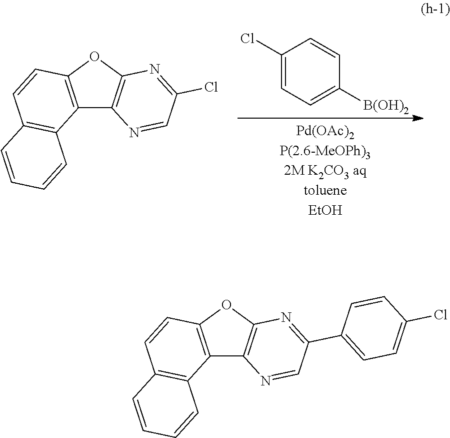 [SMILES: Clc1cnc2c(n1)oc1ccc3ccccc3c12 ; OB(O)c1ccc(Cl)cc1 ; Clc1ccc(-c2cnc3c(n2)oc2ccc4ccccc4c23)cc1]

### Step 2: Synthesis of 9pPCCzPNfpr

Next, into a three-neck flask were put 1.39 g of 9-(4-chlorophenyl)naphtho[1′,2′:4,5]furo[2,3-b]pyrazine obtained in Step 1, 1.72 g of 9′-phenyl-3,3′-bi-9H-carbazole, and 21 mL of mesitylene, and the air in the flask was replaced with nitrogen. The mixture in the flask was degassed by being stirred under reduced pressure, and then 0.81 g of sodium tert-butoxide, 0.024 g of tris(dibenzylideneacetone)dipalladium(0) (abbreviation: Pd₂(dba)₃), and 0.034 g of 2-dicyclohexylphosphino-2′,6′-dimethoxybiphenyl (abbreviation: S-Phos) were added thereto. The mixture was stirred at 150° C. for 6 hours to be reacted.

After a predetermined time elapsed, the reaction solution was subjected to extraction with toluene. The solid obtained by concentrating the extract solution was purified by silica gel column chromatography using toluene as a developing solvent, and then recrystallized with toluene three times, so that 1.84 g of a target yellow solid was obtained in a yield of 62%.

By a train sublimation method, 1.81 g of the obtained yellow solid was purified by sublimation. In the purification by sublimation, the solid was heated at 380° C. under a pressure of 2.7 Pa with an argon gas flow rate of 18 mL/min. After the purification by sublimation, 1.35 g of a target yellow solid was obtained in a yield of 75%. A synthesis scheme of Step 2 is shown in (h-2) below.

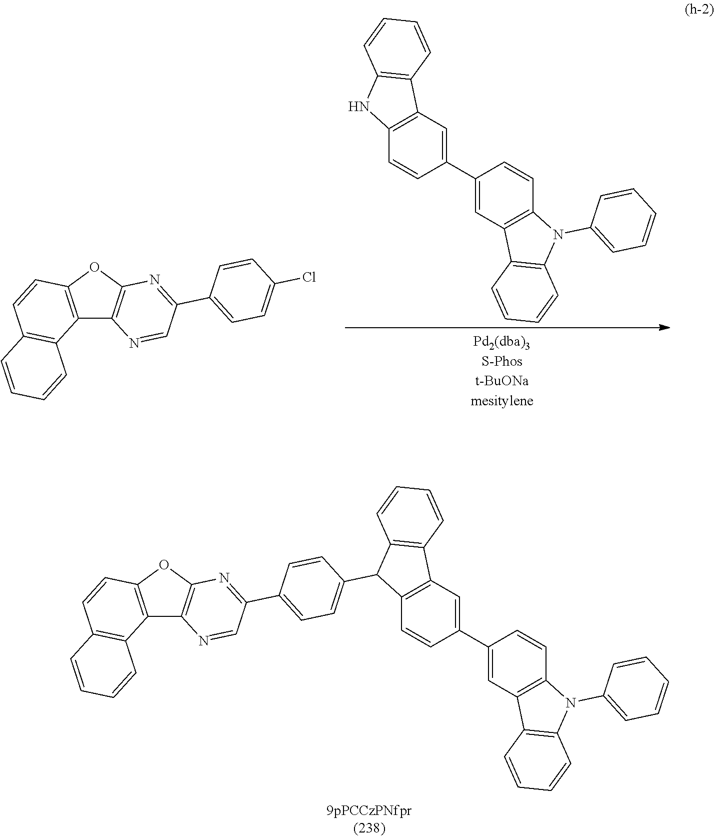 [SMILES: Clc1ccc(-c2cnc3c(n2)oc2ccc4ccccc4c23)cc1 ; c1ccc(-n2c3ccccc3c3cc(-c4ccc5[nH]c6ccccc6c5c4)ccc32)cc1 ; c1ccc(-n2c3ccccc3c3cc(-c4ccc5c(c4)-c4ccccc4C5c4ccc(-c5cnc6c(n5)oc5ccc7ccccc7c56)cc4)ccc32)cc1]

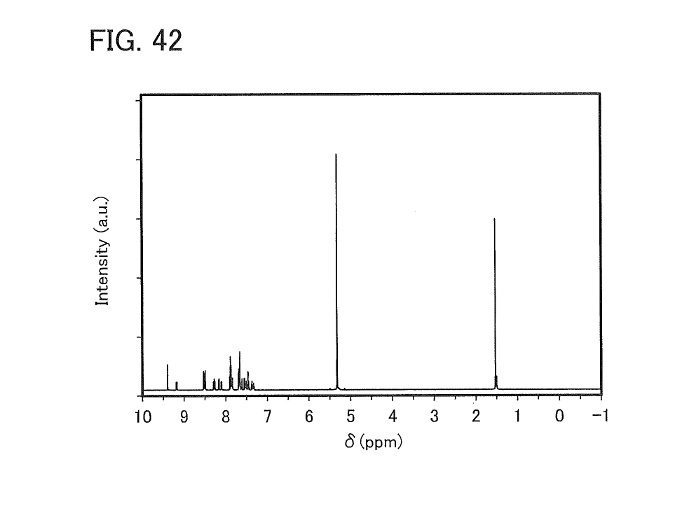

Analysis results by nuclear magnetic resonance (¹H-NMR) spectroscopy of the yellow solid obtained in Step 2 are shown below. FIG. 42 is the ¹H-NMR chart. The results revealed that 9pPCCzPNfpr, the organic compound represented by Structural Formula (238), was obtained in this example.

¹H-NMR. δ (CD₂Cl₂): 7.32-7.39 (m, 2H), 7.44-7.56 (m, 5H), 7.61 (d, 1H), 7.64-7.69 (m, 6H), 7.83-7.91 (m, 6H), 8.11 (d, 1H), 8.17 (d, 1H), 8.28 (d, 2H), 8.49-8.53 (m, 4H), 9.18 (d, 1H), 9.40 (s, 1H).

### Example 13

### Synthesis Example 9

This example describes a method for synthesizing 9-[4-(9′-phenyl-2,3′-bi-9H-carbazol-9-yl)phenyl]naphtho[1′,2′:4,5]furo[2,3-b]pyrazine (abbreviation: 9pPCCzPNfpr-02), which is the organic compound of one embodiment of the present invention represented by Structural Formula (239) in Embodiment 1. The structure of 9pPCCzPNfpr-02 is shown below.

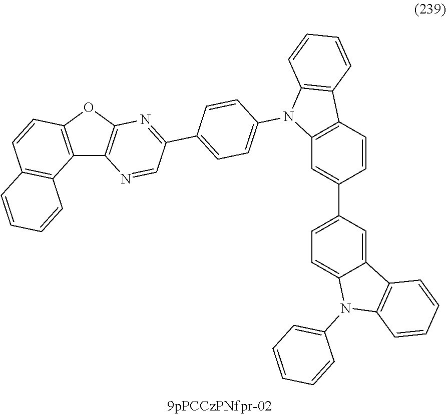 [SMILES: c1ccc(-n2c3ccccc3c3cc(-c4ccc5c6ccccc6n(-c6ccc(-c7cnc8c(n7)oc7ccc9ccccc9c78)cc6)c5c4)ccc32)cc1]

Into a three-neck flask were put 1.76 g of 9-(4-chlorophenyl)naphtho[1′,2′:4,5]furo[2,3-b]pyrazine whose synthesis method is described in Step 1 in Example 12, 2.22 g of 9′-phenyl-2,3′-bi-9H-carbazole, and 27 mL of mesitylene, and the air in the flask was replaced with nitrogen. The mixture in the flask was degassed by being stirred under reduced pressure, and then 1.09 g of sodium tert-butoxide, 0.031 g of tris(dibenzylideneacetone)dipalladium(0) (abbreviation: Pd₂(dba)₃), and 0.045 g of 2-dicyclohexylphosphino-2′,6′-dimethoxybiphenyl (abbreviation: S-Phos) were added thereto. The mixture was stirred at 150° C. for 6 hours to be reacted.

After a predetermined time elapsed, the obtained suspension was subjected to suction filtration and the residue was washed with water and ethanol. The obtained solid was purified by silica gel column chromatography using toluene as a developing solvent, and then recrystallized with a mixed solvent of toluene and hexane, so that 1.95 g of a target yellow solid was obtained in a yield of 52%.

By a train sublimation method, 1.94 g of the obtained yellow solid was purified by sublimation. In the purification by sublimation, the solid was heated at 380° C. under a pressure of 2.7 Pa with an argon gas flow rate of 18 mL/min. After the purification by sublimation, 1.62 g of a target yellow solid was obtained in a yield of 84%. A synthesis scheme is shown in (i-1) below.

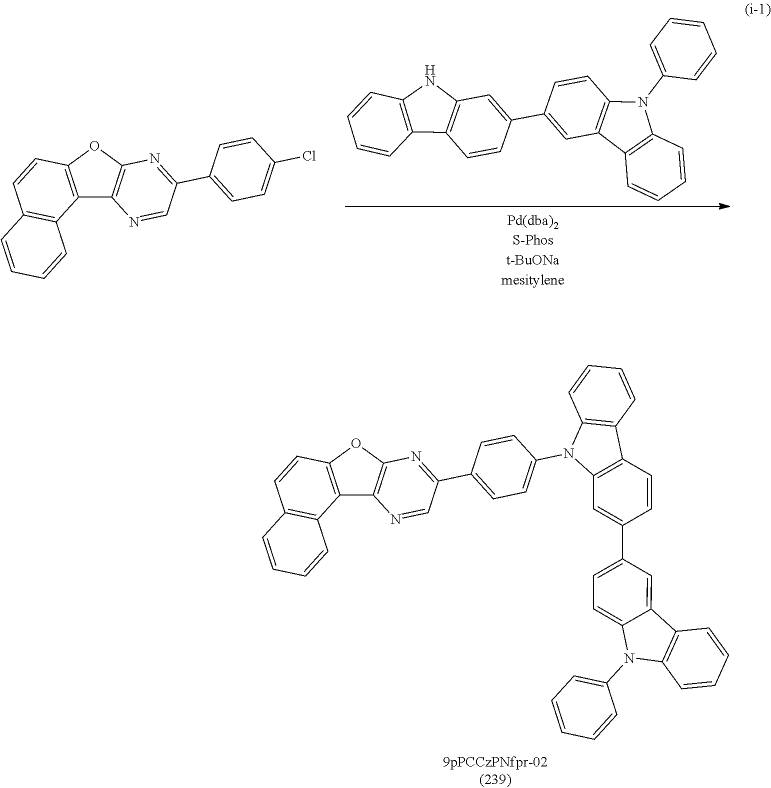 [SMILES: Clc1ccc(-c2cnc3c(n2)oc2ccc4ccccc4c23)cc1 ; c1ccc(-n2c3ccccc3c3cc(-c4ccc5c(c4)[nH]c4ccccc45)ccc32)cc1 ; c1ccc(-n2c3ccccc3c3cc(-c4ccc5c6ccccc6n(-c6ccc(-c7cnc8c(n7)oc7ccc9ccccc9c78)cc6)c5c4)ccc32)cc1]

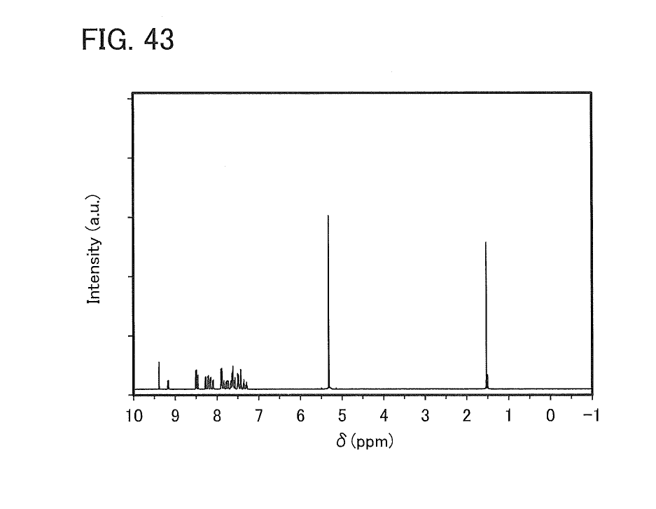

Analysis results by nuclear magnetic resonance (¹H-NMR) spectroscopy of the yellow solid obtained above are shown below. FIG. 43 is the ¹H-NMR chart. The results revealed that 9pPCCzPNfpr-02, the organic compound represented by Structural Formula (239), was obtained in this example.

¹H-NMR. δ (CD₂Cl₂): 7.28-7.31 (m, 1H), 7.36 (t, 1H), 7.40-7.44 (m, 2H), 7.46-7.51 (m, 3H), 7.57-7.69 (m, 6H), 7.74 (d, 1H), 8.78 (d, 1H), 7.84 (t, 1H), 7.81-7.88 (m, 4H), 8.10 (d, 1H), 8.16 (d, 1H), 8.22 (d, 2H), 8.28 (d, 1H), 8.46 (s, 1H), 8.50 (d, 2H), 9.17 (d, 1H), 9.38 (s, 1H).

### Example 14

### Synthesis Example 10

This example describes a method for synthesizing 9-[3′-(6-phenylbenzo[b]naphtho[1,2-d]furan-8-yl)biphenyl-3-yl]naphtho[1′,2′:4,5]furo[2,3-b]pyrazine (abbreviation: 9mBnfBPNfpr), which is the organic compound of one embodiment of the present invention represented by Structural Formula (244) in Embodiment 1. The structure of 9mBnfBPNfpr is shown below.

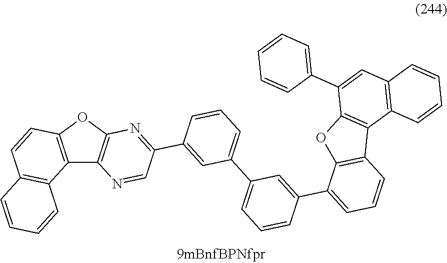 [SMILES: c1ccc(-c2cc3ccccc3c3c2oc2c(-c4cccc(-c5cccc(-c6cnc7c(n6)oc6ccc8ccccc8c67)c5)c4)cccc23)cc1]

Into a three-neck flask were put 1.28 g of 9-(3-chlorophenyl)naphtho[1′,2′:4,5]furo[2,3-b]pyrazine whose synthesis method is described in Step 1 in Example 7, 2.26 g of 3-(6-phenylbenzo[b]naphtho[1,2-d]furan-8-yl)phenylboronic acid pinacol ester, 2.53 g of tripotassium phosphate, 0.89 g of tert-butyl alcohol, and 32 mL of diethylene glycol dimethyl ether (abbreviation: diglyme), and the air in the flask was replaced with nitrogen. The mixture in the flask was degassed by being stirred under reduced pressure, and then 8.8 mg of palladium(II) acetate (abbreviation: Pd(OAc)₂) and 28 mg of di(1-adamantyl)-n-butylphosphine (abbreviation: CataCXium A) were added thereto. The mixture was stirred at 140° C. for 8.5 hours to be reacted.

After a predetermined time elapsed, the obtained suspension was subjected to suction filtration and was washed with water and ethanol. The obtained solid was purified by silica gel column chromatography using toluene as a developing solvent, and then recrystallized with toluene, so that 0.66 g of a target yellow solid was obtained in a yield of 25%. A synthesis scheme is shown in (j-1) below.

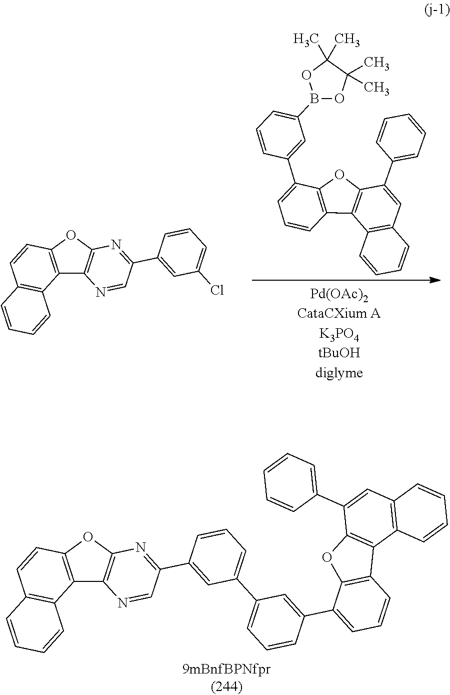 [SMILES: Clc1cccc(-c2cnc3c(n2)oc2ccc4ccccc4c23)c1 ; CC1(C)OB(c2cccc(-c3cccc4c3oc3c(-c5ccccc5)cc5ccccc5c34)c2)OC1(C)C ; c1ccc(-c2cc3ccccc3c3c2oc2c(-c4cccc(-c5cccc(-c6cnc7c(n6)oc6ccc8ccccc8c67)c5)c4)cccc23)cc1]

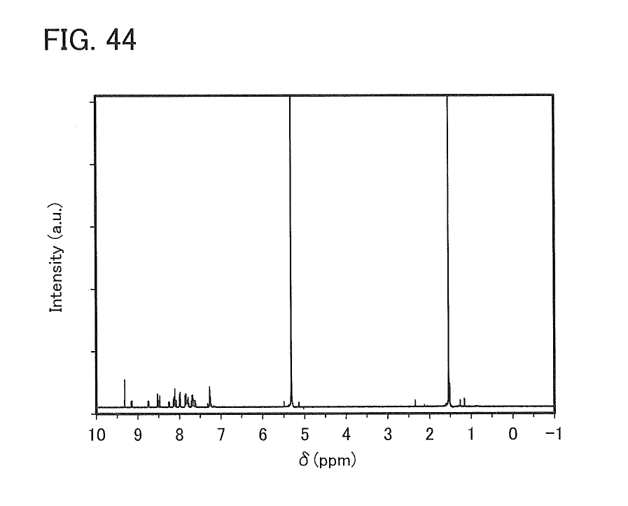

Analysis results by nuclear magnetic resonance (¹H-NMR) spectroscopy of the yellow solid obtained above are shown below. FIG. 44 is the ¹H-NMR chart. The results revealed that 9mBnfBPNfpr, the organic compound represented by Structural Formula (244), was obtained in this example.

¹H-NMR. δ (CD₂Cl₂): 7.24-7.28 (m, 3H), 7.61-7.72 (m, 5H), 7.78-7.87 (m, 6H), 7.98-8.00 (m, 3H), 8.08 (d, 1H), 8.11-8.15 (m, 3H), 8.25 (d, 1H), 8.48 (s, 1H), 8.51-8.53 (m, 2H), 8.75 (d, 1H), 9.15 (d, 1H), 9.32 (s, 1H).

### Example 15

### Synthesis Example 11

This example describes a method for synthesizing 9-[3′-(6-phenyldibenzothiophen-4-yl)biphenyl-3-yl]naphtho[1′,2′:4,5]furo[2,3-b]pyrazine (abbreviation: 9mDBtBPNfpr-02), which is the organic compound of one embodiment of the present invention represented by Structural Formula (245) in Embodiment 1. The structure of 9mDBtBPNfpr-02 is shown below.

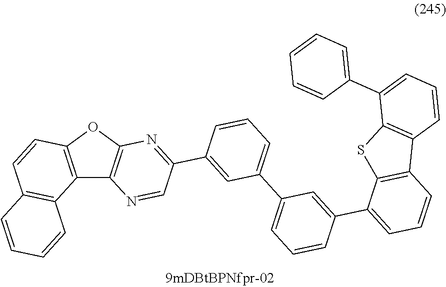 [SMILES: c1ccc(-c2cccc3c2sc2c(-c4cccc(-c5cccc(-c6cnc7c(n6)oc6ccc8ccccc8c67)c5)c4)cccc23)cc1]

Into a three-neck flask were put 1.19 g of 9-(3-chlorophenyl)naphtho[1′,2′:4,5]furo[2,3-b]pyrazine whose synthesis method is described in Step 1 in Example 7, 1.97 g of 3-(6-phenyldibenzothiophen-4-yl)phenylboronic acid pinacol ester, 2.29 g of tripotassium phosphate, 0.82 g of tert-butyl alcohol, and 29 mL of diethylene glycol dimethyl ether (abbreviation: diglyme), and the air in the flask was replaced with nitrogen. The mixture in the flask was degassed by being stirred under reduced pressure, and then 16 mg of palladium(II) acetate (abbreviation: Pd(OAc)₂) and 52 mg of di(1-adamantyl)-n-butylphosphine (abbreviation: CataCXium A) were added thereto. The mixture was stirred at 140° C. for 15 hours to be reacted.

After a predetermined time elapsed, the obtained suspension was subjected to suction filtration and was washed with water and ethanol. The obtained solid was purified by silica gel column chromatography using toluene as a developing solvent, and then recrystallized with toluene, so that 1.17 g of a target yellowish white solid was obtained in a yield of 52%. A synthesis scheme is shown in (k-1) below.

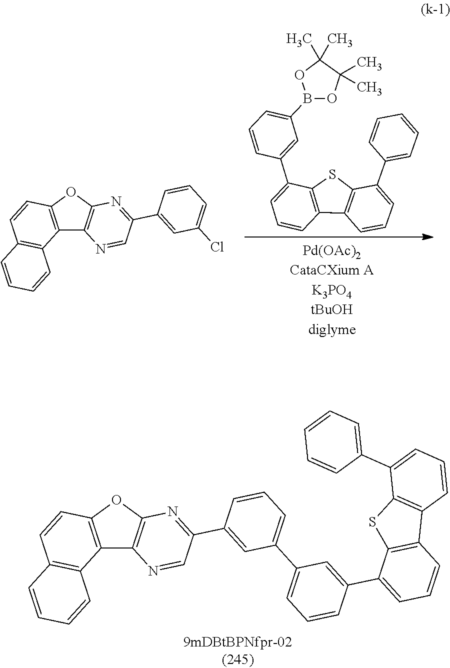 [SMILES: Clc1cccc(-c2cnc3c(n2)oc2ccc4ccccc4c23)c1 ; CC1(C)OB(c2cccc(-c3cccc4c3sc3c(-c5ccccc5)cccc34)c2)OC1(C)C ; c1ccc(-c2cccc3c2sc2c(-c4cccc(-c5cccc(-c6cnc7c(n6)oc6ccc8ccccc8c67)c5)c4)cccc23)cc1]

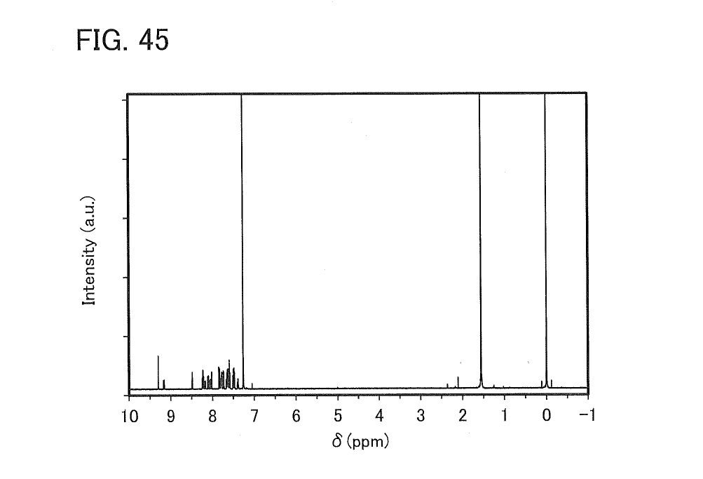

Analysis results by nuclear magnetic resonance (¹H-NMR) spectroscopy of the yellowish white solid obtained above are shown below. FIG. 45 is the ¹H-NMR chart. The results revealed that 9mDBtBPNfpr-02, the organic compound represented by Structural Formula (245), was obtained in this example.

¹H-NMR. δ (CD₂Cl₂): 7.39 (t, 1H), 7.47-7.51 (m, 3H), 7.58-7.67 (m, 6H), 7.73 (d, 2H), 7.78-7.85 (m, 5H), 8.02 (s, 1H), 8.06 (d, 1H), 8.10 (d, 1H), 8.18 (d, 1H), 8.23 (t, 2H), 8.49 (s, 1H), 9.17 (d, 1H), 9.30 (s, 1H).

### Example 16

### Synthesis Example 12

This example describes a method for synthesizing 9-{3-[6-(9,9-dimethylfluoren-2-yl)dibenzothiophen-4-yl]phenyl}naphtho[1′,2′:4,5]furo[2,3-b]pyrazine (abbreviation: 9mFDBtPNfpr), which is the organic compound of one embodiment of the present invention represented by Structural Formula (246) in Embodiment 1. The structure of 9mFDBtPNfpr is shown below.

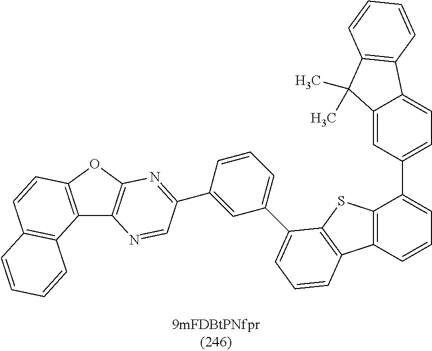 [SMILES: CC1(C)c2ccccc2-c2ccc(-c3cccc4c3sc3c(-c5cccc(-c6cnc7c(n6)oc6ccc8ccccc8c67)c5)cccc34)cc21]

Into a three-neck flask were put 1.01 g of 9-(3-chlorophenyl)naphtho[1′,2′:4,5]furo[2,3-b]pyrazine whose synthesis method is described in Step 1 in Example 7, 1.46 g of 3-[6-(9,9-dimethylfluoren-2-yl)dibenzothiophen-4-yl]phenylboronic acid, 1.89 g of tripotassium phosphate, 0.67 g of tert-butyl alcohol, and 24 mL of diethylene glycol dimethyl ether (abbreviation: diglyme), and the air in the flask was replaced with nitrogen. The mixture in the flask was degassed by being stirred under reduced pressure, and then 27 mg of palladium(II) acetate (abbreviation: Pd(OAc)₂) and 88 mg of di(1-adamantyl)-n-butylphosphine (abbreviation: CataCXium A) were added thereto. The mixture was stirred at 140° C. for 30 hours to be reacted.

After a predetermined time elapsed, the obtained suspension was subjected to suction filtration and was washed with water and ethanol. The obtained solid was purified by silica gel column chromatography using toluene as a developing solvent, and then recrystallized with a mixed solvent of toluene and hexane, so that 0.75 g of a target yellowish white solid was obtained in a yield of 37%. A synthesis scheme is shown in (l-1) below.

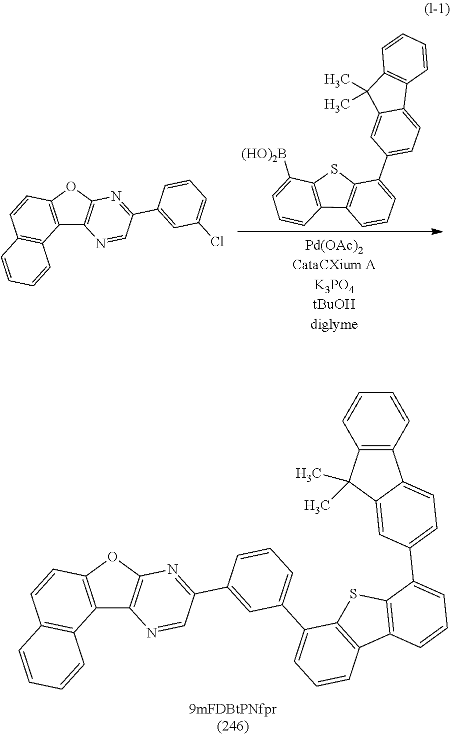 [SMILES: Clc1cccc(-c2cnc3c(n2)oc2ccc4ccccc4c23)c1 ; CC1(C)c2ccccc2-c2ccc(-c3cccc4c3sc3c(B(O)O)cccc34)cc21 ; CC1(C)c2ccccc2-c2ccc(-c3cccc4c3sc3c(-c5cccc(-c6cnc7c(n6)oc6ccc8ccccc8c67)c5)cccc34)cc21]

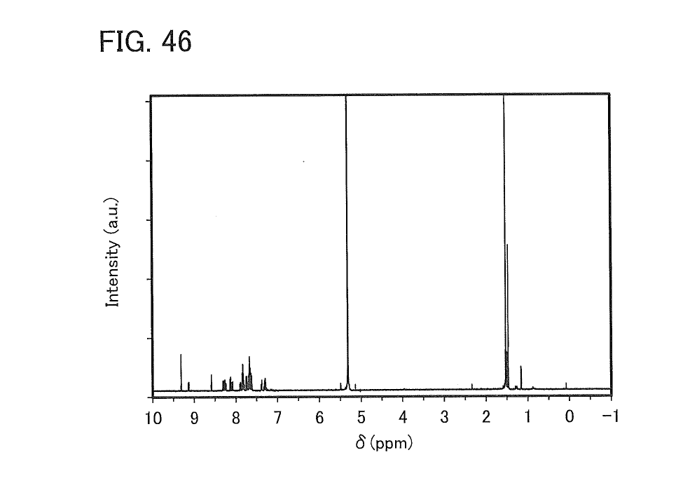

Analysis results by nuclear magnetic resonance (¹H-NMR) spectroscopy of the yellowish white solid obtained above are shown below. FIG. 46 is the ¹H-NMR chart. The results revealed that 9mFDBtPNfpr, the organic compound represented by Structural Formula (246), was obtained in this example.

¹H-NMR. δ (CD₂Cl₂): 1.47 (s, 6H), 7.27-7.32 (m, 2H), 7.38 (d, 1H), 7.61-7.76 (m, 8H), 7.79-7.85 (m, 4H), 7.89 (d, 1H), 8.08 (d, 1H), 8.13 (d, 1H), 8.24-8.31 (m, 3H), 8.59 (s, 1H), 9.14 (d, 1H), 9.31 (s, 1H).

### Example 17

### Synthesis Example 13

This example describes a method for synthesizing 11-(3-naphtho[1′,2′:4,5]furo[2,3-b]pyrazin-9-yl-phenyl)-12-phenylindolo[2,3-a]carbazole (abbreviation: 9mIcz(II)PNfpr), which is the organic compound of one embodiment of the present invention represented by Structural Formula (247) in Embodiment 1. The structure of 9mIcz(II)PNfpr is shown below.

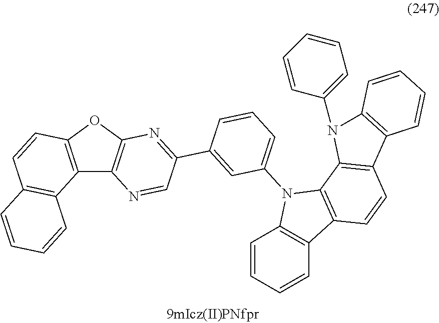 [SMILES: c1ccc(-n2c3ccccc3c3ccc4c5ccccc5n(-c5cccc(-c6cnc7c(n6)oc6ccc8ccccc8c67)c5)c4c32)cc1]

A synthesis method of 9mIcz(II)PNfpr is shown by a synthesis scheme (m-1) below.

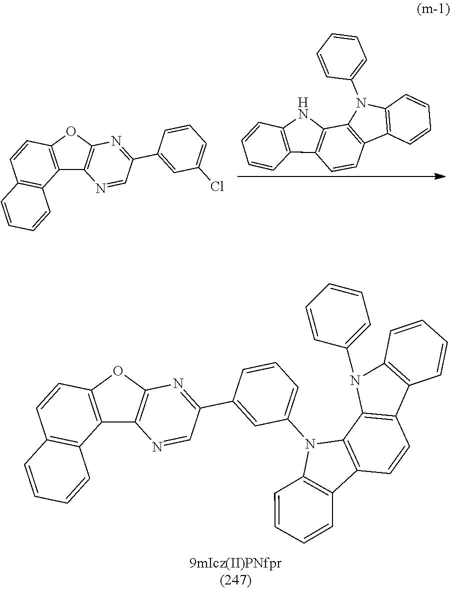

### Example 18

### Synthesis Example 14

This example describes a method for synthesizing 3-naphtho[1′,2′:4,5]furo[2,3-b]pyrazin-9-yl-N,N-diphenylbenzenamine (abbreviation: 9mTPANfpr), which is the organic compound of one embodiment of the present invention represented by Structural Formula (248) in Embodiment 1. The structure of 9mTPANfpr is shown below.

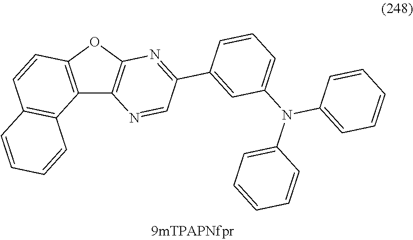 [SMILES: c1ccc(N(c2ccccc2)c2cccc(-c3cnc4c(n3)oc3ccc5ccccc5c34)c2)cc1]

A synthesis method of 9mTPANfpr is shown by a synthesis scheme (n-1) below.

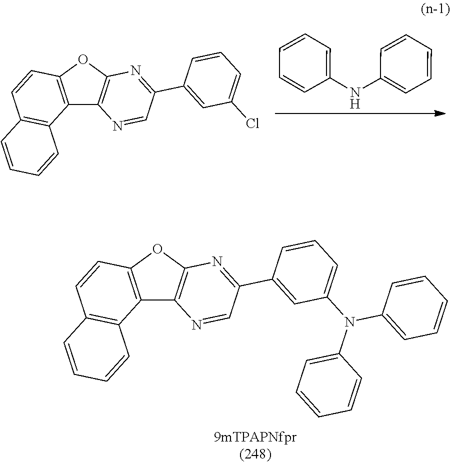

### Example 19

In this example, a light-emitting element 8 using 10mDBtBPNfpr (Structural Formula (133), Example 9) in its light-emitting layer was fabricated as a light-emitting element of one embodiment of the present invention. The measured characteristic results of the light-emitting element 8 will be described below.

The element structure of the light-emitting element 8 fabricated in this example was similar to the element structure described in Example 2 with reference to FIG. 11. Table 12 shows specific structures of layers in the element structure. Chemical formulae of materials used in this example are shown below.

TABLE 12

| | | | Hole- | Light- | | | Electron- | |
|---|---|---|---|---|---|---|---|---|
| | First | Hole-injection | transport | emitting | | | injection | Second |
| | electrode | layer | layer | layer | Electron-transport layer | | layer | electrode |
| | 901 | 911 | 912 | 913 | 914 | | 915 | 903 |
| Light- | ITSO | DBT3P-II:MoOx | PCBBi1BP | * | 10mDBtBPNfpr | NBphen | LiF | Al |
| emitting | (70 nm) | (2:1, 75 nm) | (20 nm) | | (30 nm) | (15 nm) | (1 nm) | (200 nm) |
| element 8 | | | | | | | | |

* 10mDBtBPNfpr:PCBBiF:[Ir(dmpqn)₂(acac)] (0.75:0.25:0.1, 40 nm)

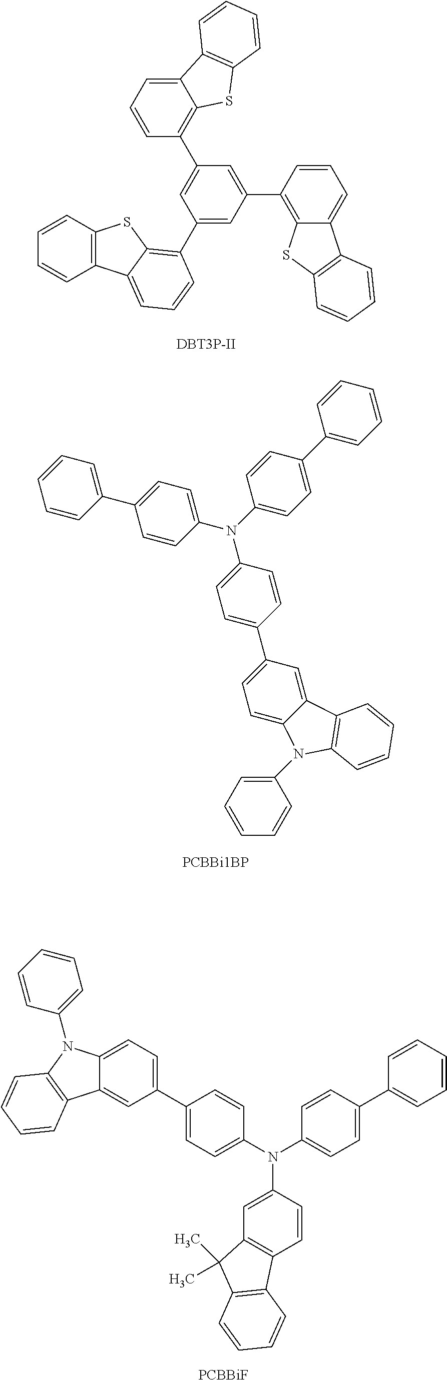

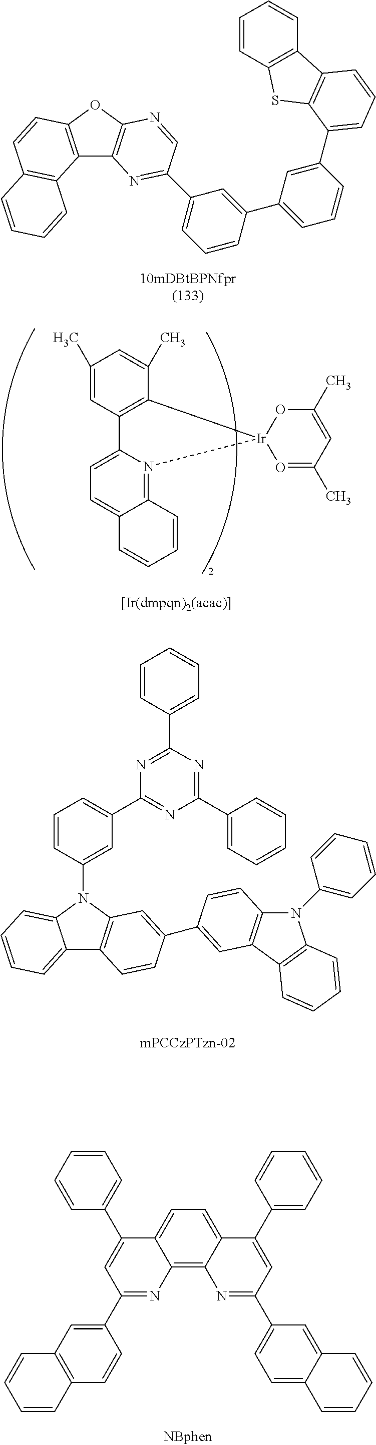

### <<Operation Characteristics of Light-Emitting Element 8>>

Operation characteristics of the fabricated light-emitting element 8 were measured. Note that the measurement was performed at room temperature (in an atmosphere kept at 25° C.).

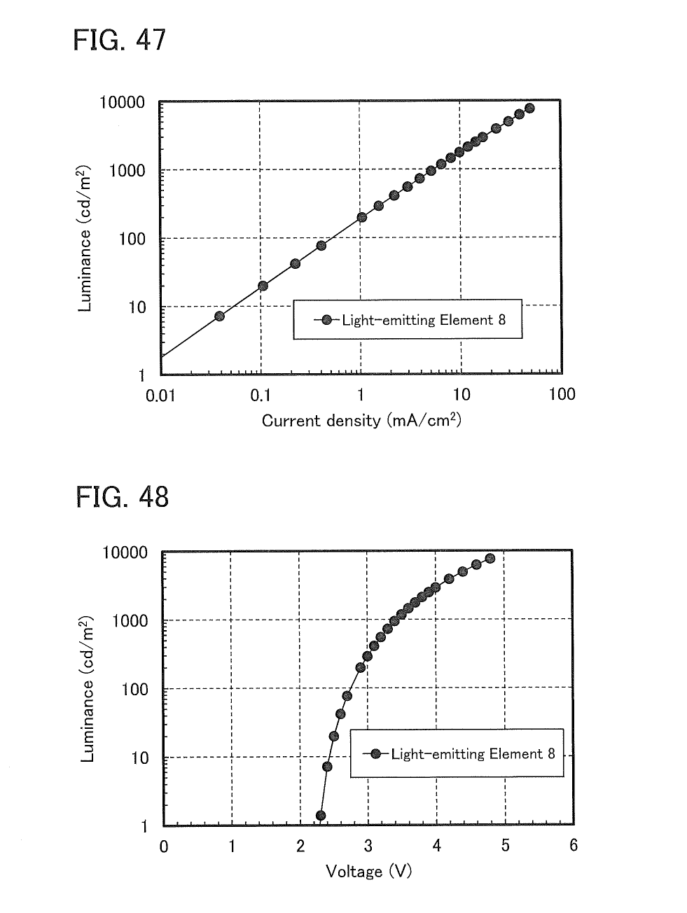

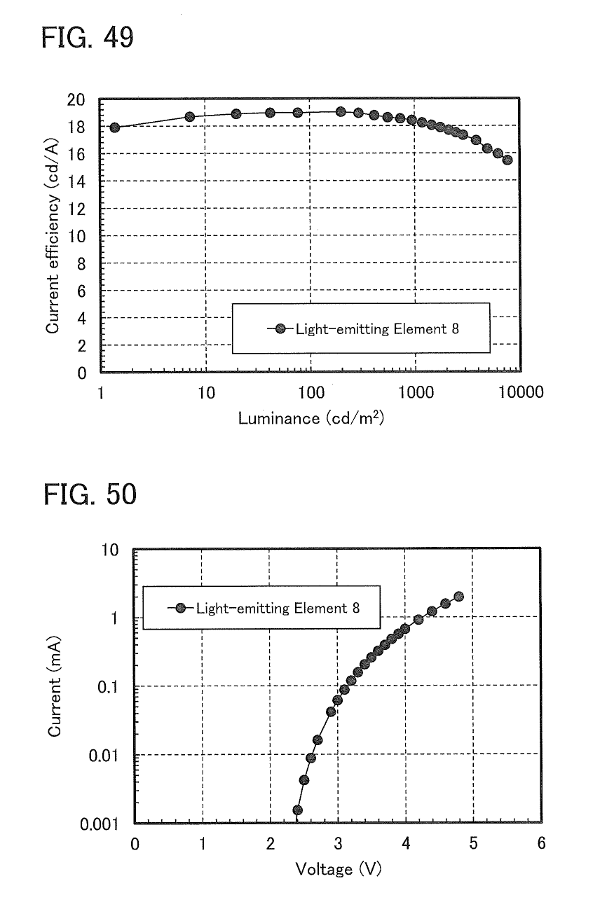

FIG. 47, FIG. 48, FIG. 49, and FIG. 50 show the current density-luminance characteristics, the voltage-luminance characteristics, the luminance-current efficiency characteristics, and the voltage-current characteristics, respectively, of the light-emitting element 8.

Table 13 shows initial values of main characteristics of the light-emitting element 8 at around 1000 cd/m².

TABLE 13

| | | | | | | | | External |
|---|---|---|---|---|---|---|---|---|
| | | | Current | | | Current | Power | quantum |
| | Voltage | Current | density | Chromaticity | Luminance | efficiency | efficiency | efficiency |
| | (V) | (mA) | (mA/cm²) | (x, y) | (cd/m²) | (cd/A) | (lm/W) | (%) |
| Light- | 3.4 | 0.21 | 5.1 | (0.68, 0.32) | 950 | 18 | 17 | 19 |
| emitting | | | | | | | | |
| element 8 | | | | | | | | |

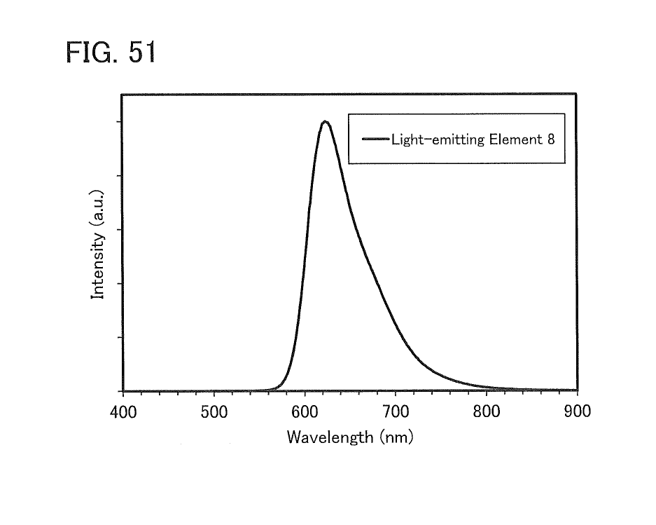

FIG. 51 shows an emission spectrum when current at a current density of 2.5 mA/cm² was applied to the light-emitting element 8. As shown in FIG. 51, the emission spectrum of the light-emitting element has a peak at around 626 nm that is probably derived from light emission of [Ir(dmpqn)₂(acac)] contained in the light-emitting layer 913.

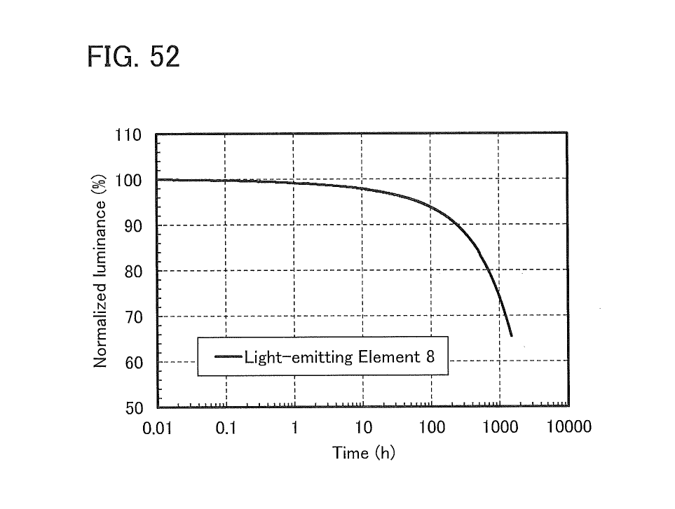

Next, a reliability test was performed on the light-emitting element 8. FIG. 52 shows results of the reliability test. In FIG. 52, the vertical axis represents normalized luminance (%) with an initial luminance of 100%, and the horizontal axis represents driving time (h) of the element. As the reliability test, a constant current driving test at a constant current density of 75 mA/cm² was performed.

The results of the reliability test show that the light-emitting element 8 including 10mDBtBPNfpr, which is the organic compound of one embodiment of the present invention, has high reliability. This indicates that the use of the organic compound of one embodiment of the present invention is effective in improving the reliability of a light-emitting element.

### Example 20

In this example, a light-emitting element 9 using 12mDBtBPPnfpr (Structural Formula (208), Example 11) in its light-emitting layer was fabricated as a light-emitting element of one embodiment of the present invention. The measured characteristic results of the light-emitting element 9 will be described below.

The element structure of the light-emitting element 9 fabricated in this example was similar to the element structure described in Example 2 with reference to FIG. 11. Table 14 shows specific structures of layers in the element structure. Chemical formulae of materials used in this example are shown below.

TABLE 14

| | | | Hole- | Light- | | | Electron- | |
|---|---|---|---|---|---|---|---|---|
| | First | Hole-injection | transport | emitting | | | injection | Second |
| | electrode | layer | layer | layer | Electron-transport layer | | layer | electrode |
| | 901 | 911 | 912 | 913 | 914 | | 915 | 903 |
| Light- | ITSO | DBT3P-II:MoOx | PCBBi1BP | * | 12mDBtBPPnfpr | NBphen | LiF | Al |
| emitting | (70 nm) | (2:1, 70 nm) | (20 nm) | | (30 nm) | (15 nm) | (1 nm) | (200 nm) |
| element 9 | | | | | | | | |

* 12mDBtBPPnfpr:PCBBiF:[Ir(dmpqn)₂(acac)] (0.75:0.25:0.1, 40 nm)

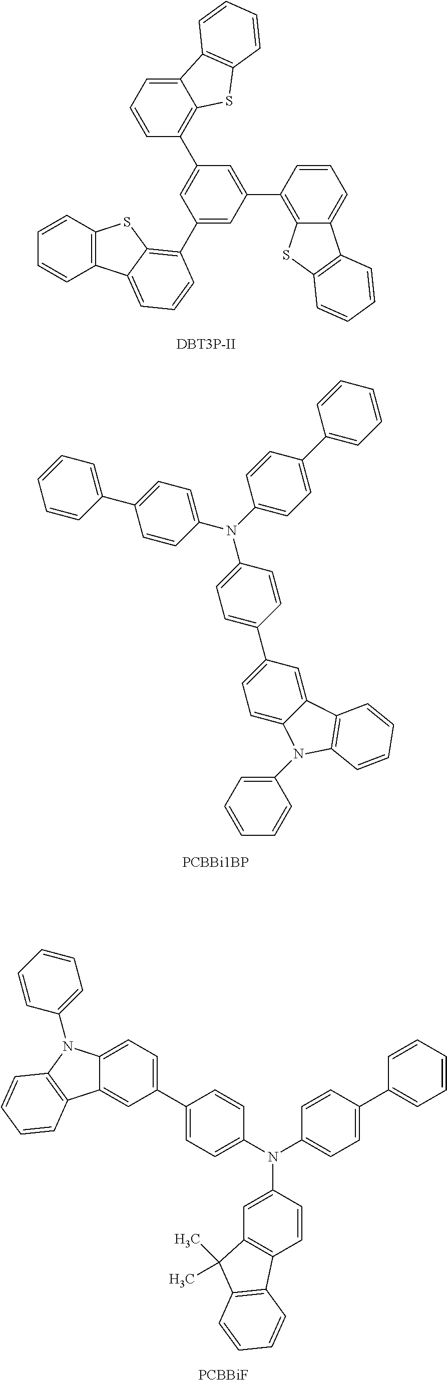

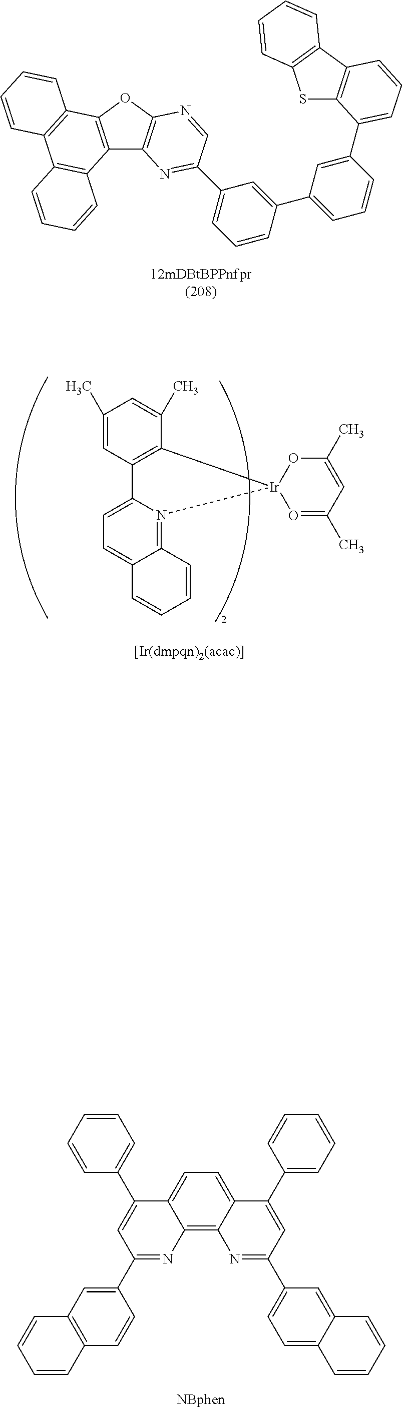

### <<Operation Characteristics of Light-Emitting Element 9>>

Operation characteristics of the fabricated light-emitting element 9 were measured. Note that the measurement was performed at room temperature (in an atmosphere kept at 25° C.).

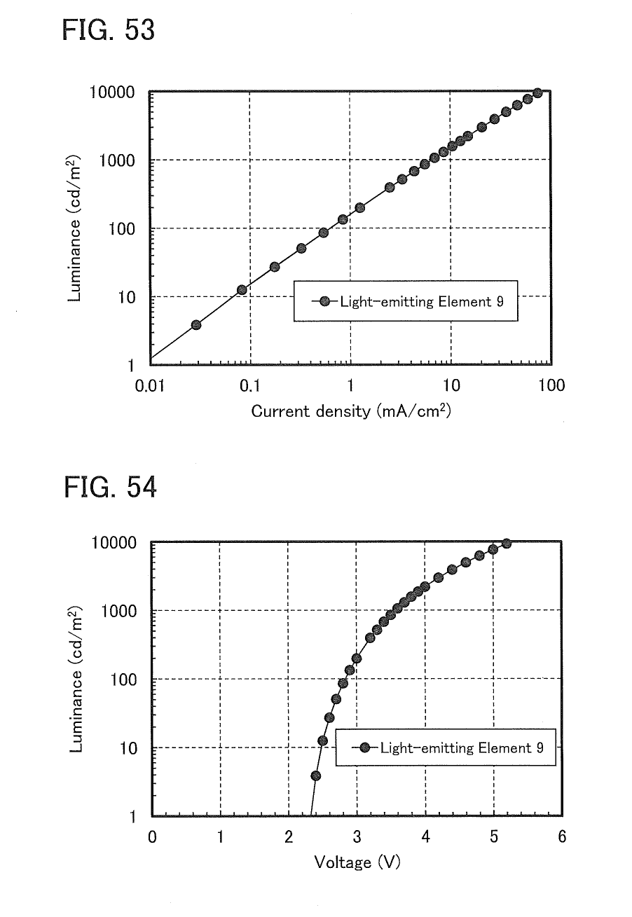

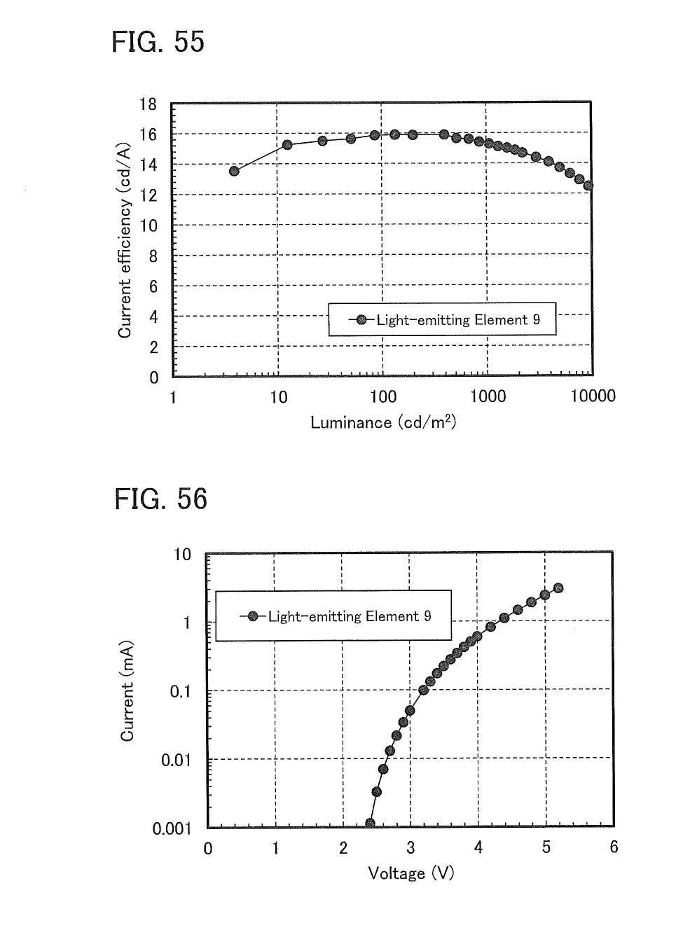

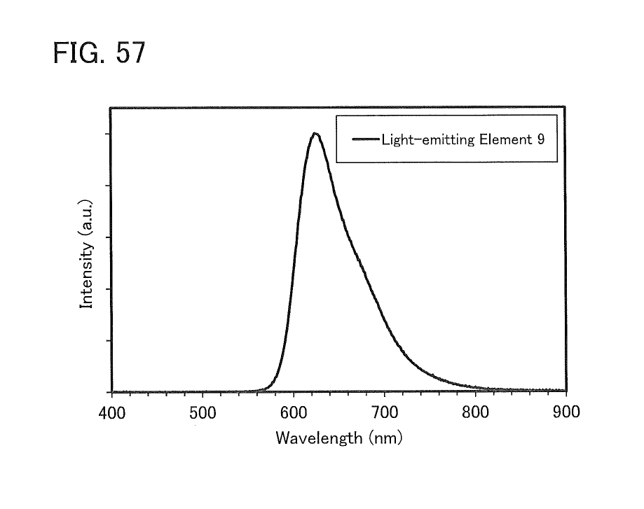

FIG. 53, FIG. 54, FIG. 55, and FIG. 56 show the current density-luminance characteristics, the voltage-luminance characteristics, the luminance-current efficiency characteristics, and the voltage-current characteristics, respectively, of the light-emitting element 9.

Table 15 shows initial values of main characteristics of the light-emitting element 9 at around 1000 cd/m².

TABLE 15

| | | | | | | | | External |
|---|---|---|---|---|---|---|---|---|
| | | | Current | | | Current | Power | quantum |
| | Voltage | Current | density | Chromaticity | Luminance | efficiency | efficiency | efficiency |
| | (V) | (mA) | (mA/cm²) | (x, y) | (cd/m²) | (cd/A) | (lm/W) | (%) |
| Light- | 3.6 | 0.28 | 7.0 | (0.68, 0.32) | 1100 | 15 | 13 | 17 |
| emitting | | | | | | | | |
| element 9 | | | | | | | | |

FIG. 57 shows an emission spectrum when current at a current density of 2.5 mA/cm² was applied to the light-emitting element 9. As shown in FIG. 57, the emission spectrum of the light-emitting element has a peak at around 626 nm that is probably derived from light emission of [Ir(dmpqn)₂(acac)] contained in the light-emitting layer 913.

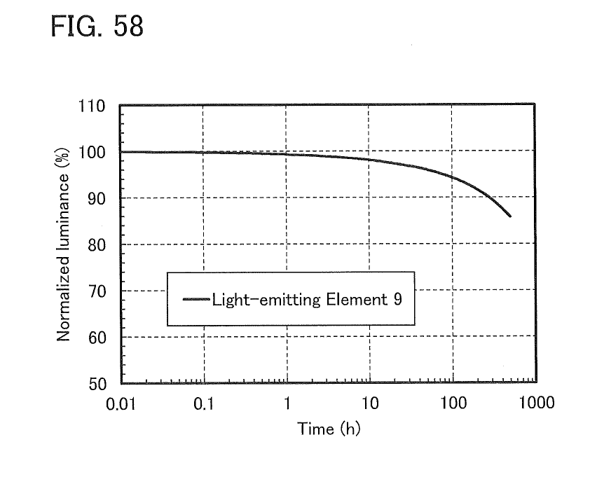

Next, a reliability test was performed on the light-emitting element 9. FIG. 58 shows results of the reliability test. In FIG. 58, the vertical axis represents normalized luminance (%) with an initial luminance of 100%, and the horizontal axis represents driving time (h) of the element. As the reliability test, a constant current driving test at a constant current density of 75 mA/cm² was performed.

The results of the reliability test show that the light-emitting element 9 including 12mDBtBPPnfpr, which is the organic compound of one embodiment of the present invention, has high reliability. This indicates that the use of the organic compound of one embodiment of the present invention is effective in improving the reliability of a light-emitting element.

### Example 21

In this example, light-emitting elements 10 to 15 were fabricated as light-emitting elements of embodiments of the present invention. The light-emitting element 10 was fabricated using 9PCCzNfpr (Structural Formula (123), Example 6) in its light-emitting layer. The light-emitting element 11 was fabricated using 10PCCzNfpr (Structural Formula (156), Example 10) in its light-emitting layer. The light-emitting element 12 was fabricated using 9mPCCzPNfpr (Structural Formula (125), Example 7) in its light-emitting layer. The light-emitting element 13 was fabricated using 9mPCCzPNfpr-02 (Structural Formula (126), Example 8) in its light-emitting layer. The light-emitting element 14 was fabricated using 9pPCCzPNfpr (Structural Formula (238), Example 12) in its light-emitting layer. The light-emitting element 15 was fabricated using 9pPCCzPNfpr-02 (Structural Formula (239), Example 13) in its light-emitting layer. The measured characteristic results of the light-emitting elements 10 to 15 will be described below.

The element structures of the light-emitting elements 10 to 15 fabricated in this example were similar to the element structure of the light-emitting element 3 described in Example 3. Table 16 shows specific structures of layers in the element structures. Chemical formulae of materials used in this example are shown below.

TABLE 16

| | | | Hole- | Light- | | | Electron- | |
|---|---|---|---|---|---|---|---|---|
| | First | Hole-injection | transport | emitting | | | injection | Second |
| | electrode | layer | layer | layer | Electron-transport layer | | layer | electrode |
| | 901 | 911 | 912 | 913 | 914 | | 915 | 903 |
| Light- | ITSO | DBT3P-II:MoOx | PCBBi1BP | * | 9PCCzNfpr | NBphen | LiF | Al |
| emitting | (70 nm) | (2:1, 70 nm) | (20 nm) | | (30 nm) | (15 nm) | (1 nm) | (200 nm) |
| element 10 | | | | | | | | |
| Light- | ITSO | DBT3P-II:MoOx | PCBBi1BP | ** | 10PCCzNfpr | NBphen | LiF | Al |
| emitting | (70 nm) | (2:1, 70 nm) | (20 nm) | | (30 nm) | (15 nm) | (1 nm) | (200 nm) |
| element 11 | | | | | | | | |
| Light- | ITSO | DBT3P-II:MoOx | PCBBi1BP | *** | 9mPCCzPNfpr | NBphen | LiF | Al |
| emitting | (70 nm) | (2:1, 70 nm) | (20 nm) | | (30 nm) | (15 nm) | (1 nm) | (200 nm) |
| element 12 | | | | | | | | |
| Light- | ITSO | DBT3P-II:MoOx | PCBBi1BP | **** | 9mPCCzPNfpr-02 | NBphen | LiF | Al |
| emitting | (70 nm) | (2:1, 70 nm) | (20 nm) | | (30 nm) | (15 nm) | (1 nm) | (200 nm) |
| element 13 | | | | | | | | |
| Light- | ITSO | DBT3P-II:MoOx | PCBBi1BP | ***** | 9pPCCzPNfpr | NBphen | LiF | Al |
| emitting | (70 nm) | (2:1, 70 nm) | (20 nm) | | (30 nm) | (15 nm) | (1 nm) | (200 nm) |
| element 14 | | | | | | | | |
| Light- | ITSO | DBT3P-II:MoOx | PCBBi1BP | ****** | 9pPCCzPNfpr-02 | NBphen | LiF | Al |
| emitting | (70 nm) | (2:1, 70 nm) | (20 nm) | | (30 nm) | (15 nm) | (1 nm) | (200 nm) |
| element 15 | | | | | | | | |

* 9PCCzNfpr:[Ir(dmpqn)₂(acac)] (1.0:0.1, 40 nm)
** 10PCCzNfpr:[Ir(dmpqn)₂(acac)] (1.0:0.1, 40 nm)
*** 9mPCCzPNfpr:[Ir(dmpqn)₂(acac)] (1.0:0.1, 40 nm)
**** 9mPCCzPNfpr-02:[Ir(dmpqn)₂(acac)] (1.0:0.1, 40 nm)
***** 9pPCCzPNfpr:[Ir(dmpqn)₂(acac)] (1.0:0.1, 40 nm)
****** 9pPCCzPNfpr-02:[Ir(dmpqn)₂(acac)] (1.0:0.1, 40 nm)

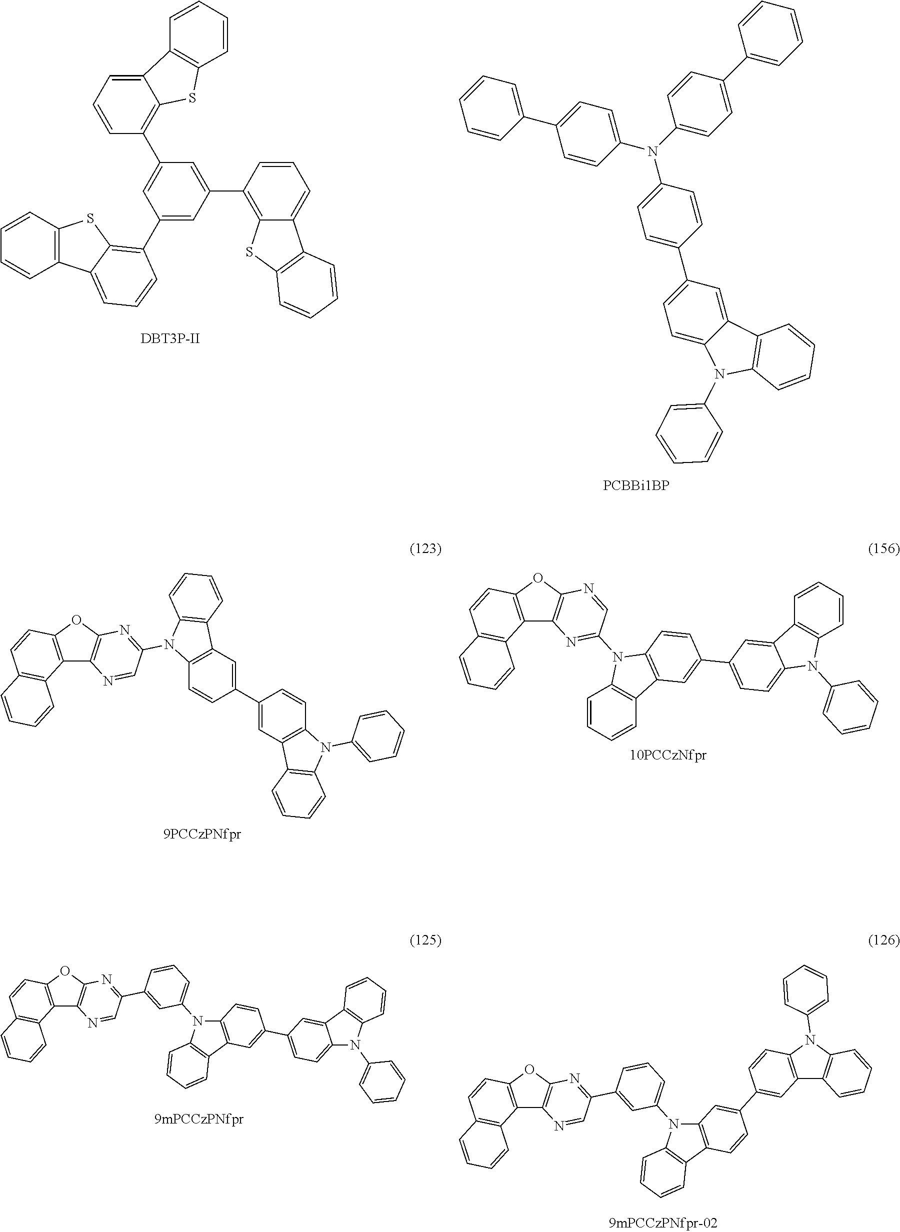

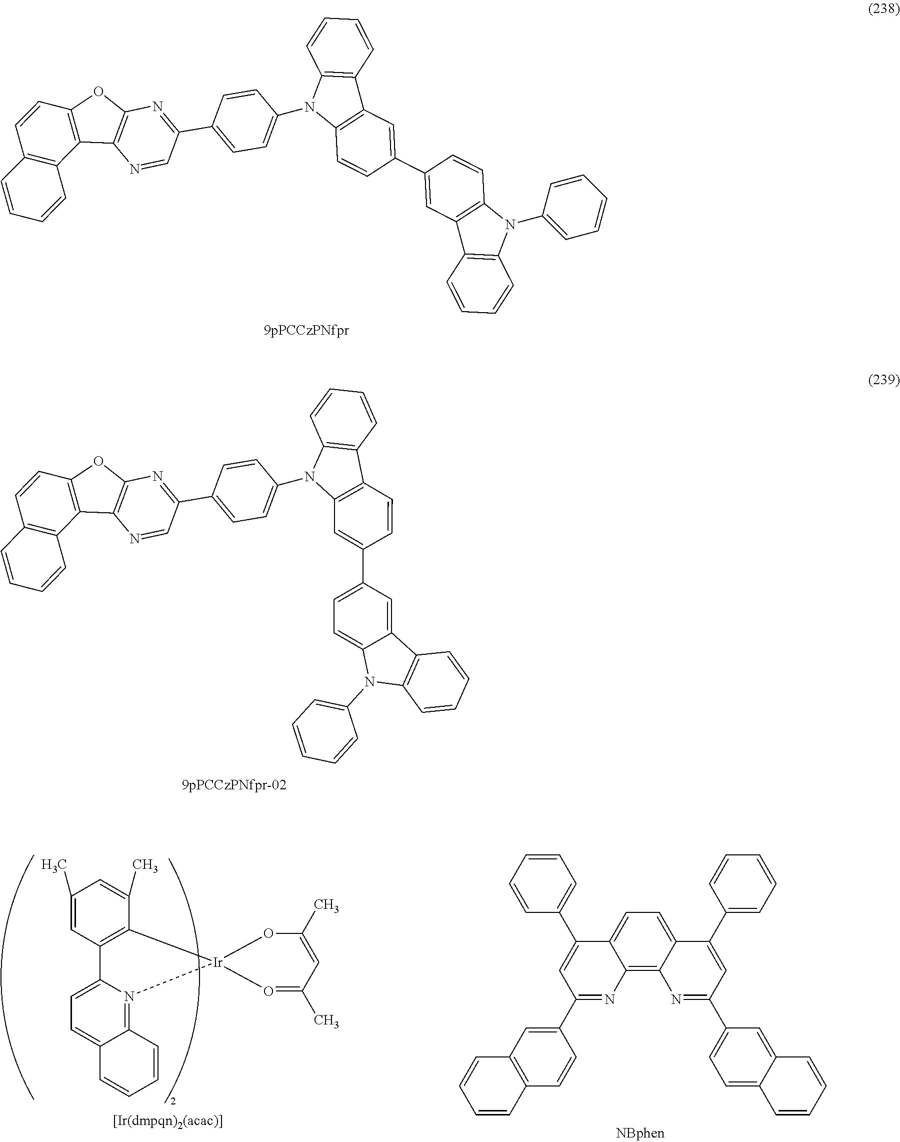

### <<Operation Characteristics of Light-Emitting Elements>>

Operation characteristics of the fabricated light-emitting elements 10 to 15 were measured. Note that the measurement was performed at room temperature (in an atmosphere kept at 25° C.).

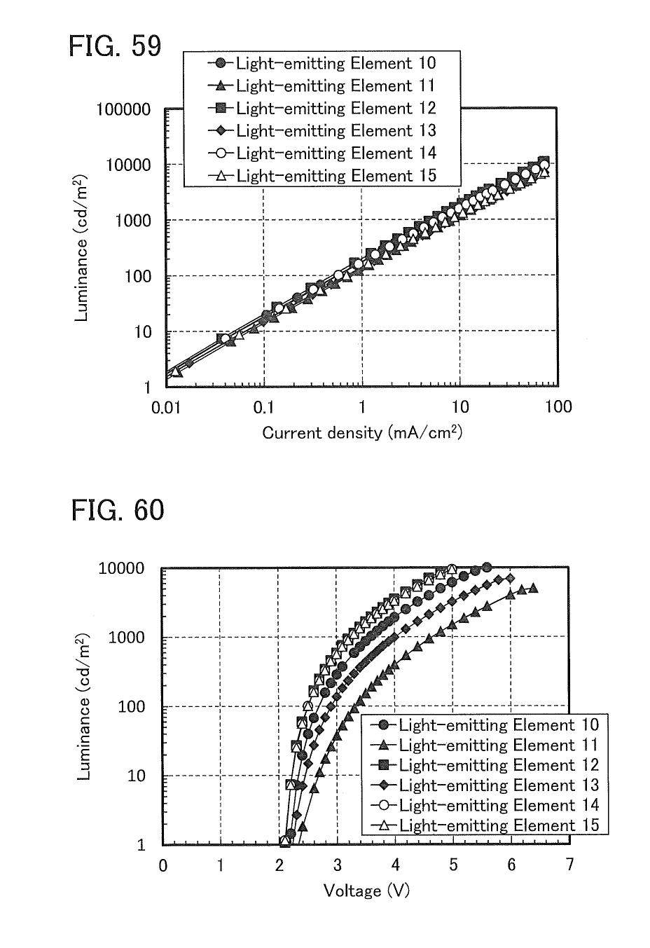

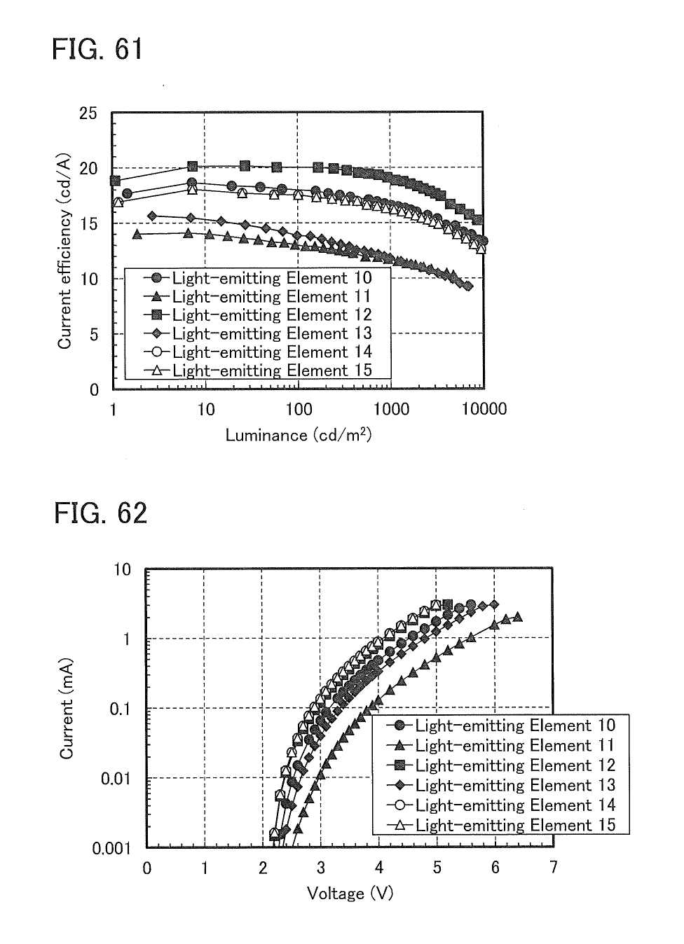

FIG. 59, FIG. 60, FIG. 61, and FIG. 62 show the current density-luminance characteristics, the voltage-luminance characteristics, the luminance-current efficiency characteristics, and the voltage-current characteristics, respectively, of the light-emitting elements.

Table 17 shows initial values of main characteristics of the light-emitting elements at around 1000 cd/m².

TABLE 17

| | | | | | | | | External |
|---|---|---|---|---|---|---|---|---|
| | | | Current | | | Current | Power | quantum |
| | Voltage | Current | density | Chromaticity | Luminance | efficiency | efficiency | efficiency |
| | (V) | (mA) | (mA/cm²) | (x, y) | (cd/m²) | (cd/A) | (lm/W) | (%) |
| Light- | 3.6 | 0.25 | 6.2 | (0.68, 0.32) | 1000 | 17 | 14 | 18 |
| emitting | | | | | | | | |
| element 10 | | | | | | | | |
| Light- | 4.6 | 0.32 | 8.0 | (0.68, 0.32) | 940 | 12 | 8.0 | 14 |
| emitting | | | | | | | | |
| element 11 | | | | | | | | |
| Light- | 3.2 | 0.20 | 4.9 | (0.68, 0.32) | 930 | 19 | 19 | 21 |
| emitting | | | | | | | | |
| element 12 | | | | | | | | |
| Light- | 4.0 | 0.33 | 8.4 | (0.68, 0.32) | 990 | 12 | 9.3 | 14 |
| emitting | | | | | | | | |
| element 13 | | | | | | | | |
| Light- | 3.3 | 0.27 | 6.8 | (0.68, 0.32) | 1100 | 16 | 15 | 19 |
| emitting | | | | | | | | |
| element 14 | | | | | | | | |
| Light- | 3.3 | 0.29 | 7.2 | (0.68, 0.32) | 900 | 13 | 12 | 15 |
| emitting | | | | | | | | |
| element 15 | | | | | | | | |

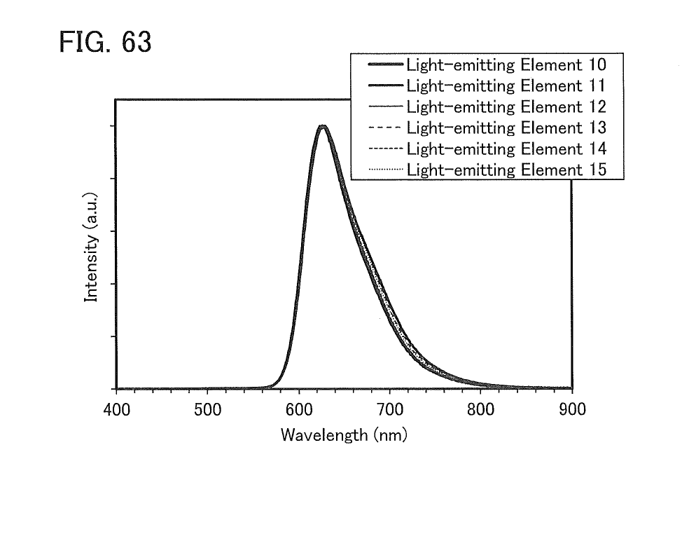

FIG. 63 shows emission spectra when current at a current density of 2.5 mA/cm² was applied to the light-emitting elements. As shown in FIG. 63, the emission spectrum of each light-emitting element has a peak at around 629 nm that is probably derived from light emission of [Ir(dmpqn)₂(acac)] contained in the light-emitting layer 913.

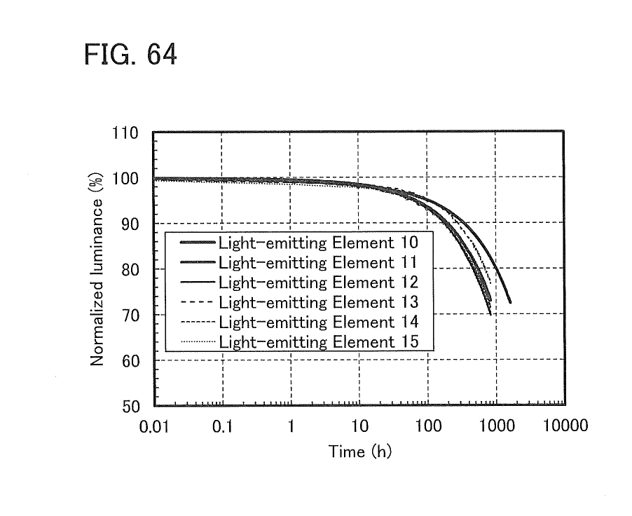

Next, reliability tests were performed on the light-emitting elements. FIG. 64 shows results of the reliability tests. In FIG. 64, the vertical axis represents normalized luminance (%) with an initial luminance of 100%, and the horizontal axis represents driving time (h) of the elements. As the reliability tests, constant current driving tests at a constant current density of 75 mA/cm² were performed.

The results of the reliability tests show that the light-emitting elements 10 to 15 including 9PCCzNfpr, 10PCCzNfpr, 9mPCCzPNfpr, 9mPCCzPNfpr-02, 9pPCCzPNfpr, and 9pPCCzPNfpr-02, respectively, which are the organic compounds of embodiments of the present invention, in the light-emitting layers have high reliability. This indicates that the use of the organic compound of one embodiment of the present invention is effective in improving the reliability of a light-emitting element.

### Example 22

### Synthesis Example 15

This example describes a method for synthesizing 10-[4-(9′-phenyl-3,3′-bi-9H-carbazol-9-yl)phenyl]naphtho[1′,2′:4,5]furo[2,3-b]pyrazine (abbreviation: 10mPCCzPNfpr), which is the organic compound of one embodiment of the present invention represented by Structural Formula (158) in Embodiment 1. The structure of 10mPCCzPNfpr is shown below.

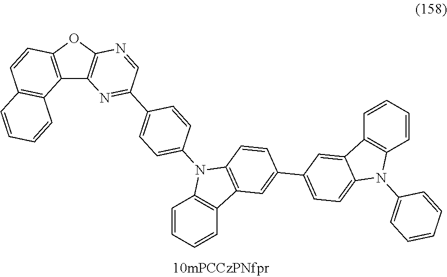 [SMILES: c1ccc(-n2c3ccccc3c3cc(-c4ccc5c(c4)c4ccccc4n5-c4ccc(-c5cnc6oc7ccc8ccccc8c7c6n5)cc4)ccc32)cc1]

A synthesis method of 10mPCCzPNfpr is shown by synthesis schemes (o-1) to (o-4) below.

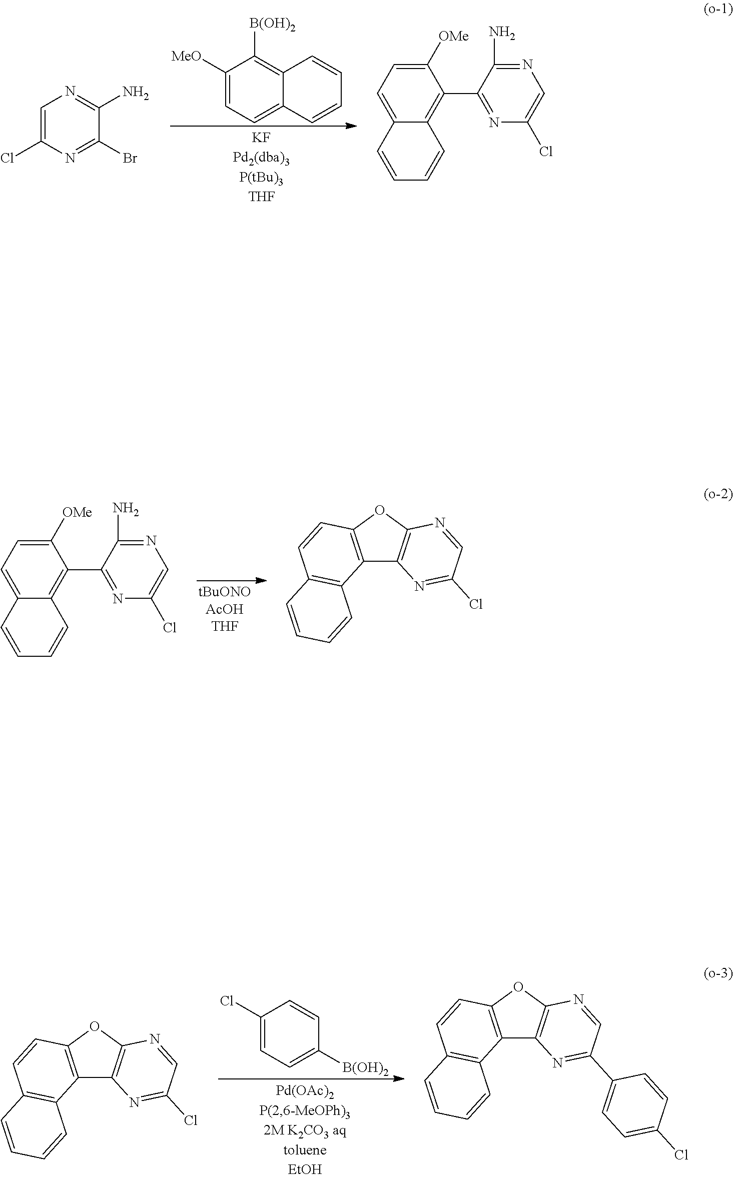

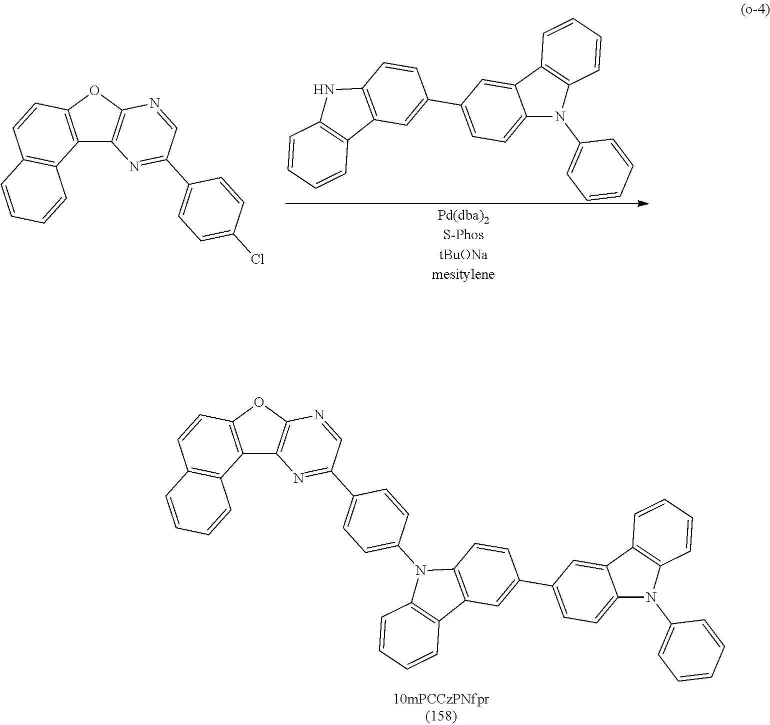 [SMILES: Clc1ccc(-c2cnc3oc4ccc5ccccc5c4c3n2)cc1 ; c1ccc(-n2c3ccccc3c3cc(-c4ccc5[nH]c6ccccc6c5c4)ccc32)cc1 ; c1ccc(-n2c3ccccc3c3cc(-c4ccc5c(c4)c4ccccc4n5-c4ccc(-c5cnc6oc7ccc8ccccc8c7c6n5)cc4)ccc32)cc1]

### Example 23

### Synthesis Example 16

This example describes a method for synthesizing 11-[(3′-(dibenzothiophen-4-yl)biphenyl-3-yl]phenanthro[9′,10′:4,5]furo[2,3-b]pyrazine (abbreviation: 11mDBtBPPnfpr), which is the organic compound of one embodiment of the present invention represented by Structural Formula (178) in Embodiment 1. The structure of 11 mDBtBPPnfpr is shown below.

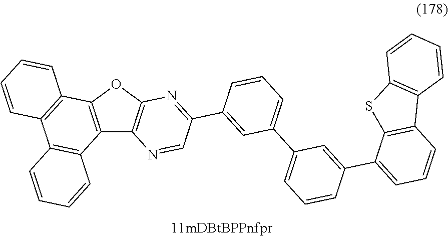 [SMILES: c1cc(-c2cccc(-c3cccc4c3sc3ccccc34)c2)cc(-c2cnc3c(n2)oc2c4ccccc4c4ccccc4c32)c1]

A synthesis method of 11mDBtBPPnfpr is shown by synthesis schemes (p-1) to (p-7) below.

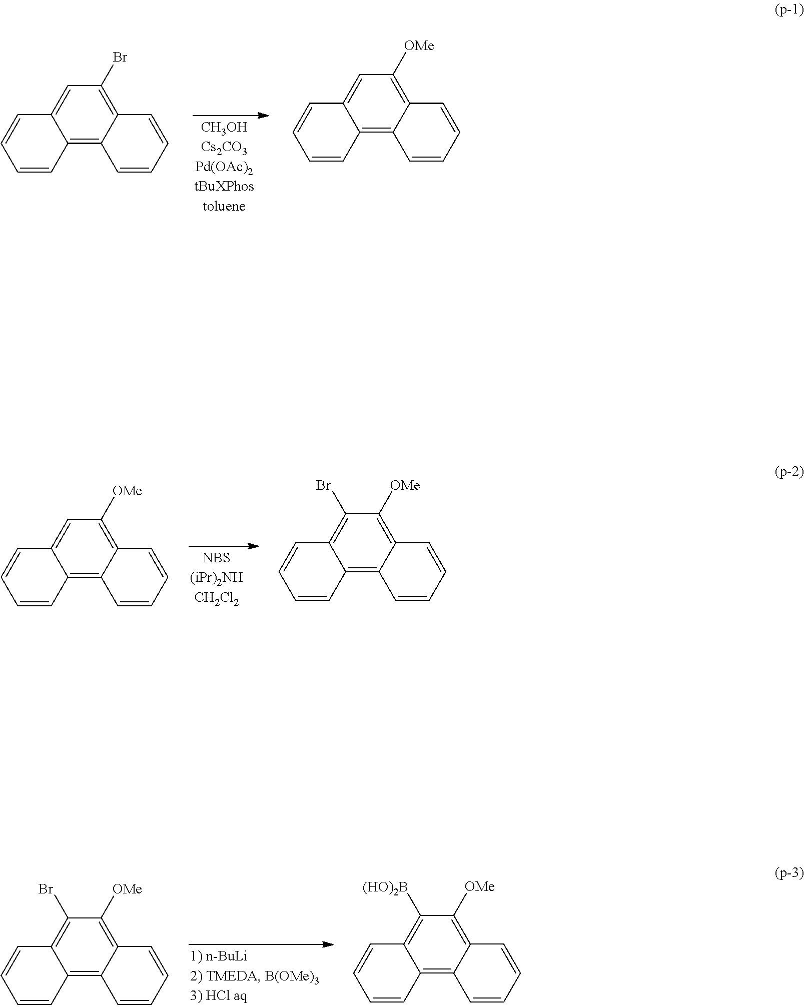 [SMILES: Brc1cc2ccccc2c2ccccc12 ; COc1cc2ccccc2c2ccccc12 ; COc1cc2ccccc2c2ccccc12 ; COc1c(Br)c2ccccc2c2ccccc12 ; COc1c(Br)c2ccccc2c2ccccc12 ; COc1c(B(O)O)c2ccccc2c2ccccc12]

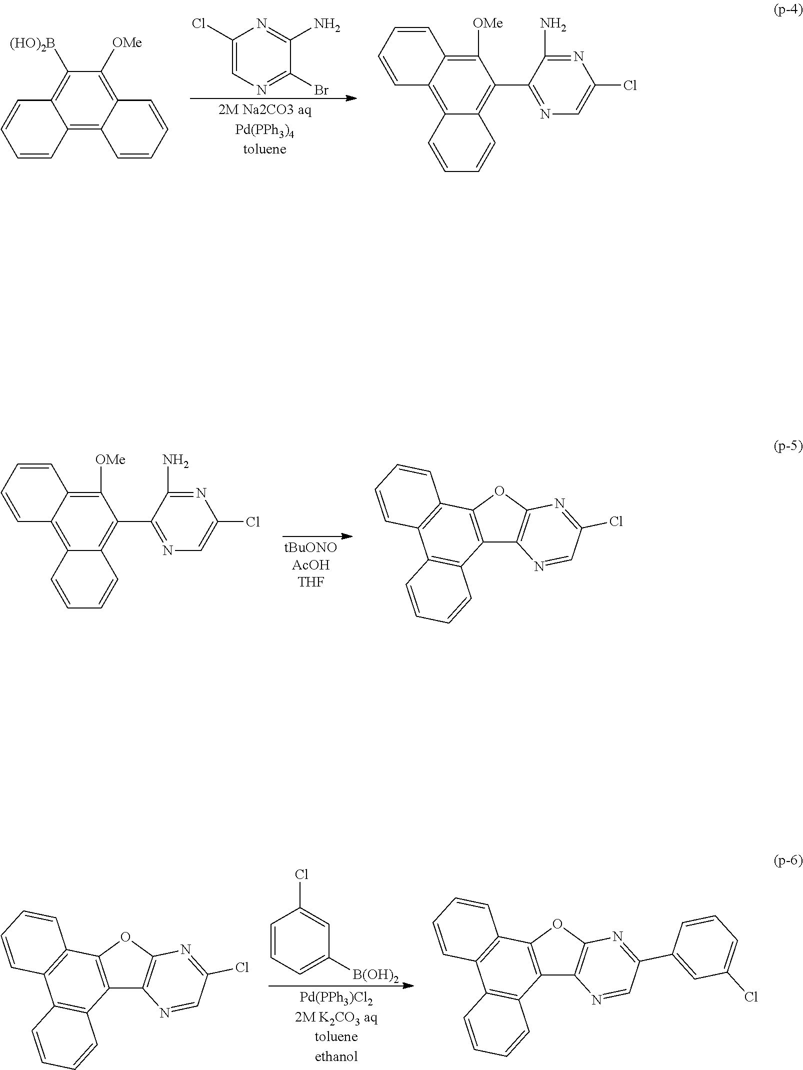

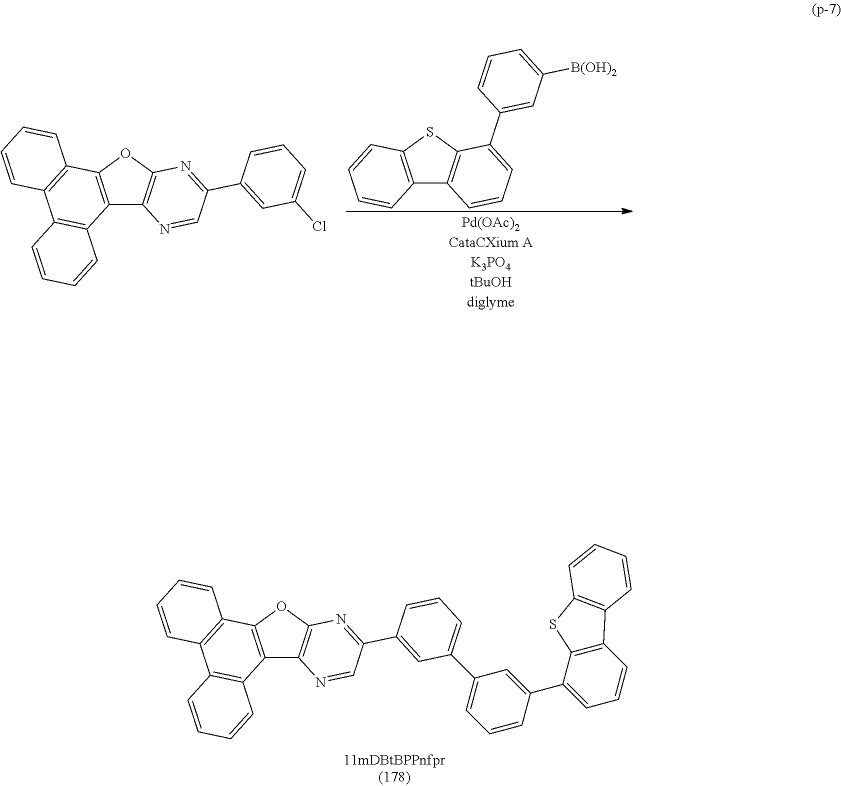

### Example 24

### Synthesis Example 17

This example describes a method for synthesizing 10-[3-(9′-phenyl-3,3′-bi-9H-carbazol-9-yl)phenyl]naphtho[1′,2′:4,5]furo[2,3-b]pyrazine (abbreviation: 10pPCCzPNfpr), which is the organic compound of one embodiment of the present invention represented by Structural Formula (240) in Embodiment 1. The structure of 10pPCCzPNfpr is shown below.

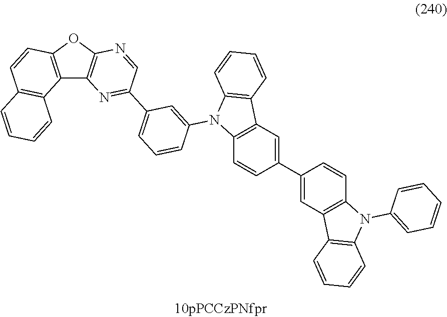 [SMILES: c1ccc(-n2c3ccccc3c3cc(-c4ccc5c(c4)c4ccccc4n5-c4cccc(-c5cnc6oc7ccc8ccccc8c7c6n5)c4)ccc32)cc1]

A synthesis method of 10pPCCzPNfpr is shown by synthesis schemes (q-1) to (q-4) below.

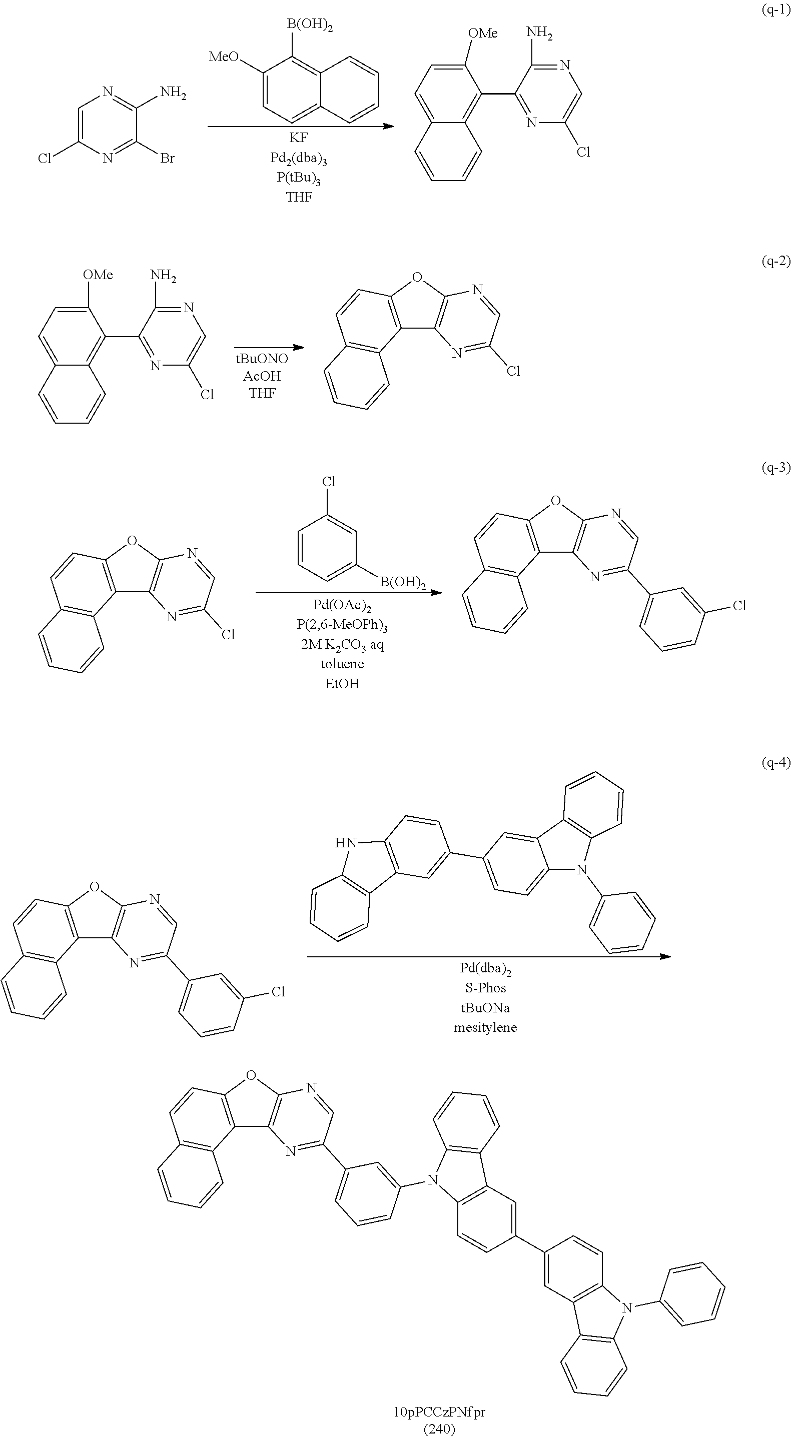

### Example 25

### Synthesis Example 18

This example describes a method for synthesizing 9-[3-(7H-dibenzo[c,g]carbazol-7-yl)phenyl]naphtho[1′,2′:4,5]furo[2,3-b]pyrazine (abbreviation: 9mcgDBCzPNfpr), which is the organic compound of one embodiment of the present invention represented by Structural Formula (242) in Embodiment 1. The structure of 9mcgDBCzPNfpr is shown below.

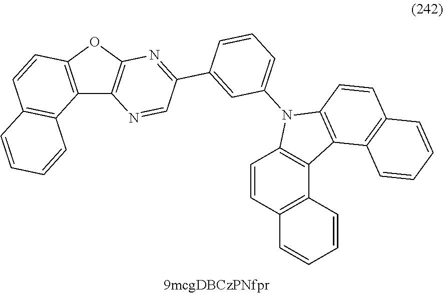 [SMILES: c1cc(-c2cnc3c(n2)oc2ccc4ccccc4c23)cc(-n2c3ccc4ccccc4c3c3c4ccccc4ccc32)c1]

A synthesis method of 9mcgDBCzPNfpr is shown by synthesis schemes (r-1) to (r-4) below.

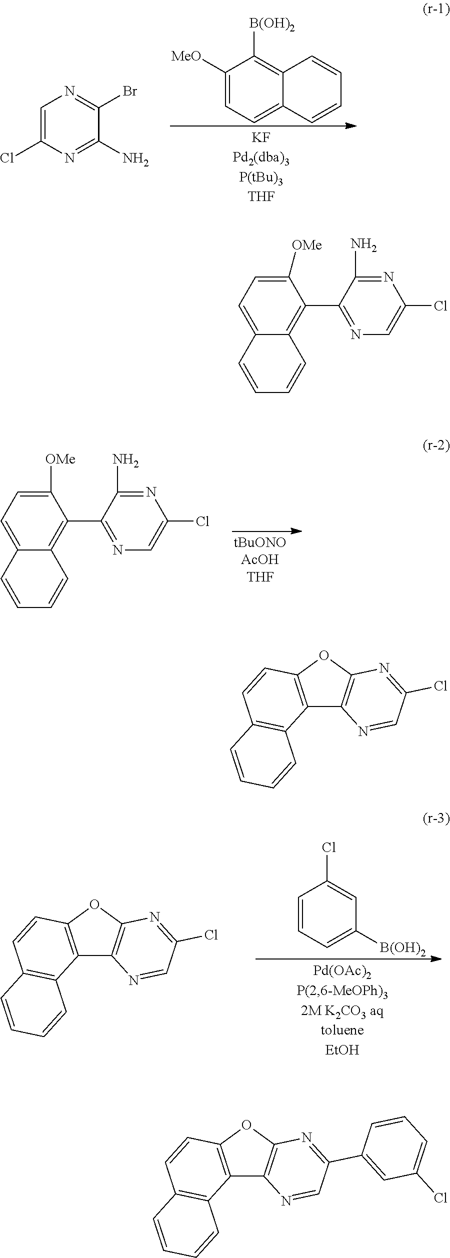

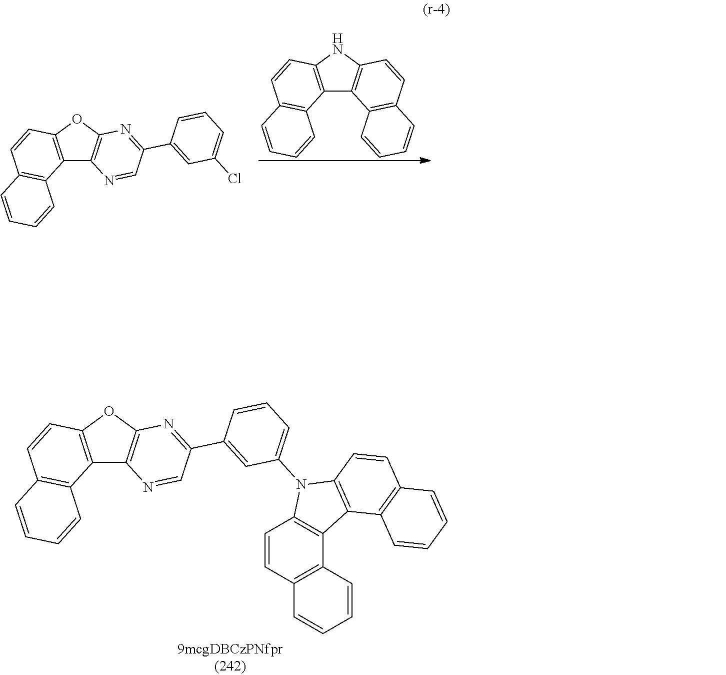

### Example 26

### Synthesis Example 19

This example describes a method for synthesizing 9-{3′-[6-(biphenyl-3-yl)dibenzothiophen-4-yl]biphenyl-3-yl}naphtho[1′,2′:4,5]furo[2,3-b]pyrazine (abbreviation: 9mDBtBPNfpr-03), which is the organic compound of one embodiment of the present invention represented by Structural Formula (249) in Embodiment 1. The structure of 9mDBtBPNfpr-03 is shown below.

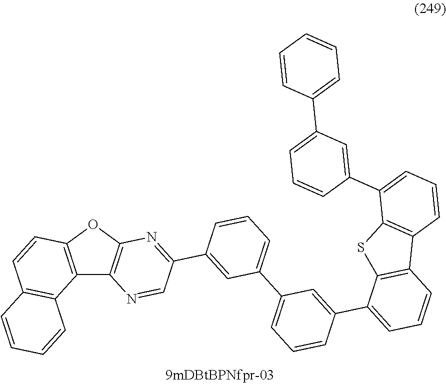 [SMILES: c1ccc(-c2cccc(-c3cccc4c3sc3c(-c5cccc(-c6cccc(-c7cnc8c(n7)oc7ccc9ccccc9c78)c6)c5)cccc34)c2)cc1]

A synthesis method of 9mDBtBPNfpr-03 is shown by synthesis schemes (s-1) to (s-4) below.

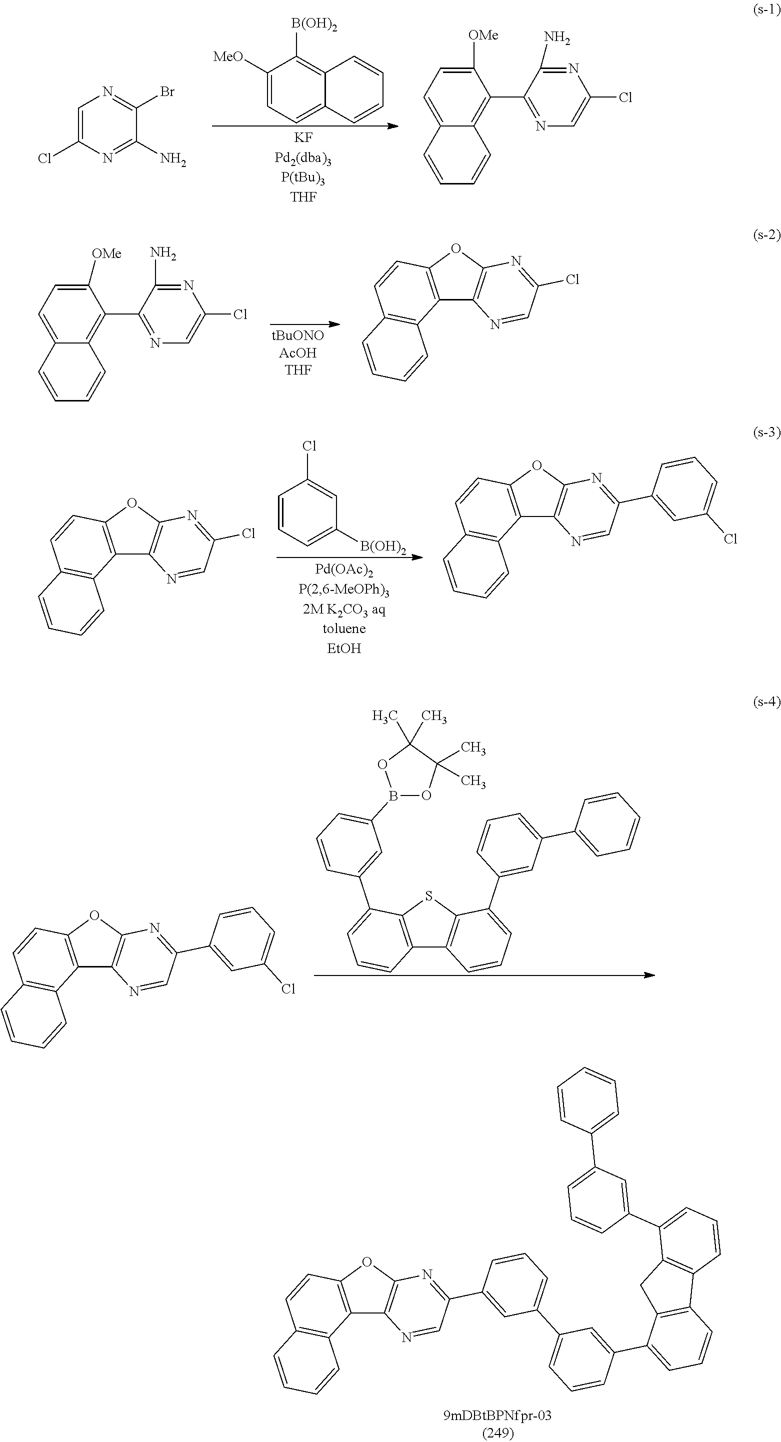

### Example 27

### Synthesis Example 20

This example describes a method for synthesizing 9-{3′-[6-(biphenyl-4-yl)dibenzothiophen-4-yl]biphenyl-3-yl}naphtho[1′,2′:4,5]furo[2,3-b]pyrazine (abbreviation: 9mDBtBPNfpr-04), which is the organic compound of one embodiment of the present invention represented by Structural Formula (250) in Embodiment 1. The structure of 9mDBtBPNfpr-04 is shown below.

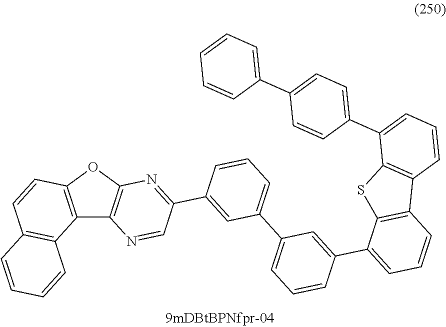 [SMILES: c1ccc(-c2ccc(-c3cccc4c3sc3c(-c5cccc(-c6cccc(-c7cnc8c(n7)oc7ccc9ccccc9c78)c6)c5)cccc34)cc2)cc1]

A synthesis method of 9mDBtBPNfpr-04 is shown by synthesis schemes (t-1) to (t-4) below.

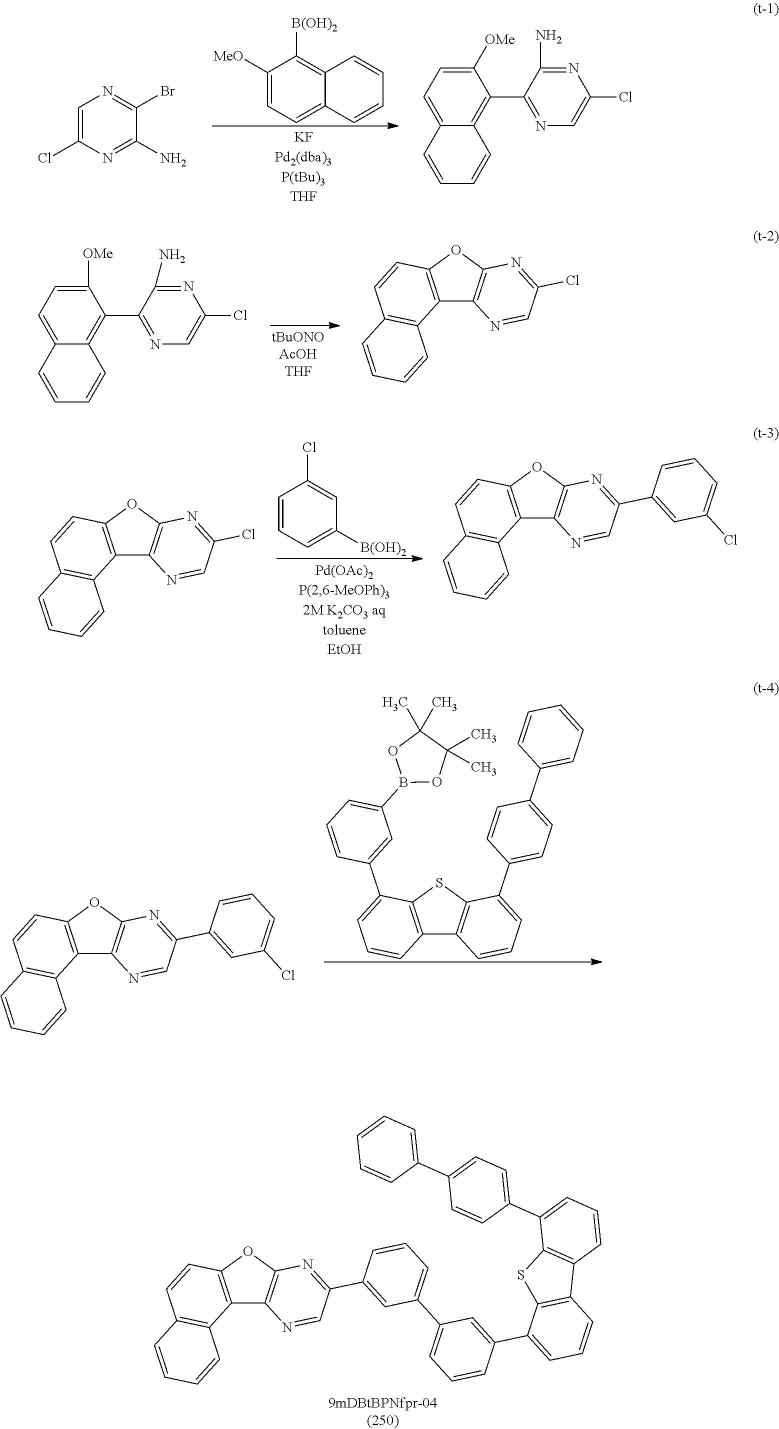

### Example 28

### Synthesis Example 21

This example describes a method for synthesizing 11-[3′-(6-phenyldibenzothiophen-4-yl)biphenyl-3-yl]phenanthro[9′,10′:4,5]furo[2,3-b]pyrazine (abbreviation: 11mDBtBPPnfpr-02), which is the organic compound of one embodiment of the present invention represented by Structural Formula (251) in Embodiment 1. The structure of 11mDBtBPPnfpr-02 is shown below.

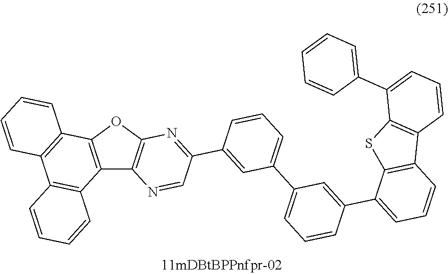 [SMILES: c1ccc(-c2cccc3c2sc2c(-c4cccc(-c5cccc(-c6cnc7c(n6)oc6c8ccccc8c8ccccc8c76)c5)c4)cccc23)cc1]

A synthesis method of 11 mDBtBPPnfpr-02 is shown by synthesis schemes (u-1) to (u-7) below.

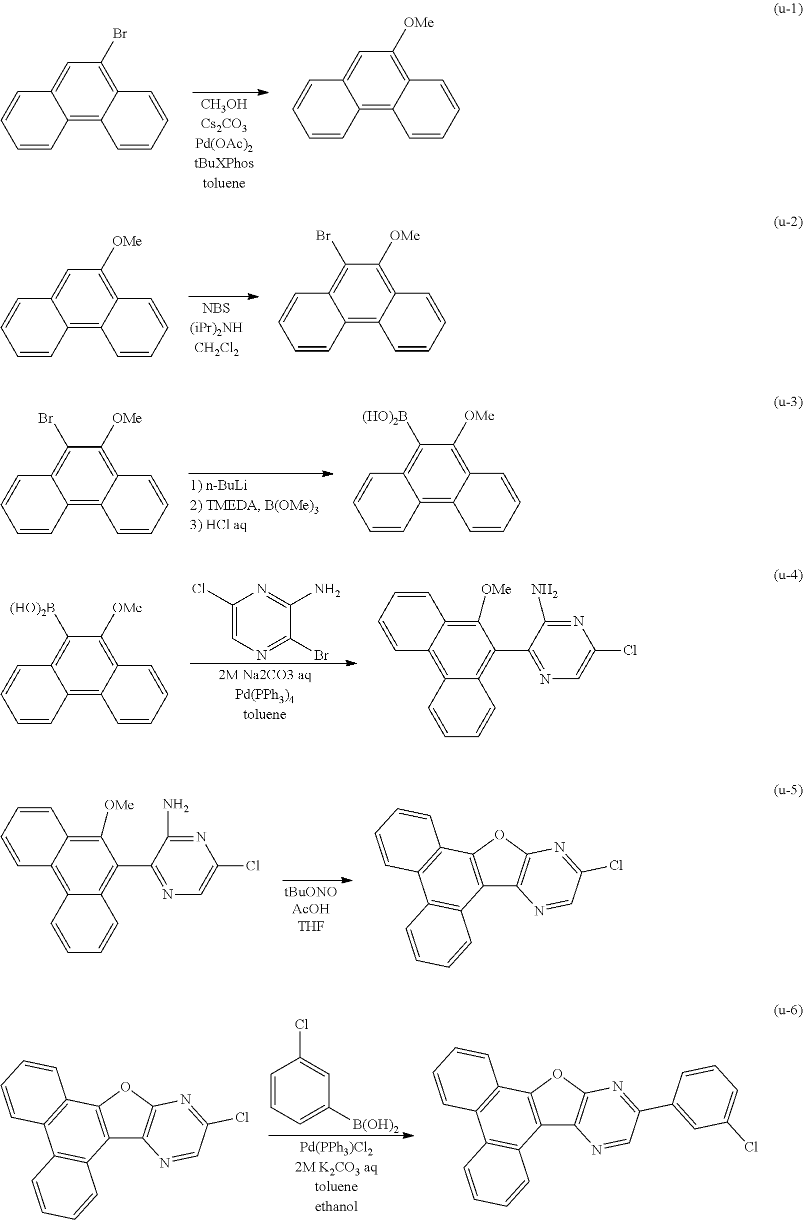

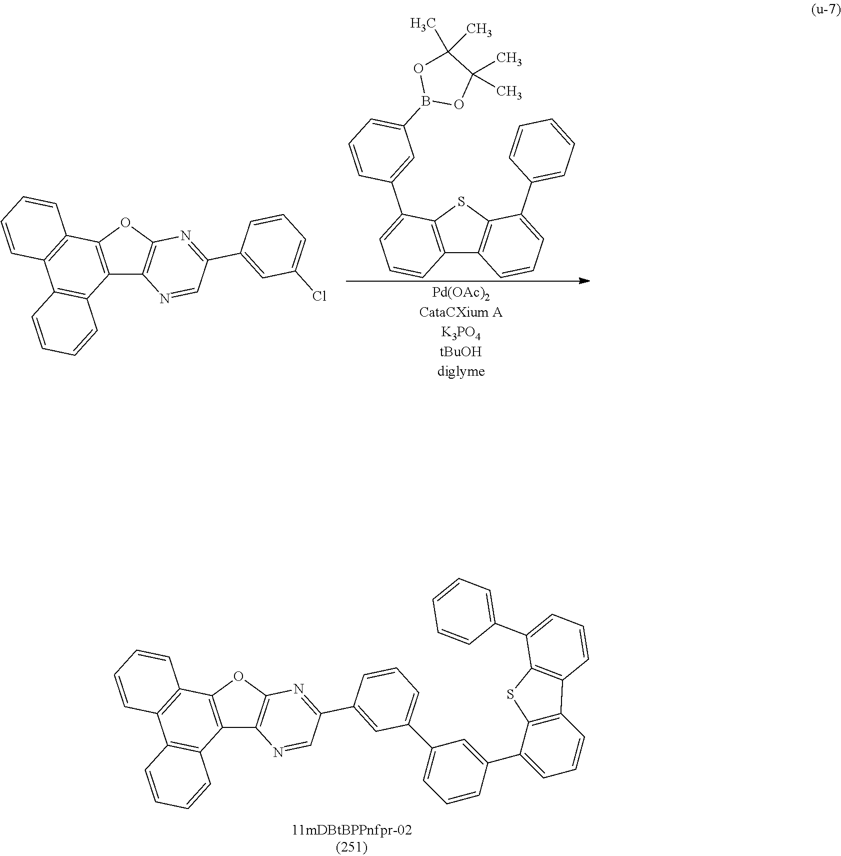 [SMILES: Clc1cccc(-c2cnc3c(n2)oc2c4ccccc4c4ccccc4c32)c1 ; CC1(C)OB(c2cccc(-c3cccc4c3sc3c(-c5ccccc5)cccc34)c2)OC1(C)C ; c1ccc(-c2cccc3c2sc2c(-c4cccc(-c5cccc(-c6cnc7c(n6)oc6c8ccccc8c8ccccc8c76)c5)c4)cccc23)cc1]

### Example 29

In this example, a light-emitting element 16 (light-emitting element of one embodiment of the present invention) was fabricated using 12mDBtBPPnfpr (Structural Formula (208), Example 11) in its light-emitting layer and a comparative light-emitting element 17 was fabricated using 2-[3′-(dibenzothiophen-4-yl)biphenyl-3-yl]dibenzo[f,h]quinoxaline (abbreviation: 2mDBTBPDBq-II) in its light-emitting layer. The measured characteristic results of these light-emitting elements will be described below.

The element structures of the light-emitting element 16 and the comparative light-emitting element 17 fabricated in this example were similar to the element structure described in Example 2 with reference to FIG. 11. Table 18 shows specific structures of layers in the element structures. Chemical formulae of materials used in this example are shown below.

TABLE 18

| | | | Hole- | Light- | | | Electron- | |
|---|---|---|---|---|---|---|---|---|
| | First | Hole-injection | transport | emitting | | | injection | Second |
| | electrode | layer | layer | layer | Electron-transport layer | | layer | electrode |
| | 901 | 911 | 912 | 913 | 914 | | 915 | 903 |
| Light- | ITSO | DBT3P-II:MoOx | PCBBiF | * | 12mDBtBPPnfpr | NBphen | LiF | Al |
| emitting | (70 nm) | (2:1, 60 nm) | (20 nm) | | (25 nm) | (15 nm) | (1 nm) | (200 nm) |
| element 16 | | | | | | | | |
| Comparative | ITSO | DBT3P-II:MoOx | PCBBiF | ** | 2mDBTBPDBq-II | NBphen | LiF | Al |
| light-emitting | (70 nm) | (2:1, 60 nm) | (20 nm) | | (25 nm) | (15 nm) | (1 nm) | (200 nm) |
| element 17 | | | | | | | | |

* 12mDBtBPPnfpr:PCBBiF:[Ir(dppm)₂(acac)] (0.75:0.25:0.075, 40 nm)
** 2mDBTBPDBq-II:PCBBiF:[Ir(dppm)₂(acac)] (0.75:0.25:0.075, 40 nm)

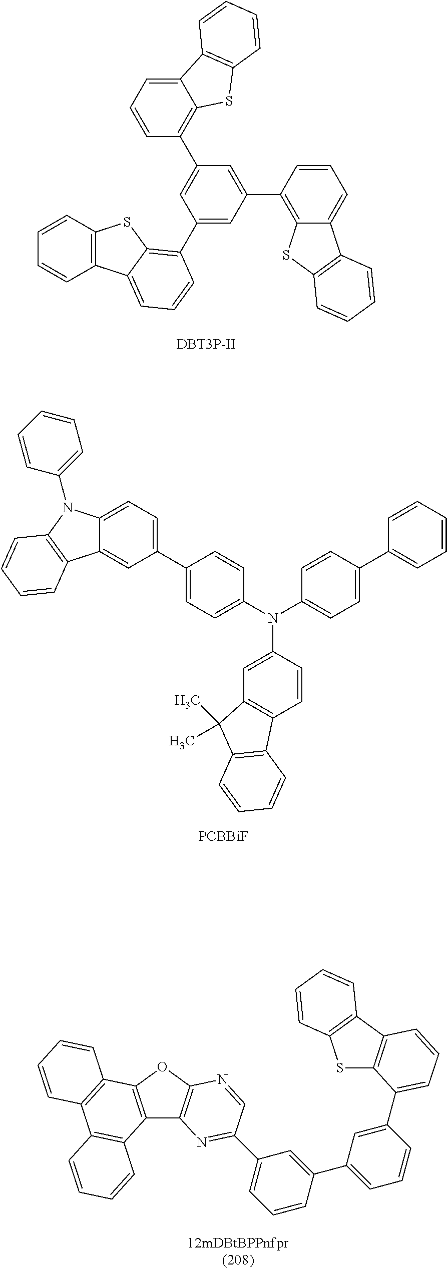

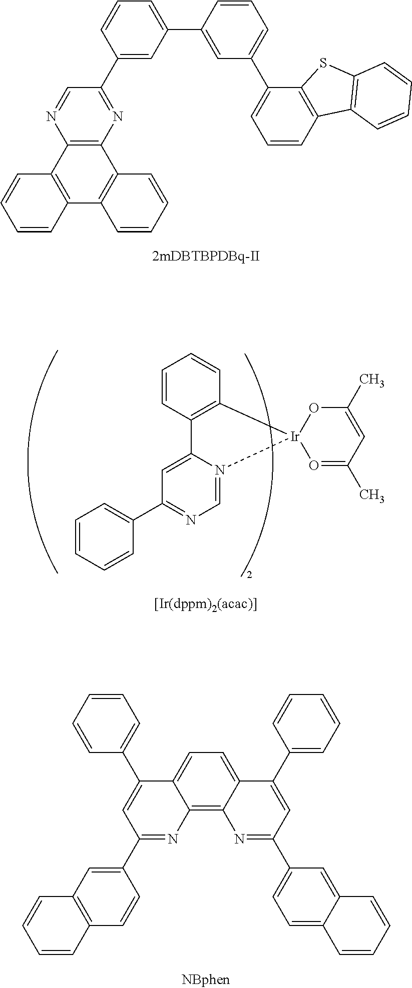

### <<Operation Characteristics of Light-Emitting Elements>>

Operation characteristics of the fabricated light-emitting element 16 and comparative light-emitting element 17 were measured. Note that the measurement was performed at room temperature (in an atmosphere kept at 25° C.).

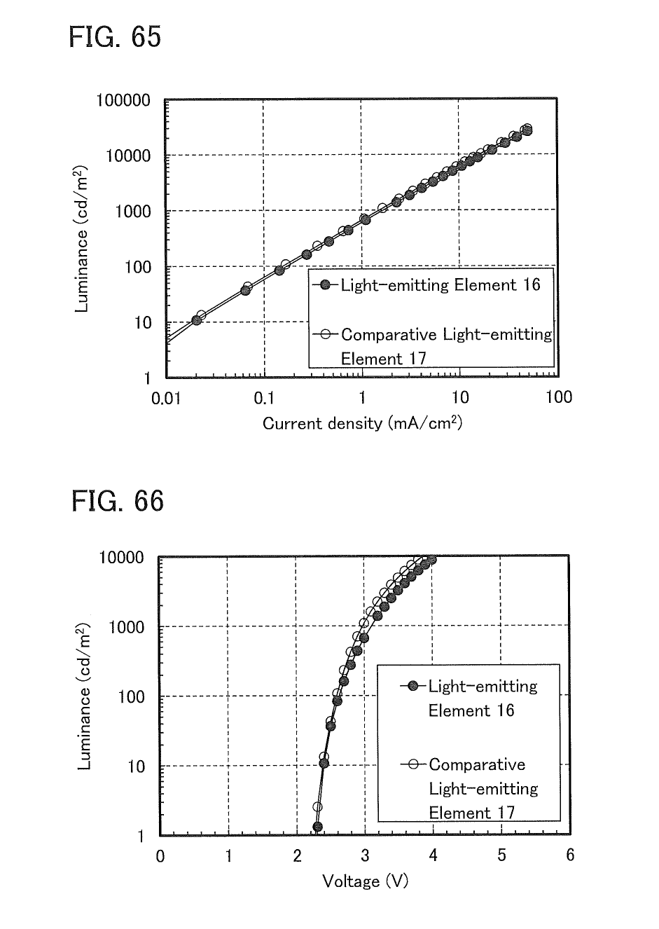

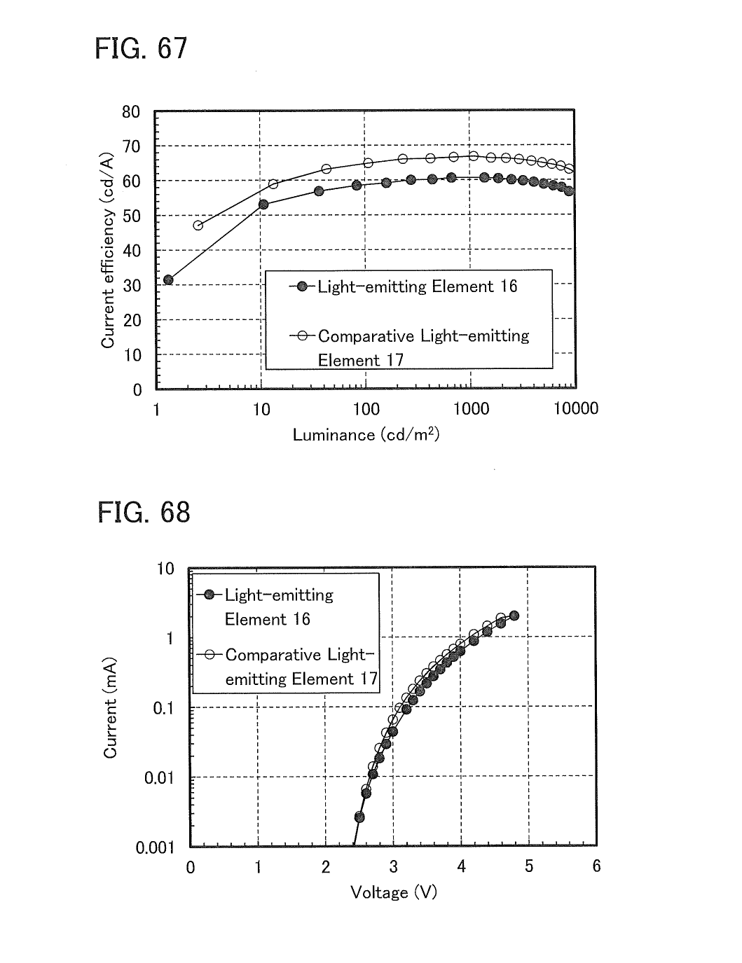

FIG. 65, FIG. 66, FIG. 67, and FIG. 68 show the current density-luminance characteristics, the voltage-luminance characteristics, the luminance-current efficiency characteristics, and the voltage-current characteristics, respectively, of the light-emitting element 16 and the comparative light-emitting element 17.

Table 19 shows initial values of main characteristics of the light-emitting element 16 and the comparative light-emitting element 17 at around 1000 cd/m².

TABLE 19

| | | | | | | | | External |
|---|---|---|---|---|---|---|---|---|
| | | | Current | | | Current | Power | quantum |
| | Voltage | Current | density | Chromaticity | Luminance | efficiency | efficiency | efficiency |
| | (V) | (mA) | (mA/cm²) | (x, y) | (cd/m²) | (cd/A) | (lm/W) | (%) |
| Light- | 3.0 | 0.04 | 1.1 | (0.56, 0.44) | 670 | 61 | 64 | 26 |
| emitting | | | | | | | | |
| element 16 | | | | | | | | |
| Comparative | 3.0 | 0.07 | 1.6 | (0.56, 0.43) | 1100 | 67 | 70 | 28 |
| light-emitting | | | | | | | | |
| element 17 | | | | | | | | |

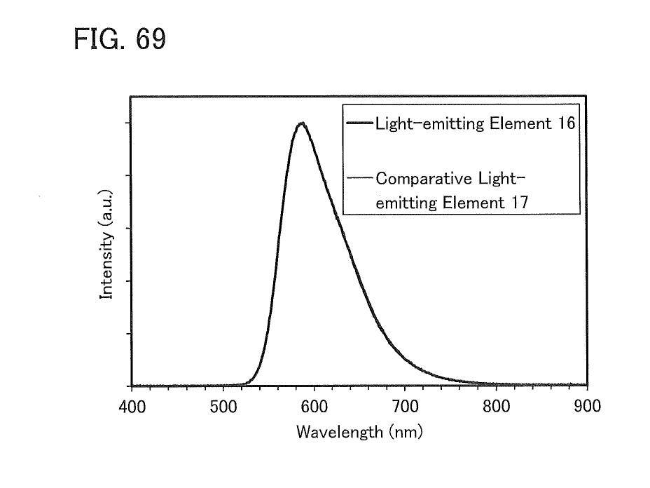

FIG. 69 shows emission spectra when current at a current density of 2.5 mA/cm² was applied to the light-emitting elements. As shown in FIG. 69, the emission spectrum of each light-emitting element has a peak at around 586 nm that is probably derived from light emission of [Ir(dppm)₂(acac)] contained in the light-emitting layer 913.

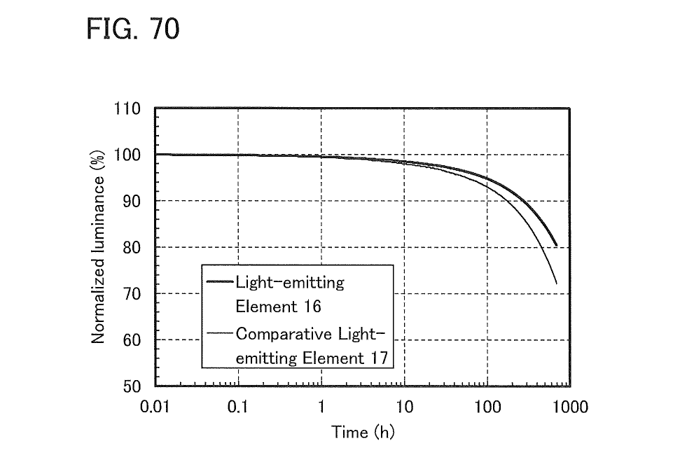

Next, reliability tests were performed on the light-emitting elements. FIG. 70 shows results of the reliability tests. In FIG. 70, the vertical axis represents normalized luminance (%) with an initial luminance of 100%, and the horizontal axis represents driving time (h) of the elements. As the reliability tests, constant current driving tests at a constant current density of 75 mA/cm² were performed.

The results of the reliability tests show that the light-emitting element 16 including 12mDBtBPPnfpr, which is the organic compound of one embodiment of the present invention, has higher reliability than the comparative light-emitting element 17 including 2mDBTBPDBq-II. This is probably derived from a difference in molecular structures between 12mDBtBPPnfpr and 2mDBTBPDBq-II, that is, a difference between a phenanthrofuropyrazine skeleton and a dibenzoquinoxaline skeleton, thus showing robustness of a furopyrazine derivative of one embodiment of the present invention. Accordingly, it is indicated that the use of the organic compound of one embodiment of the present invention is effective in improving the reliability of a light-emitting element.

This application is based on Japanese Patent Application Serial No. 2017-145790 filed with Japan Patent Office on Jul. 27, 2017 and Japanese Patent Application Serial No. 2017-231510 filed with Japan Patent Office on Dec. 1, 2017, the entire contents of which are hereby incorporated by reference.

What is claimed is:

1. An organic compound represented by General Formula (G1): 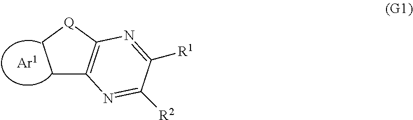
wherein Q represents oxygen or sulfur,
wherein Ar¹ represents a substituted or unsubstituted condensed aromatic ring,
wherein R¹ and R² independently represent hydrogen or a group having 1 to 100 total carbon atoms, and
wherein at least one of R¹ and R² comprises a hole-transport skeleton.

2. The organic compound according to claim 1, wherein Ar¹ represents any one of substituted or unsubstituted naphthalene, substituted or unsubstituted phenanthrene, and substituted or unsubstituted chrysene.

3. The organic compound according to claim 1,
wherein Ar¹ in General Formula (G1) is any one of General Formulae (t1) to (t3): 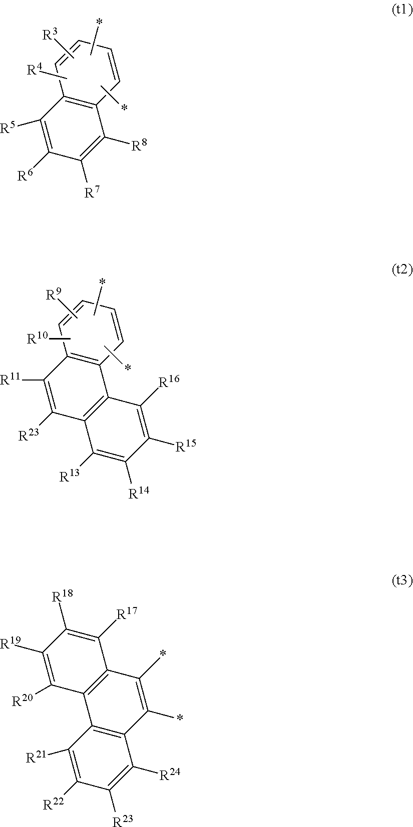
wherein R³ to R²⁴ independently represent any one of hydrogen, a substituted or unsubstituted alkyl group having 1 to 6 carbon atoms, a substituted or unsubstituted cycloalkyl group having 3 to 7 carbon atoms, and a substituted or unsubstituted aryl group having 6 to 30 carbon atoms, and
wherein * represents a bonding portion in General Formula (G1).

4. The organic compound according to claim 1,
wherein General Formula (G1) is any one of General Formulae (G1-1) to (G1-4): 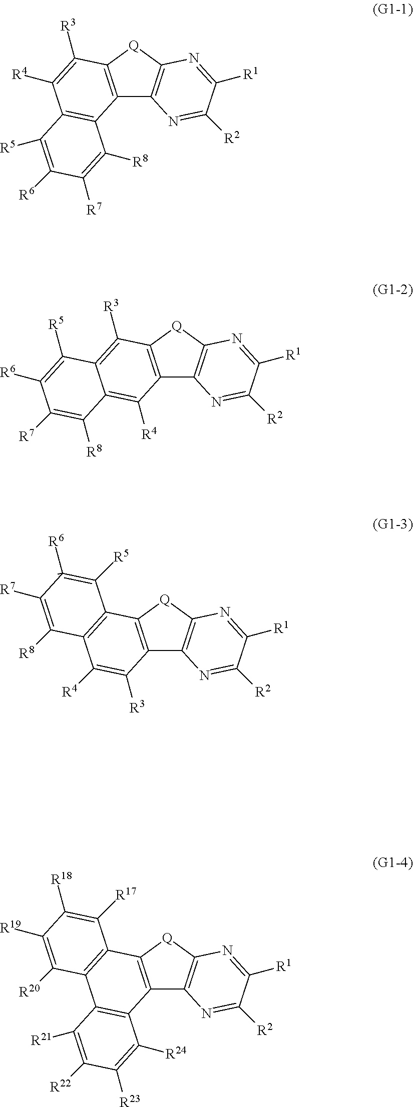
wherein R³ to R⁸ and R¹⁷ to R²⁴ independently represent any one of hydrogen, a substituted or unsubstituted alkyl group having 1 to 6 carbon atoms, a substituted or unsubstituted cycloalkyl group having 3 to 7 carbon atoms, and a substituted or unsubstituted aryl group having 6 to 30 carbon atoms.

5. The organic compound according to claim 1, wherein the hole-transport skeleton is any one of a substituted or unsubstituted diarylamino group, a substituted or unsubstituted condensed aromatic hydrocarbon ring, and a substituted or unsubstituted π-electron rich condensed heteroaromatic ring.

6. A light-emitting element comprising the organic compound according to claim 1.

7. A light-emitting element comprising an EL layer between a pair of electrodes, wherein the EL layer comprises the organic compound according to claim 1.

8. A light-emitting element comprising an EL layer between a pair of electrodes,
wherein the EL layer comprises a light-emitting layer, and
wherein the light-emitting layer comprises the organic compound according to claim 1.

9. An organic compound represented by General Formula (G1): 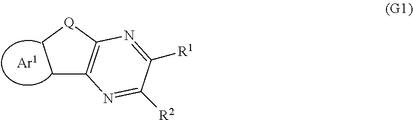
wherein Q represents oxygen or sulfur,
wherein Ar¹ represents a substituted or unsubstituted condensed aromatic ring,
wherein R¹ and R² independently represent hydrogen or a group having 1 to 100 total carbon atoms, and
wherein at least one of R¹ and R² is a group comprising a condensed ring.

10. The organic compound according to claim 9, wherein Ar¹ represents any one of substituted or unsubstituted naphthalene, substituted or unsubstituted phenanthrene, and substituted or unsubstituted chrysene.

11. The organic compound according to claim 9,
wherein Ar¹ in General Formula (G1) is any one of General Formulae (t1) to (t3): 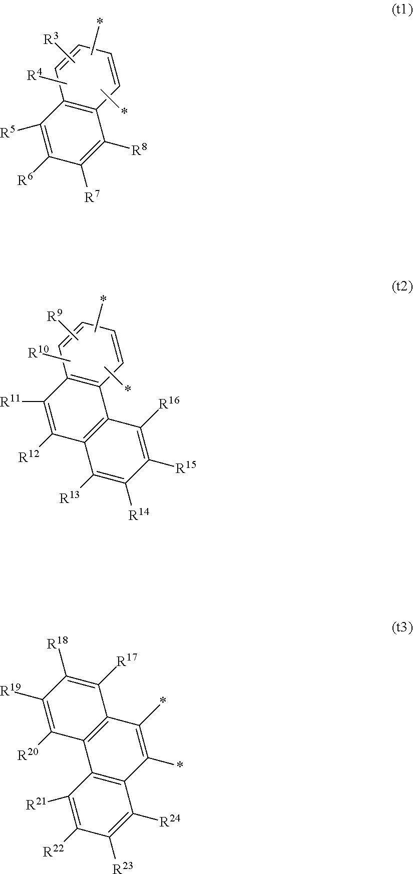
wherein R³ to R²⁴ independently represent any one of hydrogen, a substituted or unsubstituted alkyl group having 1 to 6 carbon atoms, a substituted or unsubstituted cycloalkyl group having 3 to 7 carbon atoms, and a substituted or unsubstituted aryl group having 6 to 30 carbon atoms, and
wherein * represents a bonding portion in General Formula (G1).

12. The organic compound according to claim 9,
wherein General Formula (G1) is any one of General Formulae (G1-1) to (G1-4): 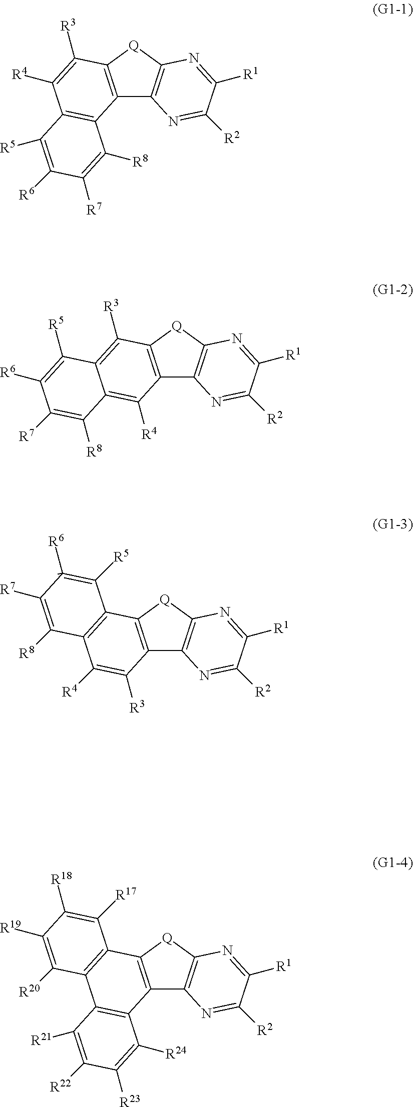
wherein R³ to R⁸ and R¹⁷ to R²⁴ independently represent any one of hydrogen, a substituted or unsubstituted alkyl group having 1 to 6 carbon atoms, a substituted or unsubstituted cycloalkyl group having 3 to 7 carbon atoms, and a substituted or unsubstituted aryl group having 6 to 30 carbon atoms.

13. The organic compound according to claim 9, wherein the condensed ring is any one of a substituted or unsubstituted condensed aromatic hydrocarbon ring and a substituted or unsubstituted π-electron rich condensed heteroaromatic ring.

14. The organic compound according to claim 9, wherein the condensed ring is a substituted or unsubstituted condensed heteroaromatic ring comprising any one of a dibenzothiophene skeleton, a dibenzofuran skeleton, and a carbazole skeleton.

15. The organic compound according to claim 9, wherein the condensed ring is a substituted or unsubstituted condensed aromatic hydrocarbon ring comprising any one of a naphthalene skeleton, a fluorene skeleton, a triphenylene skeleton, and a phenanthrene skeleton.

16. The organic compound according to claim 9, the organic compound is represented by any one of Structural Formulae (100), (123), (125), (126), (133), (156), (208), (238), (239), (244), (245), and (246).

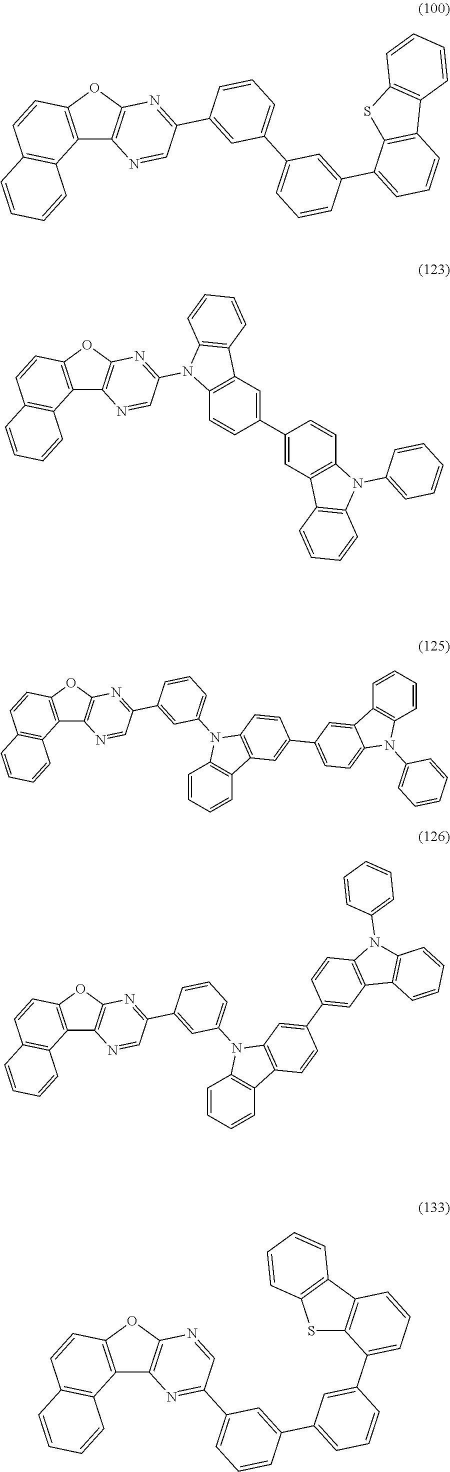 [SMILES: c1cc(-c2cccc(-c3cccc4c3sc3ccccc34)c2)cc(-c2cnc3c(n2)oc2ccc4ccccc4c23)c1 ; c1ccc(-n2c3ccccc3c3cc(-c4ccc5c(c4)c4ccccc4n5-c4cnc5c(n4)oc4ccc6ccccc6c45)ccc32)cc1 ; c1ccc(-n2c3ccccc3c3cc(-c4ccc5c(c4)c4ccccc4n5-c4cccc(-c5cnc6c(n5)oc5ccc7ccccc7c56)c4)ccc32)cc1 ; c1ccc(-n2c3ccccc3c3cc(-c4ccc5c6ccccc6n(-c6cccc(-c7cnc8c(n7)oc7ccc9ccccc9c78)c6)c5c4)ccc32)cc1 ; c1cc(-c2cccc(-c3cccc4c3sc3ccccc34)c2)cc(-c2cnc3oc4ccc5ccccc5c4c3n2)c1]

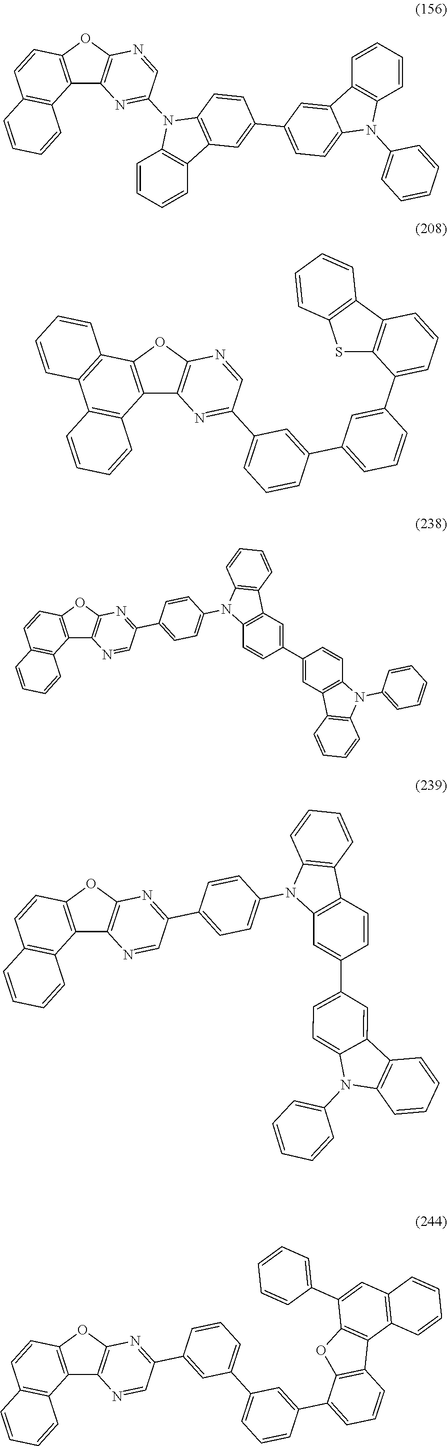 [SMILES: c1ccc(-n2c3ccccc3c3cc(-c4ccc5c(c4)c4ccccc4n5-c4cnc5oc6ccc7ccccc7c6c5n4)ccc32)cc1 ; c1cc(-c2cccc(-c3cccc4c3sc3ccccc34)c2)cc(-c2cnc3oc4c5ccccc5c5ccccc5c4c3n2)c1 ; c1ccc(-n2c3ccccc3c3cc(-c4ccc5c(c4)c4ccccc4n5-c4ccc(-c5cnc6c(n5)oc5ccc7ccccc7c56)cc4)ccc32)cc1 ; c1ccc(-n2c3ccccc3c3cc(-c4ccc5c6ccccc6n(-c6ccc(-c7cnc8c(n7)oc7ccc9ccccc9c78)cc6)c5c4)ccc32)cc1 ; c1ccc(-c2cc3ccccc3c3c2oc2c(-c4cccc(-c5cccc(-c6cnc7c(n6)oc6ccc8ccccc8c67)c5)c4)cccc23)cc1]

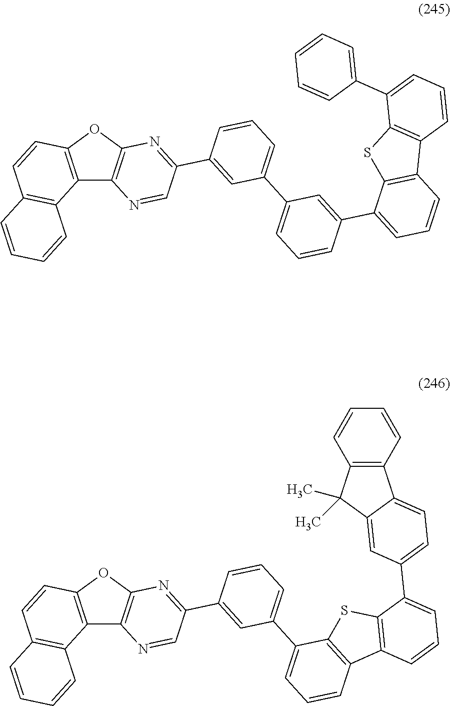 [SMILES: c1ccc(-c2cccc3c2sc2c(-c4cccc(-c5cccc(-c6cnc7c(n6)oc6ccc8ccccc8c67)c5)c4)cccc23)cc1 ; CC1(C)c2ccccc2-c2ccc(-c3cccc4c3sc3c(-c5cccc(-c6cnc7c(n6)oc6ccc8ccccc8c67)c5)cccc34)cc21]

17. A light-emitting element comprising the organic compound according to claim 9.

18. A light-emitting element comprising an EL layer between a pair of electrodes, wherein the EL layer comprises the organic compound according to claim 9.

19. A light-emitting element comprising an EL layer between a pair of electrodes,
wherein the EL layer comprises a light-emitting layer, and
wherein the light-emitting layer comprises the organic compound according to claim 9.

20. An organic compound represented by General Formula (G1): 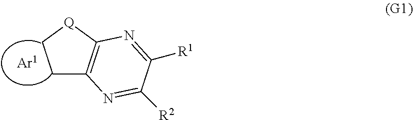
wherein Q represents oxygen or sulfur,
wherein Ar¹ represents a substituted or unsubstituted condensed aromatic ring,
wherein R¹ and R² independently represent hydrogen or a group having 1 to 100 total carbon atoms,
wherein at least one of R¹ and R² comprises a hole-transport skeleton,
wherein the at least one of R¹ and R² is a group represented by General Formula (u1):
A¹-(α)ₙ-* (u1)
wherein α represents a substituted or unsubstituted arylene group having 6 to 25 carbon atoms,
wherein n represents an integer of 0 to 4, and
wherein A¹ represents a substituted or unsubstituted aryl group having 6 to 30 total carbon atoms or a substituted or unsubstituted heteroaryl group having 3 to 30 total carbon atoms.

21. The organic compound according to claim 20,
wherein A¹ in General Formula (u1) is any one of General Formulae (A¹-1) to (A¹-17): 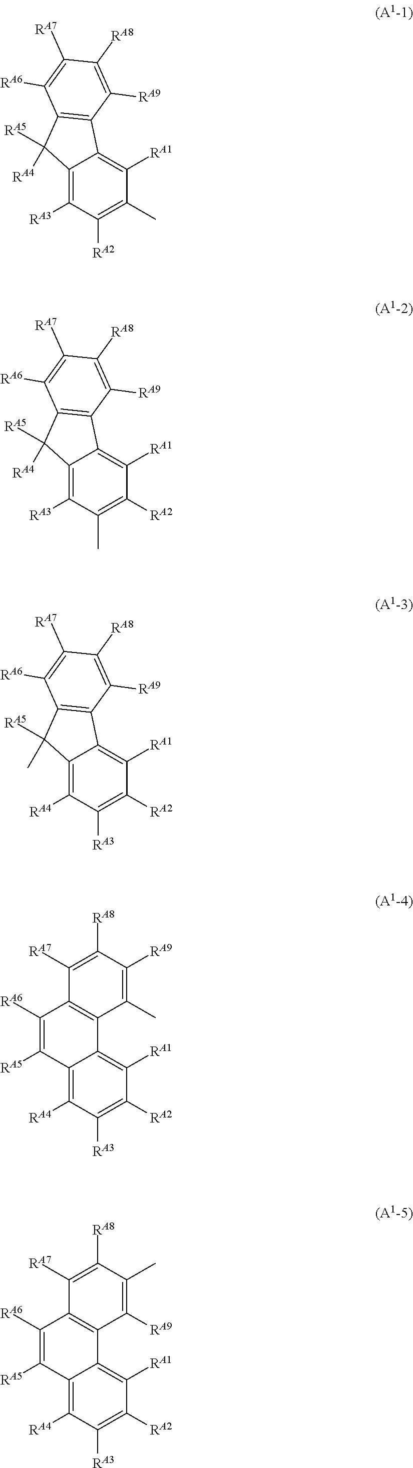 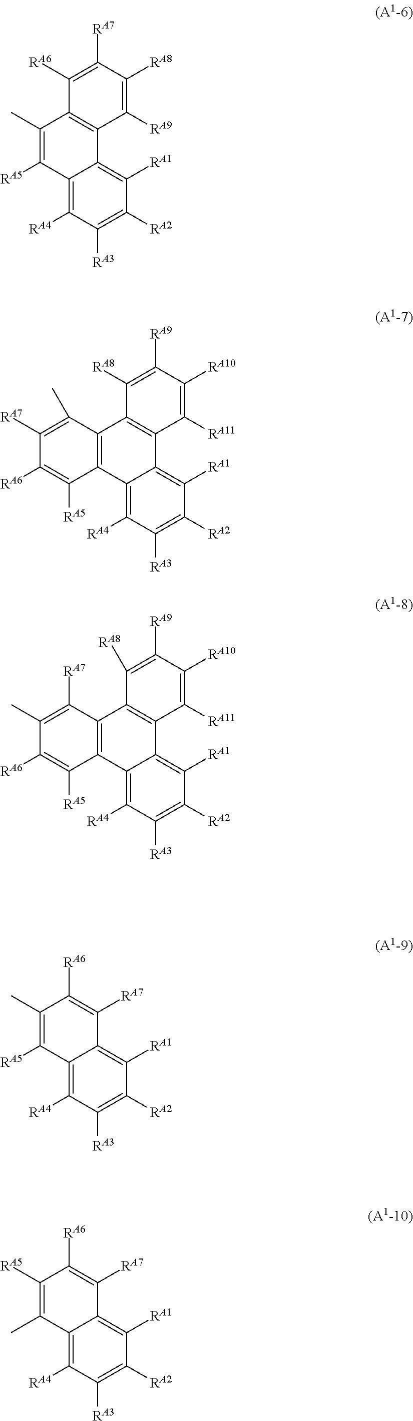 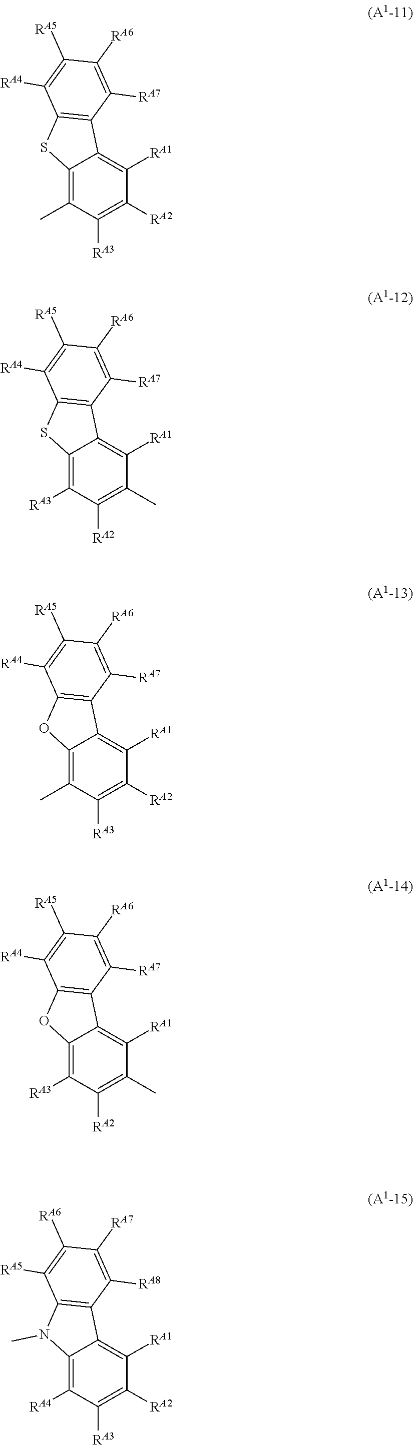 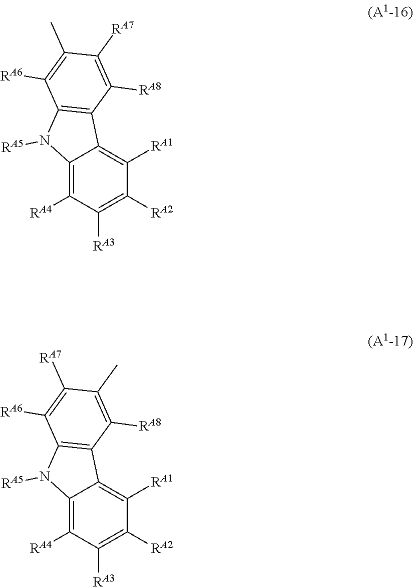
wherein R^{A1} to R^{A11} independently represent any one of hydrogen, a substituted or unsubstituted alkyl group having 1 to 6 carbon atoms, a substituted or unsubstituted cycloalkyl group having 3 to 7 carbon atoms, and a substituted or unsubstituted aryl group having 6 to 30 carbon atoms.

22. The organic compound according to claim 20,
wherein α in General Formula (u1) is any one of General Formulae (Ar-1) to (Ar-14): 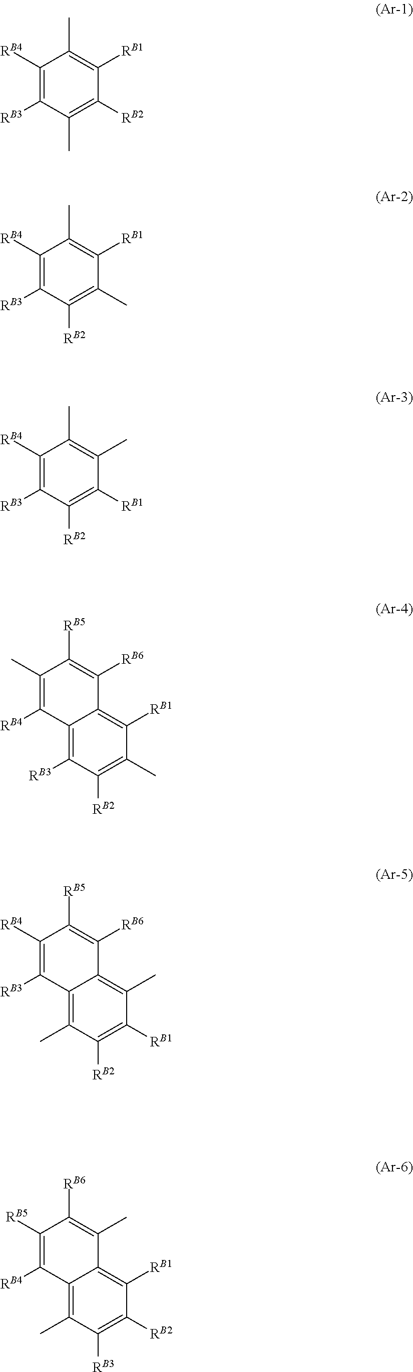 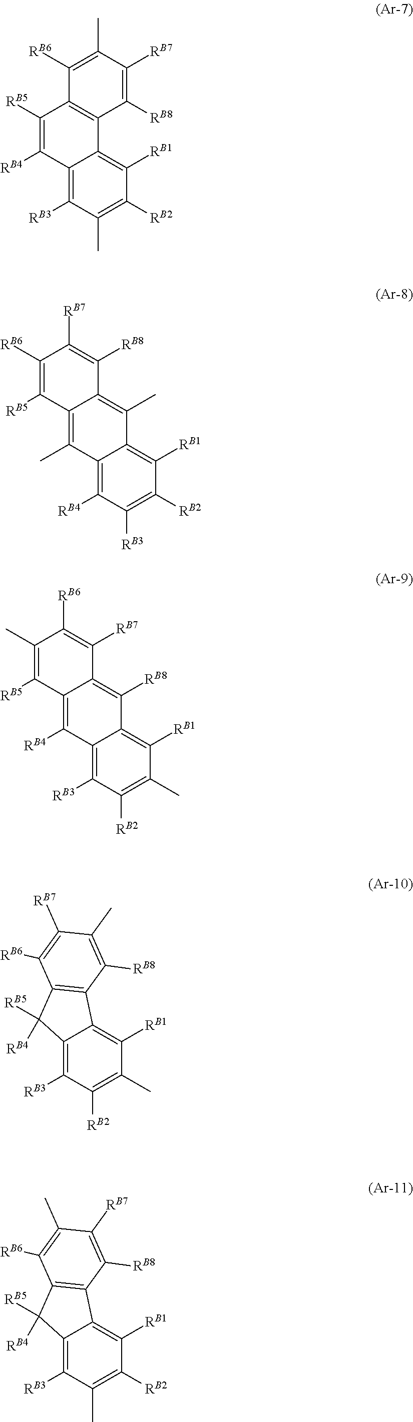 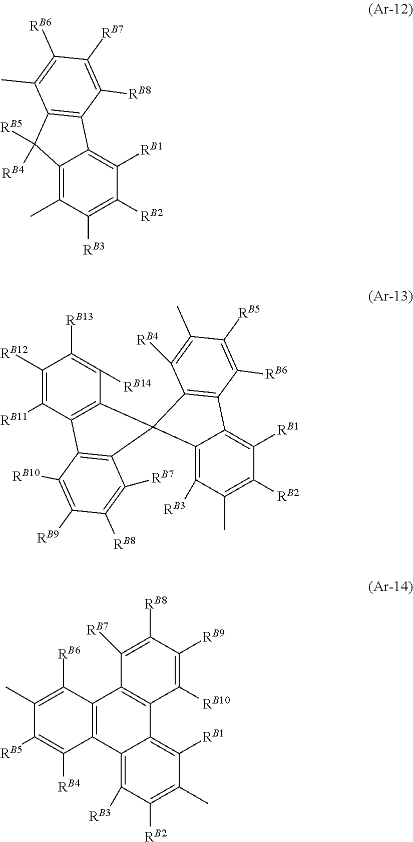
wherein R^{B1} to R^{B14} independently represent any one of hydrogen, a substituted or unsubstituted alkyl group having 1 to 6 carbon atoms, a substituted or unsubstituted cycloalkyl group having 3 to 7 carbon atoms, and a substituted or unsubstituted aryl group having 6 to 30 carbon atoms.

23. A light-emitting element comprising the organic compound according to claim 20.

24. A light-emitting element comprising an EL layer between a pair of electrodes, wherein the EL layer comprises the organic compound according to claim 20.

25. A light-emitting element comprising an EL layer between a pair of electrodes,
wherein the EL layer comprises a light-emitting layer, and
wherein the light-emitting layer comprises the organic compound according to claim 20.
